(19) United States
(12) Patent Application Publication
(10) Pub. No.: US 2025/0289887 A1
(43) Pub. Date: Sep. 18, 2025

(54) INTEIN-BASED SORTING SYSTEM AND MODULAR CHIMERIC POLYPEPTIDES

(71) Applicants: Memorial Sloan-Kettering Cancer Center, New York, NY (US); Sloan-Kettering Institute for Caqncer Research, New York, NY (US); Memorial Hospital for Cancer and Allied Diseases, New York, NY (US)

(72) Inventors: Scott JAMES, San Marino, CA (US); Marcel Van Den Brink, New York, NY (US); Brandon D. NG, Monroe Township, NJ (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 19/196,390

(22) Filed: May 1, 2025

Related U.S. Application Data
(63) Continuation of application No. PCT/US2023/078362, filed on Nov. 1, 2023.
(60) Provisional application No. 63/381,921, filed on Nov. 1, 2022.

(51) Int. Cl. C07K 16/28; A61K 40/11; A61K 40/31; A61K 40/42

(52) U.S. Cl. CPC C07K 16/2803; A61K 40/11; A61K 40/31; A61K 40/4211; A61K 40/4221; C07K 16/2887; C07K 2317/53; C07K 2317/622; C07K 2319/02; C07K 2319/03; C07K 2319/20

(57) ABSTRACT

The present application is directed to multiplex intein-based methods and compositions for the generation engineered cells expressing modular polypeptides, for example CARs and CCRs.

### 1. CROSS-REFERENCE TO RELATED APPLICATIONS

This application is a continuation of International Patent Application No. PCT/US2023/078362, filed Nov. 1, 2023, which claims priority to U.S. Provisional Patent Application No. 63/381,921, filed on Nov. 1, 2022, the entire contents of both of which are incorporated by reference in their entireties, and to which priority is claimed.

### 2. SEQUENCE LISTING

The instant application contains a Sequence Listing which has been submitted electronically in XML format and is hereby incorporated by reference in its entirety. Said XML copy, created on Feb. 9, 2024, is named 072734.1764SL.xml and is 331,710 bytes in size.

### 3. BACKGROUND OF THE INVENTION

Cell-based immunotherapies have established curative potential for the treatment of a variety of cancers. In many of such approaches, T cells and/or other immune cells are modified to target specific antigens via the expression of polypeptides, e.g., Chimeric Antigen Receptors (CARs) and Chimeric Costimulatory Receptors (CCRs), specific to the antigen(s) of interest. For example, targeted T cell therapies using CARs specific for the cancer-associated antigen CD19 have shown clinical success in treating hematologic malignancies. However, to ensure potent cancer eradication with minimal toxicity, cell-based immunotherapies can involve targeting multiple antigens. The targeting of multiple antigens generally requires the delivery and integration of large recombinant constructs into the appropriate T cells and/or other immune cells. It is difficult, however, to stably integrate large amounts of genetic information into primary T cells with the required efficiency. Both retroviruses and lentiviruses, for example, exhibit a significant decrease in viral titer as the viral vector insert approaches and exceeds the packaging limit of the virus (about 6-8 kb for retroviruses and about 10-12 kb for lentiviruses). Low viral titers result in low transduction efficiency and low copy number integrations per cell, leading to inferior levels of gene construct expression. Accordingly, there is a need for novel genetic engineering strategies to express the necessary polypeptides, and for therapeutic strategies capable of inducing potent cancer eradication with minimal toxicity and off-target activity.

### 4. SUMMARY OF THE INVENTION

In certain embodiments, the present disclosure is directed to cells, systems, and methods relating to the production of modular polypeptides, e.g., chimeric antigen receptors (CARs) and other cell surface transmembrane polypeptides, in which orthogonal split-intein tags are used to post-translationally trans-splice polypeptide subunits to generate functional, mature CARs or other transmembrane constructs. In certain embodiments, the trans-splicing peptide subunits include, but are not limited to: antigen binding domains (which can include one or more affinity tag(s)); surface presented membrane-bound cytokines and chemokines; spacers and transmembrane domains (which can include one or more affinity tag(s)); positive and/or negative costimulatory molecule extracellular domains; intracellular signaling domains; and intracellular non-signaling domains. In certain embodiments, antigen-binding and signaling intein-tagged subunits may be multiplexed to generate mixed populations of mature trans-spliced inteins inside individual cells. In certain embodiments, antigen binding domain polypeptide interaction with spacer-transmembrane domain polypeptide.

In certain embodiments, the present disclosure is directed to systems of affinity-tagged modular CARs or extracellular and transmembrane domain-containing polypeptides utilizing orthogonal inteins and endoplasmic reticulum (ER) retention motifs to constitute a cell sorting system. In certain embodiments, the ER retention motif retains the transmembrane domain containing construct inside the cell in the non-spliced state. In certain embodiments, the ER retention motif is cleaved off during trans-splicing with a cognate orthogonal intein pair, allowing the mature spliced intein-tagged transmembrane polypeptide to traffic to the cell surface. In certain embodiments, ER retention motifs can be appended to multiple, distinct intracellular polypeptides and by utilizing orthogonal inteins, multiple rounds of trans-splicing and ER retention motif cleavage can be engineered to either retain trans-spliced transmembrane polypeptides in the ER or allow traffic to the cell membrane. In certain embodiments, multiplexed genetic modification of cells with multiple vectors, in which each vector encodes single subunits of modular intein-tagged polypeptides that when combined reconstitute a mature affinity-tagged transmembrane polypeptide expressed on the cell surface. In certain embodiments, magnetic bead-based sorting (MACS) targeting a specific affinity tag enables selective enrichment of cells that have correctly trans-spliced the mature affinity-tagged polypeptide product, resulting in selective isolation of cells that have integrated at least one copy of each vector that encodes the intein-tagged polypeptide subunits (molecular coincidence detector). For example, this process can enable selective sorting of cells integrating at least four unique vectors. In certain embodiments, additional transgenes can be incorporated into each vector, enabling enhanced capacity for genetic information transfer into cells. In addition, in certain embodiments, trans-splicing of degenerate modular peptides enables generation of increased numbers of mature, trans-spliced polypeptides from a given set of available precursor polypeptides.

In certain embodiments, the present disclosure is directed to systems for the production of affinity-tagged modular CARs utilizing drug-regulated polypeptide degradation systems of the intracellular intein-tagged polypeptide. In certain embodiments, unstable degrons are appended to an intracellular intein-tagged polypeptide, resulting in selective drug-regulated degradation of the intracellular polypeptide and therefore, drug-regulated trans-splicing with the intein-tagged transmembrane polypeptide. In certain embodiments, the intein-tagged transmembrane polypeptide contains an ER retention motif, retaining the polypeptide intracellularly when the degron-containing intracellular intein-tagged polypeptide is degraded. In certain embodiments, the intein-tagged transmembrane polypeptide trans-splices with the degron-containing, intein-tagged intracellular polypeptide when in the presence of a small molecule, which promotes increased concentrations of the intracellular intein-tagged polypeptide, cleavage of the ER retention motif, and cell surface transit of the mature trans-spliced polypeptide (drug-on system). In certain embodiments, the intein-tagged transmembrane polypeptide trans-splices with the degron-containing, intein-tagged intracellular polypeptide when in the absence of a small molecule, which promotes increased concentrations of the intracellular intein-tagged polypeptide, cleavage of the ER retention motif, and cell surface transit of the mature trans-spliced polypeptide (drug-off system). In certain embodiments, multiplexing of orthogonal drug destabilizing motifs enables selective splicing of multiple, distinct intein-tagged intracellular domain polypeptides by inducing selective, orthogonal degradation or stabilization. In certain embodiments, MACS sorting enables selective enrichment of cells that have correctly spliced the mature, surface-expressed, affinity-tagged, trans-spliced product, resulting in selective isolation of cells that have integrated at least one copy of each vector that encodes an intein-tagged polypeptide subunit.

In certain embodiments, the present disclosure is directed to systems for producing affinity-tagged modular CARs utilizing low-affinity orthogonal inteins requiring small molecule-regulated dimerization of appended heterodimerizing polypeptide substituents. In certain embodiments, small molecule-induced dimerization of intein-tagged polypeptide subunits promotes trans-splicing to form a mature polypeptide. In certain embodiments of this system, trans-splicing results in cleavage of the ER retention motif, resulting relocation of the mature trans-spliced polypeptide from the ER to the cell surface. In certain embodiments of the system, the receptors do not contain ER retention motifs, and transit to the cell surface independent of trans-splicing. In certain embodiments, combinations of the above embodiments may be generated using the orthogonality principle of intein trans-splicing to enforce recombination of specific molecules. For example, specific antigen binding domains can splice with intracellular domain inteins containing CD3z while other antigen binding domains splice with costimulatory domain inteins (e.g., 4-1BB and CD28). In addition, a subset of orthogonal inteins and polypeptide subunits can be arranged into a sorting system, while other orthogonal inteins are used to enable small molecule induced splicing of CAR subunits.

In certain non-limiting embodiments, the presently disclosed subject matter is directed to methods of modifying a cell comprising delivering to the cell, the system of the present disclosure. In certain of such methods, the cell is a mammalian cell. In certain of such methods, the mammalian cell is an immune cell. In certain of such methods, the immune cell is a T cell.

In certain non-limiting embodiments, the presently disclosed subject matter is directed to methods for enriching a population of modified cells where the enrichment comprises: modifying a population of cells as disclosed herein; culturing the population of cells; and enriching for the population of modified cells by selecting for the surface expression of an extein. In certain of such methods, the cell is a mammalian cell. In certain of such methods, the mammalian cell is an immune cell. In certain of such methods, the immune cell is a T cell.

In certain non-limiting embodiments, the presently disclosed subject matter is directed to methods for treating a disease comprising providing to a subject in need thereof, a population of modified cells comprising a system as described herein, or a cell modified according to a method of modification disclosed herein, or an enriched population of cells enriched according to a method disclosed herein. In certain of such embodiments, the subject is a human subject. In certain of such embodiments, the disease is a cancer, an autoimmune disease, an inflammatory disease, or a graft versus-host disease. In certain of such embodiments, the cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma. In certain of such embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain of such embodiments, the lymphoma is Hodgkin's lymphoma or non-Hodgkin's lymphoma. In certain of such embodiments, the non-Hodgkin's lymphoma is B-cell non-Hodgkin's lymphoma or T-cell non-Hodgkin's lymphoma. In certain of such embodiments, the cancer comprises cells expressing CD19 or CD20. In certain of such embodiments, the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

### 5. BRIEF DESCRIPTION OF THE DRAWINGS

FIGS. 1A-1D depict exemplary split intein mediated trans-splicing reactions. FIG. 1A illustrates an exemplary intein trans-splicing reaction a first polypeptide comprising a split intein fused at the N terminus of the intein and a second polypeptide comprising the complementary split intein fused at the C terminus of the intein. Upon association of the split inteins, trans-splicing of first and second polypeptides (exteins) is catalyzed, resulting in covalent linkage of the first and second polypeptides via a peptide bond and release of the non-covalently associated inteins. FIG. 1B illustrates degeneracy of intein based trans-splicing reactions, which enables generation of a combination of mature, trans-spliced polypeptides from a given set of available precursor polypeptides. FIG. 1C provides an exemplary illustration of intein degeneracy using anti-CD19 scFv or anti-CD20 scFv fused at the N terminus of a split intein, and co-stimulatory molecules CD28z or 4-1BB at the C terminus of a complementary split intein. FIG. 1D illustrates a comparison between encoding necessary for transducing exogenous genes through conventional versus intein-based gene expression (e.g., CAR). The degeneracy of intein based trans-splicing reactions provides a distinct advantage by enabling expression of large constructs with higher efficiency than using conventional approaches and enables encoding of a larger number of receptors using the same amount of DNA.

FIGS. 2A-2B depict an exemplary two-vector intein sorting system. FIG. 2A illustrates that in this exemplary approach, Vector 1 encodes a secreted affinity-tagged molecule fused to an intein N terminal domain. Vector 2 encodes a C terminus intein capture domain fused to a spacer, transmembrane domain, linker, and non-signaling cytoplasmic domain. Trans-splicing of the two intein-tagged polypeptides results in cell selective surface display of the affinity tag on dual-vector transduced cells resulting from selective intracellular splicing. This enables selective affinity-tag based MACS sorting of cells incorporating both vectors and completing the trans-splicing reaction. Each vector can carry additional transgenes to boost engineered cell functionality. FIG. 2B depicts the results of an exemplary experiment employing a two-vector intein sorting system making use of a secreted affinity tag with intein tag, and a transmembrane intein capture stalk.

FIGS. 3A-3I depict an exemplary approach for two-vector intein endoplasmic reticulum retention-based sorting and post-translational CAR assembly. FIG. 3A illustrates that in this exemplary approach, Vector 1 encodes an intein-tagged antigen binding construct comprised of an scFv fused to an affinity tag, a spacer, a transmembrane domain, an intein N terminus, and an endoplasmic reticulum (ER) retention motif, and Vector 2 encodes a cytoplasmic signaling domain shuttle construct comprised of an intein C terminus fused to a costimulatory domain, and a T cell receptor signaling domain (e.g., CD3 zeta). Trans-splicing of the two the intein-tagged polypeptides results in an intracellular splice junction and a recapitulates a fully functional chimeric antigen receptor (CAR). Surface expression of the affinity-tagged antigen-binding domain is markedly reduced due to ER retention until the two inteins trans-splice, which removes the ER retention motif, resulting in cell surface trafficking. This enables selective affinity-tag based MACS sorting of cells incorporating both vectors and completing the trans-splicing reaction. Each vector can carry additional transgenes to boost engineered cell functionality. FIG. 3B depicts an exemplary two-vector intein modular CAR sorting system where an extracellular scFv with spacer is fused to transmembrane intein capture stalk with an inward rectifier potassium (Kir6.2) RXR ER retention motif. FIG. 3C depicts an exemplary two-vector intein modular CAR sorting system where an extracellular scFv with spacer is fused to transmembrane intein capture stalk with a E319K ER retention motif. FIG. 3D shows the efficacy of CD19-CAR T cells in killing CD19 expressing BM185 cells. FIG. 3E illustrates an exemplary three vector intein retention-based using an RXR or a E319K (KKXX) retention motifs. FIGS. 3F-3I demonstrate the ability of RXR-type ER retention motifs to function inside of a polypeptide chain.

FIGS. 4A-4G. FIG. 4A depict an exemplary two-vector intein sorting system for generating cells expressing modular dual-CARs having dual antigen specificity. In this exemplary approach, Vector 1 encodes dual affinity-tagged intein-tagged scFvs each fused to an affinity tag and degenerate intein N terminus, and Vector 2 encodes a transmembrane signaling domain shuttle comprised of an intein C terminus fused to a spacer domain, transmembrane domain, a costimulatory domain, and a T cell receptor signaling domain. Following capture and splicing of the intein domains, both antigen binding-domains are displayed on the cell surface, recapitulating full-length CAR molecules. Trans-splicing of the two intein-tagged polypeptides results in cell selective surface display of the affinity tag in dual-vector transduced cells due to selective intracellular splicing. This enables selective affinity-tag based MACS sorting of cells incorporating both vectors and completing the trans-splicing reaction. Each vector can carry additional transgenes to boost engineered cell functionality. FIG. 4B depicts the results of enrichment by MACS sorting of an exemplary modular scFv intein-CAR having dual antigen specificity with dual secreted intein-tagged affinity-tagged scFvs and a transmembrane intein capture stalk fused to a cytoplasmic signaling domain. FIG. 4C shows the efficacy of dual-targeted CD19/CD20-specific intein CAR-T cells in killing BM185-CD19 target cells. FIG. 4D shows the efficacy of dual-targeted CD19/CD20-specific intein CAR-T cells in killing BM185-CD20 target cells. FIG. 4E depicts another exemplary two-vector dual-CAR intein sorting system in which the cytoplasmic signaling domain shuttle as depicted in FIG. 3A utilizes a zeta-chain-associated protein kinase 70 (Zap70) signaling domain and a tracer protein (e.g., EGFP). FIG. 4F depicts the results of MACS sorting enrichment of the transduced cells generated using the exemplary constructs in FIG. 4E. FIG. 4G shows the efficacy of dual-targeted CD19/CD20-specific Zap70 intein CAR-T cells in killing C1498 target cells expressing either CD19 or CD20.

FIGS. 5A-5D. depict an exemplary three-vector intein ER retention-based sorting system and post-translational CAR assembly with secreted intein-tagged scFvs. FIG. 5A illustrates that in this exemplary approach, Vector 1 encodes dual antigen-binding scFvs each fused to an affinity tag and an orthogonal intein N terminus, Vector 2 encodes a dual-intein transmembrane adapter comprised of an orthogonal intein C terminus fused to extracellular and transmembrane domains, an orthogonal intein N terminus, and an ER retention motif, and Vector 3 encodes a cytoplasmic signaling domain shuttle comprised of an orthogonal intein C terminus fused to a costimulatory domain and T cell receptor signaling domain. The intein-tagged scFv molecules trans-splice with the dual-intein transmembrane adapter, which exits the ER following trans-splicing of the orthogonal inteins of the adapter and cytoplasmic shuttle constructs, thereby recapitulating a full-length CAR, with cleavage of the ER retention motif. This enables selective affinity-tag based MACS sorting of cells incorporating three vectors following completion of two orthogonal trans-splicing reactions. Each vector can carry additional transgenes to boost engineered cell functionality. FIG. 5B depicts the results of MACS enrichment of the triple vector transduced cells. FIG. 5C shows the efficacy of dual-targeted CD19/CD20-specific intein CAR-T cells in killing BM185-CD19 target cells. FIG. 5D shows the efficacy of dual-targeted CD19/CD20-specific intein CAR-T cells in killing BM185-CD19 target cells.

FIGS. 6A-6D depict an exemplary three-vector hybrid leucine zipper-intein ER retention-based sorting system and post-translational CAR assembly. FIG. 6A illustrates that in this exemplary approach, Vector 1 encodes dual antigen-binding scFvs each fused to an affinity tag and a heterodimerizing leucine zipper, Vector 2 encodes a leucine zipper-intein transmembrane adapter comprised of a heterodimerizing leucine zipper fused a short hinge domain, transmembrane domain, an intein N terminus, and an ER retention motif, and Vector 3 encodes a cytoplasmic signaling domain shuttle comprised of an intein C terminus fused to a costimulatory domain and a T cell receptor signaling domain. The leucine zipper-tagged scFv molecules bind to the capture zipper-intein transmembrane adapter in the ER, which can exit the ER following trans-splicing of inteins of the adapter and cytoplasmic shuttle constructs, which recapitulates a full-length CAR, with cleavage of the ER retention motif. This enables selective affinity-tag based MACS sorting of cells incorporating three vectors following completion of zipper heterodimerization and intein trans-splicing reactions. Each vector can carry additional transgenes to boost engineered cell functionality. FIG. 6B shows the results of MACS sorting enrichment of the transduced cells generated using the exemplary constructs in FIG. 6A. FIGS. 6C and 6D show the efficacy of the dual-targeted CD19/CD20-specific Zap70 intein CAR-T cells generated using the approach shown in FIG. 6A, BM185-CD19 and BM185-CD20 target cells respectively.

FIG. 7 depicts an exemplary three-vector-intein ER retention-based sorting system and post-translational CAR assembly with intracellular intein adapter. In this exemplary approach, Vector 1 encodes an intein-tagged antigen binding construct comprised of an scFv fused to an affinity tag, a spacer, a transmembrane domain, an orthogonal intein N terminus, and an ER retention motif, Vector 2 encodes a dual-intein cytoplasmic adapter comprised of an orthogonal intein C terminus fused to a signaling domain, an orthogonal intein N terminus, and an ER retention motif, and Vector 3 encodes a cytoplasmic signaling domain shuttle comprised of an orthogonal intein C terminus fused to a costimulatory domain and/or T cell receptor signaling domain. The orthogonal intein-tagged molecules trans-splice as depicted with serial cleavage of both ER retention motifs with recapitulation of a full-length CAR molecule. Removal of ER retention motifs enables selective trafficking of the CAR molecule to the cell surface and selective affinity-tag based MACS sorting of cells incorporating three vectors following completion of two orthogonal trans-splicing reactions. Each vector can carry additional transgenes to boost engineered cell functionality.

FIGS. 8A-8D depict an exemplary four-vector intein ER retention-based sorting system and post-translational CAR assembly with combined use of transmembrane and intracellular intein adapters. FIG. 8A illustrates that in this exemplary approach, Vector 1 encodes dual antigen-binding scFv fused to an affinity tag and an orthogonal intein N terminus, Vector 2 encodes a dual-intein transmembrane adapter comprised of an orthogonal intein C terminus fused to extracellular and transmembrane domains, an orthogonal intein N terminus, and an ER retention motif, and Vector 3 encodes a dual-intein cytoplasmic adapter comprised of an orthogonal intein C terminus fused to a signaling domain, an orthogonal intein N terminus, and an ER retention motif, and Vector 4 encodes a cytoplasmic signaling domain shuttle comprised of an orthogonal intein C terminus fused to a costimulatory domain and/or T cell receptor signaling domain. The orthogonal intein-tagged molecules trans-splice as depicted with serial cleavage of both ER retention motifs with recapitulation of a full-length CAR molecule. Sequential removal of ER retention motifs enables selective trafficking of the CAR or sorting stalk molecule to the cell surface and selective affinity-tag-based MACS sorting of cells incorporating four vectors following completion of three orthogonal trans-splicing reactions. Each vector can carry additional transgenes to boost engineered cell functionality. FIG. 8B shows the results of exemplary experiments using the four-vector intein sorting system having a FLAG tag, which enables MACS sorting enrichment of the transduced cells. FIG. 8C shows the results of exemplary experiments using the four-vector intein sorting system having a human CD34 tag, which enables MACS sorting enrichment of the transduced cells. FIG. 8D shows characterization of transduced cells prepared using all 4 vectors or, omitting Vector 3 in FIG. 8A.

FIGS. 9A-9C depict an exemplary two-vector intein sorting system that employs a drug regulatable CAR expression-based approach. FIG. 9A illustrates that in this exemplary approach, Vector 1 encodes an intein-tagged antigen binding construct comprised of an scFv fused to an affinity tag, a spacer, a transmembrane domain, a low-affinity intein N terminus, a drug-dimerizable domain (e.g., FKBP12), and an ER retention motif, and Vector 2 encodes a drug-dimerizable domain (e.g., FRB*) fused to a low-affinity intein C terminus, a costimulatory domain, and a T cell receptor signaling domain (e.g., CD3 zeta). Trans-splicing of the two the low-affinity intein-tagged polypeptides occurs maximally in the presence of a dimerizer drug due to enhanced association of the polypeptides mediated by the drug-dimerizable domains. Trans-splicing results in an intracellular splice junction and a recapitulates a fully functional CAR. Surface expression of the affinity-tagged antigen-binding domain polypeptide is markedly reduced due to ER retention until the two inteins trans-splice, which removes the ER retention motif, resulting in cell surface trafficking. This system enables drug-dependent post-translational CAR assembly and selective affinity-tag-based MACS sorting of cells incorporating both vectors and completing the trans-splicing reaction. Each vector can carry additional transgenes to boost engineered cell functionality. FIG. 9B shows the results of exemplary flow cytometric experiments using the drug regulatable two-vector intein sorting system illustrated in FIG. 9A, characterizing expression of hCD34 in the absence or presence of an A/C heterodimerizer (drug). FIG. 9C shows selective killing of BM185-CD19 (target cells, T) by CAR-T cells (effector cells, E, generated using the approach shown in FIG. 9A) in the presence of the dimerizing drug (e.g., AP21967).

FIGS. 10A-10L depict an exemplary two-vector intein sorting system with drug-regulatable dual-CAR expression-based approach. FIG. 10A illustrates that in this exemplary approach, Vector 1 encodes two distinct antigen-binding scFv transmembrane-domain-containing fusion polypeptides with cytoplasmic low-affinity N terminus intein domains. Each scFv fusion polypeptide contains a distinct affinity tag, a spacer, a transmembrane domain, a low-affinity intein N terminus, and a drug-dimerizable domain (e.g., FKBP12), but only one contains an ER retention motif, due to the ER retention motif functioning most efficiently at the end of a vector construct (C terminus of fusion polypeptide and 2A sequence). Vector 2 encodes a drug-regulated cytoplasmic signaling domain shuttle comprised of a drug-dimerizable domain (e.g., FRB*) fused to a low-affinity intein C terminus, a costimulatory domain, and a T cell receptor signaling domain (e.g., CD3 zeta). Trans-splicing of the two the low-affinity intein-tagged polypeptides occurs maximally in the presence of a dimerizer drug due to enhanced association of the polypeptides mediated by the drug-dimerizable domains. Trans-splicing results in an intracellular splice junction and recapitulates fully functional CARs each targeting cognate antigens. Surface expression of the affinity-tagged antigen-binding domain polypeptide with an ER retention motif is markedly reduced due to ER retention until the two inteins trans-splice, which removes the ER retention motif, resulting in cell surface trafficking. This system enables drug-dependent post-translational dual-CAR assembly and selective affinity-tag based MACS sorting of cells incorporating both vectors and completing the trans-splicing reaction. Each vector can carry additional transgenes to boost engineered cell functionality. FIG. 10B shows the results of exemplary flow cytometric experiments using the drug regulatable two-vector intein sorting system illustrated in FIG. 10A, demonstrating enhanced surface expression of an hCD34-tagged CAR in the presence of an A/C heterodimerizer or rapamycin, resulting from cleavage of its ER retention motif by the cytoplasmic signaling shuttle intein when brought into proximity to the antigen binding stalk molecules by either drug. FIG. 10C shows the efficacy of the CAR-T cells (effector cells, E) generated using the approach shown in FIG. 10A, in killing CD19 expressing BM185 cells (target cells, T). FIG. 10D shows the efficacy of the CAR-T cells generated using the approach shown in FIG. 10A, in killing CD20 expressing BM185 cells. FIGS. 10E-10L show live cell imaging of BM185-CD19 and BM185-CD20 tumor cell lines co-cultured with CD19/CD20 dual-targeting intein CAR T cells in the presence or absence of activating small molecules.

FIGS. 11A-11C depict an exemplary two-vector intein sorting system with drug regulatable trans-presented cell surface cytokine expression-based approach. FIG. 11A illustrates that in this exemplary approach, Vector 1 encodes a cytokine fused to an affinity tag, a spacer, a transmembrane domain, a low-affinity intein N terminus, a drug-dimerizable domain (e.g., FKBP12), and an endoplasmic reticulum (ER) retention motif, Vector 2 encodes a drug-dimerizable domain (e.g., FRB*) fused to a low-affinity intein C terminus and a costimulatory domain containing or non-signaling intracellular domain. Trans-splicing of the two low-affinity intein-tagged polypeptides occurs maximally in the presence of a dimerizer drug due to enhanced association of the polypeptides mediated by the drug-dimerizable domains. Trans-splicing results in excision of the ER retention motif. Surface expression of the affinity-tagged cytokine trans-presentation polypeptide is markedly reduced due to ER retention until the two inteins trans-splice, which removes the retention motif, resulting in cell surface trafficking. This system enables drug-dependent graded expression of a cell surface trans-presented cytokine and selective affinity-tag-based MACS sorting of cells incorporating both vectors and completing the trans-splicing reaction. Each vector can carry additional transgenes to boost engineered cell functionality. FIG. 11B shows the results of constitutive and drug-regulated expression of IL-7 on the cell surface. FIG. 11C shows the results of T cell expansion for constitutive and drug-regulated expression of IL-7.

FIGS. 12A-12C depict an exemplary two-vector degenerate intein trans-splicing to enable dual-CAR formation with a drug-stabilized cytoplasmic signaling domain shuttle expression-based approach. FIG. 12A illustrates that in this exemplary approach, Vector 1 encodes two distinct antigen-binding scFv transmembrane-domain-containing fusion polypeptides with degenerate cytoplasmic N terminus intein domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes a cytoplasmic signaling domain shuttle comprised of an intein C terminus, a costimulatory domain, a T cell receptor signaling domain (e.g., CD3 zeta), and a drug-stabilized degron domain (e.g., E coli DHFR destabilization domain). Surface expression of the affinity-tagged antigen-binding domain polypeptide with an ER retention motif is markedly reduced due to ER retention until trans-splicing with the cytoplasmic signaling domain shuttle, which removes the retention motif, resulting in cell surface trafficking. Maximum expression of the cytoplasmic signaling domain shuttle occurs in the presence of the stabilizing drug, facilitating trans-splicing reaction. This system enables drug-dependent post-translational dual-CAR and assembly combined with selective affinity-tag-based MACS sorting of cells incorporating both vectors and completing the trans-splicing reaction. Each vector can carry additional transgenes to boost engineered cell functionality. FIG. 12B depicts the results of exemplary experiments employing the two-vector intein-based modular CAR approach illustrated in FIG. 12A in the absence and presence of trimethoprim. FIG. 12C shows the efficacy of the CAR-T cells generated using the approach illustrated in FIG. 12A, in killing CD19 expressing BM185 cells in the absence and presence of trimethoprim.

FIGS. 13A-13G depicts an exemplary two-vector-degenerate intein trans-splicing to enable dual-CAR formation with drug-destabilized cytoplasmic signaling domain shuttle expression-based approach. FIG. 13A illustrates that in this exemplary approach, Vector 1 encodes two distinct antigen-binding scFv transmembrane-domain-containing fusion polypeptides with degenerate cytoplasmic N terminus intein domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes a cytoplasmic signaling domain shuttle comprised of an intein C terminus, a costimulatory domain, a T cell receptor signaling domain (e.g., CD3 zeta), and a drug-activated degron domain (e.g., SMASh tag). Surface expression of the affinity-tagged antigen-binding domain polypeptide with an ER retention motif is markedly reduced due to ER retention until trans-splicing with the cytoplasmic signaling domain shuttle, which removes the retention motif, resulting in cell surface trafficking. Expression of the cytoplasmic signaling domain shuttle is reduced in the presence of the inhibitor drug, which promotes proteasomal degradation, resulting in inhibition of the trans-splicing reaction and reduction in CAR formation. This system enables drug-dependent post-translational dual-CAR assembly combined with selective affinity-tag-based MACS sorting of cells incorporating both vectors and completing the trans-splicing reaction. Each vector can carry additional transgenes to boost engineered cell functionality. FIG. 13B depicts characterization of transfected cells precultured in the absence or presence of asunaprevir using a hCD34 affinity tag. FIG. 13C depicts characterization of transfected cells precultured in the absence or presence of asunaprevir using a FLAG affinity tag. FIG. 13D shows the efficacy of the CAR-T cells precultured in the absence or presence of asunaprevir in killing CD19 expressing BM185 cells. FIG. 13E depicts characterization of dual-CAR transfected cells precultured in the absence or presence of asunaprevir using a hCD34 or FLAG affinity tag. FIG. 13F shows the efficacy of the dual-CAR-T cells precultured in the absence or presence of asunaprevir in killing CD19 expressing BM185 cells. FIG. 13G shows the efficacy of the dual-CAR-T cells precultured in the absence or presence of asunaprevir in killing CD20 expressing BM185 cells.

FIGS. 14A-14C depict an exemplary two-vector dual-CAR intein sorting system comprising a drug regulatable Zap70 domain shuttle FIG. 14A illustrates that in this exemplary approach, Vector 1 encodes two distinct antigen-binding scFv transmembrane-domain-containing fusion polypeptides (e.g., anti-CD19, anti-CD20) with degenerate cytoplasmic N terminus intein (I_{N}) domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes a drug regulatable Zap70 domain shuttle comprised of a Non-structural 3 (NS3) protease and flanking 4A/4B and 5A/B HCV protease cleavage recognition sequences and an intein C terminus (Ic). FIG. 14B illustrates the process where, in the absence of an HCV protease inhibitor (e.g., grazoprevir), the NS3 protease cleaves the intein away from the NS3-Zap70 fusion protein, abrogating generation of a signaling competent Zap70-based intein CAR. FIG. 14C illustrates the efficacy of T cells transfected with a dual CD19/CD20 CAR Zap70 construct in killing CD19 or CD20 expressing BM185 cells in the presence of grazoprevir (GZP).

FIGS. 15A-15D depict exemplary intein-assisted NS3-based drug-regulated CAR. FIG. 15A illustrates that in this exemplary approach, where drug-regulated dual-Zap70 intein-CAR T cells demonstrate killing both BM185-CD19 and BM185-CD20 tumor cell targets in the presence of grazoprevir (GZP). FIG. 15B illustrates the efficacy of drug-regulated CD19-targeting NS3 CD28z intein-CAR T cells with either two flanking HCV protease cut sites (5A/5B and 4A/4B) or either alone enable selective, drug induced killing of BM185-CD19 tumor cell targets in the presence of grazoprevir (GZP), without leaky killing of targets in the non-drug-induced state. FIGS. 15C-15D illustrate live cell imaging microscopy showing selective killing of BM185-CD19 and BM185-CD20 by drug-regulated CD19/CD20 dual-targeting NS3 CD28z intein-CAR T cells in the presence of grazoprevir without non-induced “leaky” target killing.

FIGS. 16A-16C depict exemplary intein-assisted NS3-based drug-regulated CARs generated by a nested intein approach, whereby nested inteins are used to sequentially slice out drug-regulated cleavage cassette. FIG. 16A illustrates that in this exemplary approach. The low-affinity AES split intein only splices once brought into proximity by the initial splicing and peptide fusion between the antigen binding stalk and cytoplasmic signaling shuttle by the high-affinity Cfa intein. This second splicing step generates a trans-spliced mature CAR molecule without intervening NS3 sequences, which may impair CAR signaling. FIG. 16B illustrates the efficacy of CAR T cells generated by the non-nested (FIG. 15A) and nested approaches in killing target cell expressing a high density of CD19 antigen. FIG. 16C illustrates the efficacy of CAR T cells generated by the non-nested (FIG. 15A) and nested approaches in killing target cells expressing a low density of CD19 antigen.

FIGS. 17A-17H depicts an exemplary two-vector degenerate intein trans-splicing to enable multiplexed post-translational CAR and chimeric costimulatory receptor (CCR) formation. FIG. 17A illustrates that in this exemplary approach, Vector 1 encodes two distinct antigen-binding scFv transmembrane-domain-containing fusion polypeptides with degenerate cytoplasmic N terminus intein domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes two cytoplasmic signaling domain shuttles each comprised of a degenerate intein C terminus, and a distinct costimulatory domain and/or a T cell receptor signaling domain (e.g., CD3 zeta). Trans-splicing of combinations of two degenerate intein-tagged antigen-binding polypeptides with the two cytoplasmic signaling domain shuttles enables generation of two CAR and two CCR molecules totaling four mature trans-sliced polypeptides. Surface expression of the affinity-tagged antigen-binding domain polypeptide with an ER retention motif is markedly reduced due to ER retention until the two inteins trans-splice, which removes the retention motif, resulting in cell surface trafficking. This system enables post-translational dual-CAR and dual-CCR assembly combined with selective affinity-tag-based MACS sorting of cells incorporating both vectors and completing the trans-splicing reaction. Further modification to the cytoplasmic signaling domain shuttles to include orthogonal drug-destabilization or selective degron domains can enable selective drug-dependent regulation of each signaling domain. Each vector can carry additional transgenes to boost engineered cell functionality. FIG. 17B illustrates exemplary flow cytometric analysis of CD19 CAR and CCR transduced cells before and after MACS sorting. FIG. 17C illustrates exemplary flow cytometric analysis of CD19 CAR only transduced cells before and after MACS sorting. FIG. 17D shows the results for expansion of CD19 CAR/CCR expressing cells and CD19 CAR only expressing cells after exposure to CD19 target cells. FIG. 17E shows the results of flow cytometric analysis of an exemplary dual-CAR (CD19/CD20)+dual-CCR (dual stalk+CD28z+4-1BB shown in FIG. 17F. FIG. 17G illustrates the efficacy of dual-CAR T cells to eliminate BM185-CD19 or BM185-CD20 cell targets at the indicated E:T ratios. FIG. 17H shows the results of expansion studies for dual-CAR, dual-CCR T cells after exposure to BM185-CD19 or BM185-CD20 target cells at the indicated E:T ratios.

FIGS. 18A-18C depict an exemplary two-vector dual-CAR intein sorting system comprising degenerate Zap70 domain shuttle and 4-1BB domain shuttle sequences, which can combine in four possible combinations: CD19-Zap70-CAR, CD20-Zap70-CAR, CD19-CCR, CD20-CCR. This configuration represents a data compression algorithm whereby re-use of degenerate encoded DNA elements enables post-translational assembly of a greater number of receptors than could be encoded with the same amount of DNA. FIG. 18A illustrates that in this exemplary approach, Vector 1 encodes two distinct antigen-binding scFv transmembrane-domain-containing fusion polypeptides (e.g., anti-CD19, anti-CD20) with degenerate cytoplasmic N terminus intein (I_{N}) domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct. Vector 2 encodes Zap70 domain shuttle and a 4-1BB domain sequence each attached at their N terminus to an intein C terminus (Ic). FIGS. 18B and 18C demonstrate enhanced killing of BM185-CD19 and BM185-CD20 targets by CD19/CD20 dual-targeting Zap70+BB intein-CAR/CCR T cells and enhanced T cell proliferation compared with intein CARs utilizing only the Zap70 cytoplasmic shuttle (CARs, but no CCRs formed).

### 6. DETAILED DESCRIPTION

The present application is directed to multiplex intein-based methods and compositions for the generation and sorting of modular polypeptides, e.g., CARs and CCRs. For example, but not by way of limitation, the present application is directed to multiplexed pairs of inteins, where each intein of a pair is a fused to an extein, e.g., a modular polypeptide sequence, and where the paired inteins catalyze trans polypeptide splicing (“trans-splicing”), thereby fusing the corresponding exteins to facilitate the production of a desired mature modular polypeptide. In certain embodiments, orthogonal pairs of inteins can be employed to allow for multiplexed trans-splicing. Because certain intein pairs, e.g., split intein pairs, only trans-splice with their paired sequence, multiplexing of such pairs allows for the trans-splicing of distinct exteins, e.g., distinct antibody binding domains, to paired exteins, e.g., particular transmembrane domains, while the unrelated split-intein pairs simultaneously allow for the trans-splicing of distinct extein pairs, e.g., intracellular signaling domains. As described herein, exemplary potential trans-splicing exteins include: antigen binding domains (which may include affinity tag(s)); surface presented membrane-bound cytokines; spacer and transmembrane domains (which may include affinity tag(s)); positive and negative costimulatory molecule extracellular domains; intracellular signaling domains; and intracellular non-signaling domains.

In certain embodiments, the subject matter of the present disclosure comprises methods and compositions relating to cell sorting systems. For example, but not by way of limitation, a cell sorting system according to the present disclosure can comprise affinity-tagged modular extracellular and transmembrane domain-containing polypeptides, e.g., CARs and CCRs, utilizing orthogonal inteins and endoplasmic reticulum (ER) retention motifs. In certain embodiments, the ER retention motif retains the transmembrane domain containing construct inside the cell in the non-spliced state. In certain embodiments, the ER retention motif can be cleaved off during trans-splicing with a cognate orthogonal intein pair, allowing the mature spliced intein-tagged transmembrane polypeptide to traffic to the cell surface. In certain embodiments, ER retention motifs can be appended to multiple, distinct intracellular polypeptides and by utilizing orthogonal inteins, multiple rounds of trans-splicing and ER retention motif cleavage can be engineered to either retain trans-spliced transmembrane polypeptides in the ER or allow traffic to the cell membrane. In certain embodiments, multiplexed genetic modification of cells with multiple vectors, where each vector encodes single subunits of modular intein-tagged polypeptides that when combined reconstitute a mature affinity-tagged transmembrane polypeptide expressed on the cell surface.

In certain embodiments, magnetic bead-based sorting (MACS) targeting a specific affinity tag enables selective enrichment of cells that have correctly trans-spliced the mature affinity-tagged polypeptide product, resulting in selective isolation of cells that have integrated at least one copy of each vector that encodes the intein-tagged polypeptide subunits (molecular coincidence detector). In certain embodiments, the process enables selective sorting of cells integrating at least four unique vectors. In certain embodiments, additional transgenes can be incorporated into each vector, enabling enhanced capacity for genetic information transfer into cells. In certain embodiments, trans-splicing of degenerate modular peptides enables generation of increased numbers of mature, trans-spliced polypeptides from a given set of available precursor polypeptides.

In certain embodiments, the subject matter of the present disclosure comprises methods and compositions relating to systems for the drug-regulated generation and sorting of modular intracellular and transmembrane domain-containing polypeptides, e.g., CARs and CCRs. In certain embodiments, unstable degrons are appended to an intracellular intein-tagged polypeptides, resulting in selective drug-regulated degradation of the intracellular polypeptides and therefore, drug-regulated trans-splicing with the intein-tagged transmembrane polypeptides. In certain embodiments, the intein-tagged transmembrane polypeptide contains an ER retention motif, retaining the polypeptide intracellularly when the degron-containing intracellular intein-tagged polypeptide is degraded. In certain embodiments, the intein-tagged transmembrane polypeptide trans-splices with the degron-containing, intein-tagged intracellular polypeptide when in the presence of a small molecule, which promotes increased concentrations of the intracellular intein-tagged polypeptide, cleavage of the ER retention motif, and cell surface transit of the mature trans-spliced polypeptide (a “drug-on” system). In certain embodiments, the intein-tagged transmembrane polypeptide trans-splices with the degron-containing, intein-tagged intracellular polypeptide when in the absence of a small molecule, which promotes increased concentrations of the intracellular intein-tagged polypeptide, cleavage of the ER retention motif, and cell surface transit of the mature trans-spliced polypeptide (a “drug-off” system). In certain embodiments, multiplexing of orthogonal drug destabilizing motifs enables selective splicing of multiple, distinct intein-tagged intracellular domain polypeptides by inducing selective, orthogonal degradation or stabilization. In certain embodiments, MACS sorting enables selective enrichment of cells that have correctly spliced the mature, surface-expressed, affinity-tagged, trans-spliced product, resulting in selective isolation of cells that have integrated at least one copy of each vector that encodes an intein-tagged polypeptide subunit.

In certain embodiments, the subject matter of the present disclosure comprises methods and compositions relating to systems for the generation of affinity-tagged modular polypeptides utilizing low-affinity orthogonal inteins requiring small molecule-regulated dimerization of appended heterodimerizing polypeptide substituents. In certain embodiments, small molecule-induced dimerization of intein-tagged polypeptide subunits promote trans-splicing to form a mature modular polypeptide. In certain embodiments, trans-splicing results in cleavage of the ER retention motif, resulting relocation of the mature trans-spliced polypeptide from the ER to the cell surface. In certain embodiments, the modular polypeptides, e.g., CARs and CCRs, do not contain ER retention motifs, and transit to the cell surface independent of trans-splicing.

In certain embodiments, the subject matter of the present disclosure comprises methods and compositions relating to systems comprising combinations of the embodiments described herein using the orthogonality principle of intein trans-splicing to enforce recombination of specific molecules. For example, but not by way of limitation, specific antigen binding domains can splice with intracellular domain inteins, e.g., those containing CD3z, while other antigen binding domains splice with costimulatory domain inteins (e.g., 4-1BB and CD28). In certain embodiments, a subset of orthogonal inteins and polypeptide subunits are arranged into a sorting system, while other orthogonal inteins are used, e.g., to enable small molecule or other “drug”-induced splicing of modular polypeptide, e.g., CAR or CCR, subunits.

### 6.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “intein” refers to any of a variety of auto-catalytic polypeptide sequences capable of self-excision from a larger polypeptide sequence via polypeptide splicing. As used herein, “polypeptide splicing” refers to the auto-catalytic process by which an intein self-excises from a larger polypeptide and which results in the ligation of the intein-flanking sequences (“exteins”) via a new peptide bond. Inteins exist as bifunctional sequences, where the endonuclease domain is present within the splicing domain allowing for cis polypeptide splicing, as well as “split inteins” which are present in separate polypeptide sequences that can self-associate non-covalently to catalyze trans polypeptide splicing. Exemplary inteins are described in Pinto et al., Nature Comm., 11:1529 (2020) and Stevens et al., J. Am. Chem. Soc., 138:2162-2165 (2016), which are incorporated by reference in their entireties for all purposes.

As used herein, an “orthogonal system” is a network of components (e.g., polypeptides, RNAs, DNAs, and small molecules) that interact with each other to achieve a specific function without impeding or being impeded by the native functions of the host cell. Accordingly, “orthogonal split-intein tags” will interact with each other to facilitate trans polypeptide splicing of the exteins to which they are fused without impeding or being impeded by the native functions of the host cell. Orthogonality of inteins derives from specificity of split N terminal and C terminal polypeptide sequences that exhibit high binding affinity and high specificity. As a result, unrelated inteins can be co-expressed in the same cell without high background levels of non-specific trans-splicing of unrelated inteins. This enables co-expression of multiple intein-tagged peptide constructs inside a cell, utilizing orthogonal inteins to specify intended regions of different polypeptides that will splice with one another in a defined configuration. This orthogonal intein strategy allows intracellular construction of complex, mature, trans-spliced proteins from a set of functional domain-containing proteins. Non-limiting examples of these domains include: antigen binding domains, spacers, transmembrane domains, signaling domains, and cytokines. Orthogonality of inteins enables the intein-tagged subunits to assemble in an engineered pre-defined configuration.

As used herein, “degeneracy” in intein-based intracellular polypeptide assembly refers to re-use of specific protein domains in multiple constructs produced within a cell. For example, and not bound by any limitation, an intracellular signaling domain tagged with a specific intein (e.g., Cfa C terminus) can trans-splice with multiple antigen binding domain/spacer/transmembrane-domain/Cfa-N terminus intein constructs. Similarly, multiple Cfa C terminal intein-tagged signaling domains can also trans-splice in parallel with these antigen binding domain constructs, yielding multiple mature and modular trans-splice CAR constructs.

As used herein, the term “molecular coincidence detector” refers to beneficial features of the system in enabling selective enrichment of select cells from a mixed population of cells, wherein the select cells express all proteins encoded by a designated set of vectors. The sorting system aspect of the intein system utilizes combinations of intein-conjugated molecules that are also tagged with affinity-tags or endoplasmic reticulum (ER) retention motifs. In the non-spliced state, cells expressing individual components of the intein sorting system (i.e. transduced with fewer than the number necessary to complete a set of co-transduced vectors) will have minimal amounts of the affinity tag will be expressed on the surface of the cell. However, in cells co-transduced with a complete set of intein sorting system vectors, trans-splicing removes all ER retention motifs from trans-spliced polypeptides and appends an affinity tag to be displayed on the surface of the cells. Consequently, a sorting method, for example, but not limited to MACS sorting using anti-affinity-tag beads selectively purifies only the cells with coincident co-transduction with the necessary vector set to enable surface expression. Thus, in this example, the system enables MACS beads to use the affinity tag as a “molecular coincidence detector” to designate cells that have incorporated all the vectors in a designated vector set. The multi-vector sorting implementation of the intein sorting system thus enables introduction of large amounts of DNA into cells followed by selective purification of cells incorporating all the delivered vectors.

As used herein the term “modular polypeptide” refers to any polypeptide comprised of subunits that when combined reconstitute a mature polypeptide. Non-limiting examples of such modular polypeptide include extracellular domain-containing, intracellular domain-containing, and transmembrane domain-containing polypeptides, as well as polypeptides comprising two or more of such domains e.g., CARs and CCRs.

As used herein, a “linker” refers to a functional group (e.g., chemical or polypeptide) that covalently attaches two or more polypeptides or nucleic acids so that they are connected to one another. In certain embodiments, the linker comprises one or more amino acids used to couple two polypeptides together (e.g., to couple V_{H} and V_{L} domains or to couple two dimerization domains). The linker can be usually rich in glycine for flexibility, as well as serine or threonine for solubility.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence operably linked to appropriate nucleic acid sequences necessary for the expression of the coding sequence in a particular host organism. Nucleic acid sequences necessary for expression in prokaryotes usually include a promoter, an operator (optional), and a ribosome binding site, often along with other sequences. Nucleic acid sequences necessary for expression in eukaryotic cells can include, but are not limited to, promoters, enhancers, and termination and polyadenylation signals.

In certain embodiments, nucleic acid molecules useful in the presently disclosed subject matter include nucleic acid molecules that encode an antibody or an antigen-binding fragment thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule.

As used herein, the term “disease” refers to any condition or disorder that damages or interferes with the normal function of a cell, tissue, or organ. Examples of diseases include neoplasia or pathogenic infection of a cell, tissue, or organ.

An “effective amount” (or “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The dose comprising an effective amount is generally determined by the physician on a case-by-case basis and making such a determination is within the level of ordinary skill in the art. Several factors are typically taken into account when determining an appropriate dosage to achieve an effective amount. These factors include age, sex and weight of the subject, the condition being treated, the severity of the condition and the form and effective concentration of the cells (e.g., engineered immune cells) administered.

As used herein, the term “neoplasm” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of skin, bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, spinal cord, spleen, stomach, testes, thymus, thyroid, trachea, urogenital tract, ureter, urethra, uterus, and vagina, or a tissue or cell type thereof. Neoplasia include cancers, such as melanoma, sarcomas, carcinomas, or plasmacytomas (malignant tumor of the plasma cells).

As used herein, the term “immunoresponsive cell” refers to a cell that functions in an immune response, and includes a progenitor of such cell, and a progeny of such cell.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” polypeptide is sufficiently free of other materials such that any impurities do not materially affect the biological properties of the polypeptide or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or polypeptide gives rise to essentially one band in an electrophoretic gel. For a polypeptide that can be subjected to modifications, for example, phosphorylation or glycosylation, different modifications may give rise to different isolated polypeptides, which can be separately purified.

As used herein, the term “secreted” refers to a polypeptide that is released from a cell via the secretory pathway through the endoplasmic reticulum, Golgi apparatus, and as a vesicle that transiently fuses at the cell plasma membrane, releasing the polypeptides outside of the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, bispecific antibodies; chimeric antibodies; Fab, Fab′, single chain V region fragments (scFv), fusion polypeptides, and unconventional antibodies. In certain embodiments, an antibody is a glycopolypeptide comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FRI, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, the term “single-chain variable fragment” or “scFv” is a fusion polypeptide of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}.

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

In certain non-limiting embodiments, an intracellular signaling domain of a CAR or a comprises a CD3ζ polypeptide, which can activate or stimulate a cell (e.g., a cell of the lymphoid lineage, e.g., a T cell). CD3ζ comprises 3 immunoreceptor tyrosine-based activation motifs (ITAMs), and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain non-limiting embodiments, a CAR can also comprise a spacer/hinge region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and fragments of CD3, a fragment of a CD28 polypeptide, a fragment of a CD8 polypeptide, a variant thereof, or a synthetic spacer sequence.

As used herein, “costimulatory molecules” refer to cell surface molecules other than antigen receptors or their ligands that are required for a response of lymphocytes to antigen. The at least one co-stimulatory signaling region can include a CD28 polypeptide (e.g., intracellular domain of CD28 or a fragment thereof), a 4-1BB polypeptide (e.g., intracellular domain of 4-1BB or a fragment thereof), an OX40 polypeptide (e.g., intracellular domain of OX40 or a fragment thereof), an ICOS polypeptide (e.g., intracellular domain of ICOS or a fragment thereof), a DAP-10 polypeptide (e.g., intracellular domain of DAP10 or a fragment thereof), or a combination thereof. The co-stimulatory molecule can bind to a co-stimulatory ligand. As used herein, the term a “co-stimulatory ligand” refers to a polypeptide expressed on cell surface that upon binding to its receptor produces a co-stimulatory response, i.e., an intracellular response that effects the stimulation provided by an activating signaling domain (e.g., a CD3ζ signaling domain). Non-limiting examples of co-stimulatory ligands include tumor necrosis factor (TNF) family members, immunoglobulin (Ig) superfamily members, or combination thereof, the co-stimulatory ligand is selected from the group consisting of tumor necrosis factor (TNF) family members, immunoglobulin (Ig) superfamily members, and combinations thereof. Non-limiting examples of TNF family member include 4-1BBL, OX40L, CD70, GITRL, CD40L, and CD30L. Non-limiting examples of Ig superfamily member include CD80, CD86, and ICOSLG. For example, 4-1BBL may bind to 4-1BB for providing an intracellular signal that in combination with a CAR signal induces an effector cell function of the CAR⁺ T cell. CARs comprising an intracellular signaling domain that comprises a co-stimulatory signaling region comprising a 4-1BB, ICOS or DAP-10 co-stimulatory signaling domain are disclosed in U.S. Pat. No. 7,446,190, which is herein incorporated by reference in its entirety.

As used herein, the term “multimerization” refers to the formation of multimers (including dimers). Multimerization includes dimerization.

As used herein, the term “a conservative sequence modification” refers to an amino acid modification that does not significantly affect or alter the binding characteristics of the presently disclosed polypeptide (e.g., the extracellular antigen-binding domain of the polypeptide) comprising the amino acid sequence. Conservative modifications can include amino acid substitutions, additions and deletions. Modifications can be introduced into the human scFv of the presently disclosed polypeptide by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal polypeptide processing (e.g., removal of amino acids from the nascent polypeptide that are not required for biological activity or removal of amino acids by alternative mRNA splicing or alternative polypeptide processing events).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST polypeptide searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

### 6.2. Expression & Sorting Systems Utilizing Intein Trans-Splicing Reactions

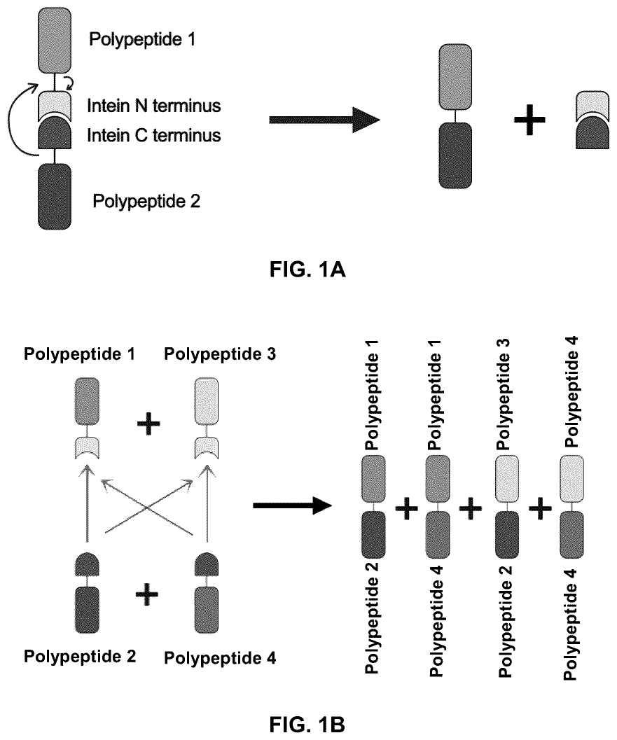

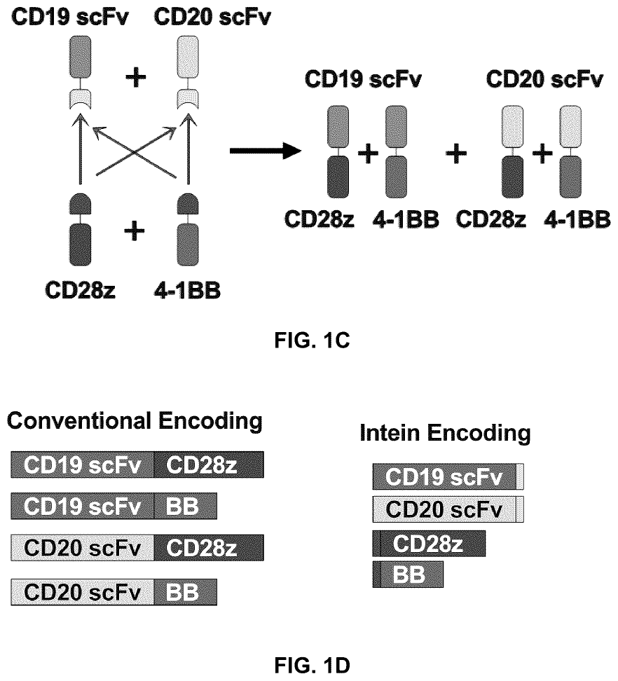

In a first aspect, the present disclosure is directed to expression and sorting systems utilizing intein trans-splicing reactions in connection with the production of modular polypeptides. FIG. 1 depicts a split intein mediated trans-splicing reaction. In this exemplary reaction, a first polypeptide (extein) comprises a split intein fused at the N terminus of the intein and a second polypeptide (extein) comprises the complementary split intein fused at the C terminus of the intein. Upon association of the split inteins, trans-splicing of first and second polypeptides is catalyzed, resulting in covalent linkage of the first and second polypeptides (exteins) via a peptide bond and the release of the non-covalently associated inteins. FIG. 1D illustrates a comparison between encoding necessary for transducing exogenous genes through conventional versus intein-based gene expression using CD19 and CD20 CAR as an exemplary polypeptide. These constructs can include additional genes encoding for domains and/or motifs, for example, but not limited to spacer, linker, hinge, transmembrane, ER retention and kinases. The degeneracy of intein based trans-splicing reactions provides a distinct advantage by enabling expression of large constructs with higher efficiency than using conventional approaches and enables encoding of a larger number of receptors using the same amount of DNA.

Orthogonality and degeneracy may be combined to enable trans-splicing and combination of a large number of receptors with shared binding and signaling domains (degeneracy) or to designate specific binding domains splice with specific signaling domain (orthogonality). Drug-regulation domains can be combined with different signaling domains to enable drug-regulation of degenerate components (e.g., Zap70 and 4-1BB).

In certain embodiments, and not by way of any limitation, the N-terminus and C-terminus complementary split inteins include, a Cfa intein, a gp41-1 intein, a gp41-8 intein, an Aes intein, and a Nrdj-1 intein. Table 1 lists exemplary intein amino acid sequences. Amino acid motifs involved in splicing (e.g., CLS, CFN, CLD, HNS, SVV, SVYLN, CLV), are underlined.

TABLE 1
Exemplary Intein sequences

| | | SEQ |
|---|---|---|
| Name | Sequence | ID NO. |
| Cfa intein N- | AEYCLSYDTEILTVEYGFLPIG | 1 |
| terminus | KIVEERIECTVYTVDKNGFVYT | |
| | QPIAQWHNRGEQEVFEYCLEDG | |
| | SIIRATKDHKFMTTDGQMLPID | |
| | EIFERGLDLKQVDGLP | |
| Cfa intein C- | MVKIISRKSLGTQNVYDIGVEK | 2 |
| terminus | DHNFLLKNGLVASNCFN | |
| gp41-1 intein | TRSGYCLDLKTQVQTPQGMKEI | 3 |
| N-terminus | SNIQVGDLVLSNTGYNEVLNVF | |
| | PKSKKKSYKITLEDGKEIICSE | |
| | EHLFPTQTGEMNISGGLKEGMC | |
| | LYVKE | |
| gp41-1 intein | MMLKKILKIEELDERELIDIEV | 4 |
| C-terminus | SGNHLFYANDILTHNSSSDV | |
| gp41-8 intein | SQLNRCLSLDTMVVTNGKAIEI | 5 |
| N-terminus | RDVKVGDWLESECGPVQVTEVL | |
| | PIIKQPVFEIVLKSGKKIRVSA | |
| | NHKFPTKDGLKTINSGLKVGDF | |
| | LRSRAK | |
| gp41-8 intein | MCEIFENEIDWDEIASIEYVGV | 6 |
| C-terminus | EETIDINVTNDRLFFANGILTH | |
| | NSSAVEE | |
| Aes intein N- | YIDTDSVVGDTIIDVSGKKMTI | 7 |
| terminus | AEFYDSTPDG | |
| Aes intein C- | EARDWVKRVGGKTSLSVNTYSG | 8 |
| terminus | EVERKNINYIMKHTVKKRMFKI | |
| | KAGGKEVIVTADHSVMVKRDGK | |
| | IIDVKPTEMKQTDRVVKWMLTG | |
| | SHMIEFIEFEIEDLGVMEIDVY | |
| | DIEVDGNHNFFGNDILVHNSVY | |
| | LN | |
| Nrdj-1 N- | GTNPCCLVGSSEIITRNYGKTT | 9 |
| terminus | IKEVVEIFDNDKNIQVLAFNTH | |
| | TDNIEWAPIKAAQLTRPNAELV | |
| | ELEIDTLHGVKTIRCTPDHPVY | |
| | TKNRGYVRADELTDDDELVVAI | |
| Nrdj-1 C- | MEAKTYIGKLKSRKIVSNEDTY | 10 |
| terminus | DIQTSTHNFFANDILVHNSSEI | |
| | VL | |

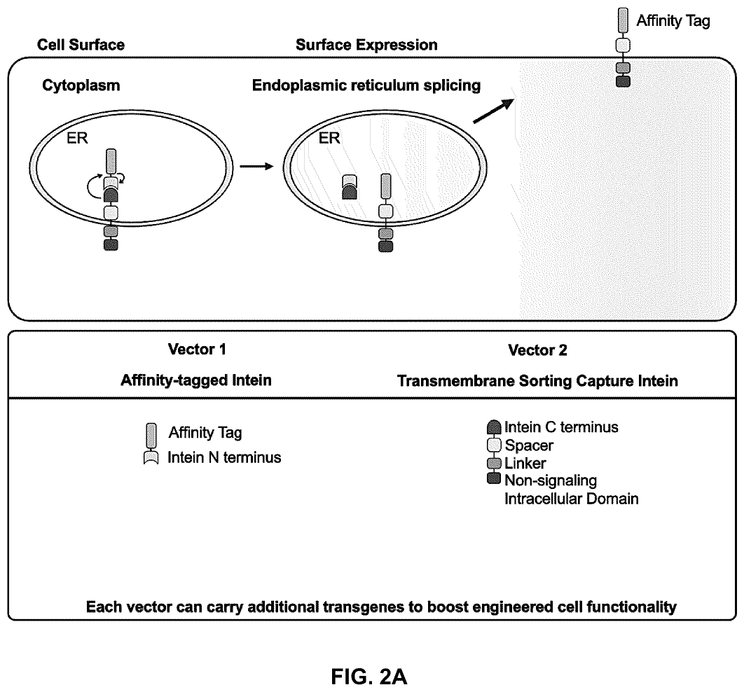

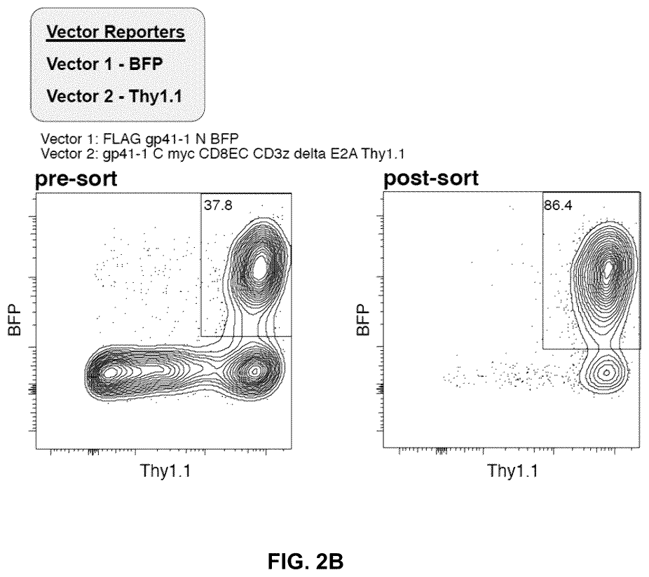

In certain embodiments, the present disclosure is directed to a two-vector intein sorting system. For example, FIG. 2A depicts an exemplary two-vector intein sorting system. In this exemplary approach, Vector 1 encodes a secreted affinity-tagged molecule fused to an intein N terminal domain. Vector 2 encodes a C terminus intein capture domain fused to a spacer, transmembrane domain, linker, and non-signaling cytoplasmic domain. Trans-splicing of the two intein-tagged polypeptides results in cell selective surface display of the affinity tag on dual-vector transduced cells resulting from selective intracellular splicing. In certain embodiments, this approach enables selective affinity-tag based MACS sorting of cells incorporating both vectors and completing the trans-splicing reaction. In certain embodiments, each vector can carry additional transgenes to boost engineered cell functionality.

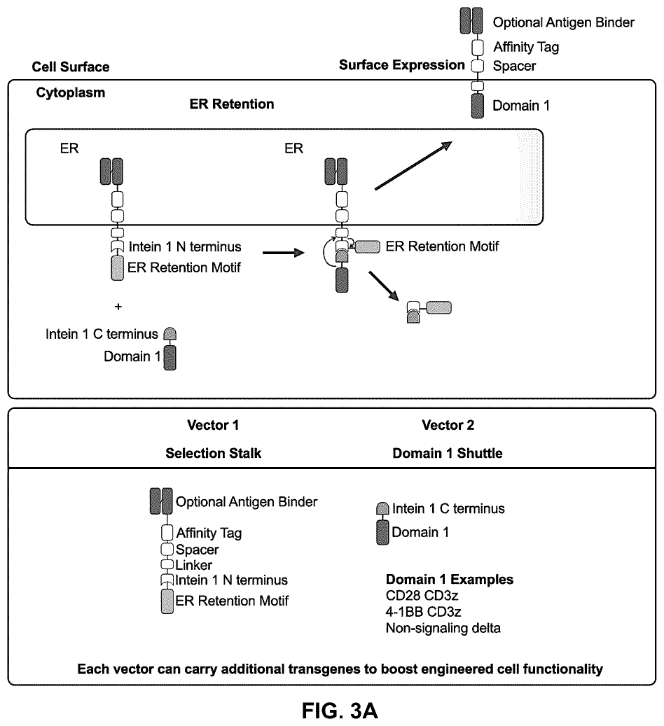

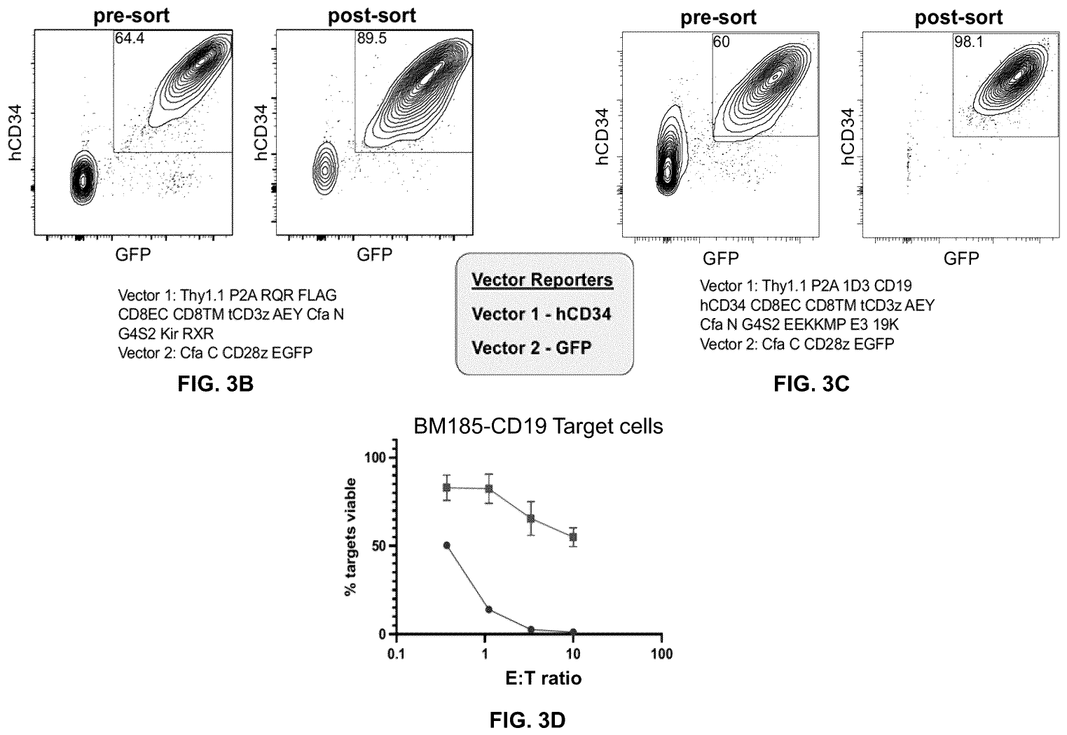

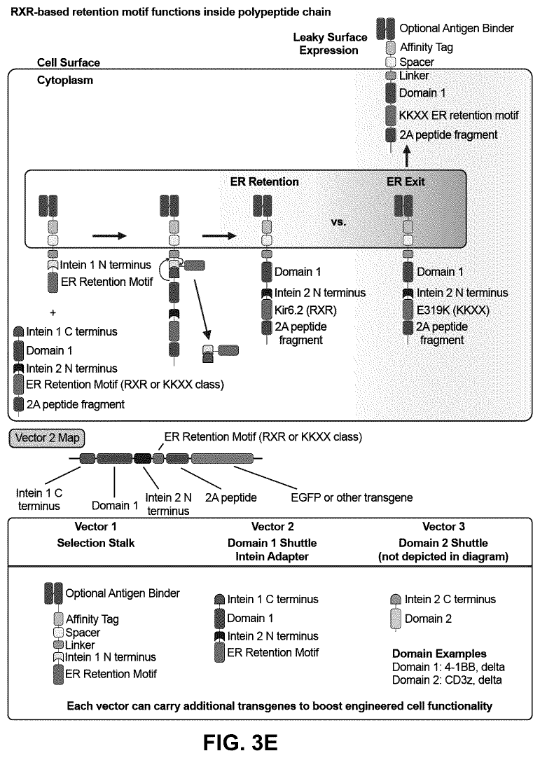

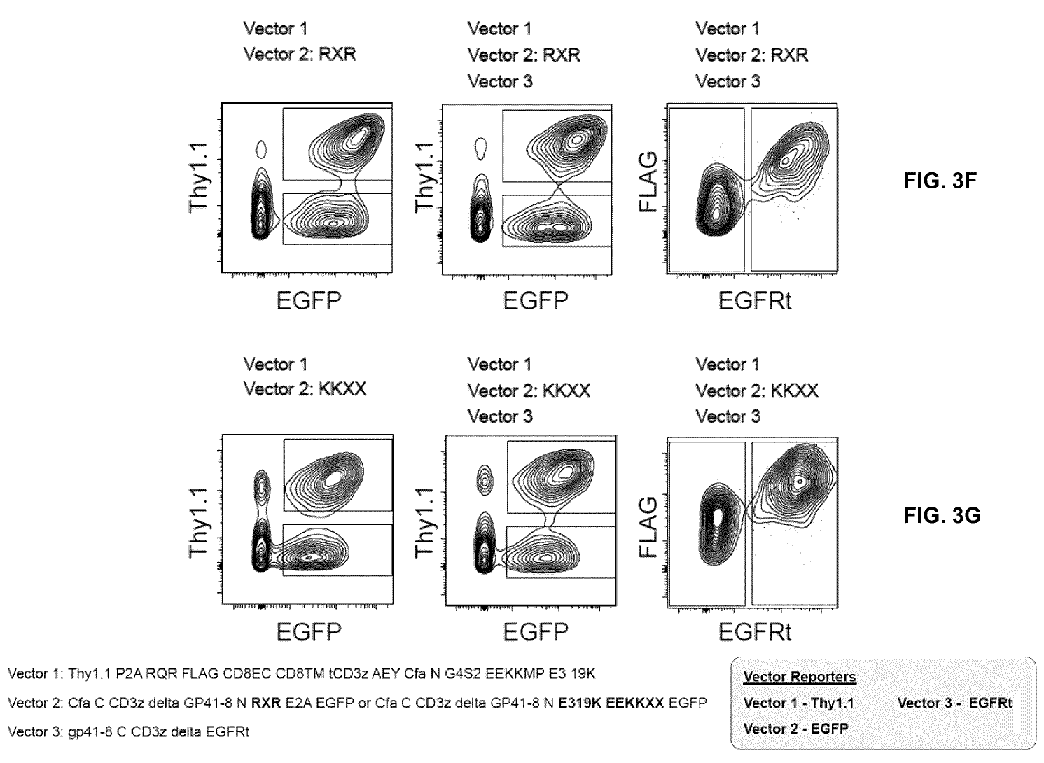

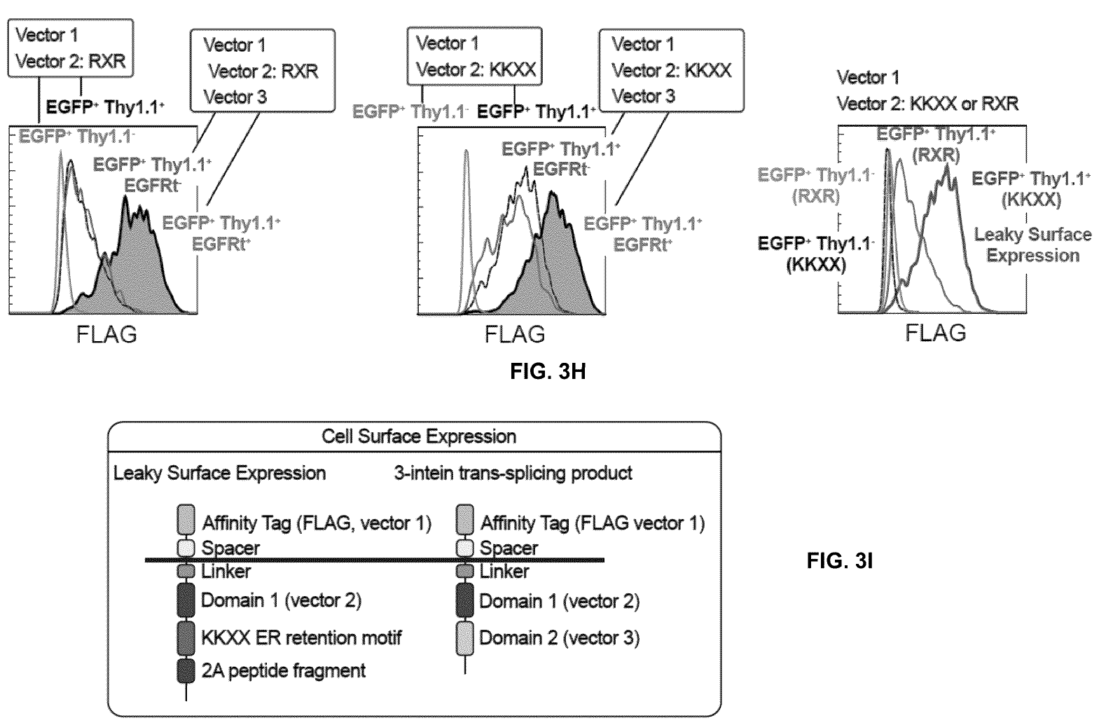

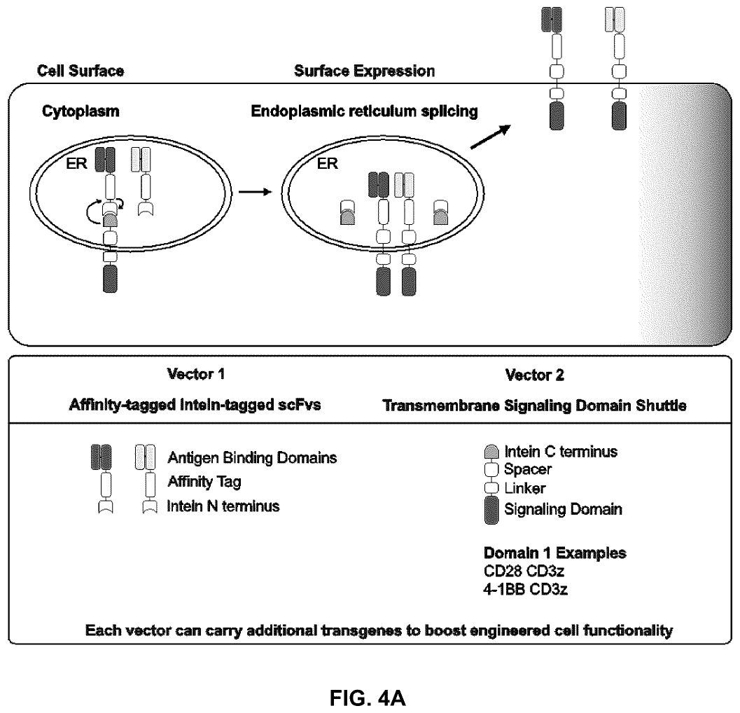

In certain embodiments, the present disclosure is directed to two-vector intein endoplasmic reticulum retention-based sorting and post-translational CAR assembly. For example, FIG. 3A depicts an exemplary approach for two-vector intein endoplasmic reticulum retention-based sorting and post-translational CAR assembly. In this exemplary approach, Vector 1 encodes an intein-tagged antigen binding construct comprised of an scFv fused to an affinity tag, a spacer, a transmembrane domain, an intein N terminus, and an endoplasmic reticulum (ER) retention motif, and Vector 2 encodes a cytoplasmic signaling domain shuttle construct comprised of an intein C terminus fused to a costimulatory domain, and a T cell receptor signaling domain (e.g., CD3 zeta). Trans-splicing of the two the intein-tagged polypeptides results in an intracellular splice junction and a recapitulates a fully functional chimeric antigen receptor (CAR). Surface expression of the affinity-tagged antigen-binding domain is markedly reduced due to ER retention until the two inteins trans-splice, which removes the ER retention motif, resulting in cell surface trafficking. In certain embodiments, this approach enables selective affinity-tag based MACS sorting of cells incorporating both vectors and completing the trans-splicing reaction. In certain embodiments, each vector can carry additional transgenes to boost engineered cell functionality.

In certain embodiments, any ER retention motif can be employed in the context of the compositions, systems, and methods of the present disclosure. In certain embodiments, e.g., where the ER retention motif is occupies the end of the polypeptide chain, either an E3 19K KKXX-type motif or a RXR-type ER retention motif can be employed (FIG. 3E). In certain embodiments, e.g., when the ER retention motif occupies a position within a polypeptide chain, an RXR-type ER retention motifs is employed. For example, but not by way of limitation, FIGS. 3E-3H demonstrate the ability of RXR-type ER retention motifs to function inside of a polypeptide chain (in this case a 2A peptide remnant remaining attached to the RXR retention motif). In contrast, the E3 19K KKXX-type motif fails in this setting due to the requirement of this retention motif to occupy the end of the polypeptide chain. As illustrated in FIGS. 3F-3I, cells transduced with Vector 1 and Vector 2 encoding either RXR or KKXX motifs, co-transduction with Vector 3 encoding a gp41-1 C intein removes the ER retention motif and promotes strong surface trafficking of the fully spliced sorting stalk molecule. However, the RXR-type ER retention motif version enables high expression of this construct only in triple-transduced cells, enabling selective MACS sorting of triple-transduced cells. This methodology enables single-step MACS purification of cells incorporating two or more vectors with enhanced overall vector payload to generate effector cells with enhanced functionality. Furthermore, use of RXR-type ER retention motifs enables placement of ER-retained constructs in 5′ or 3′ locations in 2A peptide flanked polycistronic vectors containing multiple encoded proteins

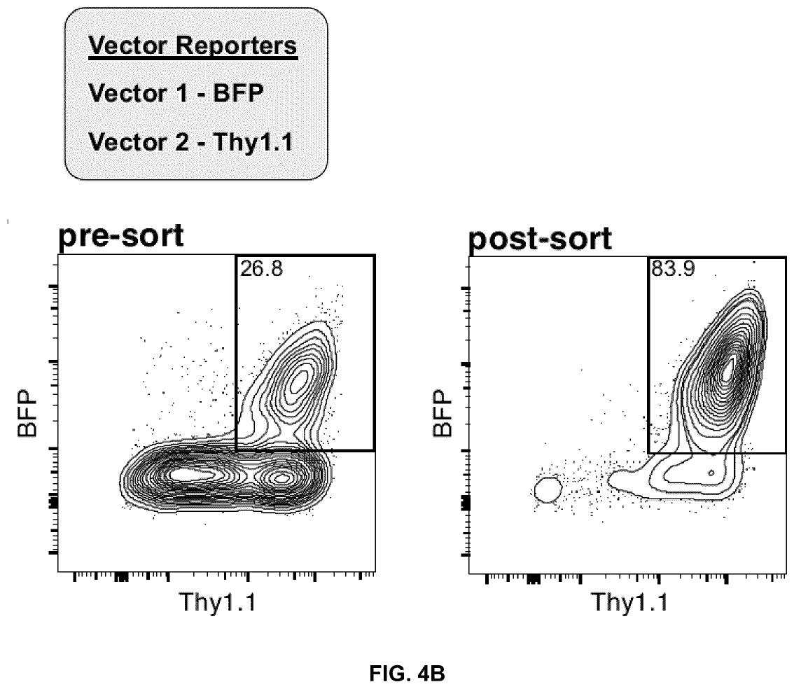

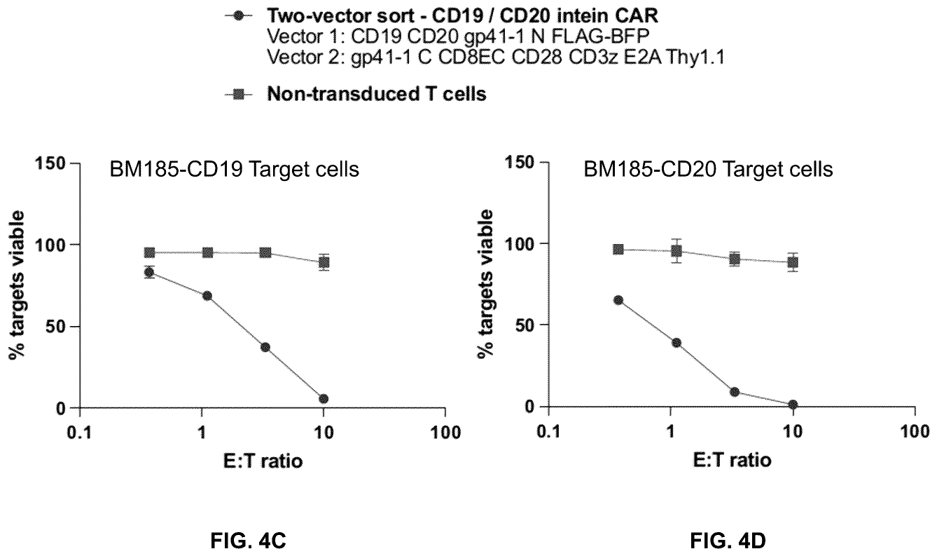

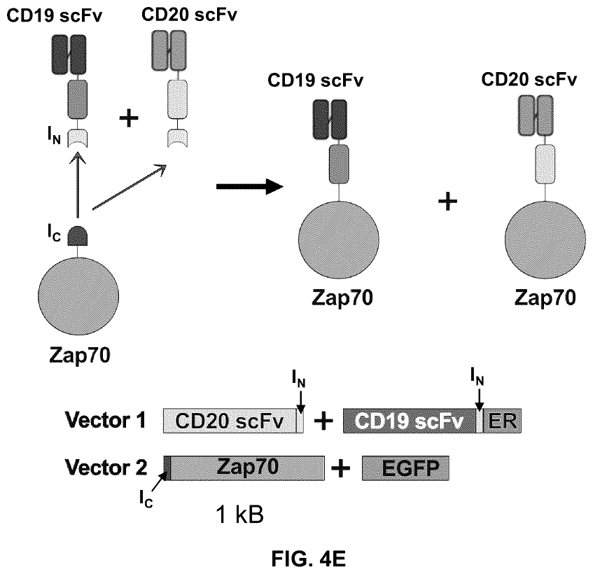

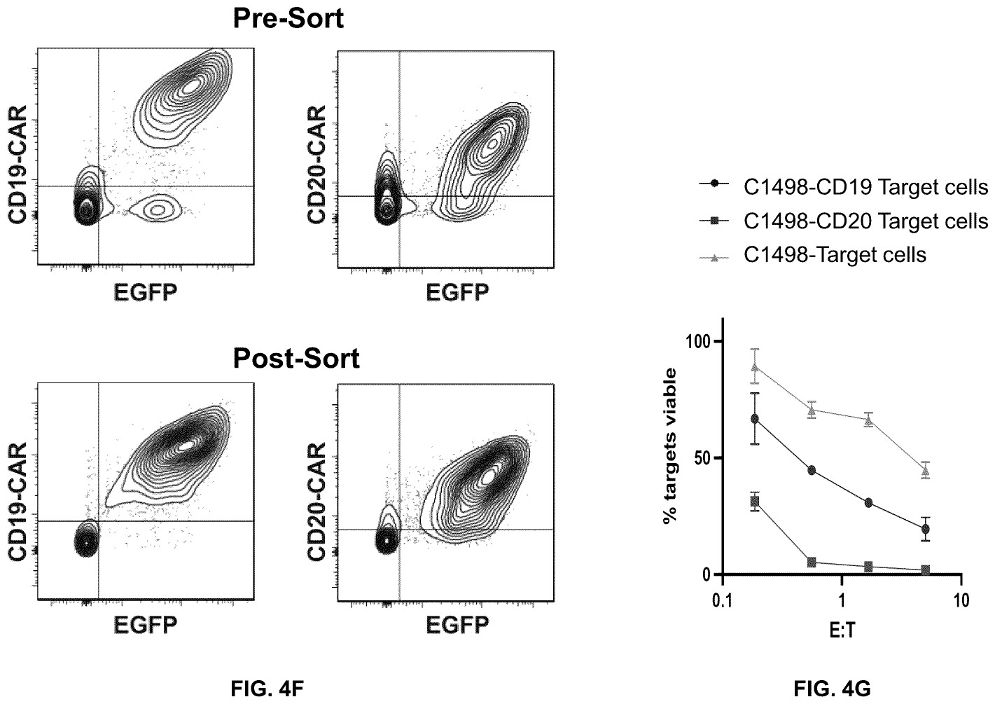

In certain embodiments, the present disclosure is directed to a two-vector dual secreted intein-tagged scFv sorting system and post-translational CAR assembly. For example, FIG. 4A depicts an exemplary approach for two-vector dual secreted intein-tagged scFv sorting and post-translational CAR assembly. In this exemplary approach, Vector 1 encodes dual affinity-tagged intein-tagged scFvs each fused to an affinity tag and degenerate intein N terminus and Vector 2 encodes a transmembrane signaling domain shuttle comprised of an intein C terminus fused to a spacer domain, transmembrane domain, a costimulatory domain, and a T cell receptor signaling domain. Following capture and splicing of the intein domains, both antigen binding-domains are displayed on the cell surface, recapitulating full-length CAR molecules. Trans-splicing of the two intein-tagged polypeptides results in cell selective surface display of the affinity tag in dual-vector transduced cells due to selective intracellular splicing. In certain embodiments, this enables selective affinity-tag based MACS sorting of cells incorporating both vectors and completing the trans-splicing reaction. In certain embodiments, each vector can carry additional transgenes to boost engineered cell functionality.

In certain embodiments, the present disclosure is directed to zeta-chain-associated protein kinase 70 (Zap70) based intein CAR expression. For example, FIG. 4E depicts an exemplary approach for two-vector degenerate intein trans-splicing to enable expression of both, a Zap70 based CD19 scFv and a Zap70 based CD20 scFv. In this exemplary approach, Vector 1 encodes two distinct antigen-binding scFv domain containing fusion polypeptides with degenerate cytoplasmic N terminus intein domains. Each scFv fusion polypeptide contains a distinct affinity tag and may additionally comprise an ER retention motif at the end of a vector construct at the C terminus of fusion polypeptide (see FIG. 3A). Vector 2 encodes a Zap70 domain shuttle comprised of a degenerate intein C terminus, and protein tag (e.g., EGFP tag). Trans-splicing of combinations of two degenerate intein-tagged antigen-binding polypeptides with the Zap70 domain shuttle enables generation of two CAR molecules totaling two mature trans-sliced polypeptides. Presence of an ER retention motif minimizes surface expression of the affinity-tagged antigen-binding domain polypeptide until the two inteins trans-splice, which removes the retention motif, resulting in cell surface trafficking. This system enables post-translational dual-CAR assembly combined with selective affinity-tag-based MACS sorting of cells incorporating both vectors and completing the trans-splicing reaction. Each vector can carry additional transgenes to boost engineered cell functionality.

In certain embodiments, the compositions, systems, and methods of the present disclosure can be facilitated by the degenerate splicing of inteins. For example, but not by way of limitation, degenerate splicing of inteins sharing a common cytoplasmic signaling domain shuttle enables reuse of DNA encoding large structural elements (e.g., Zap70 interdomain and kinase). In certain embodiments, such use can serve as a data compression algorithm as it enables a greater number of receptors to be generated per the amount of DNA delivered by the vector. For example, but not by way of limitation, the use of degenerate inteins tagged to domains used in common for multiple CAR constructs (e.g., Zap70 signaling domain and 4-1BB costimulatory domain) can be multiplexed with multiple antigen binding domain-containing molecules to generate a greater number of trans-spliced CAR and CCR molecules than could be encoded with the same amount of DNA. DNA “data” savings increases with the size of the encoded domains and as the domains are reused in a greater number of molecules. In certain embodiments, the quantity of DNA, Q_{C}, required to encode a series of conventionally encoded molecules, e.g., CAR and CCR molecules, with common binding domains of size Bi and signaling domains of size Dⱼ can be described by Equation 1;

$${Q}_{C} = d \sum {B}_{i} + b \sum {D}_{j} \qquad Equation 1$$

where d is the number of distinct signaling domains with size Dⱼ used and b is the number of distinct binding domains with size Bi used. Whereas the quantity of DNA, Q_{I}, required to encode a series of intein-encoded CAR and CCR molecules can be described by Equation 2;

$${Q}_{I} = \sum {B}_{i} + \sum {D}_{j} + {bI}_{N} + {dI}_{C} \qquad Equation 2$$

where I_{N} is the size of DNA required to encode the N terminal split intein and I_{C} is the size of DNA required to encode the C terminal split intein. The DNA savings (Δ=Q_{c}−Q_{I}) resulting from intein-based compression encoding can be determined by Equation 3

$$Δ = ( d - 1 ) \sum {B}_{i} + ( b - 1 ) \sum {D}_{j} - {bI}_{N} - {dI}_{C} \qquad Equation 3$$

where d is the number of signaling domains encoded, b is the number of binding stalk molecules encoded, Bᵢ is the size of each binding domain, Dⱼ is the size of each signaling domain, I_{N} is the size of the N terminal split intein, and I_{C} is the size of the C terminal intein. This is illustrated by one example of conventional encoding of two CAR and two CCR molecules utilizing an NS3 drug-regulated Zap70 signaling domain and a 4-1BB costimulatory domain with domain sizes; B1=1 kB, B2=1 kB, D1=2.2 kB, D2=0.1 kB. This would require per Equation 1:

$$Equation 4$$

$$2 * ( B 1 + B 2 ) + 2 * ( D 1 + D 2 ) = 2 * ( 1 + 1 ) + 2 * ( 2.2 + 0.1 ) = 8.6 kB$$

Rearranging by CAR and CCR molecules:

$$Equation 5$$

$${( B 1 + D 1 )}_{CAR 1} + {( B 2 + D 1 )}_{CAR 2} + {( B 1 + D 2 )}_{CCR 1} + {( B 2 + D 2 )}_{CCR 1} = 3.2 + 3.2 + 1.1 + 1.1 = 8.6 kB$$

In contrast, intein encoding where I_{N}=0.3 kB and I_{C}=0.1, would require the following DNA to sum encode:

$$Equation 5$$

$$( B 1 + B 2 ) + ( D 1 + D 2 ) + 2 ( {I}_{N} ) + 2 ( {I}_{C} ) = 2 + 2.3 + 0.6 + 0.2 = 5.1 kB$$

which yields a 8.6 kB−5.1 kB=3.5 KB savings of DNA. Confirming with Equation 3, data savings is:

$$1 * 2 + 1 * 2.3 - 0.6 - 0.2 = 3.5 kB \qquad Equation 6$$

This data savings increases the number of antigen binding domains B and signaling domains D, or the size of each domain increases. For example, but not by way of limitation, use of three signaling domains with sizes (2 kB, 0.3 kB, and 0.1 kB) and three antigen binding domains with size 1 kB, where I_{N}=0.3 kB and I_{C}=0.1 kB, would encode nine receptors and would yield the following DNA savings in intein encoding compared with conventional encoding:

$$Equation 7$$

$$2 * ( 1 + 1 + 1 ) + 2 * ( 2 + 0.3 + 0.1 ) - 3 * 0.3 - 3 * 0.1 = 9.6 kB .$$

In certain embodiments, the selective sequestration of the encoded protein, e.g., a FLAG-tagged CAR, by the ER retention motif and subsequent surface trafficking following trans-splicing with the appropriate cytoplasmic signaling domain shuttle and removal of the ER retention motif enables selective MACS sorting of dual-transduced cells. In certain embodiments, such an approach can act as a molecular coincidence detector.

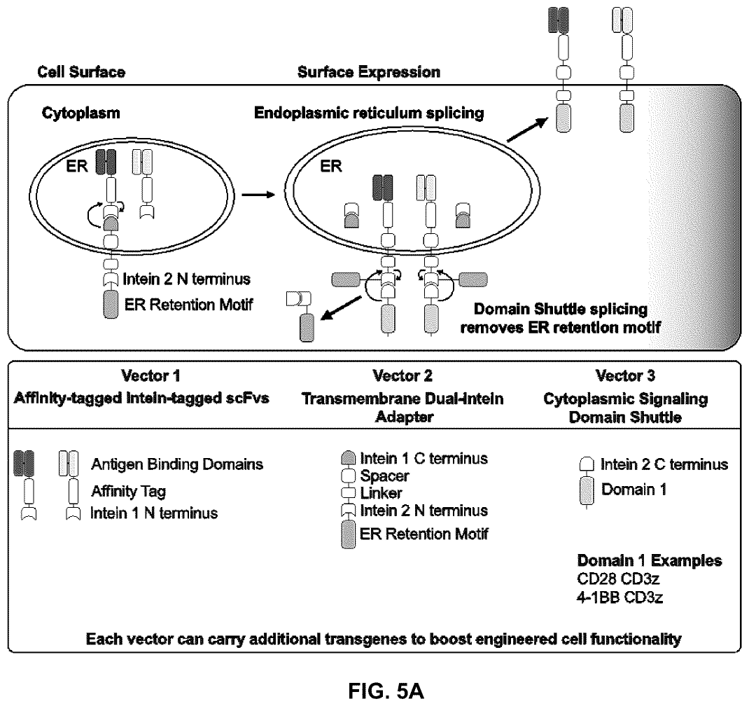

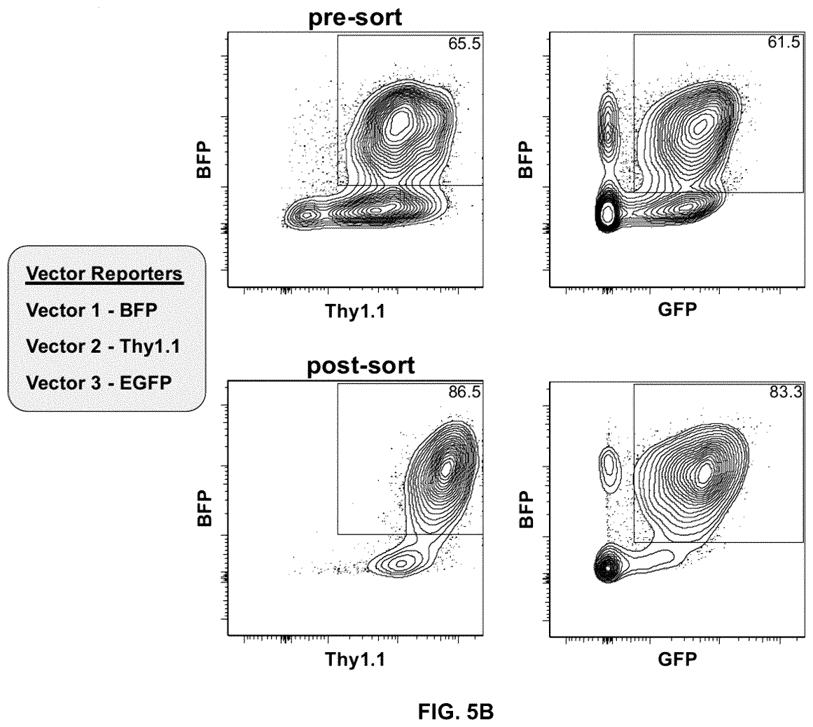

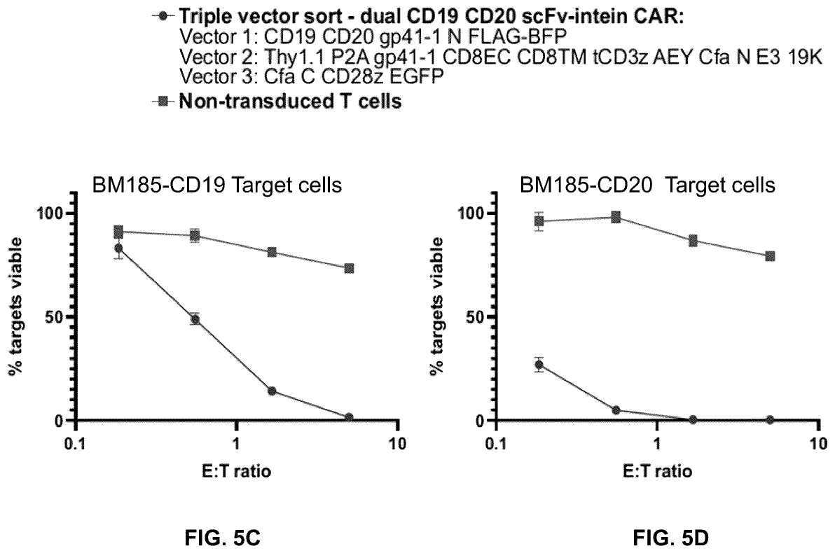

In certain embodiments, the present disclosure is directed to a three-vector intein ER retention-based sorting system and post-translational CAR assembly with secreted intein-tagged scFvs. For example, FIG. 5A depicts an exemplary approach for three-vector intein ER retention-based sorting and post-translational CAR assembly with secreted intein-tagged scFvs. In this exemplary approach, Vector 1 encodes dual antigen-binding scFvs each fused to an affinity tag and an orthogonal intein N terminus and Vector 2 encodes a dual-intein transmembrane adapter comprised of an orthogonal intein C terminus fused to extracellular and transmembrane domains, an orthogonal intein N terminus, and an ER retention motif, and Vector 3 encodes a cytoplasmic signaling domain shuttle comprised of an orthogonal intein C terminus fused to a costimulatory domain and T cell receptor signaling domain. The intein-tagged scFv molecules trans-splice with the dual-intein transmembrane adapter, which can exit the ER following trans-splicing of the orthogonal inteins of the adapter and cytoplasmic shuttle constructs, which recapitulates a full-length CAR, with cleavage of the ER retention motif. In certain embodiments, this enables selective affinity-tag based MACS sorting of cells incorporating three vectors following completion of two orthogonal trans-splicing reactions. In certain embodiments, each vector can carry additional transgenes to boost engineered cell functionality.

In certain embodiments, the compositions, systems, and methods of the present disclosure can be facilitated using orthogonal two-intein, tri-molecular trans-splicing methodologies. For example, but not by way of limitation, such methodologies, e.g., as illustrated in FIGS. 5A-5D, enable selective MACS sorting of T cells incorporating three unique vectors, which can further encode additional transgenes to enhance effector cell functionality.

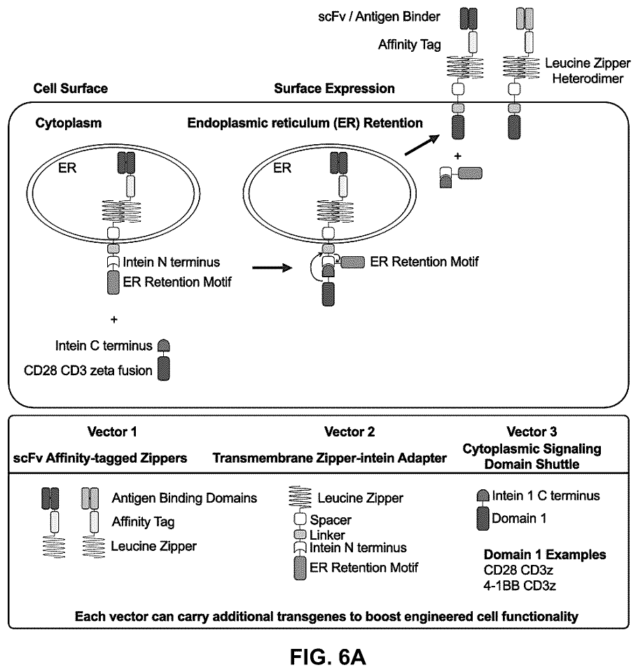

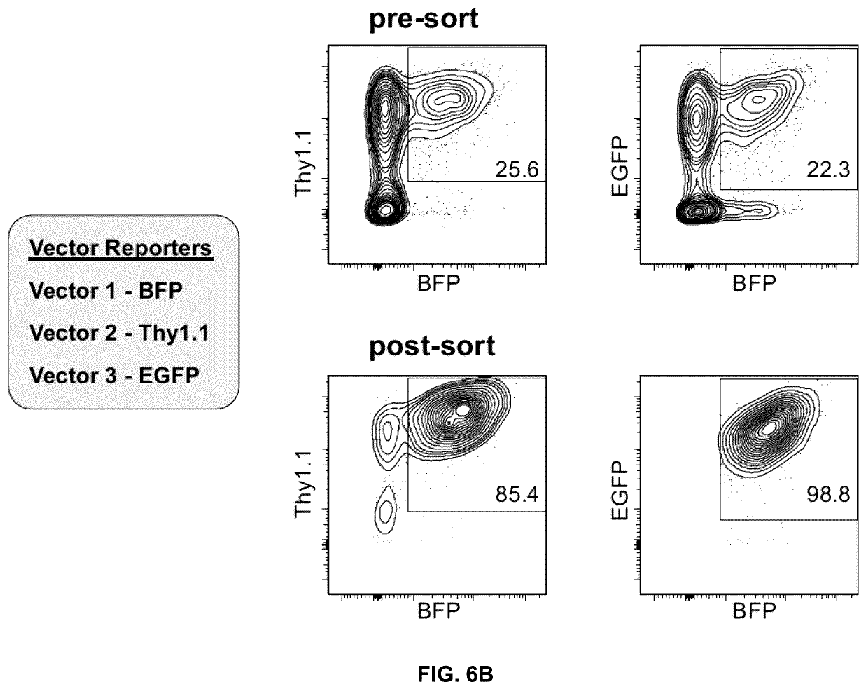

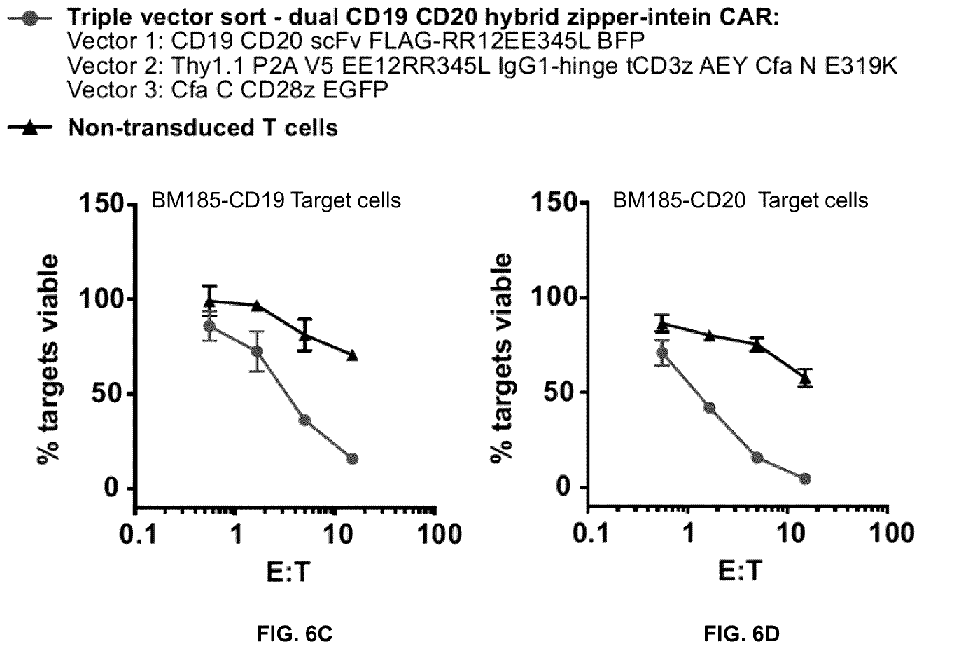

In certain embodiments, the present disclosure is directed to three-vector hybrid leucine zipper-intein ER retention-based sorting system and post-translational CAR assembly. For example, FIG. 6A depicts an exemplary approach for three-vector-hybrid leucine zipper-intein ER retention-based sorting and post-translational CAR assembly. In this exemplary approach, Vector 1 encodes dual antigen-binding scFvs each fused to an affinity tag and a heterodimerizing leucine zipper, Vector 2 encodes a leucine zipper-intein transmembrane adapter comprised of a heterodimerizing leucine zipper fused a short hinge domain, transmembrane domain, an intein N terminus, and an ER retention motif, and Vector 3 encodes a cytoplasmic signaling domain shuttle comprised of an intein C terminus fused to a costimulatory domain and a T cell receptor signaling domain. The leucine zipper-tagged scFv molecules bind to the capture zipper-intein transmembrane adapter in the ER, which can exit the ER following trans-splicing of inteins of the adapter and cytoplasmic shuttle constructs, which recapitulates a full-length CAR, with cleavage of the ER retention motif. In certain embodiments, this enables selective affinity-tag based MACS sorting of cells incorporating three vectors following completion of zipper heterodimerization and intein trans-splicing reactions. In certain embodiments, each vector can carry additional transgenes to boost engineered cell functionality.

In certain embodiments, the compositions, systems, and methods of the present disclosure can be facilitated using orthogonal inteins and leucine zippers to enable selective MACS sorting. For example, but not by way of limitation, such methodologies, e.g., as illustrated in FIGS. 6A-6D, can facilitate the selective MACS sorting of T cells incorporating three unique vectors, which can further encode additional transgenes to enhance effector cell functionality.

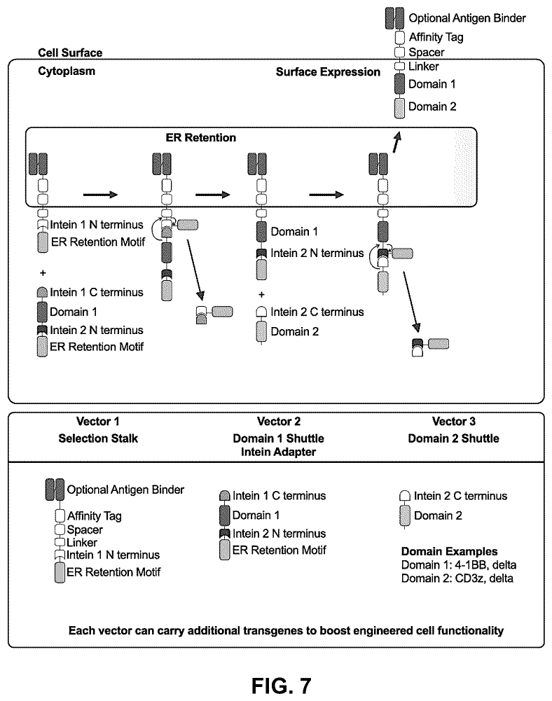

In certain embodiments, the present disclosure is directed to a three-vector intein ER retention-based sorting system and post-translational CAR assembly with intracellular intein adapters. For example, FIG. 7 depicts an exemplary approach for three-vector intein ER retention-based sorting and post-translational CAR assembly with intracellular intein adapters. In this exemplary approach, Vector 1 encodes an intein-tagged antigen binding construct comprised of an scFv fused to an affinity tag, a spacer, a transmembrane domain, an orthogonal intein N terminus, and an ER retention motif, Vector 2 encodes a dual-intein cytoplasmic adapter comprised of an orthogonal intein C terminus fused to an optional signaling domain, an orthogonal intein N terminus, and an ER retention motif, and Vector 3 encodes a cytoplasmic signaling domain shuttle comprised of an orthogonal intein C terminus fused to a costimulatory domain and/or T cell receptor signaling domain. The orthogonal intein-tagged molecules trans-splice as depicted with serial cleavage of both ER retention motifs with recapitulation of a full-length CAR molecule. In certain embodiments, removal of ER retention motifs enables selective trafficking of the CAR molecule to the cell surface and selective affinity-tag based MACS sorting of cells incorporating three vectors following completion of two orthogonal trans-splicing reactions. In certain embodiments, each vector can carry additional transgenes to boost engineered cell functionality.

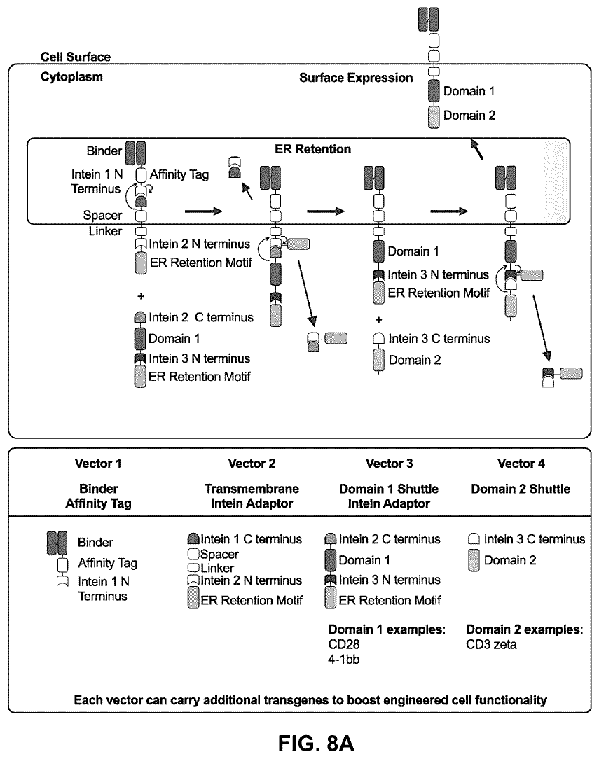

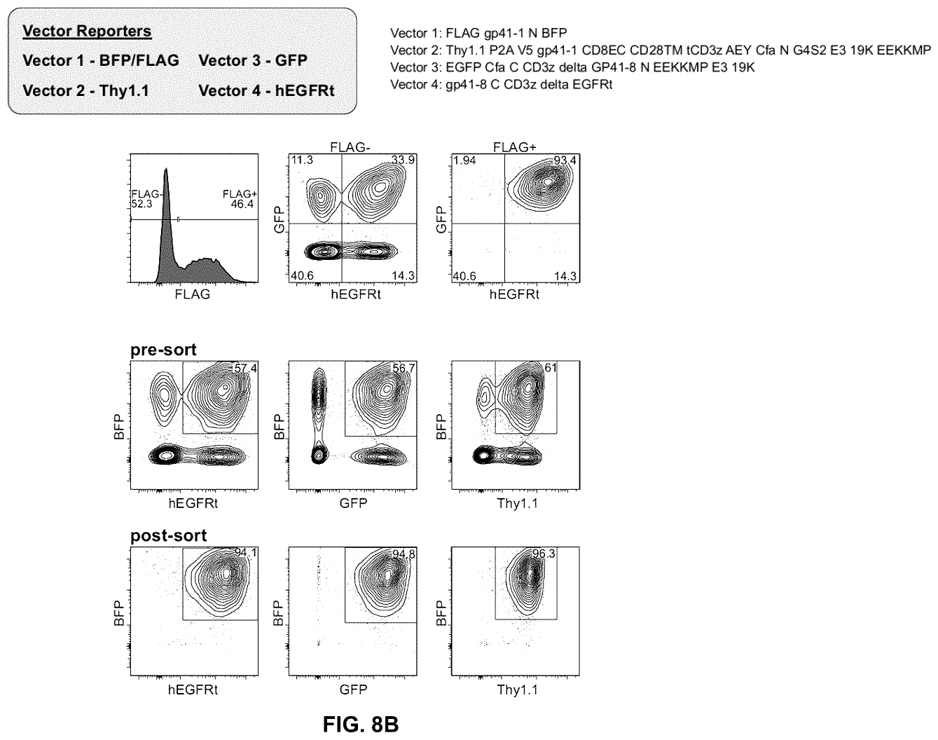

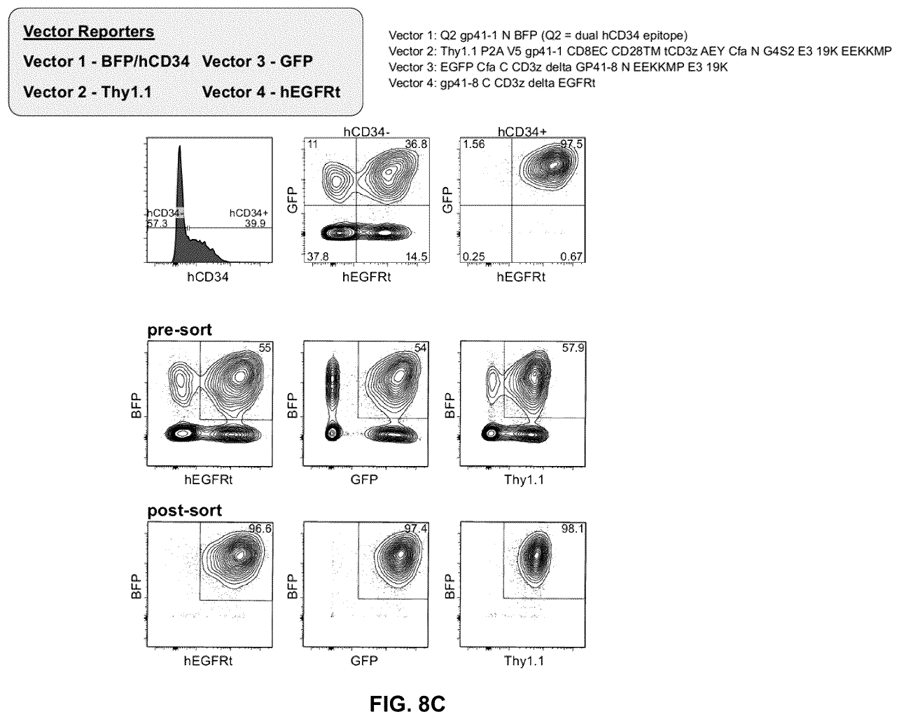

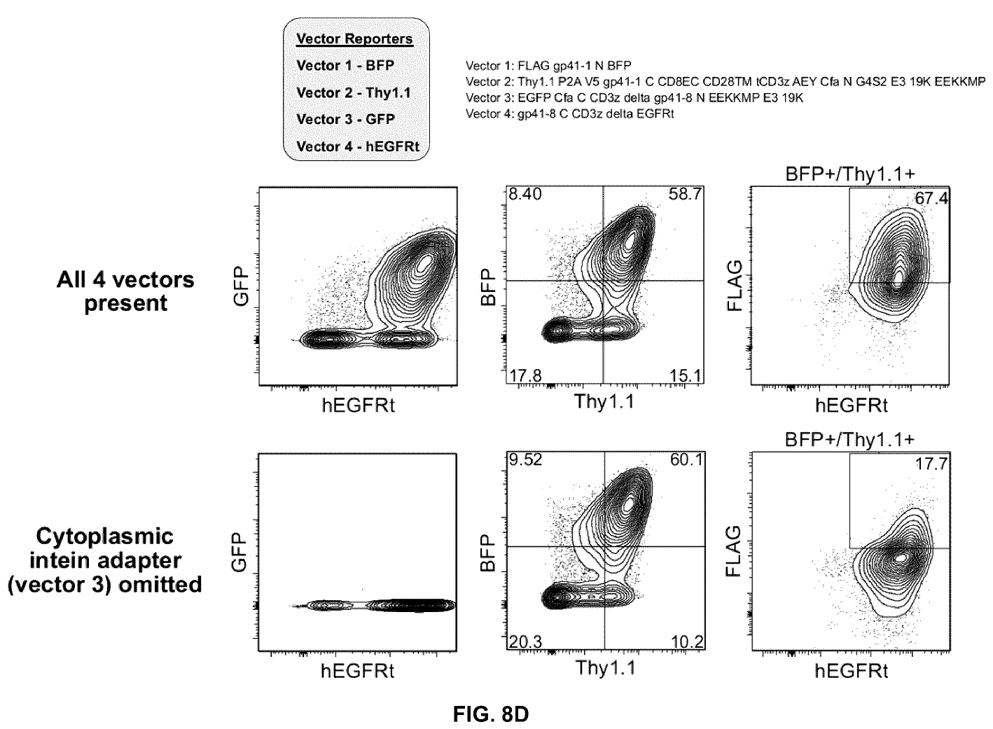

In certain embodiments, the present disclosure is directed to a four-vector intein ER retention-based sorting system and post-translational CAR assembly with combined use of transmembrane and intracellular intein adapters. For example, FIG. 8A depicts an exemplary approach for four-vector intein ER retention-based sorting and post-translational CAR assembly with combined use of transmembrane and intracellular intein adapters. In this exemplary approach, Vector 1 encodes dual antigen-binding scFv fused to an affinity tag and an orthogonal intein N terminus, Vector 2 encodes a dual-intein transmembrane adapter comprised of an orthogonal intein C terminus fused to extracellular and transmembrane domains, an orthogonal intein N terminus, and an ER retention motif, Vector 3 encodes a dual-intein cytoplasmic adapter comprised of an orthogonal intein C terminus fused to an optional signaling domain, an orthogonal intein N terminus, and an ER retention motif, and Vector 4 encodes a cytoplasmic signaling domain shuttle comprised of an orthogonal intein C terminus fused to a costimulatory domain and/or T cell receptor signaling domain. The orthogonal intein-tagged molecules trans-splice as depicted with serial cleavage of both ER retention motifs with recapitulation of a full-length CAR molecule. In certain embodiments, removal of ER retention motifs enables selective trafficking of the CAR molecule to the cell surface and selective affinity-tag-based MACS sorting of cells incorporating four vectors following completion of three orthogonal trans-splicing reactions. In certain embodiments, each vector can carry additional transgenes to boost engineered cell functionality.

In certain embodiments, the compositions, systems, and methods of the present disclosure can be facilitated using orthogonal three-intein splicing methodologies that specifically generate a trans-spliced CAR or sorting stalk molecule in cells incorporating four unique vectors. For example, but not by way of limitation, this recursive strategy of nesting orthogonal inteins tagged with ER retention motifs can be extended to greater than 4 vectors by adding additional orthogonal inteins. Moreover, in certain embodiments, this strategy can increase the number of transgenes delivered to engineered cells due to the increased number of vectors integrated.

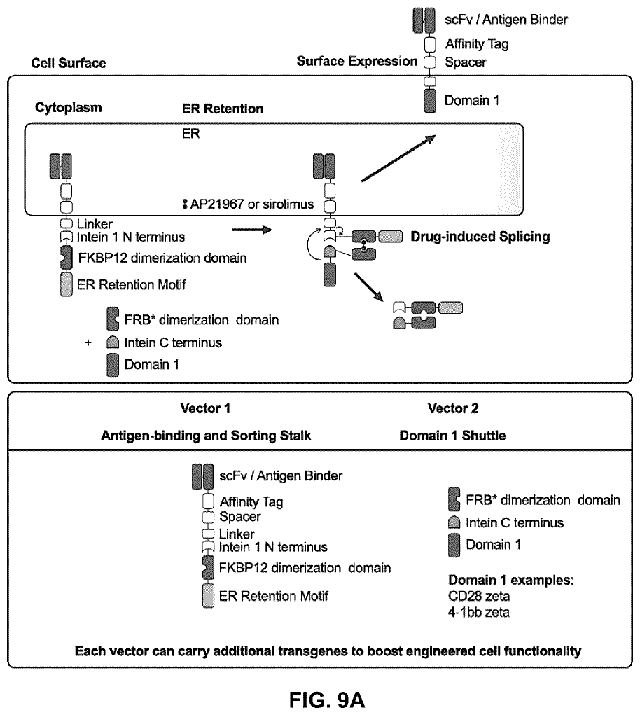

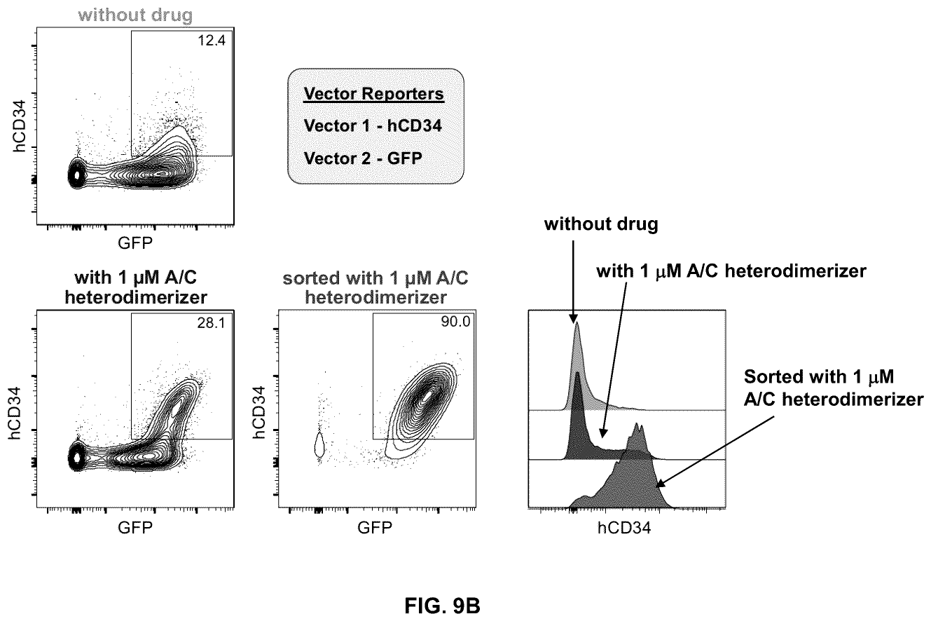

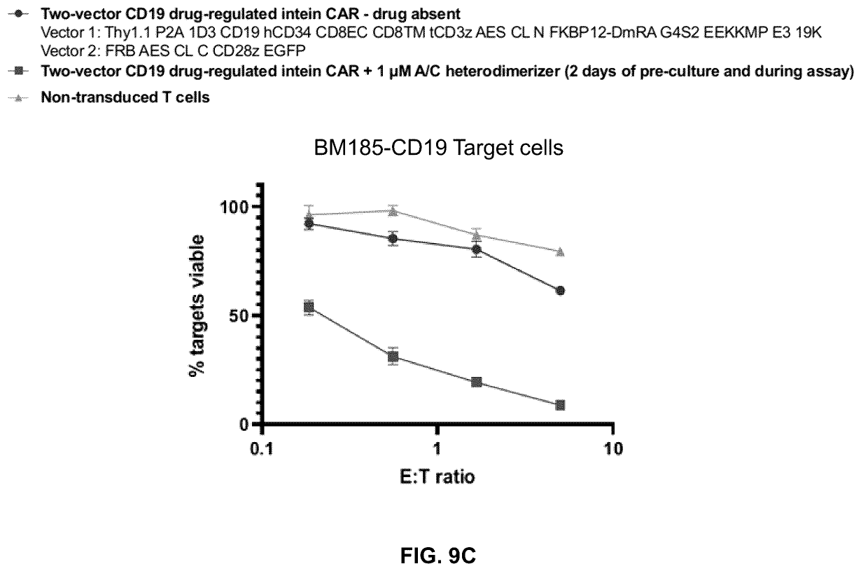

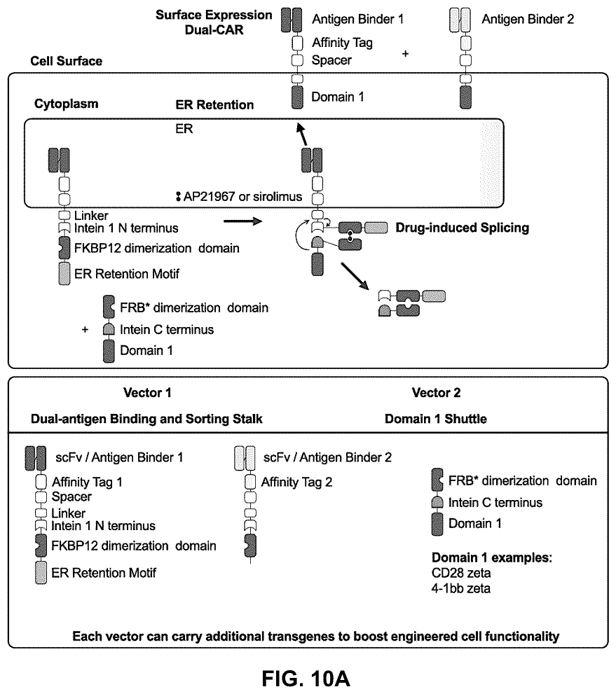

In certain embodiments, the present disclosure is directed to a two-vector intein sorting system employing drug-regulated CAR expression. For example, FIG. 9A depicts an exemplary approach for two-vector intein sorting employing drug-regulated CAR expression. In this exemplary approach, Vector 1 encodes an intein-tagged antigen binding construct comprised of an scFv fused to an affinity tag, a spacer, a transmembrane domain, a low-affinity intein N terminus, a drug-dimerizable domain (e.g., FKBP12), and an ER retention motif, and Vector 2 encodes a drug-dimerizable domain (e.g., FRB*) fused to a low-affinity intein C terminus, a costimulatory domain, and a T cell receptor signaling domain (e.g., CD3 zeta). Trans-splicing of the two the low-affinity intein-tagged polypeptides occurs maximally in the presence of a dimerizer drug due to enhanced association of the polypeptides mediated by the drug-dimerizable domains. Trans-splicing results in an intracellular splice junction and a recapitulates a fully functional CAR. Surface expression of the affinity-tagged antigen-binding domain polypeptide is markedly reduced due to ER retention until the two inteins trans-splice, which removes the ER retention motif, resulting in cell surface trafficking. In certain embodiments, this system enables drug-dependent post-translational CAR assembly and selective affinity-tag-based MACS sorting of cells incorporating both vectors and completing the trans-splicing reaction. In certain embodiments, each vector can carry additional transgenes to boost engineered cell functionality.

In certain embodiments, the compositions, systems, and methods of the present disclosure can be facilitated using a methodology that improves upon previous FKBP/FRB-based drug-regulated CAR systems by eliminating the FKBP/FRB proteins following trans-splicing and generating a mature CAR molecule. In certain embodiments, such an approach would simplify the CAR design. Additionally, in certain embodiments, the signaling domains can be brought into a membrane proximal location where signaling is favored.

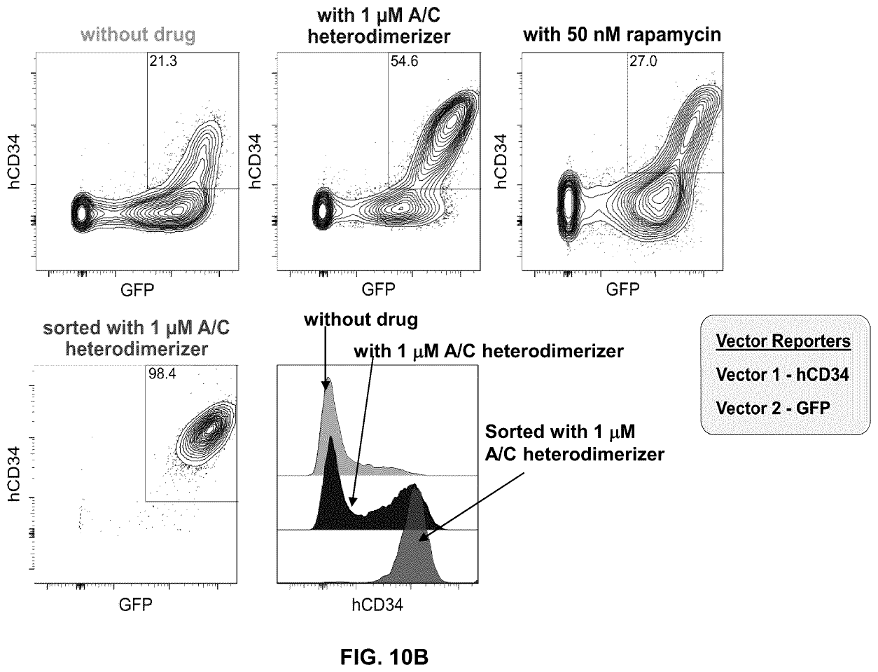

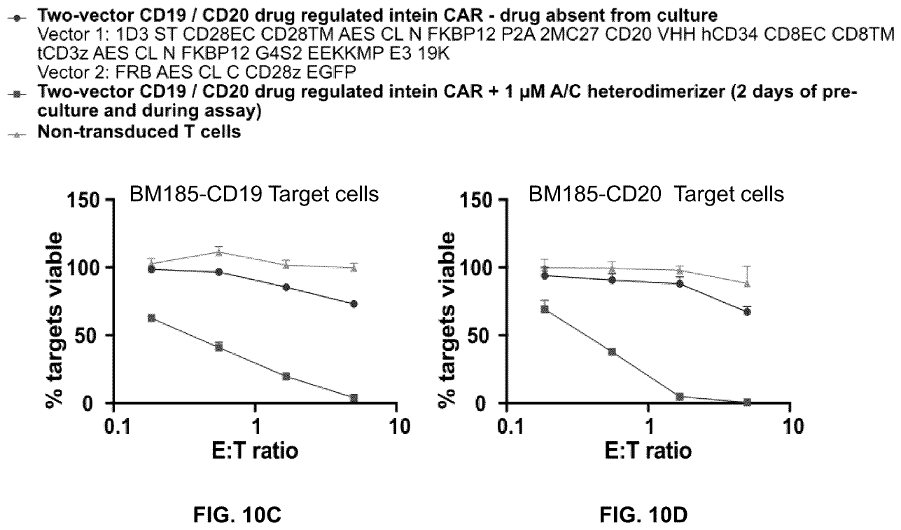

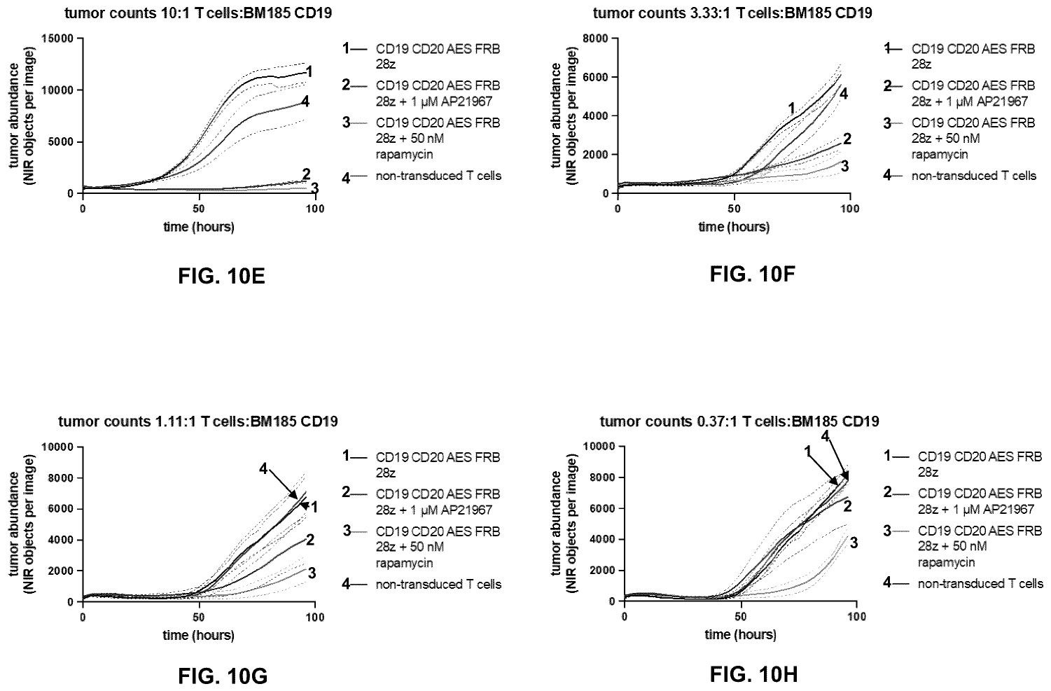

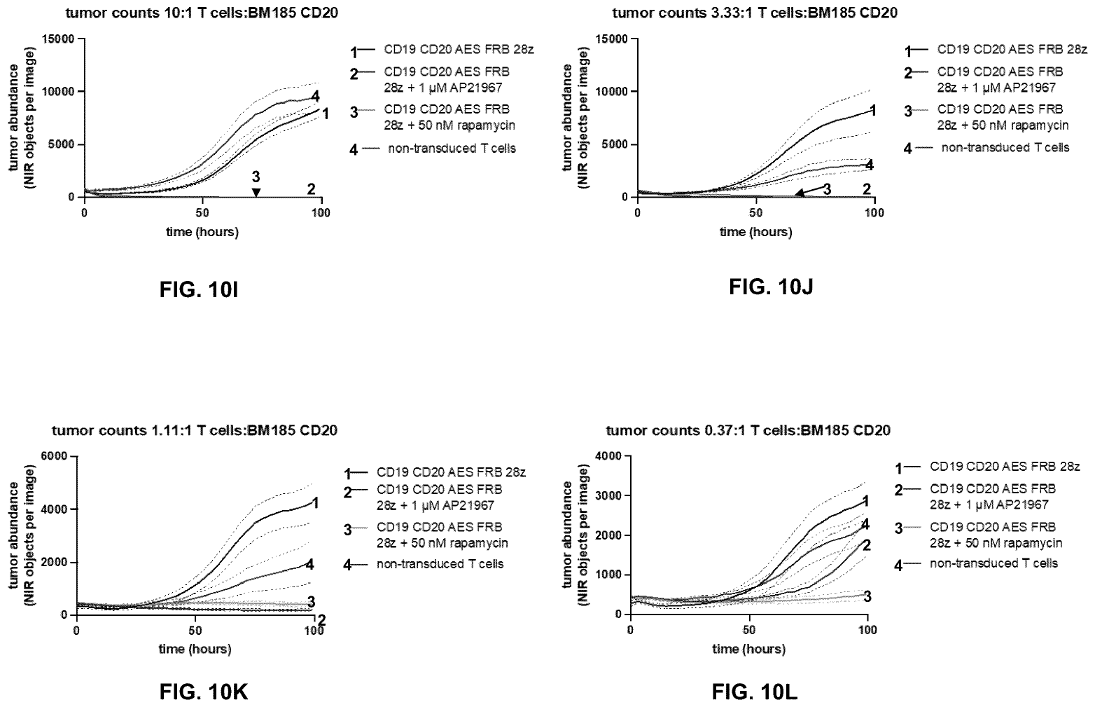

In certain embodiments, the present disclosure is directed to a two-vector intein sorting system with drug-regulated dual-CAR expression. For example, FIG. 10A depicts an exemplary approach for two-vector intein sorting with drug-regulated dual-CAR expression. In this exemplary approach, Vector 1 encodes two distinct antigen-binding scFv transmembrane-domain-containing fusion polypeptides with cytoplasmic low-affinity N terminus intein domains. Each scFv fusion polypeptide contains a distinct affinity tag, a spacer, a transmembrane domain, a low-affinity intein N terminus, and a drug-dimerizable domain (e.g., FKBP12), but only one contains an ER retention motif, due to the ER retention motif functioning most efficiently at the end of a vector construct (C terminus of fusion polypeptide and 2A sequence). Vector 2 encodes a drug-regulated cytoplasmic signaling domain shuttle comprised of a drug-dimerizable domain (e.g., FRB*) fused to a low-affinity intein C terminus, a costimulatory domain, and a T cell receptor signaling domain (e.g., CD3 zeta). Trans-splicing of the two the low-affinity intein-tagged polypeptides occurs maximally in the presence of a dimerizer drug due to enhanced association of the polypeptides mediated by the drug-dimerizable domains. Trans-splicing results in an intracellular splice junction and a recapitulates a fully functional CAR. Surface expression of the affinity-tagged antigen-binding domain polypeptide with an ER retention motif is markedly reduced due to ER retention until the two inteins trans-splice, which removes the ER retention motif, resulting in cell surface trafficking. In certain embodiments, this system enables drug-dependent post-translational dual-CAR assembly and selective affinity-tag based MACS sorting of cells incorporating both vectors and completing the trans-splicing reaction. In certain embodiments, each vector can carry additional transgenes to boost engineered cell functionality.

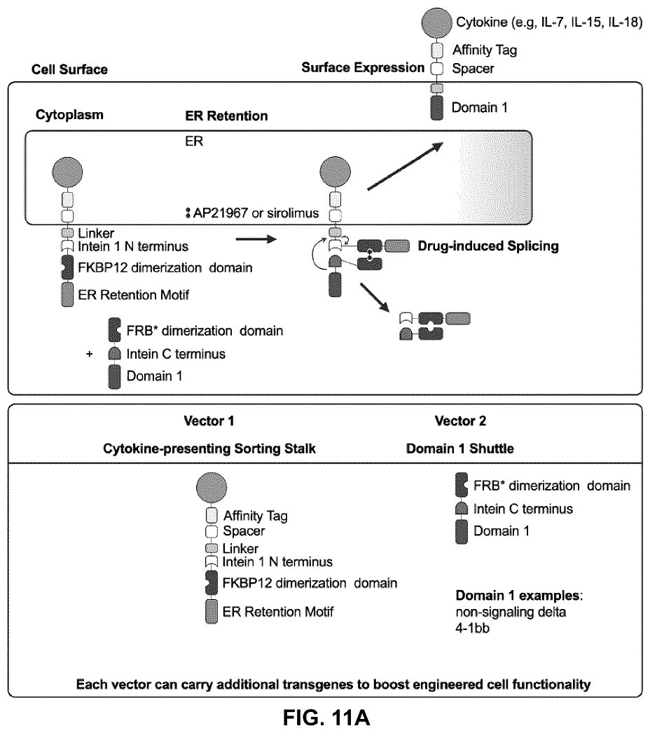

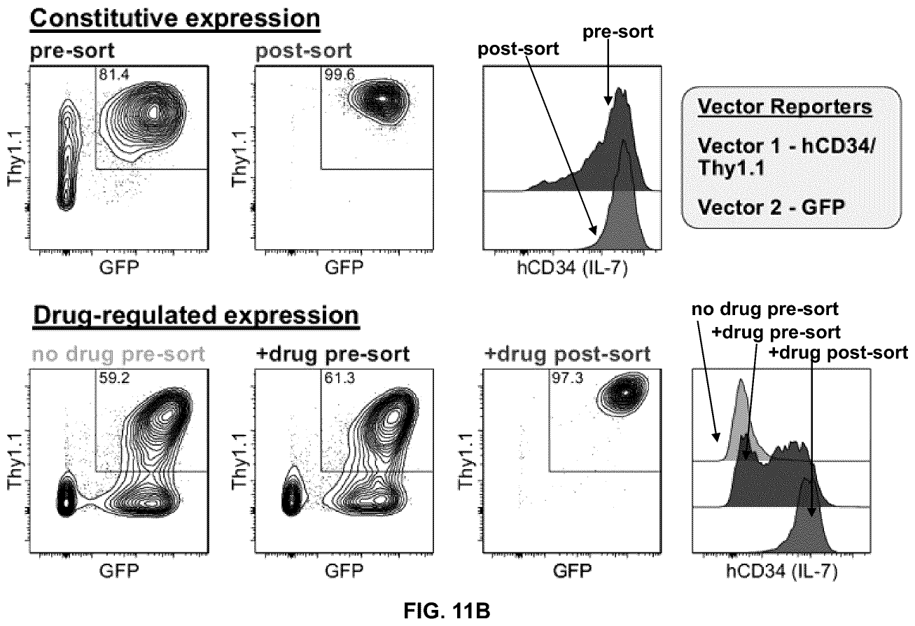

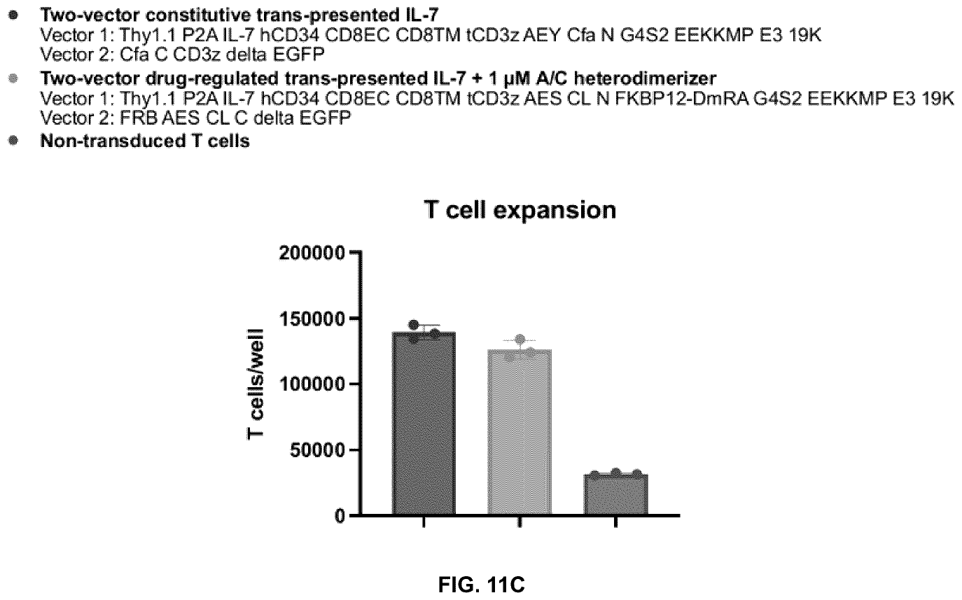

In certain embodiments, the present disclosure is directed to a two-vector intein sorting system with drug-regulated trans-presented cell surface cytokine expression. For example, FIG. 11A depicts an exemplary approach for two-vector intein sorting with drug-regulated trans-presented cell surface cytokine expression. In this exemplary approach, Vector 1 encodes a cytokine fused to an affinity tag, a spacer, a transmembrane domain, a low-affinity intein N terminus, a drug-dimerizable domain (e.g., FKBP12), and an endoplasmic reticulum (ER) retention motif, and Vector 2 encodes a drug-dimerizable domain (e.g., FRB*) fused to a low-affinity intein C terminus and a costimulatory domain containing or non-signaling intracellular domain. Trans-splicing of the two low-affinity intein-tagged polypeptides occurs maximally in the presence of a dimerizer drug due to enhanced association of the polypeptides mediated by the drug-dimerizable domains. Trans-splicing results in excision of the ER retention motif. Surface expression of the affinity-tagged cytokine trans-presentation polypeptide is markedly reduced due to ER retention until the two inteins trans-splice, which removes the retention motif, resulting in cell surface trafficking. In certain embodiments, this system enables drug-dependent graded expression of a cell surface trans-presented cytokine and selective affinity-tag-based MACS sorting of cells incorporating both vectors and completing the trans-splicing reaction. In certain embodiments, each vector can carry additional transgenes to boost engineered cell functionality.

In certain embodiments, the compositions, systems, and methods of the present disclosure can be facilitated by a methodology that utilizes ER retention motifs and drug-regulated trans-splicing to control surface expression of a tethered cytokine. For example, but not by way of limitation, such an approach can be employed to promote cis and trans stimulation of T cells and/or other effector cells to enhance immune function.

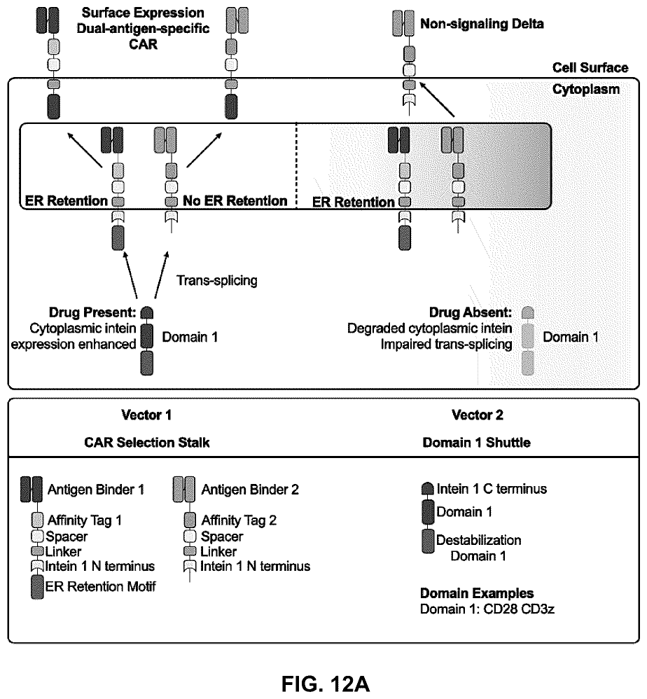

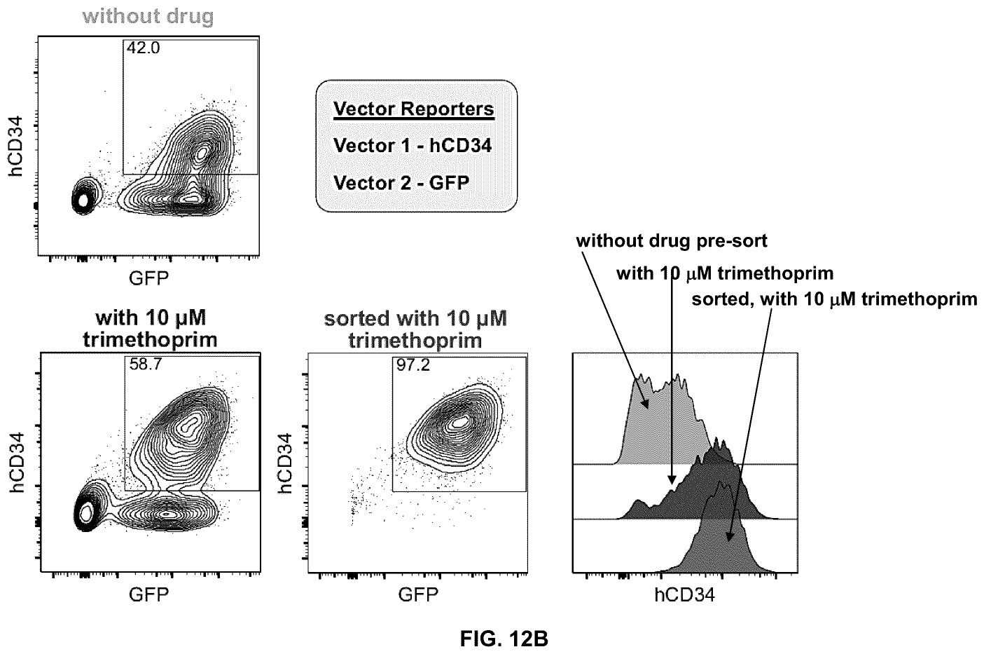

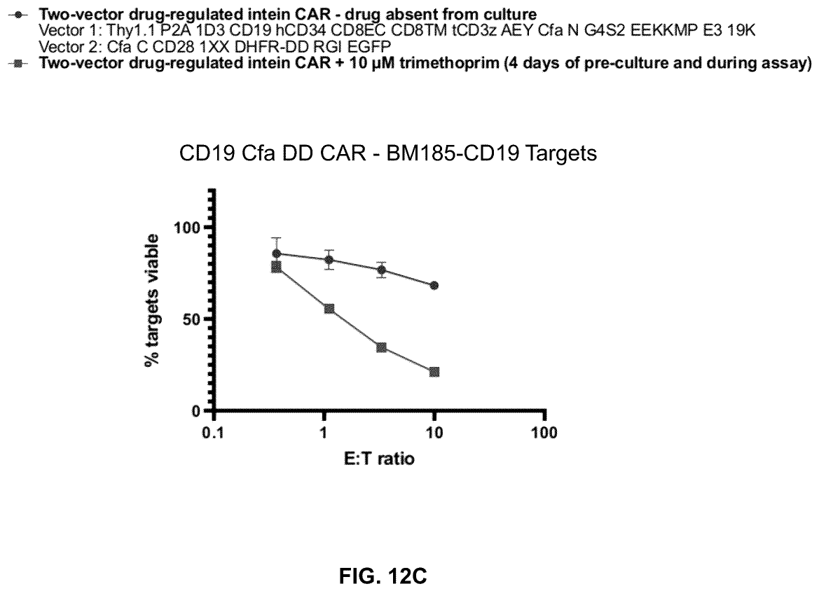

In certain embodiments, the present disclosure is directed to two-vector degenerate intein trans-splicing to enable dual-CAR formation with drug-stabilized cytoplasmic signaling domain shuttle expression. For example, FIG. 12A depicts an exemplary approach for two-vector degenerate intein trans-splicing to enable dual-CAR formation with drug-stabilized cytoplasmic signaling domain shuttle expression. In this exemplary approach, Vector 1 encodes two distinct antigen-binding scFv transmembrane-domain-containing fusion polypeptides with degenerate cytoplasmic N terminus intein domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes a cytoplasmic signaling domain shuttle comprised of an intein C terminus, a costimulatory domain, a T cell receptor signaling domain (e.g., CD3 zeta), and a drug-stabilized degron domain (e.g., E. coli DHFR destabilization domain). Surface expression of the affinity-tagged antigen-binding domain polypeptide with an ER retention motif is markedly reduced due to ER retention until trans-splicing with the cytoplasmic signaling domain shuttle, which removes the retention motif, resulting in cell surface trafficking. Maximum expression of the cytoplasmic signaling domain shuttle occurs in the presence of the stabilizing drug, facilitating trans-splicing reaction. In certain embodiments, this system enables drug-dependent post-translational dual-CAR and assembly combined with selective affinity-tag-based MACS sorting of cells incorporating both vectors and completing the trans-splicing reaction. In certain embodiments, each vector can carry additional transgenes to boost engineered cell functionality.

In certain embodiments, the compositions, systems, and methods of the present disclosure can be facilitated by a methodology that utilizes the linkage of a drug-regulated degron (destabilized protein) to the cytoplasmic signaling shuttle intein to regulate intein expression inside cells. For example, but not by way of limitation, such methodologies can thereby control trans-splicing activity and CAR signaling.

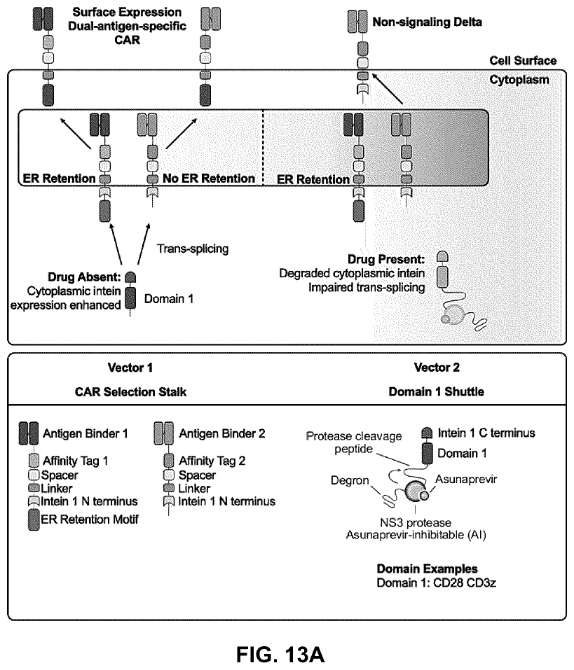

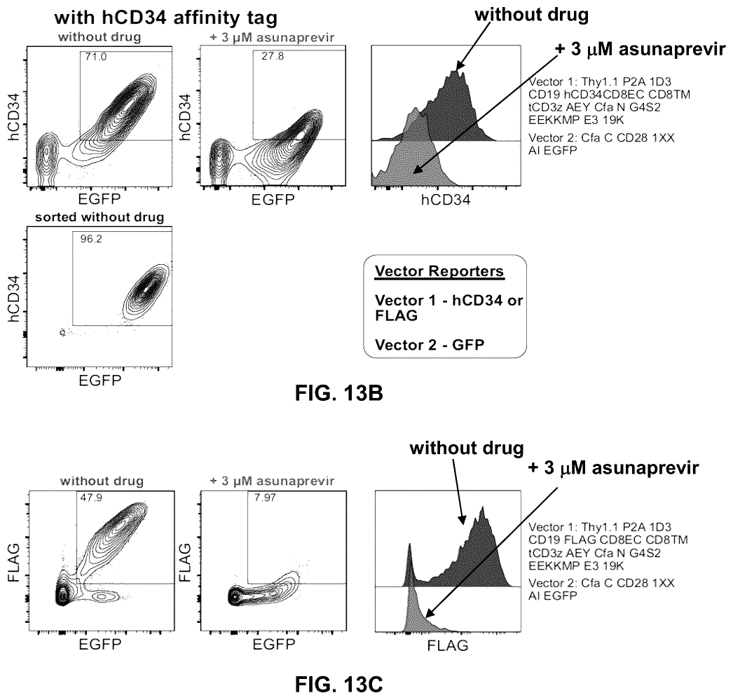

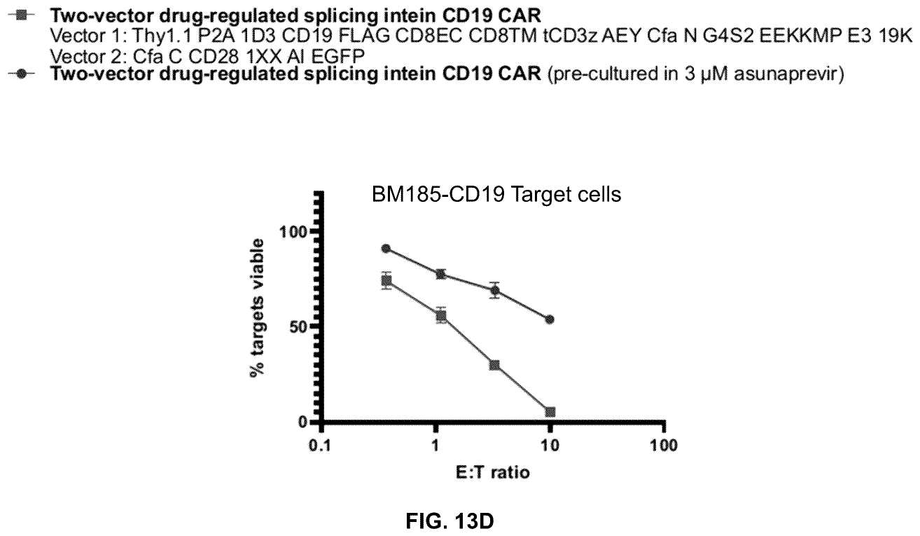

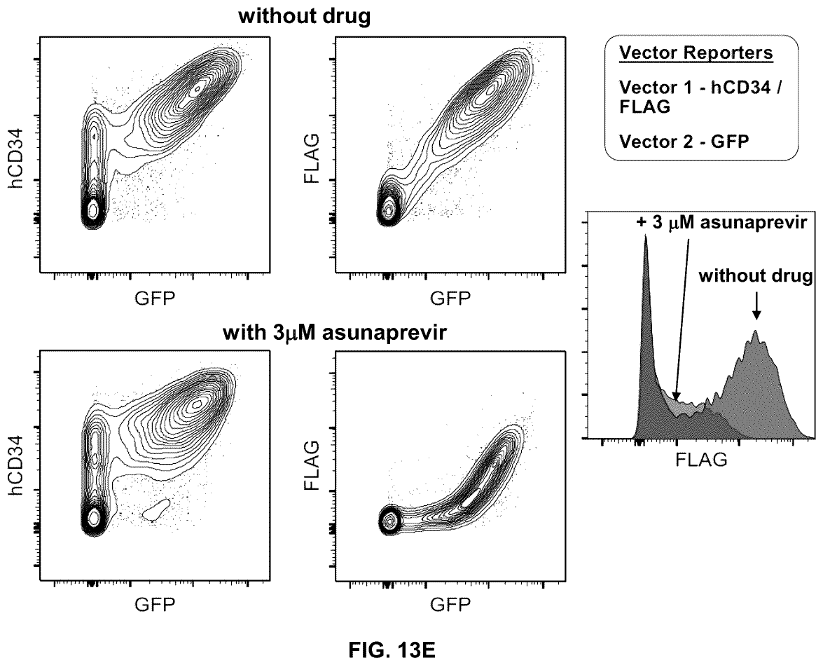

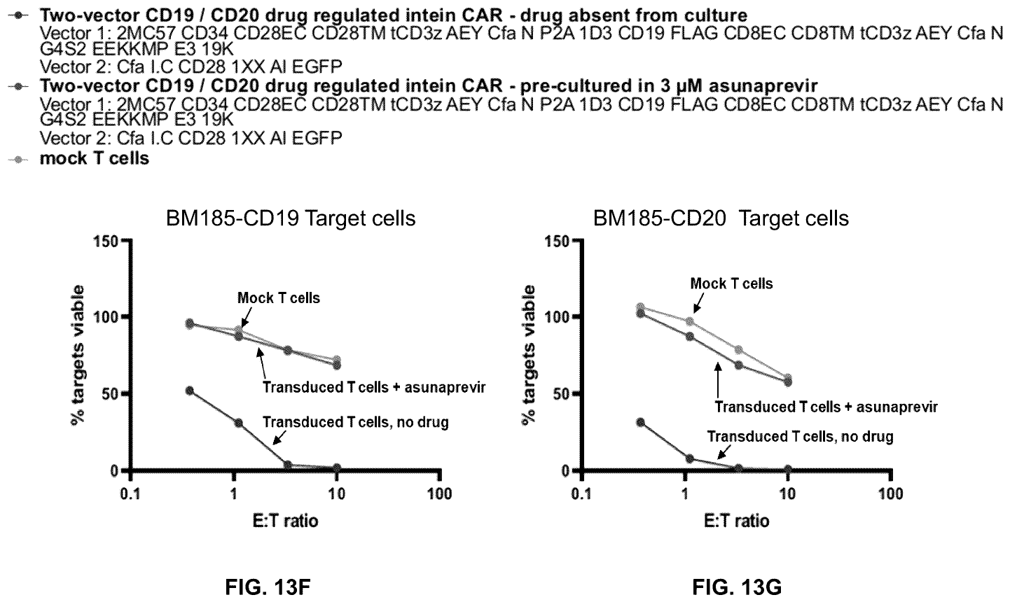

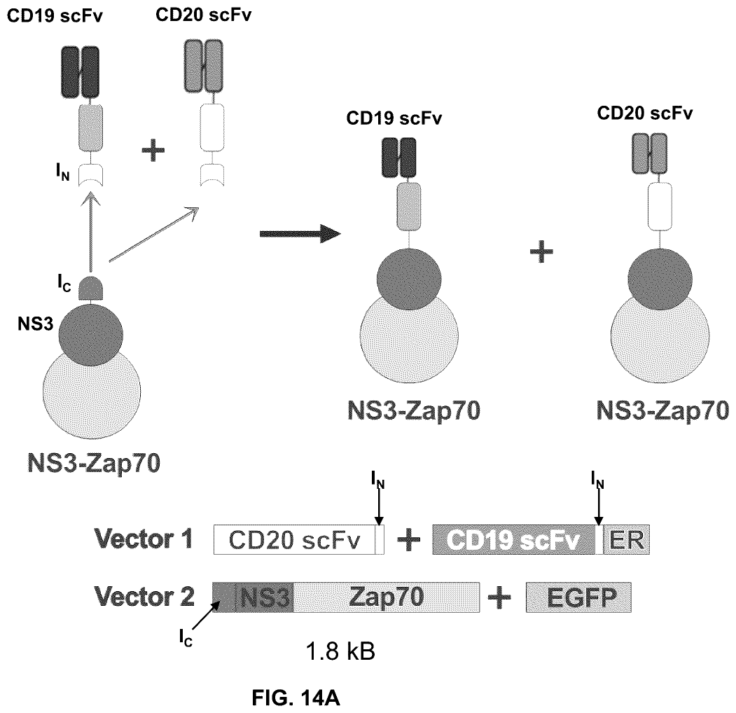

In certain embodiments, the present disclosure is directed to two-vector degenerate intein trans-splicing to enable dual-CAR formation with drug-destabilized cytoplasmic signaling domain shuttle expression. For example, FIG. 13A depicts an exemplary approach for two-vector degenerate intein trans-splicing to enable dual-CAR formation with drug-destabilized cytoplasmic signaling domain shuttle expression. In this exemplary approach, Vector 1 encodes two distinct antigen-binding scFv transmembrane-domain-containing fusion polypeptides with degenerate cytoplasmic N terminus intein domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes a cytoplasmic signaling domain shuttle comprised of an intein C terminus, a costimulatory domain, a T cell receptor signaling domain (e.g., CD3 zeta), and a drug-activated degron domain (e.g., SMASh tag). Surface expression of the affinity-tagged antigen-binding domain polypeptide with an ER retention motif is markedly reduced due to ER retention until trans-splicing with the cytoplasmic signaling domain shuttle, which removes the retention motif, resulting in cell surface trafficking. Expression of the cytoplasmic signaling domain shuttle is reduced in the presence of the inhibitor drug, which promotes proteasomal degradation, resulting in inhibition of the trans-splicing reaction and reduction in CAR formation. In certain embodiments, this system enables drug-dependent post-translational dual-CAR assembly combined with selective affinity-tag-based MACS sorting of cells incorporating both vectors and completing the trans-splicing reaction. In certain embodiments, each vector can carry additional transgenes to boost engineered cell functionality.

In certain embodiments, the compositions, systems, and methods of the present disclosure can be facilitated by a methodology, such as that exemplified in FIGS. 13A-13D, which enables generation of T cells able to target two antigens simultaneously with coordinated drug-regulation. In certain embodiments, such methodologies can enable single-step MACS purification of highly purified dual-transduced T cells incorporating two vectors.

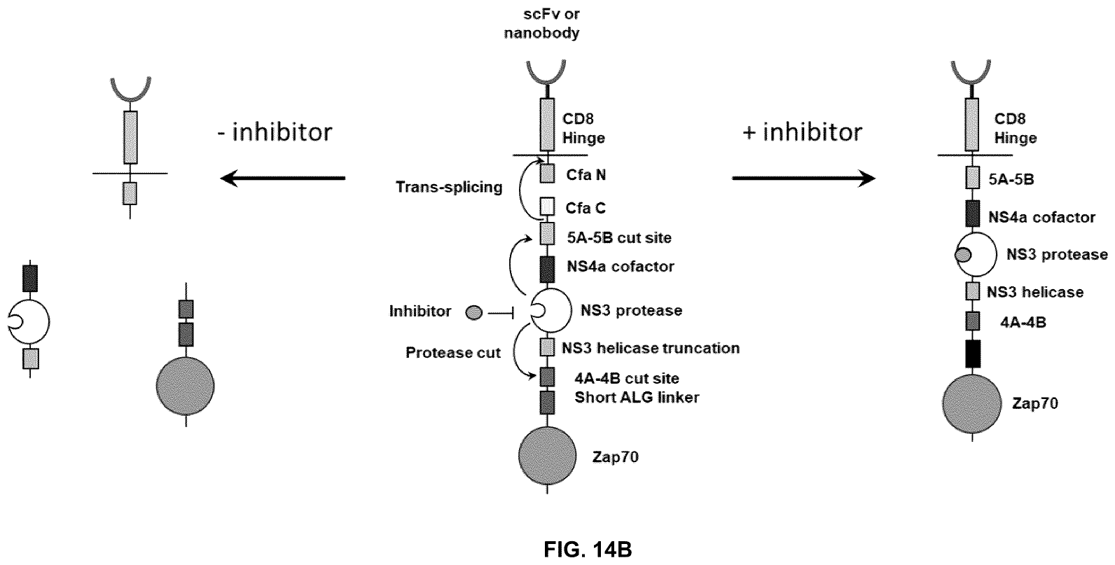

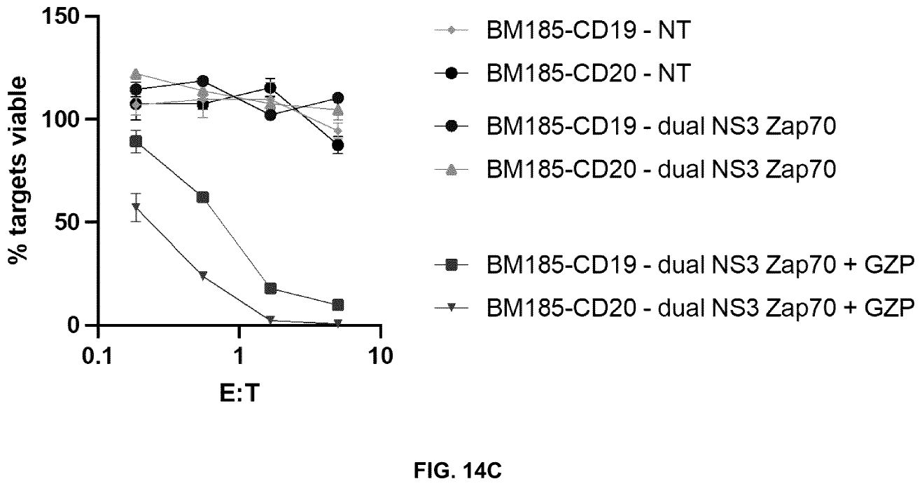

In certain embodiments, the present disclosure is directed to two-vector degenerate intein trans-splicing to enable dual-CAR formation with drug-destabilized cytoplasmic signaling domain shuttle expression. For example, FIG. 14A depicts an exemplary approach for two-vector degenerate intein trans-splicing to enable expression of both, a Zap70 based CD19 scFv and a Zap70 based CD20 scFv. In this exemplary approach, Vector 1 encodes two distinct antigen-binding scFv domain containing fusion polypeptides with degenerate cytoplasmic N terminus intein domains (I_{N}). Each scFv fusion polypeptide contains a distinct affinity tag and may additionally comprise an ER retention motif at the end of a vector construct at the C terminus of fusion polypeptide (e.g., see FIG. 3A). Vector 2 encodes a drug regulatable Zap70 domain shuttle comprised of a Non-structural 3 (NS3) protease recognition sequence and an intein C terminus (I_{C}). Surface expression of the affinity-tagged antigen-binding domain polypeptide(s) with an ER retention motif is markedly reduced due to ER retention until trans-splicing with the cytoplasmic signaling domain shuttle, which removes the retention motif, resulting in cell surface trafficking. Expression of the cytoplasmic signaling domain shuttle is reduced in the presence of the inhibitor drug, which promotes proteasomal degradation, resulting in inhibition of the trans-splicing reaction and reduction in CAR formation. In certain embodiments, this system enables drug-dependent post-translational dual-CAR assembly combined with selective affinity-tag-based MACS sorting of cells incorporating both vectors and completing the trans-splicing reaction. In certain embodiments, each vector can carry additional transgenes to boost engineered cell functionality.

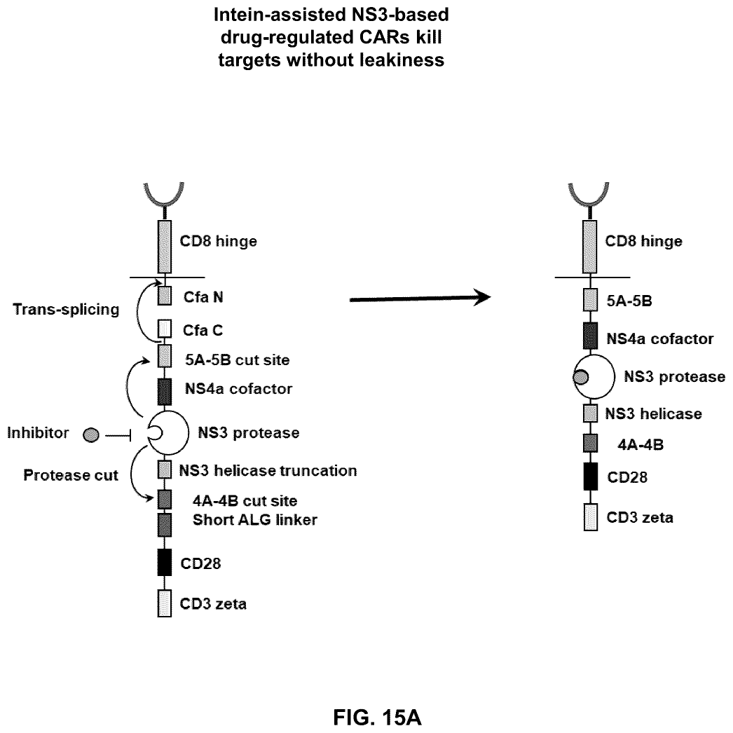

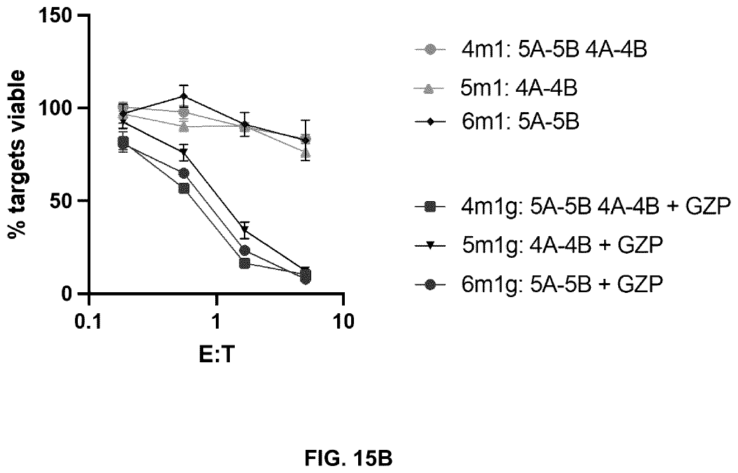

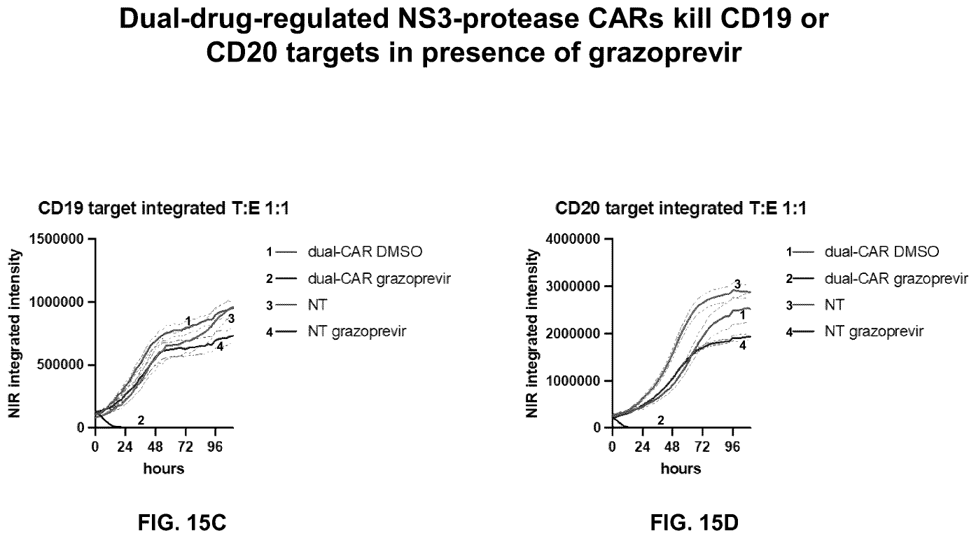

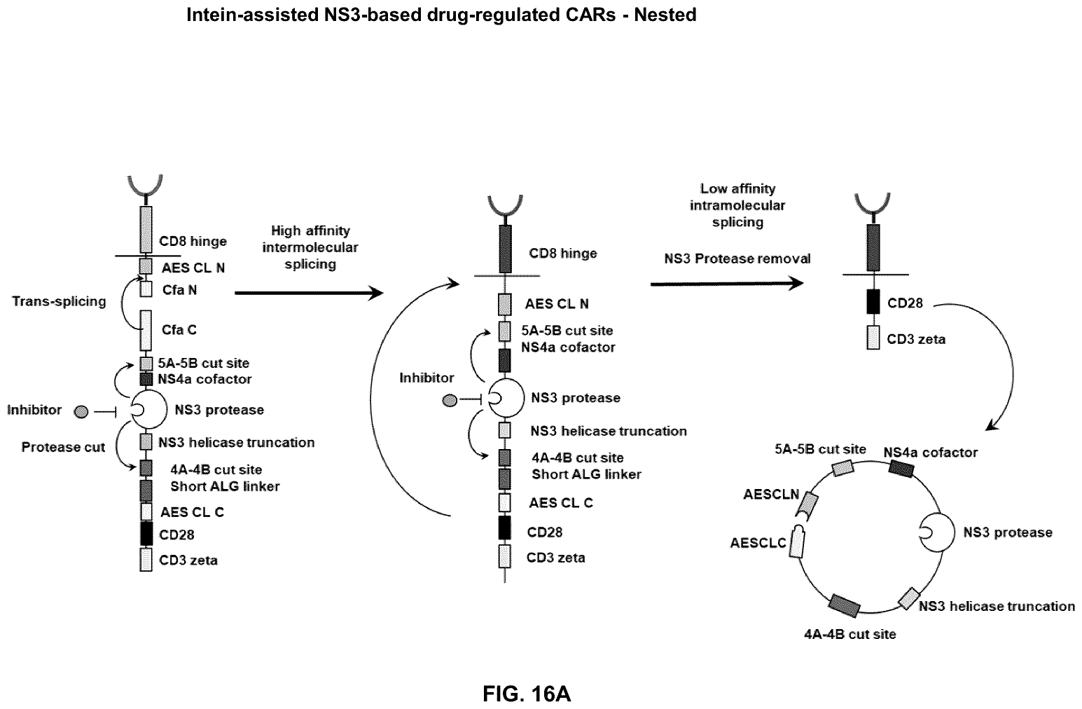

In certain embodiments, the present disclosure is directed an intein-assisted NS3-based drug-regulated CAR. For example, FIG. 15A depicts an exemplary approach for generating a drug-regulated NS3-based CAR and comprising a dual-vector-selecting cell sorting method.

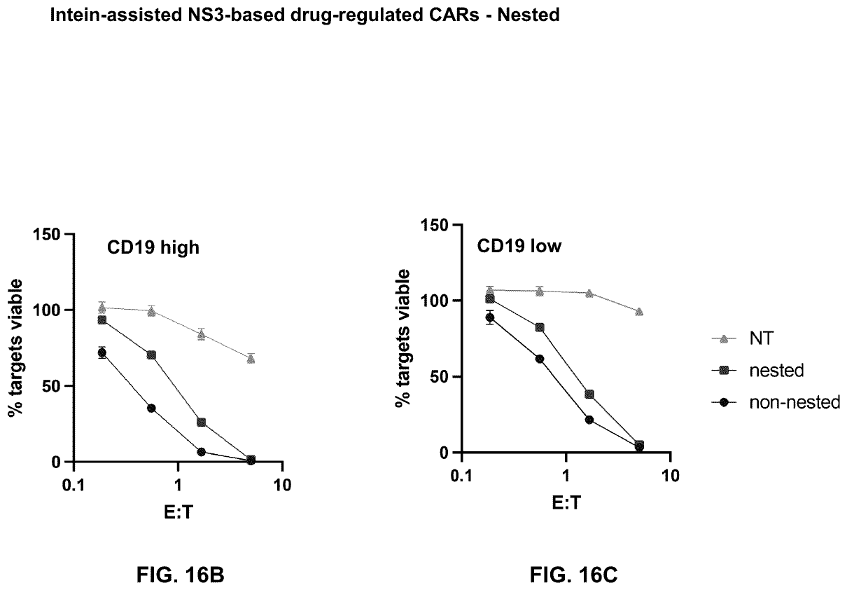

In certain embodiments, the present disclosure is directed an intein-assisted NS3-based drug-regulated CAR generated using a nested approach. For example, FIG. 16A depicts an exemplary approach for generating dual-antigen-targeting drug-regulated NS3-based CARs and comprising a dual-vector-selecting cell sorting method.

In certain embodiments, the compositions, systems, and methods of the present disclosure can be facilitated by a methodology, such as that exemplified in FIGS. 16A-16C, that enables drug-regulated generation of actively signaling CARs with removal of the drug regulation polypeptides, e.g., NS3 polypeptides, which could impair CAR signal transduction. In certain embodiments, such methodologies are accomplished by placing signaling domains in a membrane distal location as illustrated in FIGS. 16A-16C. Removal of the drug regulation polypeptides can also prolong the activity of CAR in the absence of the HCV protease inhibitor.

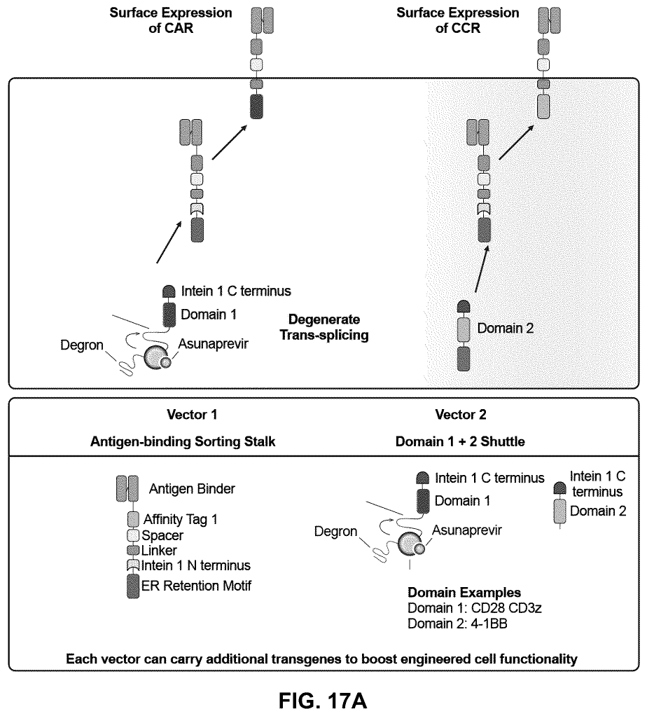

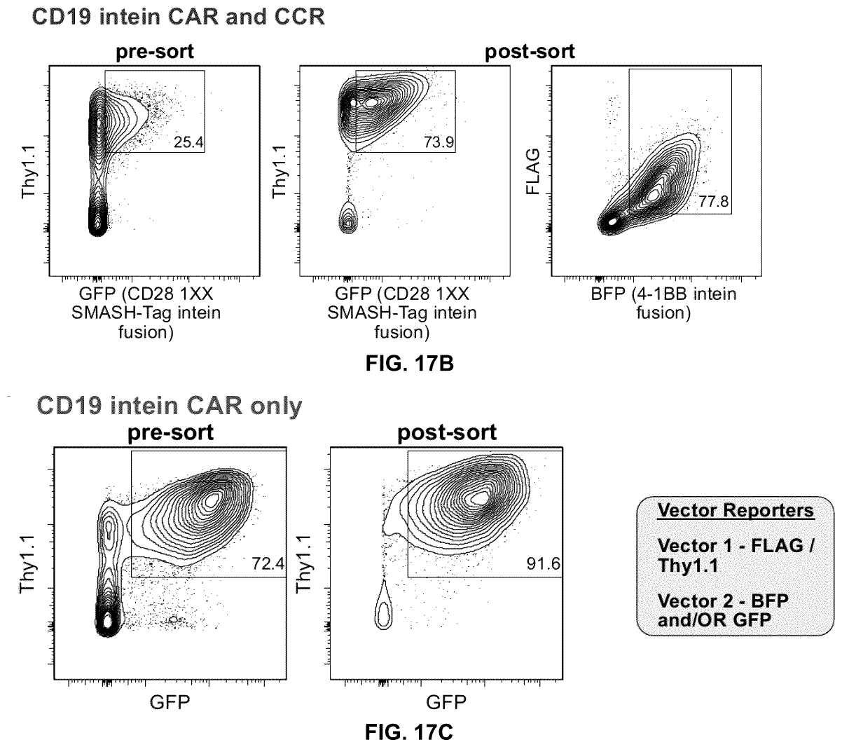

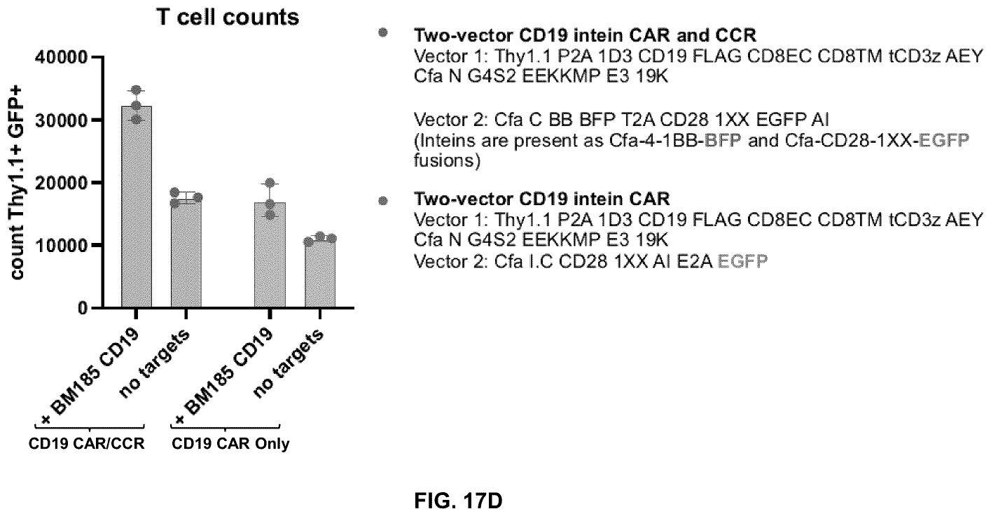

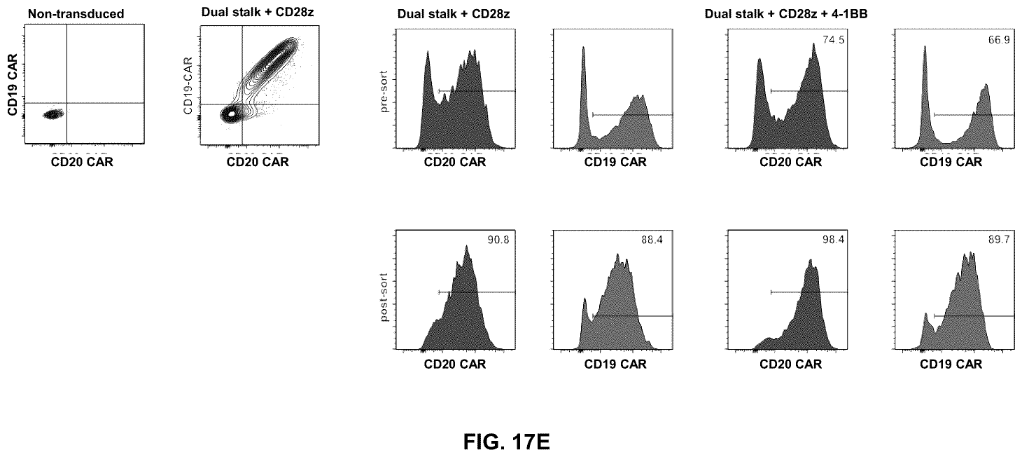

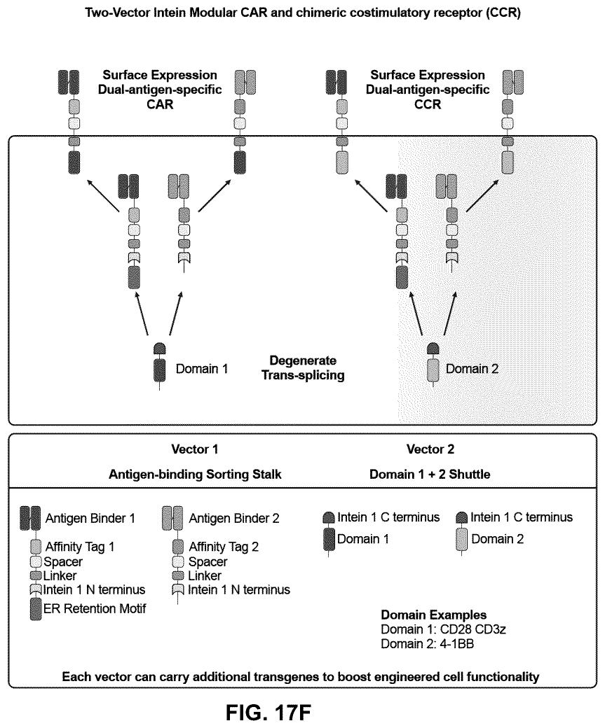

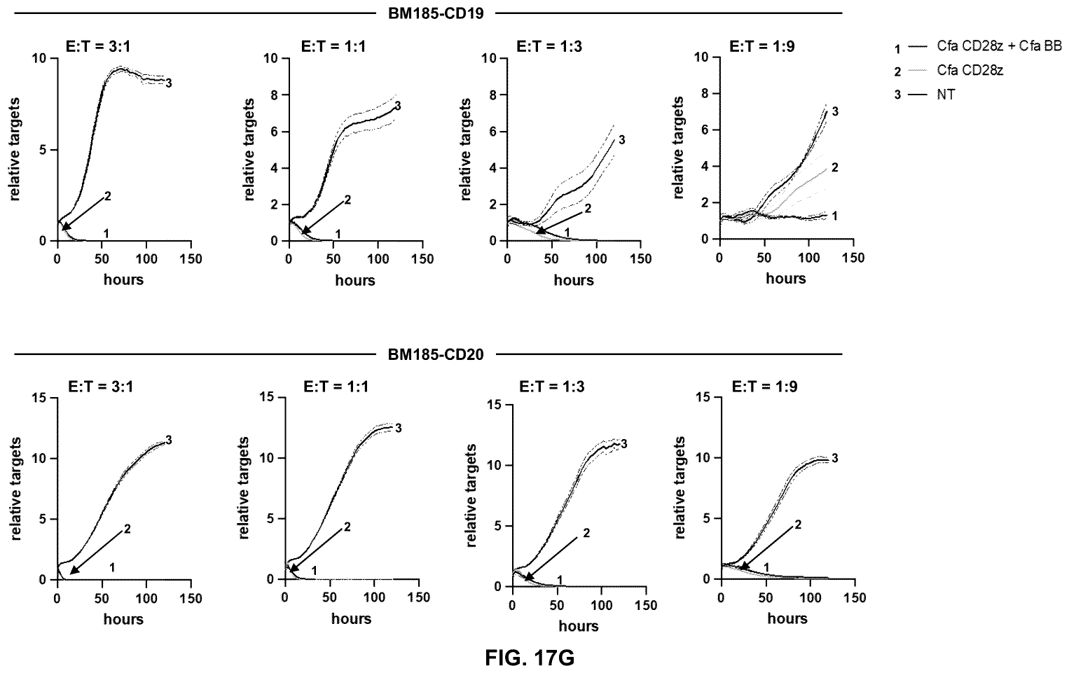

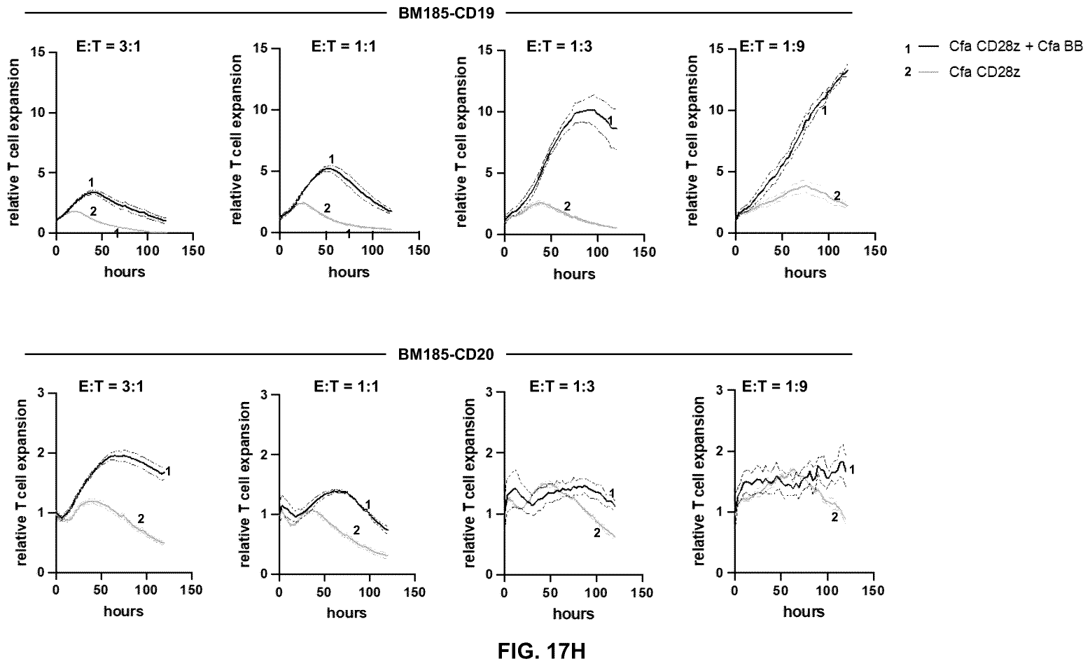

In certain embodiments, the present disclosure is directed to two-vector degenerate intein trans-splicing to enable multiplexed post-translational CAR and chimeric costimulatory receptor (CCR) formation. For example, FIG. 17A depicts an exemplary approach for two-vector degenerate intein trans-splicing to enable multiplexed post-translational CAR and CCR formation. In this exemplary approach, Vector 1 encodes an antigen-binding scFv transmembrane-domain-containing fusion polypeptides with degenerate cytoplasmic N terminus intein domains. The scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes two cytoplasmic signaling domain shuttle comprised of an intein C terminus, a costimulatory domain (e.g., CD28, 4-1BB), a T cell receptor signaling domain (e.g., CD3 zeta), and at least one drug-activated degron domain (e.g., SMASh tag). Surface expression of the affinity-tagged antigen-binding domain polypeptide with an ER retention motif is markedly reduced due to ER retention until trans-splicing with the cytoplasmic signaling domain shuttle, which removes the retention motif, resulting in cell surface trafficking. Expression of the cytoplasmic signaling domain shuttle is reduced in the presence of the inhibitor drug, which promotes proteasomal degradation, resulting in inhibition of the trans-splicing reaction and reduction in CAR formation. In certain embodiments, this system enables drug-dependent post-translational dual-CAR assembly combined with selective affinity-tag-based MACS sorting of cells incorporating both vectors and completing the trans-splicing reaction. In certain embodiments, each vector can carry additional transgenes to boost engineered cell functionality.

In certain embodiments, the present disclosure is directed to two-vector degenerate intein trans-splicing to enable multiplexed post-translational CAR and chimeric costimulatory receptor (CCR) formation. For example, FIG. 17F depicts an exemplary approach for two-vector degenerate intein trans-splicing to enable multiplexed post-translational dual CAR and dual CCR formation. In this exemplary approach, Vector 1 encodes two distinct antigen-binding scFv (CD19/CD20) transmembrane-domain-containing fusion polypeptides with degenerate cytoplasmic N terminus intein domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct (C terminus of fusion polypeptide and 2A sequence). Vector 2 encodes two cytoplasmic signaling domain shuttles each comprised of a degenerate intein C terminus, a distinct costimulatory domain (e.g., CD28z, 4-1BB). Trans-splicing of combinations of two degenerate intein-tagged antigen-binding polypeptides with the two cytoplasmic signaling domain shuttles enables generation of two CAR and two CCR molecules totaling four mature trans-sliced polypeptides. Surface expression of the affinity-tagged antigen-binding domain polypeptide with an ER retention motif is markedly reduced due to ER retention until the two inteins trans-splice, which removes the retention motif, resulting in cell surface trafficking. This system enables post-translational dual-CAR and dual-CCR assembly combined with selective affinity-tag-based MACS sorting of cells incorporating both vectors and completing the trans-splicing reaction. Further modification to the cytoplasmic signaling domain shuttles to include orthogonal drug-destabilization or selective degron domains can enable selective drug-dependent regulation of each signaling domain. Each vector can carry additional transgenes to boost engineered cell functionality.

In certain embodiments, the compositions, systems, and methods of the present disclosure can be facilitated by a methodology that enables trans-splicing combination to generate four possible combinations of CARs and/or CCRs. For example, but not by way of limitation, FIGS. 17A-17D exemplify such a methodology for generating CD19-CAR, CD20-CAR, CD19-CCR, and CD20-CCR. In certain embodiments, such methodologies represent a data compression algorithm whereby re-use of degenerate encoded DNA elements enables post-translational assembly of a greater number of receptors than could be encoded with the same amount of DNA. Moreover, in certain embodiments, the additive CAR+CCR co-stimulation promotes enhanced T cell proliferation and, in certain embodiments, enhanced target killing at low T cell E:T ratios, consistent with more active cell product.

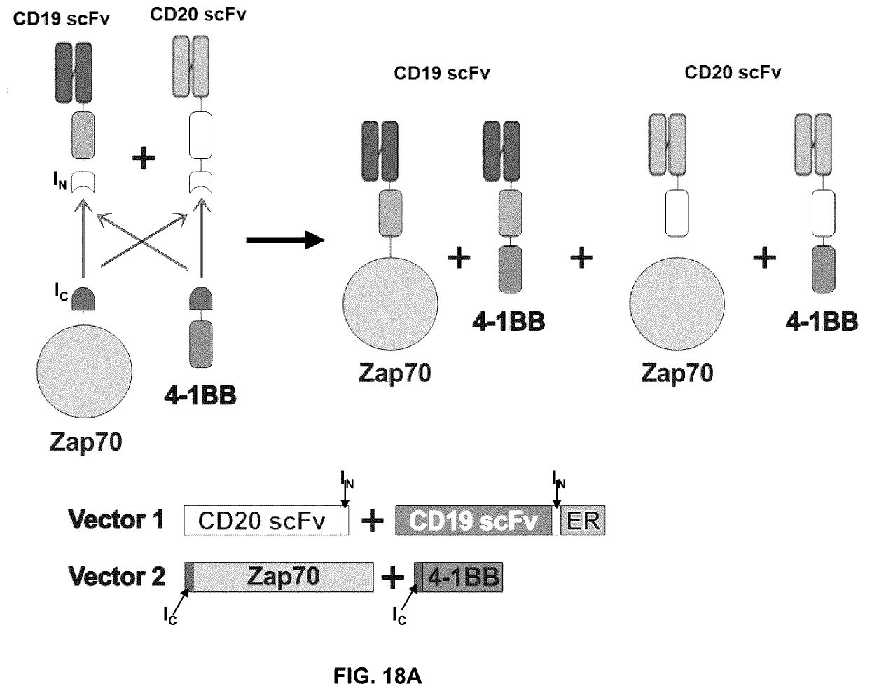

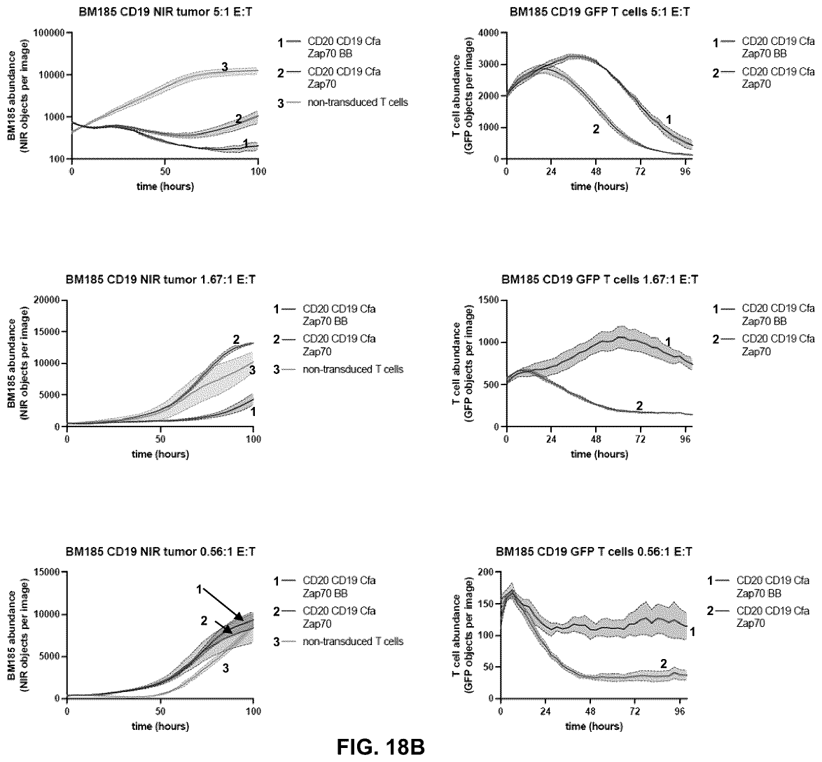

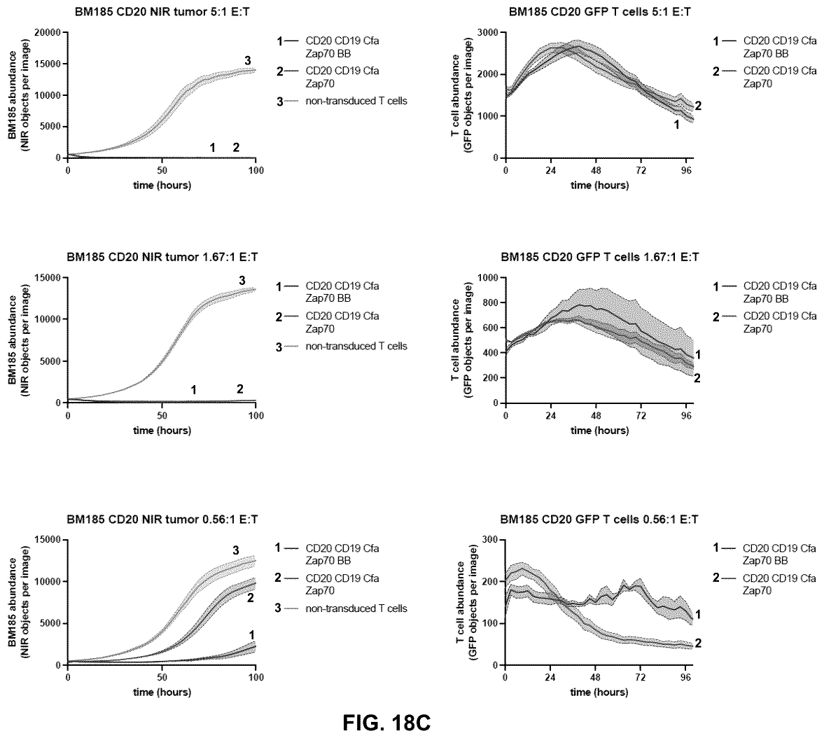

In certain embodiments, the present disclosure is directed to two-vector degenerate intein trans-splicing to enable multiplexed post-translational CAR with a Zap70 domain and chimeric costimulatory receptor (CCR). For example, FIG. 18A depicts an exemplary approach for two-vector degenerate intein trans-splicing to enable expression of both, a Zap70 based CD19 scFv and a Zap70 based CD20 scFv. In this exemplary approach, Vector 1 encodes two distinct antigen-binding scFv transmembrane-domain-containing fusion polypeptides (e.g., anti-CD19, anti-CD20) with degenerate cytoplasmic N terminus intein (I_{N}) domains. Each scFv fusion polypeptide contains a distinct affinity tag, a spacer, a transmembrane domain, and cytoplasmic degenerate intein N terminus, but only one contains an ER retention motif, due to the ER retention motif functioning most efficiently at the end of a vector construct. Vector 2 encodes Zap70 domain shuttle and a 4-1BB domain sequence each attached at their N terminus to an intein C terminus (I_{C}). Surface expression of the affinity-tagged antigen-binding domain polypeptide(s) with an ER retention motif is markedly reduced due to ER retention until trans-splicing with the cytoplasmic signaling domain shuttle, which removes the retention motif, resulting in cell surface trafficking. In certain embodiments, each vector can carry additional transgenes to boost engineered cell functionality. In certain embodiments, this configuration enables post-translational assembly of a larger number of receptors than is possible with conventional methods using the same amount of DNA nucleotides.

Table 2 shows exemplary sequences for the various elements that comprise the degenerate intein-based trans-splicing system. Table 3 lists exemplary construct sequences for the intein-based trans-splicing system.

TABLE 2
Exemplary sequences of elements comprising
the intein-based trans-splicing system

| | | SEQ |
|---|---|---|
| Name | Sequence | ID NO. |
| Linker1 | GGGGSGGGSELPTQGTFSNVSTNVSGGGGSGGGSRSG | 11 |
| Linker2 | TSGGGGSGGGSG | 12 |
| Linker3 | TSGGGGSGGGSGTG | 13 |
| Linker4 | GGGGSGGGSGTG | 14 |
| Linker5 | STGGGGSGGGS | 15 |
| Linker6 | GSGGGGSGGGS | 16 |
| Linker7 | GTSRAKRGSG | 17 |
| Linker8 | GGGGSGGGS | — |
| Linker9 | GGGGSGTG | — |
| Linker10 | GTSRAKRGS | — |
| Linker11 | ALGGSGGGS | — |
| Linker12 | GGGGSTS | — |
| Linker13 | SGGGGSD | — |
| Linker14 | GSSGSG | — |
| Linker15 | GSGGTR | — |
| Linker16 | GSGTR | — |
| Linker17 | TSGSG | — |
| Linker18 | GSGGS | — |
| Linker19 | GSS | — |
| Linker20 | GTG | — |
| Linker21 | LES | — |
| Linker22 | GSG | — |
| Linker23 | GSL | — |
| Linker24 | ALG | — |
| Furin cleavage site | RAKR | — |
| ER retention motif-E319K | GGGGSGGGSGTGKYKSRRSFIEEKKMP | 18 |
| Linker (e.g., | | |
| GGGGSGGGSGTG) | | |
| underlined | | |
| E3 19K motif in bold | | |
| ER retention motif-Kir6.2 | GGGGSGGGSGTGLLDALTLASSRGPLRKRSVAVAKA | 19 |
| RXR | KPKFSISPDSLS | |
| Linker (e.g., | | |
| GGGGSGGGSGTG) | | |
| underlined | | |
| RXR containing motif in | | |
| bold | | |
| kainite 5 RXR motif | EFIWSTRRSAESEEVSVCQEMLQELRHAVSCRKTSRSR | 20 |
| | RRRR | |
| Na+ channel Nav1.8 RXR | EVLAALGIDTTSLHSHNGSPLTSKNASERRHRIKP | 21 |
| motif | | |
| SAP97 RXR motif | RKSGRAEPDPKKKATFRAITSTLASSFKRRRSS | 22 |
| E2A | QCTNYALLKLAGDVESNPGP | 23 |
| P2A | ATNFSLLKQAGDVEENPGP | 24 |
| T2A | EGRGSLLTCGDVEENPGP | 25 |
| F2A | VKQTLNFDLLKLAGDVESNPGP | 26 |
| FLAG tag | DYKDDDDK | — |
| Tandem Strep-tag®-II | WSHPQFEKGGGSGGGSGGSAWSHPQFEK | 27 |
| V5 Tag | GKPIPNPLLGLDST | 28 |
| CD34 tag | ELPTQGTFSNVSTNVS | 29 |
| Tandem CD20 mimotope | SGGGGSDCPYSNPSLCSGGGGSDCPYSNPSLCGGGGS | 30 |
| tag | GGGS | |
| Linker (e.g., SGGGGSD, | | |
| GGGGSGGGS) underlined | | |
| Mimotope in bold | | |
| tagBFP | MSELIKENMHMKLYMEGTVDNHHFKCTSEGEGKPYE | 31 |
| | GTQTMRIKVVEGGPLPFAFDILATSFLYGSKTFINHTQG | |
| | IPDFFKQSFPEGFTWERVTTYEDGGVLTATQDTSLQDG | |
| | CLIYNVKIRGVNFTSNGPVMQKKTLGWEAFTETLYPA | |
| | DGGLEGRNDMALKLVGGSHLIANIKTTYRSKKPAKNL | |
| | KMPGVYYVDYRLERIKEANNETYVEQHEVAVARYCD | |
| | LPSKLGHKLN | |
| CD8 spacer (mouse) | STTTKPVLRTPSPVHPTGTSQPQRPEDCRPRGSVKGTG | 32 |
| | LDFACD | |
| CD28 spacer (mouse) | IEFMYPPPYLDNERSNGTIIHIKEKHLCHTQSSPKL | 33 |
| PD-1 spacer (mouse) | TERILETSTRYPSPSPKPEGRFQGM | 34 |
| IgG1 hinge (mouse) | VPRDCGCKPCICT | 35 |
| Leucine Zipper | LEIRAAFLRQRNTALRTEVAELEQEVQRLENEVSQYET | 36 |
| (RR12EE345L) | RYGPLGGGK | |
| Leucine Zipper | LEIEAAFLERENTALETRVAELRQRVQRLRNRVSQYRT | 37 |
| (EE12RR345L) | RYGPLGGGK | |
| FKBP12 | GVQVETISPGDGRTFPKRGQTCVVHYTGMLEDGKKFD | 38 |
| | SSRDRNKPFKFMLGKQEVIRGWEEGVAQMSVGQRAK | |
| | LTISPDYAYGATGHPGIIPPHATLVFDVELLKLE | |
| FRB* | MILWHEMWHEGLEEASRLYFGERNVKGMFEVLEPLH | 39 |
| | AMMERGPQTLKETSFNQAYGRDLMEAQEWCRKYMK | |
| | SGNVKDLLQAWDLYYHVFRRISK | |
| DHFR destabilization | ISLIAALAVDYVIGMENAMPWNLPADLAWFKRNTLN | 40 |
| domain | KPVIMGRHTWESIGRPLPGRKNIILSSQPSTDDRVTWV | |
| | KSVDEAIAACGDVPEIMVIGGGRVIEQFLPKAQKLYLT | |
| | HIDAEVEGDTHFPDYEPDDWESVFSEFHDADAQNSHS | |
| | YCFEILERR | |
| Degron (SMASh tag) | GTGGSGDEMEECSQHLPGAGSSGDIMGSSGTGSGSGT | 41 |
| | SAPITAYAQQTRGLLGCIITSLTGRDKNQVEGEVQIMS | |
| | TATQTFLATCINGVCWAVYHGAGTRTIASPKGPVIQM | |
| | YTNVDQDLVGWPAPQGSRSLTPCTCGSSDLYLVTRHA | |
| | DVIPVRRRGDGRGSLLSPRPISYLKGSSGGPLLCPAGH | |
| | AVGLFRAAVCTRGVAKAVDFIPVENLETTMRSPVFTD | |
| | NSSPPAVTLTHPITKIDTKYIMTCMSADLEVVTSTWVL | |
| | VGGVLAALAAYCLSTGCVVIVGRIVLSGKPAIIPDREV | |
| | LYGS | |
| HCV NS3 protease | APITAYAQQTRGLLGCIITSLTGRDKNQVEGEVQIVST | 42 |
| | ATQTFLATCINGVCWAVYHGAGTRTIASPKGPVIQMY | |
| | TNVDQDLVGWPAPQGSRSLTPCTCGSSDLYLVTRHAD | |
| | VIPVRRRGDSRGSLLSPRPISYLKGSSGGPLLCPAGHAV | |
| | GLFRAAVCTRGVAKAVDFIPVENLETTMRSPVFTD | |
| HCV NS3 protease | EDVVCCHSIYGKKKGGGGSGGGSTGCVVIVGRIVLSG | 43 |
| | SGTSAPITAYAQQTRGLLGCIITSLTGRDKNQVEGEVQI | |
| | VSTATQTFLATCINGVCWAVYHGAGTRTIASPKGPVIQ | |
| | MYTNVDQDLVGWPAPQGSRSLTPCTCGSSDLYLVTR | |
| | HADVIPVRRRGDSRGSLLSPRPISYLKGSSGGPLLCPAG | |
| | HAVGLFRAAVCTRGVAKAVDFIPVENLETTMRSPVFT | |
| | DNSSPPAVTLTHPITKIDREVLYQEFDEMEECSQH | |
| HCV NS3 protease | GTGGGGGSGGGSTGCVVIVGRIVLSGSGTSAPITAYAQ | 44 |
| | QTRGLLGCIITSLTGRDKNQVEGEVQIVSTATQTFLAT | |
| | CINGVCWAVYHGAGTRTIASPKGPVIQMYTNVDQDL | |
| | VGWPAPQGSRSLTPCTCGSSDLYLVTRHADVIPVRRR | |
| | GDSRGSLLSPRPISYLKGSSGGPLLCPAGHAVGLFRAA | |
| | VCTRGVAKAVDFIPVENLETTMRSPVFTDNSSPPAVTL | |
| | THPITKIDREVLYQEFDEMEECSQH | |
| HCV NS3 protease | EDVVCCHSIYGKKKGGGGSGGGSTGCVVIVGRIVLSG | 45 |
| | SGTSAPITAYAQQTRGLLGCIITSLTGRDKNQVEGEVQI | |
| | VSTATQTFLATCINGVCWAVYHGAGTRTIASPKGPVIQ | |
| | MYTNVDQDLVGWPAPQGSRSLTPCTCGSSDLYLVTR | |
| | HADVIPVRRRGDSRGSLLSPRPISYLKGSSGGPLLCPAG | |
| | HAVGLFRAAVCTRGVAKAVDFIPVENLETTMRSPVFT | |
| | DNSSPPAVTLTHPITKIDREVLYQEF | |
| NS4 cofactor | TGCVVIVGRIVLSG | 46 |
| HCV 5A/5B cut site | EDVVCCHSIYGKKK | 47 |
| HCV 4A/4B cut site | DEMEECSQH | — |
| Thy 1.1 mouse | QKVTSLTACLVNQNLRLDCRHENNTKDNSIQHEFSLT | 48 |
| | REKRKHVLSGTLGIPEHTYRSRVTLSNQPYIKVLTLAN | |
| | FTTKDEGDYFCELRVSGANPMSSNKSISVYRDKLVKC | |
| | GGISLLVQNTSWMLLLLLSLSLLQALDFISL | |
| Truncated epidermal growth | RKVCNGIGIGEFKDSLSINATNIKHFKNCTSISGDLHILP | 49 |
| factor receptor | VAFRGDSFTHTPPLDPQELDILKTVKEITGFLLIQAWPE | |
| | NRTDLHAFENLEIIRGRTKQHGQFSLAVVSLNITSLGLR | |
| | SLKEISDGDVIISGNKNLCYANTINWKKLFGTSGQKTKI | |
| | ISNRGENSCKATGQVCHALCSPEGCWGPEPRDCVSCR | |
| | NVSRGRECVDKCNLLEGEPREFVENSECIQCHPECLPQ | |
| | AMNITCTGRGPDNCIQCAHYIDGPHCVKTCPAGVMGE | |
| | NNTLVWKYADAGHVCHLCHPNCTYGCTGPGLEGCPT | |
| | NGPKIPSIATGMVGALLLLLVVALGIGLFM | |
| EGFP | MVSKGEELFTGVVPILVELDGDVNGHKFSVSGEGEGD | 50 |
| | ATYGKLTLKFICTTGKLPVPWPTLVTTLTYGVQCFSRY | |
| | PDHMKQHDFFKSAMPEGYVQERTIFFKDDGNYKTRA | |
| | EVKFEGDTLVNRIELKGIDFKEDGNILGHKLEYNYNSH | |
| | NVYIMADKQKNGIKVNFKIRHNIEDGSVQLADHYQQN | |
| | TPIGDGPVLLPDNHYLSTQSALSKDPNEKRDHMVLLEF | |
| | VTAAGITLGMDELYK | |
| Truncated CD3z | RAKFSRSAETAANLQDT | 51 |
| Truncated CD3z | FDALHMQTLAPR | 52 |
| Zap70 Interdomain + kinase | PNSSASAAVAAPTLPAHPSTFTQPQRRVDTLNSDGYTP | 53 |
| domain (mouse) | EPARLASSTDKPRPMPMDTSVYESPYSDPEELKDKKLF | |
| | LKRENLLVADIELGCGNFGSVRQGVYRMRKKQIDVAI | |
| | KVLKQGTEKADKDEMMREAQIMHQLDNPYIVRLIGV | |
| | CQAEALMLVMEMAGGGPLHKFLLGKKEEIPVSNVAE | |
| | LLHQVAMGMKYLEEKNFVHRDLAARNVLLVNRHYA | |
| | KISDFGLSKALGADDSYYTARSAGKWPLKWYAPECIN | |
| | FRKFSSRSDVWSYGVTMWEAFSYGQKPYKKMKGPEV | |
| | LDFIKQGKRMECPPECPPEMYALMSDCWIYKWEDRPD | |
| | FLTVEQRMRNYYYSL | |
| Zap70 Interdomain + kinase | PNSSASNASGAAAPTLPAHPSTLTHPQRRIDTLNSDGY | 54 |
| domain (human) | TPEPARITSPDKPRPMPMDTSVYESPYSDPEELKDKKL | |
| | FLKRDNLLIADIELGCGNFGSVRQGVYRMRKKQIDVAI | |
| | KVLKQGTEKADTEEMMREAQIMHQLDNPYIVRLIGVC | |
| | QAEALMLVMEMAGGGPLHKFLVGKREEIPVSNVAEL | |
| | LHQVSMGMKYLEEKNFVHRDLAARNVLLVNRHYAKI | |
| | SDFGLSKALGADDSYYTARSAGKWPLKWYAPECINFR | |
| | KFSSRSDVWSYGVTMWEALSYGQKPYKKMKGPEVM | |
| | AFIEQGKRMECPPECPPELYALMSDCWIYKWEDRPDF | |
| | LTVEQRMRACYYSL | |

TABLE 3
Exemplary Construct Sequences For The
Intein-Based Trans-Splicing System

| | | SEQ ID |
|---|---|---|
| | | NO: |
| | | (Example |
| Name | Sequence | FIGS.) |
| FLAG gp41-1 N BFP | MASPLTRFLSLNLLLLGESIILGSGEAEFTDYKDD | 55 |
| Linker (e.g., GSG) | DDKGSGTRSGYCLDLKTQVQTPQGMKEISNIQVGD | (FIGS. |
| underlined | LVLSNTGYNEVLNVFPKSKKKSYKITLEDGKEIIC | 2B, 8B) |
| Intein in bold | SEEHLFPTQTGEMNISGGLKEGMCLYVKEGSGRAK | |
| Furin cleavage | RATNFSLLKQAGDVEENPGPTRTGLESMSELIKEN | |
| site (e.g., RAKR) | MHMKLYMEGTVDNHHFKCTSEGEGKPYEGTQTMRI | |
| italicized | KVVEGGPLPFAFDILATSFLYGSKTFINHTQGIPD | |
| | FFKQSFPEGFTWERVTTYEDGGVLTATQDTSLQDG | |
| | CLIYNVKIRGVNFTSNGPVMQKKTLGWEAFTETLY | |
| | PADGGLEGRNDMALKLVGGSHLIANIKTTYRSKKP | |
| | AKNLKMPGVYYVDYRLERIKEANNETYVEQHEVAV | |
| | ARYCDLPSKLGHKLN | |
| gp41-1 C myc CD8EC | METDTLLLWVLLLWVPGSTGMMLKKILKIEELDER | 56 |
| CD3z delta E2A | ELIDIEVSGNHLFYANDILTHNSSSDVEQKLISEE | (FIG. 2B) |
| Thy1.1 | DLAAASTTTKPVLRTPSPVHPTGTSQPQRPEDCRP | |
| Intein in bold | RGSVKGTGLDFACDIYIWAPLAGICVALLLSLIIT | |
| | LICYRAKFSRSAETAANLQDTYDALHMQTLAPRGT | |
| | GQCTNYALLKLAGDVESNPGPGSMNPAISVALLLS | |
| | VLQVSRGQKVTSLTACLVNQNLRLDCRHENNTKDN | |
| | SIQHEFSLTREKRKHVLSGTLGIPEHTYRSRVTLS | |
| | NQPYIKVLTLANFTTKDEGDYFCELRVSGANPMSS | |
| | NKSISVYRDKLVKCGGISLLVQNTSWMLLLLLSLS | |
| | LLQALDFISL | |
| Thy1.1 P2A RQR | MNPAISVALLLSVLQVSRGQKVTSLTACLVNQNLR | 57 |
| FLAG CD8EC CD8TM | LDCRHENNTKDNSIQHEFSLTREKRKHVLSGTLGI | (FIG. 3B) |
| tCD3z AEY Cfa N | PEHTYRSRVTLSNQPYIKVLTLANFTTKDEGDYFC | |
| G4S2 Kir RXR | ELRVSGANPMSSNKSISVYRDKLVKCGGISLLVQN | |
| Linker (e.g., GSS, | TSWMLLLLLSLSLLQALDFISLGSGATNFSLLKQA | |
| TSGGGGSGGGSG) | GDVEENPGPEFTMETDTLLLWVLLLWVPGSTGCPY | |
| underlined | SNPSLCSGGGGSELPTQGTFSNVSTNVSPAKPTTT | |
| Intein in bold | ACPYSNPSLCGGGGSGGGSDYKDDDDKAAASTTTK | |
| Truncated | PVLRTPSPVHPTGTSQPQRPEDCRPRGSVKGTGLD | |
| CD3z (e.g., | FACDIYIWAPLAGICVALLLSLIITLICYRAKFSR | |
| RAKFSRSAETAANLQDT) | SAETAANLQDTGSSAEYCLSYDTEILTVEYGFLPI | |
| italicized | GKIVEERIECTVYTVDKNGFVYTQPIAQWHNRGEQ | |
| | EVFEYCLEDGSIIRATKDHKFMTTDGQMLPIDEIF | |
| | ERGLDLKQVDGLPTSGGGGSGGGSGTGLLDALTLA | |
| | SSRGPLRKRSVAVAKAKPKFSISPDSLS | |
| Cfa C CD28z EGFP | MVKIISRKSLGTQNVYDIGVEKDHNFLLKNGLVAS | 58 |
| Linker (e.g., GTG) | NCFNGTGNSRRNRLLQSDYMNMTPRRPGLTRKPYQ | (FIGS. |
| underlined | PYAPARDFAAYRPRAKFSRSAETAANLQDPNQLYN | 3B, |
| Intein in bold | ELNLGRREEYDVLEKKRARDPEMGGKQQRRRNPQE | 5C-5D, |
| | GVYNALQKDKMAEAYSEIGTKGERRRGKGHDGLYQ | 6C-6D) |
| | GLSTATKDTYDALHMQTLAPRGSGQCTNYALLKLA | |
| | GDVESNPGPLESMVSKGEELFTGVVPILVELDGDV | |
| | NGHKFSVSGEGEGDATYGKLTLKFICTTGKLPVPW | |
| | PTLVTTLTYGVQCFSRYPDHMKQHDFFKSAMPEGY | |
| | VQERTIFFKDDGNYKTRAEVKFEGDTLVNRIELKG | |
| | IDFKEDGNILGHKLEYNYNSHNVYIMADKQKNGIK | |
| | VNFKIRHNIEDGSVQLADHYQQNTPIGDGPVLLPD | |
| | NHYLSTQSALSKDPNEKRDHMVLLEFVTAAGITLG | |
| | MDELYK | |
| Thy1.1 P2A 1D3 | MNPAISVALLLSVLQVSRGQKVTSLTACLVNQNLR | 59 |
| CD19 hCD34 CD8EC | LDCRHENNTKDNSIQHEFSLTREKRKHVLSGTLGI | (FIGS. |
| CD8TM tCD3z AEY | PEHTYRSRVTLSNQPYIKVLTLANFTTKDEGDYFC | 3C-3D, |
| Cfa N G4S2 | ELRVSGANPMSSNKSISVYRDKLVKCGGISLLVQN | 12C) |
| EEKKMP E3 19K | TSWMLLLLLSLSLLQALDFISLGSGATNFSLLKQA | |
| Linker (e.g., GSS, | GDVEENPGPEFTMASPLTRFLSLNLLLLGESIILG | |
| TSGGGGSGGGSGTG) | SGEAEVQLQQSGAELVRPGTSVKLSCKVSGDTITF | |
| underlined | YYMHFVKQRPGQGLEWIGRIDPEDESTKYSEKFKN | |
| Intein in bold | KATLTADTSSNTAYLKLSSLTSEDTATYFCIYGGY | |
| Truncated | YFDYWGQGVMVTVSSGGGGSGGGGSGGGGSDIQMT | |
| CD3z (e.g., | QSPASLSTSLGETVTIQCQASEDIYSGLAWYQQKP | |
| RAKFSRSAETAANLQDT) | GKSPQLLIYGASDLQDGVPSRFSGSGSGTQYSLKI | |
| italicized | TSMQTEDEGVYFCQQGLTYPRTFGGGTKLELKRGG | |
| | GGSELPTQGTFSNVSTNVSGGGGSGGGSGSLSTTT | |
| | KPVLRTPSPVHPTGTSQPQRPEDCRPRGSVKGTGL | |
| | DFACDIYIWAPLAGICVALLLSLIITLICYRAKFS | |
| | RSAETAANLQDTGSSAEYCLSYDTEILTVEYGFLP | |
| | IGKIVEERIECTVYTVDKNGFVYTQPIAQWHNRGE | |
| | QEVFEYCLEDGSIIRATKDHKFMTTDGQMLPIDEI | |
| | FERGLDLKQVDGLPTSGGGGSGGGSGTGKYKSRRS | |
| | FIEEKKMP | |
| CD19 scFv gp41-1N | MASPLTRFLSLNLLLLGESIILGSGEAEVQLQQSG | 60 |
| CD20 scFv gp41-1N | AELVRPGTSVKLSCKVSGDTITFYYMHFVKQRPGQ | (FIGS. |
| FLAG-BFP Linker | GLEWIGRIDPEDESTKYSEKFKNKATLTADTSSNT | 4C-4D, |
| (e.g., GSG) | AYLKLSSLTSEDTATYFCIYGGYYFDYWGQGVMVT | 5C-5D) |
| underlined | VSSGGGGSGGGGSGGGGSDIQMTQSPASLSTSLGE | |
| Intein in bold | TVTIQCQASEDIYSGLAWYQQKPGKSPQLLIYGAS | |
| | DLQDGVPSRFSGSGSGTQYSLKITSMQTEDEGVYF | |
| | CQQGLTYPRTFGGGTKLELKREFTDYKDDDDKGSG | |
| | TRSGYCLDLKTQVQTPQGMKEISNIQVGDLVLSNT | |
| | GYNEVLNVFPKSKKKSYKITLEDGKEIICSEEHLF | |
| | PTQTGEMNISGGLKEGMCLYVKEGSGRAKRATNFS | |
| | LLKQAGDVEENPGPTRTMETDTLLLWVLLLWVPGS | |
| | TGQIVMSQSPAILSASPGEKVTMTCRARSSVSYIH | |
| | WYQQKPGSSPKPWIYATSNLASGVPGRFSGSGSGT | |
| | SYSLTITRVEAEDAATYYCQQWSSKPPTFGGGTKL | |
| | EIKRGGGGSGGGGSGGGGSQVQLQQPGAELVRPGT | |
| | SVKLSCKASGYTFTSYWMHWIKQRPGQGLEWIGVI | |
| | DPSDNYTKYNQKFKGKATLTVDTSSSTAYMQLSSL | |
| | TSEDSAVYFCAREGYYGSSPWFAYWGQGTLVTVSS | |
| | RSGDYKDDDDKGSGTRSGYCLDLKTQVQTPQGMKE | |
| | ISNIQVGDLVLSNTGYNEVLNVFPKSKKKSYKITL | |
| | EDGKEIICSEEHLFPTQTGEMNISGGLKEGMCLYV | |
| | KEGSGRAKRQCTNYALLKLAGDVESNPGPGSLGSG | |
| | LESMSELIKENMHMKLYMEGTVDNHHFKCTSEGEG | |
| | KPYEGTQTMRIKVVEGGPLPFAFDILATSFLYGSK | |
| | TFINHTQGIPDFFKQSFPEGFTWERVTTYEDGGVL | |
| | TATQDTSLQDGCLIYNVKIRGVNFTSNGPVMQKKT | |
| | LGWEAFTETLYPADGGLEGRNDMALKLVGGSHLIA | |
| | NIKTTYRSKKPAKNLKMPGVYYVDYRLERIKEANN | |
| | ETYVEQHEVAVARYCDLPSKLGHKLN | |
| gp41-1C CD8EC CD28 | METDTLLLWVLLLWVPGSTGMMLKKILKIEELDER | 61 |
| CD3z E2A Thy1.1 | ELIDIEVSGNHLFYANDILTHNSSSDVEQKLISEE | (FIGS. |
| Intein in bold | DLAAASTTTKPVLRTPSPVHPTGTSQPQRPEDCRP | 4C-4D) |
| | RGSVKGTGLDFACDIYIWAPLAGICVALLLSLIIT | |
| | LICYNSRRNRLLQSDYMNMTPRRPGLTRKPYQPYA | |
| | PARDFAAYRPRAKFSRSAETAANLQDPNQLYNELN | |
| | LGRREEYDVLEKKRARDPEMGGKQQRRRNPQEGVY | |
| | NALQKDKMAEAYSEIGTKGERRRGKGHDGLYQGLS | |
| | TATKDTYDALHMQTLAPRGTGQCTNYALLKLAGDV | |
| | ESNPGPGSMNPAISVALLLSVLQVSRGQKVTSLTA | |
| | CLVNQNLRLDCRHENNTKDNSIQHEFSLTREKRKH | |
| | VLSGTLGIPEHTYRSRVTLSNQPYIKVLTLANFTT | |
| | KDEGDYFCELRVSGANPMSSNKSISVYRDKLVKCG | |
| | GISLLVQNTSWMLLLLLSLSLLQALDFISL | |
| 2MC57 CD34 CD28EC | METDTLLLWVLLLWVPGSTGQVQLQESGGGLVQAG | 62 |
| CD28TM tCD3z AEY | GSLRLSCAASGRDFATYSMAWFRQAPGKERESVAT | (FIGS. |
| Cfa N P2A 1D3 CD19 | ISWSGQRTRYADSVKGRFTISRDNAKNTVYLQMNS | 4G, |
| FLAG CD8EC CD8TM | LKPEDTAVYYCAMPRTWGEFPPTQYDSWGQGTQVT | 13F-13G, |
| tCD3z AEY Cfa N | VSSGGGGSELPTQGTFSNVSTNVSGGGGSGGGSEA | 14A, |
| G4S2 EEKKMP E3 19K | RLIEFMYPPPYLDNERSNGTIIHIKEKHLCHTQSS | 15C-15D, |
| Linker (e.g., | PKLFWALVVVAGVLFCYGLLVTVALCVIWTRAKFS | 17E-17G) |
| GSG, GSS, | RSAETAANLQDTGSGAEYCLSYDTEILTVEYGFLP | |
| TSGGGGSGGGSGTG) | IGKIVEERIECTVYTVDKNGFVYTQPIAQWHNRGE | |
| underlined | QEVFEYCLEDGSIIRATKDHKFMTTDGQMLPIDEI | |
| Intein in bold | FERGLDLKQVDGLPGSGATNFSLLKQAGDVEENPG | |
| Truncated | PEFTMASPLTRFLSLNLLLLGESIILGSGEAEVQL | |
| CD3z (e.g., | QQSGAELVRPGTSVKLSCKVSGDTITFYYMHFVKQ | |
| RAKFSRSAETAANLQDT) | RPGQGLEWIGRIDPEDESTKYSEKFKNKATLTADT | |
| italicized | SSNTAYLKLSSLTSEDTATYFCIYGGYYFDYWGQG | |
| | VMVTVSSGGGGSGGGGSGGGGSDIQMTQSPASLST | |
| | SLGETVTIQCQASEDIYSGLAWYQQKPGKSPQLLI | |
| | YGASDLQDGVPSRFSGSGSGTQYSLKITSMQTEDE | |
| | GVYFCQQGLTYPRTFGGGTKLELKRGSGDYKDDDD | |
| | KGSLSTTTKPVLRTPSPVHPTGTSQPQRPEDCRPR | |
| | GSVKGTGLDFACDIYIWAPLAGICVALLLSLIITL | |
| | ICYRAKFSRSAETAANLQDTGSSAEYCLSYDTEIL | |
| | TVEYGFLPIGKIVEERIECTVYTVDKNGFVYTQPI | |
| | AQWHNRGEQEVFEYCLEDGSIIRATKDHKFMTTDG | |
| | QMLPIDEIFERGLDLKQVDGLPTSGGGGSGGGSGT | |
| | GKYKSRRSFIEEKKMP | |
| Cfa C Zap 70 | MVKIISRKSLGTQNVYDIGVEKDHNFLLKNGLVAS | 63 |
| E2A EGFP | NCFNGTGPNSSASAAVAAPTLPAHPSTFTQPQRRV | (FIG. 4G) |
| Linker (e.g., GTG) | DTLNSDGYTPEPARLASSTDKPRPMPMDTSVYESP | |
| underlined | YSDPEELKDKKLFLKRENLLVADIELGCGNFGSVR | |
| Intein in bold | QGVYRMRKKQIDVAIKVLKQGTEKADKDEMMREAQ | |
| | IMHQLDNPYIVRLIGVCQAEALMLVMEMAGGGPLH | |
| | KFLLGKKEEIPVSNVAELLHQVAMGMKYLEEKNFV | |
| | HRDLAARNVLLVNRHYAKISDFGLSKALGADDSYY | |
| | TARSAGKWPLKWYAPECINFRKFSSRSDVWSYGVT | |
| | MWEAFSYGQKPYKKMKGPEVLDFIKQGKRMECPPE | |
| | CPPEMYALMSDCWIYKWEDRPDFLTVEQRMRNYYY | |
| | SLGTSRAKRGSGQCTNYALLKLAGDVESNPGPLES | |
| | MVSKGEELFTGVVPILVELDGDVNGHKFSVSGEGE | |
| | GDATYGKLTLKFICTTGKLPVPWPTLVTTLTYGVQ | |
| | CFSRYPDHMKQHDFFKSAMPEGYVQERTIFFKDDG | |
| | NYKTRAEVKFEGDTLVNRIELKGIDFKEDGNILGH | |
| | KLEYNYNSHNVYIMADKQKNGIKVNFKIRHNIEDG | |
| | SVQLADHYQQNTPIGDGPVLLPDNHYLSTQSALSK | |
| | DPNEKRDHMVLLEFVTAAGITLGMDELYK | |
| Thy1.1 P2A V5 | MNPAISVALLLSVLQVSRGQKVTSLTACLVNQNLR | 64 |
| gp41-1 CD8EC | LDCRHENNTKDNSIQHEFSLTREKRKHVLSGTLGI | (FIGS. |
| CD8TM tCD3z AEY | PEHTYRSRVTLSNQPYIKVLTLANFTTKDEGDYFC | 5C/D) |
| Cfa N E3 19K | ELRVSGANPMSSNKSISVYRDKLVKCGGISLLVQN | |
| Linker (e.g., | TSWMLLLLLSLSLLQALDFISLGSGATNFSLLKQA | |
| GGGGSGGGS, | GDVEENPGPEFTMETDTLLLWVLLLWVPGSTGGKP | |
| GSL) underlined | IPNPLLGLDSTGGGGSGGGSMMLKKILKIEELDER | |
| Intein in bold | ELIDIEVSGNHLFYANDILTHNSSSDVGSLSTTTK | |
| | PVLRTPSPVHPTGTSQPQRPEDCRPRGSVKGTGLD | |
| | FACDIYIWAPLAGICVALLLSLIITLICYRAKFSR | |
| | SAETAANLQDTGSSAEYCLSYDTEILTVEYGFLPI | |
| | GKIVEERIECTVYTVDKNGFVYTQPIAQWHNRGEQ | |
| | EVFEYCLEDGSIIRATKDHKFMTTDGQMLPIDEIF | |
| | ERGLDLKQVDGLPGTGKYKSRRSFIDEKKMP | |
| CD19/CD20 scFv FLAG- | MASPLTRFLSLNLLLLGESIILGSGEAEVQLQQSG | 65 |
| RR12EE345L -BFP | AELVRPGTSVKLSCKVSGDTITFYYMHFVKQRPGQ | (FIGS. |
| Linker (e.g., GSS, | GLEWIGRIDPEDESTKYSEKFKNKATLTADTSSNT | 6C/D) |
| GSG, GSGGSG) | AYLKLSSLTSEDTATYFCIYGGYYFDYWGQGVMVT | |
| underlined | VSSGGGGSGGGGSGGGGSDIQMTQSPASLSTSLGE | |
| Leucine zippers in | TVTIQCQASEDIYSGLAWYQQKPGKSPQLLIYGAS | |
| lower case | DLQDGVPSRFSGSGSGTQYSLKITSMQTEDEGVYF | |
| | CQQGLTYPRTFGGGTKLELKREFTDYKDDDDKlei | |
| | raaflrqrntalrtevaeleqevqrlenevsqyet | |
| | rygplgggkGSGRAKRATNFSLLKQAGDVEENPGP | |
| | TRTMETDTLLLWVLLLWVPGSTGQIVMSQSPAILS | |
| | ASPGEKVTMTCRARSSVSYIHWYQQKPGSSPKPWI | |
| | YATSNLASGVPGRFSGSGSGTSYSLTITRVEAEDA | |
| | ATYYCQQWSSKPPTFGGGTKLEIKRGGGGSGGGGS | |
| | GGGGSQVQLQQPGAELVRPGTSVKLSCKASGYTFT | |
| | SYWMHWIKQRPGQGLEWIGVIDPSDNYTKYNQKFK | |
| | GKATLTVDTSSSTAYMQLSSLTSEDSAVYFCAREG | |
| | YYGSSPWFAYWGQGTLVTVSSRSGDYKDDDDKlei | |
| | raaflrqrntalrtevaeleqevqrlenevsqyet | |
| | rygplgggkGSGGSGRAKRQCTNYALLKLAGDVES | |
| | NPGPGSLGSGLESMSELIKENMHMKLYMEGTVDNH | |
| | HFKCTSEGEGKPYEGTQTMRIKVVEGGPLPFAFDI | |
| | LATSFLYGSKTFINHTQGIPDFFKQSFPEGFTWER | |
| | VTTYEDGGVLTATQDTSLQDGCLIYNVKIRGVNFT | |
| | SNGPVMQKKTLGWEAFTETLYPADGGLEGRNDMAL | |
| | KLVGGSHLIANIKTTYRSKKPAKNLKMPGVYYVDY | |
| | RLERIKEANNETYVEQHEVAVARYCDLPSKLGHKL | |
| | N | |
| Thy1.1 P2A V5 | MNPAISVALLLSVLQVSRGQKVTSLTACLVNQNLR | 66 |
| EE12RR345L IgG1- | LDCRHENNTKDNSIQHEFSLTREKRKHVLSGTLGI | (FIGS. |
| hinge tCD3z AEY | PEHTYRSRVTLSNQPYIKVLTLANFTTKDEGDYFC | 6C/D) |
| Cfa N E3 19K | ELRVSGANPMSSNKSISVYRDKLVKCGGISLLVQN | |
| Linker (e.g., | TSWMLLLLLSLSLLQALDFISLGSGATNFSLLKQA | |
| GSS, GTG) | GDVEENPGPEFTMETDTLLLWVLLLWVPGSTGGSG | |
| underlined | GKPIPNPLLGLDSTGGGGSGGGSleieaafleren | |
| Intein in bold | taletrvaeIrqrvqrlrnrvsqyrtrygplgggk | |
| Truncated | AAAVPRDCGCKPCICTGSLFWALVVVAGVLFCYGL | |
| CD3z (e.g., | LVTVALCVIWTRAKFSRSAETAANLQDTGSSAEYC | |
| RAKFSRSAETAANLQDT) | LSYDTEILTVEYGFLPIGKIVEERIECTVYTVDKN | |
| italicized | GFVYTQPIAQWHNRGEQEVFEYCLEDGSIIRATKD | |
| Leucine zipper in | HKFMTTDGQMLPIDEIFERGLDLKQVDGLPGTGKY | |
| lower case | KSRRSFIDEKKMP | |
| Thy1.1 P2A V5 | MNPAISVALLLSVLQVSRGQKVTSLTACLVNQNLR | 67 |
| gp41-1 CD8EC | LDCRHENNTKDNSIQHEFSLTREKRKHVLSGTLGI | (FIG. 8B) |
| CD28TM tCD3z AEY | PEHTYRSRVTLSNQPYIKVLTLANFTTKDEGDYFC | |
| Cfa N G4S2 E3 19K | ELRVSGANPMSSNKSISVYRDKLVKCGGISLLVQN | |
| EEKKMP | TSWMLLLLLSLSLLQALDFISLGSGATNFSLLKQA | |
| Linker (e.g., GSS, | GDVEENPGPEFTMETDTLLLWVLLLWVPGSTGGKP | |
| GSL, STGGGGSGGGS, | IPNPLLGLDSTGGGGSGGGSMMLKKILKIEELDER | |
| TSGGGGSGGGSGTG) | ELIDIEVSGNHLFYANDILTHNSSSDVGSLSTTTK | |
| underlined | PVLRTPSPVHPTGTSQPQRPEDCRPRGSVKGTGLD | |
| Intein in bold | FACDGSLFWALVVVAGVLFCYGLLVTVALCVIWTR | |
| Truncated | AKFSRSAETAANLQDTGSSAEYCLSYDTEILTVEY | |
| CD3z (e.g., | GFLPIGKIVEERIECTVYTVDKNGFVYTQPIAQWH | |
| RAKFSRSAETAANLQ) | NRGEQEVFEYCLEDGSIIRATKDHKFMTTDGQMLP | |
| italicized | IDEIFERGLDLKQVDGLPTSGGGGSGGGSGTGKYK | |
| | SRRSFIEEKKMP | |
| EGFP Cfa C CD3z | MVSKGEELFTGVVPILVELDGDVNGHKFSVSGEGE | 68 |
| delta GP41-8 N | GDATYGKLTLKFICTTGKLPVPWPTLVTTLTYGVQ | (FIG. 8B) |
| EEKKMP E3 19K | CFSRYPDHMKQHDFFKSAMPEGYVQERTIFFKDDG | |
| Linker | NYKTRAEVKFEGDTLVNRIELKGIDFKEDGNILGH | |
| (e.g., GSG, | KLEYNYNSHNVYIMADKQKNGIKVNFKIRHNIEDG | |
| GGGGSGGGSGTG) | SVQLADHYQQNTPIGDGPVLLPDNHYLSTQSALSK | |
| underlined | DPNEKRDHMVLLEFVTAAGITLGMDELYKGSGQCT | |
| Intein in bold | NYALLKLAGDVESNPGPRAKRGSGMVKIISRKSLG | |
| Truncated, | TQNVYDIGVEKDHNFLLKNGLVASNCFNGTGFDAL | |
| alternate | HMQTLAPRTSGSGSQLNRCLSLDTMVVTNGKAIEI | |
| CD3z (e.g., | RDVKVGDWLESECGPVQVTEVLPIIKQPVFEIVLK | |
| FDALHMQTLAPR) | SGKKIRVSANHKFPTKDGLKTINSGLKVGDFLRSR | |
| italicized | AKGGGGSGGGSGTGKYKSRRSFIEEKKMP | |
| gp41-8 C CD3z | MCEIFENEIDWDEIASIEYVGVEETIDINVTNDRL | 69 |
| delta EGFRt | FFANGILTHNSSAVEEGTGFDALHMQTLAPRGSGR | (FIG. 8B, |
| Linker (e.g., | AKRGSGQCTNYALLKLAGDVESNPGPLESMYSMQL | 3E-3I) |
| GTG) underlined | ASCVTLTLVLLVNSRKVCNGIGIGEFKDSLSINAT | |
| Intein in bold | NIKHFKNCTSISGDLHILPVAFRGDSFTHTPPLDP | |
| Truncated, | QELDILKTVKEITGFLLIQAWPENRTDLHAFENLE | |
| alternate | IIRGRTKQHGQFSLAVVSLNITSLGLRSLKEISDG | |
| CD3z (e.g., | DVIISGNKNLCYANTINWKKLFGTSGQKTKIISNR | |
| FDALHMQTLAPR) | GENSCKATGQVCHALCSPEGCWGPEPRDCVSCRNV | |
| italicized | SRGRECVDKCNLLEGEPREFVENSECIQCHPECLP | |
| | QAMNITCTGRGPDNCIQCAHYIDGPHCVKTCPAGV | |
| | MGENNTLVWKYADAGHVCHLCHPNCTYGCTGPGLE | |
| | GCPTNGPKIPSIATGMVGALLLLLVVALGIGLFM | |
| Q2 gp41-1 N BFP | MASPLTRFLSLNLLLLGESIILGSGEAEFTGSGEL | 70 |
| Linker (e.g., GSG, | PTQGTFSNVSTNVSGGGGSGGGSELPTQGTFSNVS | (FIG. 8C) |
| GGGGSGGGSELPTQGTFS | TNVSGGGGSGGGSRSGTRSGYCLDLKTQVQTPQGM | |
| NVSTNVSGGGGSGGGSRS | KEISNIQVGDLVLSNTGYNEVLNVFPKSKKKSYKI | |
| G) underlined | TLEDGKEIICSEEHLFPTQTGEMNISGGLKEGMCL | |
| Intein in bold | YVKEGSGRAKRATNFSLLKQAGDVEENPGPTRTGL | |
| | ESMSELIKENMHMKLYMEGTVDNHHFKCTSEGEGK | |
| | PYEGTQTMRIKVVEGGPLPFAFDILATSFLYGSKT | |
| | FINHTQGIPDFFKQSFPEGFTWERVTTYEDGGVLT | |
| | ATQDTSLQDGCLIYNVKIRGVNFTSNGPVMQKKTL | |
| | GWEAFTETLYPADGGLEGRNDMALKLVGGSHLIAN | |
| | IKTTYRSKKPAKNLKMPGVYYVDYRLERIKEANNE | |
| | TYVEQHEVAVARYCDLPSKLGHKLN | |
| Thy1.1 P2A 1D3 CD19 | MNPAISVALLLSVLQVSRGQKVTSLTACLVNQNLR | 71 |
| hCD34 CD8EC CD8TM | LDCRHENNTKDNSIQHEFSLTREKRKHVLSGTLGI | (FIG. 9C) |
| tCD3z AES CL N | PEHTYRSRVTLSNQPYIKVLTLANFTTKDEGDYFC | |
| FKBP12-DmRA G4S2 | ELRVSGANPMSSNKSISVYRDKLVKCGGISLLVQN | |
| EEKKMP E3 19K | TSWMLLLLLSLSLLQALDFISLGSGATNFSLLKQA | |
| Linker (e.g., | GDVEENPGPEFTMASPLTRFLSLNLLLLGESIILG | |
| GSSGSG, GSGGTR) | SGEAEVQLQQSGAELVRPGTSVKLSCKVSGDTITF | |
| underlined | YYMHFVKQRPGQGLEWIGRIDPEDESTKYSEKFKN | |
| Intein in bold | KATLTADTSSNTAYLKLSSLTSEDTATYFCIYGGY | |
| Truncated | YFDYWGQGVMVTVSSGGGGSGGGGSGGGGSDIQMT | |
| CD3z (e.g., | QSPASLSTSLGETVTIQCQASEDIYSGLAWYQQKP | |
| RAKFSRSAETAANLQDT) | GKSPQLLIYGASDLQDGVPSRFSGSGSGTQYSLKI | |
| italicized | TSMQTEDEGVYFCQQGLTYPRTFGGGTKLELKRGG | |
| FKBP12 in lower | GGSELPTQGTFSNVSTNVSGGGGSGGGSGSLSTTT | |
| case | KPVLRTPSPVHPTGTSQPQRPEDCRPRGSVKGTGL | |
| | DFACDIYIWAPLAGICVALLLSLIITLICYRAKFS | |
| | RSAETAANLQDTGSSGSGYIDTDSVVGDTIIDVSG | |
| | KKMTIAEFYDSTPDGSGGTRgvqvetispgdgrtf | |
| | pkrgqtcvvhytgmledgkkfdssrdrnkpfkfml | |
| | gkqevirgweegvaqmsvgqrakltispdyaygat | |
| | ghpgiipphatlvfdvellkleTSGGGGSGGGSGT | |
| | GKYKSRRSFIEEKKMP | |
| FRB AES CL C | milwhemwhegleeasrlyfgernvkgmfevlepl | 72 |
| CD28z EGFP | hammergpqtlketsfnqaygrdlmeaqewcrkym | (FIG. 9C) |
| Linker (e.g., | ksgnvkdllqawdlyyhvfrriskGSGGGGSGGGS | |
| GSGGGGSGGGS, | EARDWVKRVGGKTSLSVNTYSGEVERKNINYIMKH | |
| GGGGSGTG) | TVKKRMFKIKAGGKEVIVTADHSVMVKRDGKIIDV | |
| underlined | KPTEMKQTDRVVKWMLTGSHMIEFIEFEIEDLGVM | |
| Intein in bold | EIDVYDIEVDGNHNFFGNDILVHNSVYLNGGGGSG | |
| FRB* in lower | TGNSRRNRLLQSDYMNMTPRRPGLTRKPYQPYAPA | |
| case | RDFAAYRPRAKFSRSAETAANLQDPNQLYNELNLG | |
| | RREEYDVLEKKRARDPEMGGKQQRRRNPQEGVYNA | |
| | LQKDKMAEAYSEIGTKGERRRGKGHDGLYQGLSTA | |
| | TKDTYDALHMQTLAPRGSGQCTNYALLKLAGDVES | |
| | NPGPLESMVSKGEELFTGVVPILVELDGDVNGHKF | |
| | SVSGEGEGDATYGKLTLKFICTTGKLPVPWPTLVT | |
| | TLTYGVQCFSRYPDHMKQHDFFKSAMPEGYVQERT | |
| | IFFKDDGNYKTRAEVKFEGDTLVNRIELKGIDFKE | |
| | DGNILGHKLEYNYNSHNVYIMADKQKNGIKVNFKI | |
| | RHNIEDGSVQLADHYQQNTPIGDGPVLLPDNHYLS | |
| | TQSALSKDPNEKRDHMVLLEFVTAAGITLGMDELY | |
| | K | |
| 1D3 CD19 ST CD28EC | METDTLLLWVLLLWVPGSTGEVQLQQSGAELVRPG | 73 |
| CD28TM AES CL N | TSVKLSCKVSGDTITFYYMHFVKQRPGQGLEWIGR | (FIGS. |
| FKBP12 P2A 2MC27 | IDPEDESTKYSEKFKNKATLTADTSSNTAYLKLSS | 10C/D) |
| CD20 VHH hCD34 | LTSEDTATYFCIYGGYYFDYWGQGVMVTVSSGGGG | |
| CD8EC CD8TM tCD3z | SGGGGSGGGGSDIQMTQSPASLSTSLGETVTIQCQ | |
| AES CL N FKBP12 | ASEDIYSGLAWYQQKPGKSPQLLIYGASDLQDGVP | |
| G4S2 EEKKMP | SRFSGSGSGTQYSLKITSMQTEDEGVYFCQQGLTY | |
| E3 19K | PRTFGGGTKLELKRSAWSHPQFEKGGGSGGGSGGS | |
| Linker (e.g., | AWSHPQFEKGGGGSGGGSEARLIEFMYPPPYLDNE | |
| GSSGSG, GSGGTR) | RSNGTIIHIKEKHLCHTQSSPKLFWALVVVAGVLF | |
| underlined | CYGLLVTVALCVIWTRAKFSRSAETAANLQDTGSS | |
| Intein in bold | GSGYIDTDSVVGDTIIDVSGKKMTIAEFYDSTPDG | |
| Truncated | SGGTRgvqvetispgdgrtfpkrgqtcvvhytgml | |
| CD3z (e.g., | edgkkfdssrdrnkpfkfmlgkqevirgweegvaq | |
| RAKFSRSAETAANLQDT) | msvgqrakltispdyaygatghpgiipphatlvfd | |
| italicized | vellkleGRSSGATNFSLLKQAGDVEENPGPEFTM | |
| FKBP12 in | ASPLTRFLSLNLLLLGESIILGSGEAQVQLQESGG | |
| lower case | GLVQAGGSLRLSCAASGRDFATYSMAWFRQAPGKE | |
| | RESVATISWSGQRTRYADSVKGRFTISRDNAKNTV | |
| | YLQMNSLKPEDTAVYYCAMPRTWGEFPPTQYDSWG | |
| | QGTQVTVSSGGGGSELPTQGTFSNVSTNVSGGGGS | |
| | GGGSGSLSTTTKPVLRTPSPVHPTGTSQPQRPEDC | |
| | RPRGSVKGTGLDFACDIYIWAPLAGICVALLLSLI | |
| | ITLICYrakfsrsaetaanlqdtGSSGSGYIDTDS | |
| | VVGDTIIDVSGKKMTIAEFYDSTPDGSGGTRgvqv | |
| | etispgdgrtfpkrgqtcvvhytgmledgkkfdss | |
| | rdrnkpfkfmlgkqevirgweegvaqmsvgqrakl | |
| | tispdyaygatghpgiipphatlvfdvellkleTS | |
| | GGGGSGGGSGTGKYKSRRSFIEEKKMP | |
| Thy1.1 P2A IL-7 | MNPAISVALLLSVLQVSRGQKVTSLTACLVNQNLR | 74 |
| hCD34 CD8EC | LDCRHENNTKDNSIQHEFSLTREKRKHVLSGTLGI | (FIG. |
| CD8TM tCD3z | PEHTYRSRVTLSNQPYIKVLTLANFTTKDEGDYFC | 11C) |
| AEY Cfa N G4S2 | ELRVSGANPMSSNKSISVYRDKLVKCGGISLLVQN | |
| EEKKMP E3 19K | TSWMLLLLLSLSLLQALDFISLGSGATNFSLLKQA | |
| Linker (e.g., GSS, | GDVEENPGPEFTMFHVSFRYIFGIPPLILVLLPVT | |
| TSGGGGSGGGSGTG) | SSECHIKDKEGKAYESVLMISIDELDKMTGTDSNC | |
| underlined | PNNEPNFFRKHVCDDTKEAAFLNRAARKLKQFLKM | |
| Intein in bold | NISEEFNVHLLTVSQGTQTLVNCTSKEEKNVKEQK | |
| Truncated | KNDACFLKRLLREIKTCWNKILKGSIGGGGRSELP | |
| CD3z (e.g., | TQGTFSNVSTNVSGGGGSGSLSTTTKPVLRTPSPV | |
| RAKFSRSAETAANLQDT) | HPTGTSQPQRPEDCRPRGSVKGTGLDFACDIYIWA | |
| italicized | PLAGICVALLLSLIITLICYRAKFSRSAETAANLQ | |
| | DTGSSAEYCLSYDTEILTVEYGFLPIGKIVEERIE | |
| | CTVYTVDKNGFVYTQPIAQWHNRGEQEVFEYCLED | |
| | GSIIRATKDHKFMTTDGQMLPIDEIFERGLDLKQV | |
| | DGLPTSGGGGSGGGSGTGKYKSRRSFIEEKKMP | |
| Thy1.1 P2A IL-7 | MNPAISVALLLSVLQVSRGQKVTSLTACLVNQNLR | 75 |
| hCD34 CD8EC CD8TM | LDCRHENNTKDNSIQHEFSLTREKRKHVLSGTLGI | (FIG. |
| tCD3z AES CL N | PEHTYRSRVTLSNQPYIKVLTLANFTTKDEGDYFC | 11C) |
| FKBP12-DmRA | ELRVSGANPMSSNKSISVYRDKLVKCGGISLLVQN | |
| G4S2 EEKKMP E3 19K | TSWMLLLLLSLSLLQALDFISLGSGATNFSLLKQA | |
| Linker (e.g., | GDVEENPGPEFTMASPLTRFLSLNLLLLGESIILG | |
| GSSGSG, GSGGTR) | SGEAEVQLQQSGAELVRPGTSVKLSCKVSGDTITF | |
| underlined | YYMHFVKQRPGQGLEWIGRIDPEDESTKYSEKFKN | |
| Intein in bold | KATLTADTSSNTAYLKLSSLTSEDTATYFCIYGGY | |
| Truncated | YFDYWGQGVMVTVSSGGGGSGGGGSGGGGSDIQMT | |
| CD3z (e.g., | QSPASLSTSLGETVTIQCQASEDIYSGLAWYQQKP | |
| RAKFSRSAETAANLQDT) | GKSPQLLIYGASDLQDGVPSRFSGSGSGTQYSLKI | |
| italicized | TSMQTEDEGVYFCQQGLTYPRTFGGGTKLELKRGG | |
| FKBP12 in lower | GGSELPTQGTFSNVSTNVSGGGGSGGGSGSLSTTT | |
| case | KPVLRTPSPVHPTGTSQPQRPEDCRPRGSVKGTGL | |
| | DFACDIYIWAPLAGICVALLLSLIITLICYRAKFS | |
| | RSAETAANLQDTGSSGSGYIDTDSVVGDTIIDVSG | |
| | KKMTIAEFYDSTPDGSGGTRgvqvetispgdgrtf | |
| | pkrgqtcvvhytgmledgkkfdssrdrnkpfkfml | |
| | gkqevirgweegvaqmsvgqrakltispdyaygat | |
| | ghpgiipphatlvfdvellkleTSGGGGSGGGSGT | |
| | GKYKSRRSFIEEKKMP | |
| Cfa C CD28 1XX | MVKIISRKSLGTQNVYDIGVEKDHNFLLKNGLVAS | 76 |
| DHFR-DD RGI EGFP | NCFNGTGNSRRNRLLQSDYMNMTPRRPGLTRKPYQ | (FIG. |
| Linker (e.g., GTG, | PYAPARDFAAYRPRAKFSRSAETAANLQDPNQLYN | 12C) |
| TSGGGGSGGGSGTG) | ELNLGRREEYDVLEKKRARDPEMGGKQQRRRNPQE | |
| underlined | GVFNALQKDKMAEAFSEIGTKGERRRGKGHDGLFQ | |
| Intein in bold | GLSTATKDTFDALHMQTLAPRGTEARLisliaala | |
| DHFR-DD in lower | vdyvigmenampwnlpadlawfkrntlnkpvimgr | |
| case | htwesigrplpgrkniilssqpstddrvtwvksvd | |
| | eaiaacgdvpeimvigggrvieqflpkaqklylth | |
| | idaevegdthfpdyepddwesvfsefhdadaqnsh | |
| | sycfeilerrGSQCTNYALLKLAGDVESNPGPLES | |
| | MVSKGEELFTGVVPILVELDGDVNGHKFSVSGEGE | |
| | GDATYGKLTLKFICTTGKLPVPWPTLVTTLTYGVQ | |
| | CFSRYPDHMKQHDFFKSAMPEGYVQERTIFFKDDG | |
| | NYKTRAEVKFEGDTLVNRIELKGIDFKEDGNILGH | |
| | KLEYNYNSHNVYIMADKQKNGIKVNFKIRHNIEDG | |
| | SVQLADHYQQNTPIGDGPVLLPDNHYLSTQSALSK | |
| | DPNEKRDHMVLLEFVTAAGITLGMDELYK | |
| Cfa C CD28 1XX | MVKIISRKSLGTQNVYDIGVEKDHNFLLKNGLVAS | 77 |
| AI EGFP | NCFNGTGNSRRNRLLQSDYMNMTPRRPGLTRKPYQ | (FIGS. |
| Linker (e.g., | PYAPARDFAAYRPRAKFSRSAETAANLQDPNQLYN | 13B/C) |
| GTG, GSGTR) | ELNLGRREEYDVLEKKRARDPEMGGKQQRRRNPQE | |
| underlined | GVFNALQKDKMAEAFSEIGTKGERRRGKGHDGLFQ | |
| Intein in bold | GLSTATKDTFDALHMQTLAPRGSGTRgtggsgdem | |
| SMASh-tag in | eecsqhlpgagssgdimgssgtgsgsgtsapitay | |
| lower case | aqqtrgllgciitsltgrdknqvegevqimstatq | |
| | tflatcingvcwavyhgagtrtiaspkgpviqmyt | |
| | nvdqdlvgwpapqgsrsltpctcgssdlylvtrha | |
| | dvipvrrrgdgrgsllsprpisylkgssggpllcp | |
| | aghavglfraavctrgvakavdfipvenlettmrs | |
| | pvftdnssppavtlthpitkidtkyimtcmsadle | |
| | vvtstwvlvggvlaalaayclstgcvvivgrivls | |
| | gkpaiipdrevlyGSQCTNYALLKLAGDVESNPGP | |
| | LESMVSKGEELFTGVVPILVELDGDVNGHKFSVSG | |
| | EGEGDATYGKLTLKFICTTGKLPVPWPTLVTTLTY | |
| | GVQCFSRYPDHMKQHDFFKSAMPEGYVQERTIFFK | |
| | DDGNYKTRAEVKFEGDTLVNRIELKGIDFKEDGNI | |
| | LGHKLEYNYNSHNVYIMADKQKNGIKVNFKIRHNI | |
| | EDGSVQLADHYQQNTPIGDGPVLLPDNHYLSTQSA | |
| | LSKDPNEKRDHMVLLEFVTAAGITLGMDELYK | |
| Cfa C 5A-5B NS4a | MVKIISRKSLGTQNVYDIGVEKDHNFLLKNGLVAS | 78 |
| NS3 4A-4B Zap 70 | NCFNGTGEDVVCCHSIYGKKKGGGGSGGGSTGCVV | (FIG. |
| E2A EGFP | IVGRIVLSGSGTSAPITAYAQQTRGLLGCIITSLT | 14A) |
| Linker (e.g., | GRDKNQVEGEVQIVSTATQTFLATCINGVCWAVYH | |
| GTG, ALG, | GAGTRTIASPKGPVIQMYTNVDQDLVGWPAPQGSR | |
| GTSRAKRGSG) | SLTPCTCGSSDLYLVTRHADVIPVRRRGDSRGSLL | |
| underlined | SPRPISYLKGSSGGPLLCPAGHAVGLFRAAVCTRG | |
| Intein in bold | VAKAVDFIPVENLETTMRSPVFTDNSSPPAVTLTH | |
| HCV NS3 protease | PITKIDREVLYQEFDEMEECSQHALGpnssasaav | |
| and cleavage | aaptlpahpstftqpqrrvdtlnsdgytpeparla | |
| sites italicized | sstdkprpmpmdtsvyespysdpeelkdkklflkr | |
| Zap70 in lower | enllvadielgcgnfgsvrqgvyrmrkkqidvaik | |
| case | vlkqgtekadkdemmreaqimhqldnpyivrligv | |
| | cqaealmlvmemagggplhkfllgkkeeipvsnva | |
| | ellhqvamgmkyleeknfvhrdlaarnvllvnrhy | |
| | akisdfglskalgaddsyytarsagkwplkwyape | |
| | cinfrkfssrsdvwsygvtmweafsygqkpykkmk | |
| | gpevldfikqgkrmecppecppemyalmsdcwiyk | |
| | wedrpdfltveqrmrnyyyslGTSRAKRGSGQCTN | |
| | YALLKLAGDVESNPGPLESMVSKGEELFTGVVPIL | |
| | VELDGDVNGHKFSVSGEGEGDATYGKLTLKFICTT | |
| | GKLPVPWPTLVTTLTYGVQCFSRYPDHMKQHDFFK | |
| | SAMPEGYVQERTIFFKDDGNYKTRAEVKFEGDTLV | |
| | NRIELKGIDFKEDGNILGHKLEYNYNSHNVYIMAD | |
| | KQKNGIKVNFKIRHNIEDGSVQLADHYQQNTPIGD | |
| | GPVLLPDNHYLSTQSALSKDPNEKRDHMVLLEFVT | |
| | AAGITLGMDELYK | |
| Thy1.1 P2A 1D3 | MNPAISVALLLSVLQVSRGQKVTSLTACLVNQNLR | 79 |
| CD19 FLAG CD8EC | LDCRHENNTKDNSIQHEFSLTREKRKHVLSGTLGI | (FIG. |
| CD8TM tCD3z AEY | PEHTYRSRVTLSNQPYIKVLTLANFTTKDEGDYFC | 15B) |
| Cfa N G4S2 | ELRVSGANPMSSNKSISVYRDKLVKCGGISLLVQN | |
| EEKKMP E3 19K | TSWMLLLLLSLSLLQALDFISLGSGATNFSLLKQA | |
| Linker | GDVEENPGPEFTMASPLTRFLSLNLLLLGESIILG | |
| (e.g., GSS, | SGEAEVQLQQSGAELVRPGTSVKLSCKVSGDTITF | |
| TSGGGGSGGGSGTG) | YYMHFVKQRPGQGLEWIGRIDPEDESTKYSEKFKN | |
| underlined | KATLTADTSSNTAYLKLSSLTSEDTATYFCIYGGY | |
| Intein in bold | YFDYWGQGVMVTVSSGGGGSGGGGSGGGGSDIQMT | |
| Truncated | QSPASLSTSLGETVTIQCQASEDIYSGLAWYQQKP | |
| CD3z (e.g., | GKSPQLLIYGASDLQDGVPSRFSGSGSGTQYSLKI | |
| RAKFSRSAETAANLQDT) | TSMQTEDEGVYFCQQGLTYPRTFGGGTKLELKRGS | |
| italicized | GDYKDDDDKGSLSTTTKPVLRTPSPVHPTGTSQPQ | |
| | RPEDCRPRGSVKGTGLDFACDIYIWAPLAGICVAL | |
| | LLSLIITLICYRAKFSRSAETAANLQDTGSSAEYC | |
| | LSYDTEILTVEYGFLPIGKIVEERIECTVYTVDKN | |
| | GFVYTQPIAQWHNRGEQEVFEYCLEDGSIIRATKD | |
| | HKFMTTDGQMLPIDEIFERGLDLKQVDGLPTSGGG | |
| | GSGGGSGTGKYKSRRSFIEEKKMP | |
| Cfa C 5A-5B NS4a | MVKIISRKSLGTQNVYDIGVEKDHNFLLKNGLVAS | 80 |
| NS3 4A-4B | NCFNGTGedvvcchsiygkkkggggsgggstgcvv | (FIGS. |
| CD28z EGFP | ivgrivlsgsgtsapitayaqqtrgllgciitslt | 15B-15D) |
| Linker (e.g., | grdknqvegevqivstatqtflatcingvcwavyh | |
| GTG, ALG) | gagtrtiaspkgpviqmytnvdqdlvgwpapqgsr | |
| underlined | sltpctcgssdlylvtrhadvipvrrrgdsrgsll | |
| Intein in bold | sprpisylkgssggpllcpaghavglfraavctrg | |
| HCV NS3 protease | vakavdfipvenlettmrspvftdnssppavtlth | |
| and cleavage sites | pitkidrevlyqefdemeecsqhALGNSRRNRLLQ | |
| in lower case | SDYMNMTPRRPGLTRKPYQPYAPARDFAAYRPRAK | |
| | FSRSAETAANLQDPNQLYNELNLGRREEYDVLEKK | |
| | RARDPEMGGKQQRRRNPQEGVYNALQKDKMAEAYS | |
| | EIGTKGERRRGKGHDGLYQGLSTATKDTYDALHMQ | |
| | TLAPRGSGQCTNYALLKLAGDVESNPGPLESMVSK | |
| | GEELFTGVVPILVELDGDVNGHKFSVSGEGEGDAT | |
| | YGKLTLKFICTTGKLPVPWPTLVTTLTYGVQCFSR | |
| | YPDHMKQHDFFKSAMPEGYVQERTIFFKDDGNYKT | |
| | RAEVKFEGDTLVNRIELKGIDFKEDGNILGHKLEY | |
| | NYNSHNVYIMADKQKNGIKVNFKIRHNIEDGSVQL | |
| | ADHYQQNTPIGDGPVLLPDNHYLSTQSALSKDPNE | |
| | KRDHMVLLEFVTAAGITLGMDELYK | |
| Cfa C NS4a NS3 | MVKIISRKSLGTQNVYDIGVEKDHNFLLKNGLVAS | 81 |
| 4A-4B CD28z EGFP | NCFNgtgggggsgggstgcvvivgrivlsgsgtsa | (FIGS. |
| Linker (e.g., ALG) | pitayaqqtrgllgciitsltgrdknqvegevqiv | 15B-15D) |
| underlined | statqtflatcingvcwavyhgagtrtiaspkgpv | |
| Intein in bold | iqmytnvdqdlvgwpapqgsrsltpctcgssdlyl | |
| HCV NS3 protease | vtrhadvipvrrrgdsrgsllsprpisylkgssgg | |
| and cleavage sites | pllcpaghavglfraavctrgvakavdfipvenle | |
| in lower case | ttmrspvftdnssppavtlthpitkidrevlyqef | |
| | demeecsqhALGNSRRNRLLQSDYMNMTPRRPGLT | |
| | RKPYQPYAPARDFAAYRPRAKFSRSAETAANLQDP | |
| | NQLYNELNLGRREEYDVLEKKRARDPEMGGKQQRR | |
| | RNPQEGVYNALQKDKMAEAYSEIGTKGERRRGKGH | |
| | DGLYQGLSTATKDTYDALHMQTLAPRGSGQCTNYA | |
| | LLKLAGDVESNPGPLESMVSKGEELFTGVVPILVE | |
| | LDGDVNGHKFSVSGEGEGDATYGKLTLKFICTTGK | |
| | LPVPWPTLVTTLTYGVQCFSRYPDHMKQHDFFKSA | |
| | MPEGYVQERTIFFKDDGNYKTRAEVKFEGDTLVNR | |
| | IELKGIDFKEDGNILGHKLEYNYNSHNVYIMADKQ | |
| | KNGIKVNFKIRHNIEDGSVQLADHYQQNTPIGDGP | |
| | VLLPDNHYLSTQSALSKDPNEKRDHMVLLEFVTAA | |
| | GITLGMDELYK | |
| Cfa C 5A-5B NS4a | MVKIISRKSLGTQNVYDIGVEKDHNFLLKNGLVAS | 82 |
| NS3 CD28z EGFP | NCFNGTGedvvcchsiygkkkggggsgggstgcvv | (FIGS. |
| Linker (e.g., | ivgrivlsgsgtsapitayaqqtrgllgciitslt | 15B-15D) |
| GTG, ALG) | grdknqvegevqivstatqtflatcingvcwavyh | |
| underlined | gagtrtiaspkgpviqmytnvdqdlvgwpapqgsr | |
| Intein in bold | sltpctcgssdlylvtrhadvipvrrrgdsrgsll | |
| HCV NS3 protease | sprpisylkgssggpllcpaghavglfraavctrg | |
| and cleavage | vakavdfipvenlettmrspvftdnssppavtlth | |
| sites in lower | pitkidrevlyqefALGNSRRNRLLQSDYMNMTPR | |
| case | RPGLTRKPYQPYAPARDFAAYRPRAKFSRSAETAA | |
| | NLQDPNQLYNELNLGRREEYDVLEKKRARDPEMGG | |
| | KQQRRRNPQEGVYNALQKDKMAEAYSEIGTKGERR | |
| | RGKGHDGLYQGLSTATKDTYDALHMQTLAPRGSGQ | |
| | CTNYALLKLAGDVESNPGPLESMVSKGEELFTGVV | |
| | PILVELDGDVNGHKFSVSGEGEGDATYGKLTLKFI | |
| | CTTGKLPVPWPTLVTTLTYGVQCFSRYPDHMKQHD | |
| | FFKSAMPEGYVQERTIFFKDDGNYKTRAEVKFEGD | |
| | TLVNRIELKGIDFKEDGNILGHKLEYNYNSHNVYI | |
| | MADKQKNGIKVNFKIRHNIEDGSVQLADHYQQNTP | |
| | IGDGPVLLPDNHYLSTQSALSKDPNEKRDHMVLLE | |
| | FVTAAGITLGMDELYK | |
| Thy1.1 P2A 1D3 | MNPAISVALLLSVLQVSRGQKVTSLTACLVNQNLR | 83 |
| CD19 hCD34 CD8EC | LDCRHENNTKDNSIQHEFSLTREKRKHVLSGTLGI | (FIGS. |
| CD8TM tCD3z AEY | PEHTYRSRVTLSNQPYIKVLTLANFTTKDEGDYFC | 16A-16C) |
| AES CL N Cfa N | ELRVSGANPMSSNKSISVYRDKLVKCGGISLLVQN | |
| G4S2 EEKKMP E3 | TSWMLLLLLSLSLLQALDFISLGSGATNFSLLKQA | |
| 19K Linker (e.g., | GDVEENPGPEFTMASPLTRFLSLNLLLLGESIILG | |
| GSSGSG, GSGGS) | SGEAEVQLQQSGAELVRPGTSVKLSCKVSGDTITF | |
| underlined | YYMHFVKQRPGQGLEWIGRIDPEDESTKYSEKFKN | |
| Intein in bold | KATLTADTSSNTAYLKLSSLTSEDTATYFCIYGGY | |
| Truncated | YFDYWGQGVMVTVSSGGGGSGGGGSGGGGSDIQMT | |
| CD3z (e.g., | QSPASLSTSLGETVTIQCQASEDIYSGLAWYQQKP | |
| RAKFSRSAETAANLQDT) | GKSPQLLIYGASDLQDGVPSRFSGSGSGTQYSLKI | |
| italicized | TSMQTEDEGVYFCQQGLTYPRTFGGGTKLELKRGG | |
| | GGSELPTQGTFSNVSTNVSGGGGSGGGSGSLSTTT | |
| | KPVLRTPSPVHPTGTSQPQRPEDCRPRGSVKGTGL | |
| | DFACDIYIWAPLAGICVALLLSLIITLICYRAKFS | |
| | RSAETAANLQDTGSSGSGYIDTDSVVGDTIIDVSG | |
| | KKMTIAEFYDSTPDGSGGSGAEYCLSYDTEILTVE | |
| | YGFLPIGKIVEERIECTVYTVDKNGFVYTQPIAQW | |
| | HNRGEQEVFEYCLEDGSIIRATKDHKFMTTDGQML | |
| | PIDEIFERGLDLKQVDGLPTSGGGGSGGGSGTGKY | |
| | KSRRSFIEEKKMP | |
| Cfa C 5A-5B NS4a | MVKIISRKSLGTQNVYDIGVEKDHNFLLKNGLVAS | 84 |
| NS3 4A-4B AES CL | NCFNGTGedvvcchsiygkkkggggsgggstgcvv | (FIGS. |
| C CD28z EGFP | ivgrivlsgsgtsapitayaqqtrgllgciitslt | 16A-16C) |
| Linker (e.g., | grdknqvegevqivstatqtflatcingvcwavyh | |
| GTG, ALGGSGGGS, | gagtrtiaspkgpviqmytnvdqdlvgwpapqgsr | |
| GGGGSTS) | sltpctcgssdlylvtrhadvipvrrrgdsrgsll | |
| underlined | sprpisylkgssggpllcpaghavglfraavctrg | |
| Intein in bold | vakavdfipvenlettmrspvftdnssppavtlth | |
| HCV NS3 | pitkidrevlyqefdemeecsqhALGGSGGGSEAR | |
| protease and | DWVKRVGGKTSLSVNTYSGEVERKNINYIMKHTVK | |
| cleavage sites | KRMFKIKAGGKEVIVTADHSVMVKRDGKIIDVKPT | |
| in lower case | EMKQTDRVVKWMLTGSHMIEFIEFEIEDLGVMEID | |
| | VYDIEVDGNHNFFGNDILVHNSVYLNGGGGSTSNS | |
| | RRNRLLQSDYMNMTPRRPGLTRKPYQPYAPARDFA | |
| | AYRPRAKFSRSAETAANLQDPNQLYNELNLGRREE | |
| | YDVLEKKRARDPEMGGKQQRRRNPQEGVYNALQKD | |
| | KMAEAYSEIGTKGERRRGKGHDGLYQGLSTATKDT | |
| | YDALHMQTLAPRGSGQCTNYALLKLAGDVESNPGP | |
| | LESMVSKGEELFTGVVPILVELDGDVNGHKFSVSG | |
| | EGEGDATYGKLTLKFICTTGKLPVPWPTLVTTLTY | |
| | GVQCFSRYPDHMKQHDFFKSAMPEGYVQERTIFFK | |
| | DDGNYKTRAEVKFEGDTLVNRIELKGIDFKEDGNI | |
| | LGHKLEYNYNSHNVYIMADKQKNGIKVNFKIRHNI | |
| | EDGSVQLADHYQQNTPIGDGPVLLPDNHYLSTQSA | |
| | LSKDPNEKRDHMVLLEFVTAAGITLGMDELYK | |
| Thy1.1 P2A 1D3 | MNPAISVALLLSVLQVSRGQKVTSLTACLVNQNLR | 85 |
| CD19 FLAG CD8EC | LDCRHENNTKDNSIQHEFSLTREKRKHVLSGTLGI | (FIG. |
| CD8TM tCD3z AEY | PEHTYRSRVTLSNQPYIKVLTLANFTTKDEGDYFC | 17D) |
| Cfa N G4S2 | ELRVSGANPMSSNKSISVYRDKLVKCGGISLLVQN | |
| EEKKMP E3 19K | TSWMLLLLLSLSLLQALDFISLGSGATNFSLLKQA | |
| Linker | GDVEENPGPEFTMASPLTRFLSLNLLLLGESIILG | |
| (e.g., GSS, | SGEAEVQLQQSGAELVRPGTSVKLSCKVSGDTITF | |
| TSGGGGSGGGSGTG) | YYMHFVKQRPGQGLEWIGRIDPEDESTKYSEKFKN | |
| underlined | KATLTADTSSNTAYLKLSSLTSEDTATYFCIYGGY | |
| Intein in bold | YFDYWGQGVMVTVSSGGGGSGGGGSGGGGSDIQMT | |
| Truncated | QSPASLSTSLGETVTIQCQASEDIYSGLAWYQQKP | |
| CD3z (e.g., | GKSPQLLIYGASDLQDGVPSRFSGSGSGTQYSLKI | |
| RAKFSRSAETAANLQDT) | TSMQTEDEGVYFCQQGLTYPRTFGGGTKLELKRGS | |
| italicized | GDYKDDDDKGSLSTTTKPVLRTPSPVHPTGTSQPQ | |
| (linker underlined, | RPEDCRPRGSVKGTGLDFACDIYIWAPLAGICVAL | |
| intein bold, | LLSLIITLICYRAKFSRSAETAANLQDTGSSAEYC | |
| truncated CD3z | LSYDTEILTVEYGFLPIGKIVEERIECTVYTVDKN | |
| italicized) | GFVYTQPIAQWHNRGEQEVFEYCLEDGSIIRATKD | |
| | HKFMTTDGQMLPIDEIFERGLDLKQVDGLPTSGGG | |
| | GSGGGSGTGKYKSRRSFIEEKKMP | |
| Cfa C BB BFP T2A | MVKIISRKSLGTQNVYDIGVEKDHNFLLKNGLVAS | 86 |
| CD28 1XX EGFP AI | NCFNKRGRKKLLYIFKQPFMRPVQTTQEEDGCSCR | (FIG. |
| Linker (e.g., | FPEEEEGGCELGGGGSMSELIKENMHMKLYMEGTV | 17D) |
| GTG) underlined | DNHHFKCTSEGEGKPYEGTQTMRIKVVEGGPLPFA | |
| Intein in bold | FDILATSFLYGSKTFINHTQGIPDFFKQSFPEGFT | |
| SMASh-tag in lower | WERVTTYEDGGVLTATQDTSLQDGCLIYNVKIRGV | |
| case | NFTSNGPVMQKKTLGWEAFTETLYPADGGLEGRND | |
| | MALKLVGGSHLIANIKTTYRSKKPAKNLKMPGVYY | |
| | VDYRLERIKEANNETYVEQHEVAVARYCDLPSKLG | |
| | HKLNGSRSGGSGRAKRALGEGRGSLLTCGDVEENP | |
| | GPMVKIISRKSLGTQNVYDIGVEKDHNFLLKNGLV | |
| | ASNCFNGTGNSRRNRLLQSDYMNMTPRRPGLTRKP | |
| | YQPYAPARDFAAYRPRAKFSRSAETAANLQDPNQL | |
| | YNELNLGRREEYDVLEKKRARDPEMGGKQQRRRNP | |
| | QEGVFNALQKDKMAEAFSEIGTKGERRRGKGHDGL | |
| | FQGLSTATKDTFDALHMQTLAPRGGGGSGGGSMVS | |
| | KGEELFTGVVPILVELDGDVNGHKFSVSGEGEGDA | |
| | TYGKLTLKFICTTGKLPVPWPTLVTTLTYGVQCFS | |
| | RYPDHMKQHDFFKSAMPEGYVQERTIFFKDDGNYK | |
| | TRAEVKFEGDTLVNRIELKGIDFKEDGNILGHKLE | |
| | YNYNSHNVYIMADKQKNGIKVNFKIRHNIEDGSVQ | |
| | LADHYQQNTPIGDGPVLLPDNHYLSTQSALSKDPN | |
| | EKRDHMVLLEFVTAAGITLGMDELYKTRgtggsgd | |
| | emeecsqhlpgagssgdimgssgtgsgsgtsapit | |
| | ayaqqtrgllgciitsltgrdknqvegevqimsta | |
| | tqtflatcingvcwavyhgagtrtiaspkgpviqm | |
| | ytnvdqdlvgwpapqgsrsltpctcgssdlylvtr | |
| | hadvipvrrrgdgrgsllsprpisylkgssggpll | |
| | cpaghavglfraavctrgvakavdfipvenlettm | |
| | rspvftdnssppavtlthpitkidtkyimtcmsad | |
| | levvtstwvlvggvlaalaayclstgcvvivgriv | |
| | lsgkpaiipdrevlyGS | |
| Cfa C CD28z E2A | MVKIISRKSLGTQNVYDIGVEKDHNFLLKNGLVAS | 87 |
| Cfa BB Linker | NCFNGTGNSRRNRLLQSDYMNMTPRRPGLTRKPYQ | (FIGS. |
| (e.g., GTG, LES) | PYAPARDFAAYRPRAKFSRSAETAANLQDPNQLYN | 17E-17G) |
| underlined | ELNLGRREEYDVLEKKRARDPEMGGKQQRRRNPQE | |
| Intein in bold | GVYNALQKDKMAEAYSEIGTKGERRRGKGHDGLYQ | |
| | GLSTATKDTYDALHMQTLAPRGSGQCTNYALLKLA | |
| | GDVESNPGPLESMVKIISRKSLGTQNVYDIGVEKD | |
| | HNFLLKNGLVASNCFNKRGRKKLLYIFKQPFMRPV | |
| | QTTQEEDGCSCRFPEEEEGGCEL | |
| Cfa C CD28z E2A | MVKIISRKSLGTQNVYDIGVEKDHNFLLKNGLVAS | 88 |
| Linker (e.g., GTG) | NCFNGTGNSRRNRLLQSDYMNMTPRRPGLTRKPYQ | (FIGS. |
| underlined | PYAPARDFAAYRPRAKFSRSAETAANLQDPNQLYN | 17E-17G) |
| Intein in bold | ELNLGRREEYDVLEKKRARDPEMGGKQQRRRNPQE | |
| | GVYNALQKDKMAEAYSEIGTKGERRRGKGHDGLYQ | |
| | GLSTATKDTYDALHMQTLAPRGSGQCTNYALLKLA | |
| | GDVESNPGPLE | |
| Cfa C Zap 70 E2A | MVKIISRKSLGTQNVYDIGVEKDHNFLLKNGLVAS | 89 |
| Cfa BB | NCFNGTGpnssasaavaaptlpahpstftqpqrrv | (FIGS. |
| Linker (e.g., | dtlnsdgytpeparlasstdkprpmpmdtsvyesp | 17A-17C) |
| GTG, LES, | ysdpeelkdkklflkrenllvadielgcgnfgsvr | |
| GTSRAKRGS) | qgvyrmrkkqidvaikvlkqgtekadkdemmreaq | |
| underlined | imhqldnpyivrligvcqaealmlvmemagggplh | |
| Intein in bold | kfllgkkeeipvsnvaellhqvamgmkyleeknfv | |
| Zap70 in lower | hrdlaarnvllvnrhyakisdfglskalgaddsyy | |
| case | tarsagkwplkwyapecinfrkfssrsdvwsygvt | |
| | mweafsygqkpykkmkgpevldfikqgkrmecppe | |
| | cppemyalmsdcwiykwedrpdfltveqrmrnyyy | |
| | slGTSRAKRGSGQCTNYALLKLAGDVESNPGPLES | |
| | MVKIISRKSLGTQNVYDIGVEKDHNFLLKNGLVAS | |
| | NCFNKRGRKKLLYIFKQPFMRPVQTTQEEDGCSCR | |
| | FPEEEEGGCEL | |
| Cfa C Zap 70 E2A | MVKIISRKSLGTQNVYDIGVEKDHNFLLKNGLVAS | 90 |
| Linker (e.g., | NCFNGTGpnssasaavaaptlpahpstftqpqrrv | (FIGS. |
| GTG, GTSRAKRGS) | dtlnsdgytpeparlasstdkprpmpmdtsvyesp | 17A-17C) |
| underlined | ysdpeelkdkklflkrenllvadielgcgnfgsvr | |
| Intein in bold | qgvyrmrkkqidvaikvlkqgtekadkdemmreaq | |
| Zap70 in lower | imhqldnpyivrligvcqaealmlvmemagggplh | |
| case | kfllgkkeeipvsnvaellhqvamgmkyleeknfv | |
| | hrdlaarnvllvnrhyakisdfglskalgaddsyy | |
| | tarsagkwplkwyapecinfrkfssrsdvwsygvt | |
| | mweafsygqkpykkmkgpevldfikqgkrmecppe | |
| | cppemyalmsdcwiykwedrpdfltveqrmrnyyy | |
| | slGTSRAKRGSGQCTNYALLKLAGDVESNPGPLE | |
| Thy1.1 P2A RQR | MNPAISVALLLSVLQVSRGQKVTSLTACLVNQNLR | 91 |
| FLAG CD8EC CD8TM | LDCRHENNTKDNSIQHEFSLTREKRKHVLSGTLGI | (FIGS. |
| tCD3z AEY Cfa N | PEHTYRSRVTLSNQPYIKVLTLANFTTKDEGDYFC | 3E-3I) |
| G4S2 EEKKMP E3 | ELRVSGANPMSSNKSISVYRDKLVKCGGISLLVQN | |
| 19K Linker | TSWMLLLLLSLSLLQALDFISLGSGATNFSLLKQA | |
| (e.g., GSS, | GDVEENPGPEFTMETDTLLLWVLLLWVPGSTGCPY | |
| TSGGGGSGGGSGTG) | SNPSLCSGGGGSELPTQGTFSNVSTNVSPAKPTTT | |
| underlined | ACPYSNPSLCGGGGSGGGSDYKDDDDKAAASTTTK | |
| Intein in bold | PVLRTPSPVHPTGTSQPQRPEDCRPRGSVKGTGLD | |
| Truncated | FACDIYIWAPLAGICVALLLSLIITLICYRAKFSR | |
| CD3z (e.g., | SAETAANLQDTGSSAEYCLSYDTEILTVEYGFLPI | |
| RAKFSRSAETAANLQDT) | GKIVEERIECTVYTVDKNGFVYTQPIAQWHNRGEQ | |
| italicized | EVFEYCLEDGSIIRATKDHKFMTTDGQMLPIDEIF | |
| | ERGLDLKQVDGLPTSGGGGSGGGSGTGKYKSRRSF | |
| | IEEKKMP | |
| Cfa C CD3z delta | MVKIISRKSLGTQNVYDIGVEKDHNFLLKNGLVAS | 92 |
| GP41-8 N | NCFNGTGFDALHMQTLAPRTSGSGSQLNRCLSLDT | (FIGS. |
| RXR E2A EGFP | MVVTNGKAIEIRDVKVGDWLESECGPVQVTEVLPI | 3E-3I) |
| Linker (e.g., | IKQPVFEIVLKSGKKIRVSANHKFPTKDGLKTINS | |
| GTG, TSGSG, | GLKVGDFLRSRAKGGGGSGGGSGSTGlldaltlas | |
| GGGGSGGGSGSTG) | srgplrkrsvavakakpkfsispdslsALGGSGRA | |
| underlined | KRGSGQCTNYALLKLAGDVESNPGPLESMVSKGEE | |
| Intein in bold | LFTGVVPILVELDGDVNGHKFSVSGEGEGDATYGK | |
| Kir6.2 RXR in | LTLKFICTTGKLPVPWPTLVTTLTYGVQCFSRYPD | |
| lower case | HMKQHDFFKSAMPEGYVQERTIFFKDDGNYKTRAE | |
| | VKFEGDTLVNRIELKGIDFKEDGNILGHKLEYNYN | |
| | SHNVYIMADKQKNGIKVNFKIRHNIEDGSVQLADH | |
| | YQQNTPIGDGPVLLPDNHYLSTQSALSKDPNEKRD | |
| | HMVLLEFVTAAGITLGMDELYK | |
| Cfa C CD3z delta | MVKIISRKSLGTQNVYDIGVEKDHNFLLKNGLVAS | 93 |
| GP41-8 N | NCFNGTGFDALHMQTLAPRTSGSGSQLNRCLSLDT | (FIGS. |
| EEKKMP EGFP | MVVTNGKAIEIRDVKVGDWLESECGPVQVTEVLPI | 3E-3I) |
| Linker (e.g., | IKQPVFEIVLKSGKKIRVSANHKFPTKDGLKTINS | |
| GTG, TSGSG, | GLKVGDFLRSRAKGGGGSGGGSGTGkyksrrsfie | |
| GGGGSGGGSGTG) | ekkmpGSGRAKRGSGQCTNYALLKLAGDVESNPGP | |
| underlined | LESMVSKGEELFTGVVPILVELDGDVNGHKFSVSG | |
| Intein in bold | EGEGDATYGKLTLKFICTTGKLPVPWPTLVTTLTY | |
| E3 19K KKXX in | GVQCFSRYPDHMKQHDFFKSAMPEGYVQERTIFFK | |
| lower case | DDGNYKTRAEVKFEGDTLVNRIELKGIDFKEDGNI | |
| | LGHKLEYNYNSHNVYIMADKQKNGIKVNFKIRHNI | |
| | EDGSVQLADHYQQNTPIGDGPVLLPDNHYLSTQSA | |
| | LSKDPNEKRDHMVLLEFVTAAGITLGMDELYK | |

### 6.3. Exemplary Modular Polypeptides

As outlined above, the presently disclosed subject matter provides cells comprising modular polypeptides, systems for generating such modular polypeptides, and systems for sorting cells comprising such modular polypeptides. While the figures and certain aspects of the detailed description reference exemplary embodiments employing particular modular polypeptides, e.g., CARs and CCRs, it is appreciated that the systems outlined herein are not limited to those particularly exemplified modular polypeptides. Instead, the various systems described herein can be applied to modular polypeptides incorporating various modular domains discussed herein, e.g., antigen binding domains, transmembrane domains, linkers, spacers, affinity tags, ER retention motifs, intracellular signaling domains, non-signaling domains, co-stimulatory domains, drug-regulatory domains, cytokine sequences, and chemokine sequences.

In certain embodiments, the modular polypeptides are antigen-recognizing receptors. In certain embodiments, an antigen-recognizing receptor of the present disclosure binds a specific antigen of interest. In certain embodiments, the antigen of interest can be a tumor antigen or a pathogen antigen. In certain embodiments, the antigen-recognizing receptor is a chimeric receptor. In certain embodiments, the chimeric receptor is a chimeric antigen receptor (CAR). In certain embodiments, the antigen-recognizing receptor is a chimeric costimulatory receptor (CCR). In certain embodiments, the antigen-recognizing receptor is a T Cell Receptor (TCR). In certain embodiments, the antigen-recognizing receptor is a TCR-like fusion molecule. As described above in Section 5.2, the modular polypeptides, e.g., CARs, CCRs, TCRs, and TCR-like molecules, among other modular polypeptides, can be encoded by a variety of sequences incorporating inteins at one or more locations to facilitate formation of the desired mature modular polypeptide.

### 6.3.1. Chimeric Antigen Receptors (CARs)

In certain embodiments, one or more of the modular polypeptide(s) employed in the systems described herein is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4⁺ and CD8⁺ T cells through their CD35 chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40, NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the antigen-recognizing receptor is a CAR comprising an extracellular antigen-binding domain that binds to the antigen, and an intracellular signaling domain. In certain embodiments, the CAR further comprises a transmembrane domain. In certain embodiments, the CAR further comprises a hinger/spacer region. Non-limiting examples of antigens recognized by the antigen-binding domain of CAR include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g., a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MARTI, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PRI), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

In certain embodiments, the extracellular antigen-binding domain of the CAR (for example, an scFv) binds to the first antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹M or less, or about 1×10⁻¹⁰ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of polypeptide-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent polypeptide (GFP), blue fluorescent polypeptide (e.g., EBFP, EBFP2, Azurite, and mKalama1), cyan fluorescent polypeptide (e.g., ECFP, Cerulean, and CyPet), and yellow fluorescent polypeptide (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion polypeptide with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv. In certain embodiments, the extracellular antigen binding domain is a VHH antibody (nanobody). Table 4 shows exemplary sequences for the extracellular antigen-binding domain.

TABLE 4
Exemplary extracellular antigen-binding
domain sequences

| | | SEQ |
|---|---|---|
| Name | Sequence | ID NO. |
| CD 19 VH (1D3) | EVQLQQSGAELVRPGTSVKLSCKVSGDTITFYYMHF | 94 |
| | VKQRPGQGLEWIGRIDPEDESTKYSEKFKNKATLTA | |
| | DTSSNTAYLKLSSLTSEDTATYFCIYGGYYFDYWGQ | |
| | GVMVTVSS | |
| CD19 VL (1D3) | DIQMTQSPASLSTSLGETVTIQCQASEDIYSGLAWY | 95 |
| | QQKPGKSPQLLIYGASDLQDGVPSRFSGSGSGTQYS | |
| | LKITSMQTEDEGVYFCQQGLTYPRTFGGGTKLELKR | |
| CD19 scFv | EVQLQQSGAELVRPGTSVKLSCKVSGDTITFYYMHF | 96 |
| (VH-Spacer-VL) | VKQRPGQGLEWIGRIDPEDESTKYSEKFKNKATLTA | |
| Spacer bold | DTSSNTAYLKLSSLTSEDTATYFCIYGGYYFDYWGQ | |
| underlined | GVMVTVSSGGGGSGGGGSGGGGSDIQMTQSPASLST | |
| | SLGETVTIQCQASEDIYSGLAWYQQKPGKSPQLLIY | |
| | GASDLQDGVPSRFSGSGSGTQYSLKITSMQTEDEGV | |
| | YFCQQGLTYPRTFGGGTKLELKR | |
| CD20 VH (18B12) | QVQLQQPGAELVRPGTSVKLSCKASGYTFTSYWMHW | 97 |
| | IKQRPGQGLEWIGVIDPSDNYTKYNQKFKGKATLTV | |
| | DTSSSTAYMQLSSLTSEDSAVYFCAREGYYGSSPWF | |
| | AYWGQGTLVTVSS | |
| CD20 VL (18B12) | QIVMSQSPAILSASPGEKVTMTCRARSSVSYIHWYQ | 98 |
| | QKPGSSPKPWIYATSNLASGVPGRFSGSGSGTSYSL | |
| | TITRVEAEDAATYYCQQWSSKPPTFGGGTKLEIKR | |
| CD20 scFv | QIVMSQSPAILSASPGEKVTMTCRARSSVSYIHWYQ | 99 |
| (VL-Spacer-VH) | QKPGSSPKPWIYATSNLASGVPGRFSGSGSGTSYSL | |
| Spacer bold | TITRVEAEDAATYYCQQWSSKPPTFGGGTKLEIKRG | |
| underlined | GGGSGGGGGGGGSQVQLQQPGAELVRPGTSVKLSCK | |
| | ASGYTFTSYWMHWIKQRPGQGLEWIGVIDPSDNYTK | |
| | YNQKFKGKATLTVDTSSSTAYMQLSSLTSEDSAVYF | |
| | CAREGYYGSSPWFAYWGQGTLVTVSS | |
| CD20 VHH (2MC57) | QVQLQESGGGLVQAGGSLRLSCAASGRDFATYSMAW | 100 |
| | FRQAPGKERESVATISWSGQRTRYADSVKGRFTISR | |
| | DNAKNTVYLQMNSLKPEDTAVYYCAMPRTWGEFPPT | |
| | QYDSWGQGTQVTVSS | |

In certain embodiments, the antigen-recognizing receptor is a CAR that comprises a transmembrane domain. Different transmembrane domains result in different receptor stability. After antigen recognition, receptors cluster and a signal are transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a polypeptide associated with the immune response), or a combination thereof. Table 5 shows exemplary transmembrane and signaling domains.

TABLE 5
Exemplary Transmembrane and Signaling
Domain Sequences

| | | SEQ |
|---|---|---|
| | | ID |
| Name | Sequence | NO. |
| CD28 (1) | MLRLLLALNLFPSIQVTGNKILVKQSPMLVAYDNA | 101 |
| | VNLSCKYSYNLFSREFRASLHKGLDSAVEVCVVYG | |
| | NYSQQLQVYSKTGFNCDGKLGNESVTFYLQNLYVN | |
| | QTDIYFCKIEVMYPPPYLDNEKSNGTIIHVKGKHL | |
| | CPSPLFPGPSKPFWVLVVVGGVLACYSLLVTVAFI | |
| | IFWVRSKRSRLLHSDYMNMTPRRPGPTRKHYQPYA | |
| | PPRDFAAYRS | |
| CD28 | AGGAGTAAGAGGAGCAGGCTCCTGCACAGTGACTA | 102 |
| (1) - | CATGAACATGACTCCCCGCCGCCCCGGGCCCACCC | |
| encoding | GCAAGCATTACCAGCCCTATGCCCCACCACGCGAC | |
| amino | TTCGCAGCCTATCGCTCC | |
| acids | MTLRLLFLALNFFSVQVTENKILVKQSPLLVVDSN | 103 |
| 180-220 | EVSLSCRYSYNLLAKEFRASLYKGVNSDVEVCVGN | |
| CD28 | GNFTYQPQFRSNAEFNCDGDFDNETVTFRLWNLHV | |
| (mouse) | NHTDIYFCKIEFMYPPPYLDNERSNGTIIHIKEKH | |
| | LCHTQSSPKLFWALVVVAGVLFCYGLLVTVALCVI | |
| | WTNSRRNRLLQSDYMNMTPRRPGLTRKPYQPYAPA | |
| | RDFAAYRP | |
| 4-1BB | MGNSCYNIVATLLLVLNFERTRSLQDPCSNCPAGT | 104 |
| | FCDNNRNQICSPCPPNSFSSAGGQRTCDICRQCKG | |
| | VFRTRKECSSTSNAECDCTPGFHCLGAGCSMCEQD | |
| | CKQGQELTKKGCKDCCFGTFNDQKRGICRPWTNCS | |
| | LDGKSVLVNGTKERDVVCGPSPADLSPGASSVTPP | |
| | APAREPGHSPQIISFFLALTSTALLFLLFFLTLRF | |
| | SVVKRGRKKLLYIFKQPFMRPVQTTQEEDGCSCRF | |
| | PEEEEGGCEL | |
| CD3γ | MEQGKGLAVLILAIILLQGTLAQSIKGNHLVKVYD | 105 |
| | YQEDGSVLLTCDAEAKNITWFKDGKMIGFLTEDKK | |
| | KWNLGSNAKDPRGMYQCKGSQNKSKPLQVYYRMCQ | |
| | NCIELNAATISGFLFAEIVSIFVLAVGVYFIAGQD | |
| | GVRQSRASDKQTLLPNDQLYQPLKDREDDQYSHLQ | |
| | GNQLRRN | |
| CD3δ (1) | MEHSTFLSGLVLATLLSQVSPFKIPIEELEDRVFV | 106 |
| | NCNTSITWVEGTVGTLLSDITRLDLGKRILDPRGI | |
| | YRCNGTDIYKDKESTVQVHYRMCQSCVELDPATVA | |
| | GIIVTDVIATLLLALGVFCFAGHETGRLSGAADTQ | |
| | ALLRNDQVYQPLRDRDDAQYSHLGGNWARNK | |
| CD3δ (2) | MEHSTFLSGLVLATLLSQVSPFKIPIEELEDRVFV | 107 |
| | NCNTSITWVEGTVGTLLSDITRLDLGKRILDPRGI | |
| | YRCNGTDIYKDKESTVQVHYRTADTQALLRNDQVY | |
| | QPLRDRDDAQYSHLGGNWARNK | |
| CD3ζ | MQSGTHWRVLGLCLLSVGVWGQDGNEEMGGITQTP | 108 |
| | YKVSISGTTVILTCPQYPGSEILWQHNDKNIGGDE | |
| | DDKNIGSDEDHLSLKEFSELEQSGYYVCYPRGSKP | |
| | EDANFYLYLRARVCENCMEMDVMSVATIVIVDICI | |
| | TGGLLLLVYYWSKNRKAKAKPVTRGAGAGGRQRGQ | |
| | NKERPPPVPNPDYEPIRKGQRDLYSGLNQRRI | |
| CD3ζ (1) | MKWKALFTAAILQAQLPITEAQSFGLLDPKLCYLL | 109 |
| | DGILFIYGVILTALFLRVKFSRSADAPAYQQGQNQ | |
| | LYNELNLGRREEYDVLDKRRGRDPEMGGKPQRRKN | |
| | PQEGLYNELQKDKMAEAYSEIGMKGERRRGKGHDG | |
| | LYQGLSTATKDTYDALHMQALPPR | |
| CD3ζ (2) | RVKFSRSADAPAYQQGQNQLYNELNLGRREEYDVL | 110 |
| | DKRRGRDPEMGGKPRRKNPQEGLYNELQKDKMAEA | |
| | YSEIGMKGERRRGKGHDGLYQGLSTATKDTYDALH | |
| | MQALPPR | |
| CD3ζ (3) | RVKFSRSADAPAYQQGQNQLYNELNLGRREEYDVL | 111 |
| | DKRRGRDPEMGGKPRRKNPQEGLFNELQKDKMAEA | |
| | FSEIGMKGERRRGKGHDGLFQGLSTATKDTFDALH | |
| | MQALPPR | |
| CD3ζ (3) | AGAGTGAAGTTCAGCAGGAGCGCAGACGCCCCCGC | 112 |
| | GTACCAGCAGGGCCAGAACCAGCTCTATAACGAGC | |
| | TCAATCTAGGACGAAGAGAGGAGTACGATGTTTTG | |
| | GACAAGAGACGTGGCCGGGACCCTGAGATGGGGGG | |
| | AAAGCCGAGAAGGAAGAACCCTCAGGAAGGCCTGT | |
| | TCAATGAACTGCAGAAAGATAAGATGGCGGAGGCC | |
| | TTCAGTGAGATTGGGATGAAAGGCGAGCGCCGGAG | |
| | GGGCAAGGGGCACGATGGCCTTTTCCAGGGGCTCA | |
| | GTACAGCCACCAAGGACACCTTCGACGCCCTTCAC | |
| | ATGCAGGCCCTGCCCCCTCGC | |
| CD3ζ | MKWKVSVLACILHVRFPGAEAQSFGLLDPKLCYLL | 113 |
| (mouse) | DGILFIYGVIITALYLRAKFSRSAETAANLQDPNQ | |
| | LYNELNLGRREEYDVLEKKRARDPEMGGKQQRRRN | |
| | PQEGVYNALQKDKMAEAYSEIGTKGERRRGKGHDG | |
| | LYQGLSTATKDTYDALHMQTLAPR | |
| Zap70 | MPDPAAHLPFFYGSISRAEAEEHLKLAGMADGLFL | 114 |
| human | LRQCLRSLGGYVLSLVHDVRFHHFPIERQLNGTYA | |
| Inter- | IAGGKAHCGPAELCEFYSRDPDGLPCNLRKPCNRP | |
| domain + | SGLEPQPGVFDCLRDAMVRDYVRQTWKLEGEALEQ | |
| kinase | AIISQAPQVEKLIATTAHERMPWYHSSLTREEAER | |
| domain | KLYSGAQTDGKFLLRPRKEQGTYALSLIYGKTVYH | |
| in lower | YLISQDKAGKYCIPEGTKFDTLWQLVEYLKLKADG | |
| case | LIYCLKEACpnssasnasgaaaptlpahpstlthp | |
| under- | qrridtlnsdgytpeparitspdkprpmpmdtsvy | |
| lined | espysdpeelkdkklflkrdnlliadielgcgnfg | |
| | svrqgvyrmrkkgidvaikvlkqgtekadteemmr | |
| | eaqimhqldnpyivrligvcqaealmlvmemaggg | |
| | plhkflvgkreeipvsnvaellhqvsmgmkyleek | |
| | nfvhrdlaarnvllynrhyakisdfglskalgadd | |
| | syytarsagkwplkwyapecinfrkfssrsdvwsy | |
| | gvtmwealsyggkpykkmkgpevmafieggkrmec | |
| | ppecppelyalmsdcwiykwedrpdfltvegrmra | |
| | cyyslASKVEGPPGSTQKAEAACA | |
| Zap70 | MPDPAAHLPFFYGSISRAEAEEHLKLAGMADGLFL | 115 |
| mouse | LRQCLRSLGGYVLSLVHDVRFHHFPIERQLNGTYA | |
| Inter- | IAGGKAHCGPAELCQFYSQDPDGLPCNLRKPCNRP | |
| domain + | PGLEPQPGVFDCLRDAMVRDYVRQTWKLEGDALEQ | |
| kinase | AIISQAPQVEKLIATTAHERMPWYHSSLTREEAER | |
| domain | KLYSGQQTDGKFLLRPRKEQGTYALSLVYGKTVYH | |
| in lower | YLISQDKAGKYCIPEGTKFDTLWQLVEYLKLKADG | |
| case | LIYRLKEVCpnssasaavaaptlpahpstftqpqr | |
| under- | rvdtlnsdgytpeparlasstdkprpmpmdtsvye | |
| lined | spysdpeelkdkklflkrenllvadielgcgnfgs | |
| | vrqgvyrmrkkidvaikvlkqgtekadkdemmrea | |
| | gimhqldnpyivrligvcgaealmlvmemagggpl | |
| | hkfllgkkeeipvsnvaellhqvamgmkyleeknf | |
| | vhrdlaarnvllvnrhyakisdfglskalgaddsy | |
| | ytarsagkwplkwyapecinfrkfssrsdvwsygv | |
| | tmweafsygqkpykkmkgpevldfikqgkrmecpp | |
| | ecppemyalmsdcwiykwedrpdfltveqrmrnyy | |
| | yslASRAEGPPQCEQVAEAACG | |
| ITAM1 | QNQLYNELNLGRREEYDVLDKR | 116 |
| ITAM1 | CAGAACCAGCTCTATAACGAGCTCAATCTAGGACG | 117 |
| | AAGAGAGGAGTACGATGTTTTGGACAAGAGA | |
| ITAM1 | QNQLFNELNLGRREEFDVLDKR | 118 |
| variant | | |
| ITAM1 | CAGAACCAGCTCTTTAACGAGCTCAATCTAGGACG | 119 |
| variant | AAGAGAGGAGTTCGATGTTTTGGACAAGAGA | |
| ITAM2 | QEGLYNELQKDKMAEAYSEIGMK | 120 |
| ITAM2 | CAGGAAGGCCTGTACAATGAACTGCAGAAAGATAA | 121 |
| | GATGGCGGAGGCCTACAGTGAGATTGGGATGAAA | |
| ITAM2 | QEGLFNELQKDKMAEAFSEIGMK | 122 |
| variant | | |
| ITAM2 | CAGGAAGGCCTGTTCAATGAACTGCAGAAAGATAA | 123 |
| variant | GATGGCGGAGGCCTTCAGTGAGATTGGGATGAAA | |
| ITAM3 | HDGLYQGLSTATKDTYDALHMQ | 124 |
| ITAM3 | CACGATGGCCTTTACCAGGGTCTCAGTACAGCCAC | 125 |
| | CAAGGACACCTACGACGCCCTTCACATGCAG | |
| ITAM3 | HDGLFQGLSTATKDTFDALHMQ | 126 |
| variant | | |
| ITAM3 | CACGATGGCCTTTTCCAGGGGCTCAGTACAGCCAC | 127 |
| variant | CAAGGACACCTTCGACGCCCTTCACATGCAG | |

In certain embodiments, the signaling domain for the CAR is a kinase. Non-limiting examples of such kinases include the Src kinase family (e.g., Blk, Fgr, Fyn, Hck, Lck, Lyn, Src, Yes, Yrk), the Syk kinase family (e.g., Zap70, Syk, BTK, TEK, ITK), and the receptor tyrosine (RTK) superfamily, which includes, RTKs class I (EGF receptor family or ErbB family), class II (Insulin receptor family), class III (PDGF receptor family), class IV (VEGF receptors family), class V (FGF receptor family), class VI (CCK receptor family), class VII (NGF receptor family), class VIII (HGF receptor family), class IX (Eph receptor family), class X (AXL receptor family), class XI (TIE receptor family), class XII (RYK receptor family), class XIII (DDR receptor family), class XIV (RET receptor family), class XV (ROS receptor family), class XVI (LTK receptor family), class XVII (ROR receptor family), class XVIII (MuSK receptor family), class XIX (LMR receptor), class XX (Undetermined), class XVIII (MuSK receptor family), class XIX (LMR receptor), class XX and orthologs thereof. In certain embodiments, the signaling domain for the CAR comprises a kinase domain from one or more of PDGFR, KIT, Abl, Arg, EGFR, Raf, VEGFR, PDGFR, Flt3, Abl, Arg, or ErbB2, or orthologs thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 101), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 103. In certain embodiments, the transmembrane domain of the CAR comprises a CD28 polypeptide that comprises or consists of amino acids 153 to 179 of SEQ ID NO: 101 (Table 5).

In certain embodiments, the antigen-recognizing receptor is a CAR that further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a polypeptide associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 101), a portion of a CD8 polypeptide, or a synthetic spacer sequence.

In certain embodiments, the antigen-recognizing receptor is a CAR that further comprises a hinge/spacer region comprising a native or modified hinge region of a CD28 polypeptide. In certain embodiments, the hinge/spacer region of the first antigen-recognizing receptor (e.g., a CAR) comprises a CD28 polypeptide comprising or consisting of amino acids 114 to 152 of SEQ ID NO: 101.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3 ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a polypeptide associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a polypeptide associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide, and the transmembrane domain comprises a CD28 polypeptide. In certain embodiments, the hinge/spacer region comprises a CD28 polypeptide, and the transmembrane domain comprises a CD28 polypeptide. In certain embodiments, the hinge/spacer region comprises a CD84 polypeptide, and the transmembrane domain comprises a CD84 polypeptide. In certain embodiments, the hinge/spacer region comprises a CD166 polypeptide, and the transmembrane domain comprises a CD166 polypeptide. In certain embodiments, the hinge/spacer region comprises a CD8a polypeptide, and the transmembrane domain comprises a CD8a polypeptide. In certain embodiments, the hinge/spacer region comprises a CD8b polypeptide, and the transmembrane domain comprises a CD8b polypeptide. In certain embodiments, the hinge/spacer region comprises a CD28 polypeptide, and the transmembrane domain comprises an ICOS polypeptide.

In certain embodiments, the antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 109) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 109, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 109. In certain embodiments, the intracellular signaling domain of the CAR comprises a native CD3ζ comprising or consisting of the amino acid sequence of amino acids 52 to 164 of SEQ ID NO: 109 (Table 5).

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 110 (Table 5)

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 116 (Table 5).

An exemplary nucleic acid sequence encoding the amino acid sequence of SEQ ID NO: 34 is set forth in SEQ ID NO: 117 (Table 5).

In certain embodiments, the modified CD3 polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 118 (Table 5).

An exemplary nucleic acid sequence encoding the amino acid sequence of SEQ ID NO: 118 is set forth in SEQ ID NO: 119 (Table 5).

In certain embodiments, the modified CD3ζ polypeptide comprises a native ITAM2. In certain embodiments, the native ITAM2 comprises or consists of the amino acid sequence set forth in SEQ ID NO: 120 (Table 5).

An exemplary nucleic acid sequence encoding the amino acid sequence of SEQ ID NO: 120 is set forth in SEQ ID NO: 121 (Table 5).

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 122 (Table 5).

An exemplary nucleic acid sequence encoding the amino acid sequence of SEQ ID NO: 122 is set forth in SEQ ID NO: 123 (Table 3).

In certain embodiments, the modified CD3ζ polypeptide comprises a native ITAM3. In certain embodiments, the native ITAM3 comprises or consists of the amino acid sequence set forth in SEQ ID NO: 124 (Table 5).

An exemplary nucleic acid sequence encoding the amino acid sequence of SEQ ID NO: 124 is set forth in SEQ ID NO: 125 (Table 5).

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 126 (Table 3).

An exemplary nucleic acid sequence encoding the amino acid sequence of SEQ ID NO: 126 is set forth in SEQ ID NO: 127 (Table 5).

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 116, an ITAM2 variant consisting of the amino acid sequence set forth in SEQ ID NO: 122, and an ITAM3 variant consisting of the amino acid sequence set forth in SEQ ID NO: 126. In certain embodiments, the CAR is designated as “1XX”. In certain embodiments, the modified CD3 polypeptide comprises or consists of the amino acid sequence set forth in SEQ ID NO: 111 (Table 5).

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 111 or a fragment thereof, and/or may optionally comprise up to one or up to two or up to three conservative amino acid substitutions.

An exemplary nucleic acid sequence encoding the amino acid sequence of SEQ ID NO: 111 is set forth in SEQ ID NO: 112 (Table 3).

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the first antigen-recognizing receptor comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 101 or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 101, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 101. In certain embodiments, the intracellular signaling domain of the CAR comprises a co-stimulatory signaling region that comprises a CD28 polypeptide comprising or consisting of amino acids 180 to 220 of SEQ ID NO: 101.

An exemplary nucleic acid sequence encoding the amino acid sequence of amino acids 180 to 220 of SEQ ID NO: 101 is set forth in SEQ ID NO: 102 (Table 5).

In certain embodiments, the intracellular signaling domain of the first antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 103) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 103, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 103. In certain embodiments, the co-stimulatory signaling region of the CAR comprises a CD28 polypeptide that comprises or consists of amino acids 178 to 218 of SEQ ID NO: 103 (Table 5).

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 104) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 104, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 104. In certain embodiments, the co-stimulatory signaling region of the CAR comprises a 4-1BB polypeptide comprising or consisting of the amino acid sequence of amino acids 214 to 255 of SEQ ID NO: 104 (Table 5).

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In addition, the extracellular antigen-binding domain of the CAR can comprise a leader or a signal peptide that directs the nascent polypeptide into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized polypeptides that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human IL-2 signal sequence comprising or consisting of the amino acid sequence set forth in SEQ ID NO: 128), a mouse IL-2 signal sequence (e.g., a mouse IL-2 signal sequence comprising or consisting of the amino acid sequence set forth in SEQ ID NO: 129); a human kappa leader sequence (e.g., a human kappa leader sequence comprising or consisting of the amino acid sequence set forth in SEQ ID NO: 130), a mouse kappa leader sequence (e.g., a mouse kappa leader sequence comprising or consisting of the amino acid sequence set forth in SEQ ID NO: 131); a human CD8 leader sequence (e.g., a human CD8 leader sequence comprising or consisting of the amino acid sequence set forth in SEQ ID NO: 132); a truncated human CD8 signal peptide (e.g., a truncated human CD8 signal peptide comprising or consisting of the amino acid sequence set forth in SEQ ID NO: 133); a human albumin signal sequence (e.g., a human albumin signal sequence comprising or consisting of the amino acid sequence set forth in SEQ ID NO: 134); and a human prolactin signal sequence (e.g., a human prolactin signal sequence comprising or consisting of the amino acid sequence set forth in SEQ ID NO: 135). These exemplary signal sequences are shown in Table 6.

TABLE 6
Exemplary Signal Sequences

| | | SEQ |
|---|---|---|
| | | ID |
| Name | Sequence | NO. |
| Signal sequence 1 | MYRMQLLSCIALSLALVINS | 128 |
| Signal sequence 2 | MYSMQLASCVTLTLVLLVNS | 129 |
| Signal sequence 3 | METPAQLLFLLLLWLPDTTG | 130 |
| Signal sequence 4 | METDTLLLWVLLLWVPGSTG | 131 |
| Signal sequence 5 | MALPVTALLLPLALLLHAARP | 132 |
| Signal sequence 6 | MALPVTALLLPLALLLHA | 133 |
| Signal sequence 7 | MKWVTFISLLFSSAYS | 134 |
| Signal sequence 8 | MDSKGSSQKGSRLLLLLVVSN | 135 |
| | LLLCQGVVS | |

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 132.

### 6.3.2. Chimeric Co-Stimulating Receptors (CCRs)

In certain embodiments, one or more of the modular polypeptide(s) employed in the systems described herein is a CCR. The term “chimeric co-stimulating receptor” or “CCR” refers to a chimeric receptor that binds to an antigen and provides a co-stimulatory signal but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD3 polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31 (1): 71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the signaling domain for the CCR is a kinase. Non-limiting examples of such kinases include the Src kinase family (e.g., Blk, Fgr, Fyn, Hck, Lck, Lyn, Src, Yes, Yrk), the Syk kinase family (e.g., Zap70, Syk, BTK, TEK, ITK), and the receptor tyrosine (RTK) superfamily, which includes, RTKs class I (EGF receptor family or ErbB family), class II (Insulin receptor family), class III (PDGF receptor family), class IV (VEGF receptors family), class V (FGF receptor family), class VI (CCK receptor family), class VII (NGF receptor family), class VIII (HGF receptor family), class IX (Eph receptor family), class X (AXL receptor family), class XI (TIE receptor family), class XII (RYK receptor family), class XIII (DDR receptor family), class XIV (RET receptor family), class XV (ROS receptor family), class XVI (LTK receptor family), class XVII (ROR receptor family), class XVIII (MuSK receptor family), class XIX (LMR receptor), class XX (Undetermined), class XVIII (MuSK receptor family), class XIX (LMR receptor), class XX and orthologs thereof. In certain embodiments, the signaling domain for the CAR comprises a kinase domain from one or more of PDGFR, KIT, Abl, Arg, EGFR, Raf, VEGFR, PDGFR, Flt3, Abl, Arg, or ErbB2, or orthologs thereof.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both a first antigen (e.g., antigen targeted by a CAR) and the second antigen is restricted to the targeted cells (e.g., cancerous tissue or cancerous cells, or LSCs, or AML HSPCs). Similar to a CAR, the extracellular antigen-binding domain can be an scFv, a Fab, a F (ab) 2, or a fusion polypeptide with a heterologous sequence to form the extracellular antigen-binding domain.

In certain embodiments, the cell comprising a first antigen-recognizing receptor (e.g., a CAR) and a second antigen-recognizing receptor (e.g., a CAR, a TCR, or a TCR-like fusion molecule) exhibits a greater degree of cytolytic activity against cells that are positive for both the first antigen and the second antigen as compared to against cells that are singly positive for the first antigen. In certain embodiments, the cell comprising the first antigen-recognizing receptor (e.g., a CAR) and the second antigen-recognizing receptor (e.g., a CAR, a TCR, or a TCR-like fusion molecule) exhibits substantially no or negligible cytolytic activity against cells that are singly positive for the first antigen.

In certain embodiments, the first antigen recognizing receptor (e.g., a CAR, a TCR, or a TCR-like fusion molecule) binds to the first antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the first antigen recognizing receptor (e.g., a CAR, a TCR, or a TCR-like fusion molecule) binds to the first antigen with a low binding avidity. In certain embodiments, the first antigen recognizing receptor (e.g., a CAR, a TCR, or a TCR-like fusion molecule) binds to the first antigen at an epitope of low accessibility. In certain embodiments, the first antigen recognizing receptor (e.g., a CAR, a TCR, or a TCR-like fusion molecule) binds to the first antigen with a binding affinity that is lower compared to the binding affinity with which the second antigen-recognizing receptor (e.g., a CCR) binds to the second antigen. In certain embodiments, the CCR binds to the second antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 6.3.3. T Cell Receptors (TCRs)

In certain embodiments, one or more of the modular polypeptide(s) employed in the systems described herein is a TCR. A TCR is a disulfide-linked heterodimeric polypeptide consisting of two variable chains expressed as part of a complex with the invariant CD3 chain molecules. A TCR is found on the surface of T cells and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the antigen-recognizing receptor is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 6.3.4. TCR-Like Fusion Molecules

In certain embodiments, one or more of the modular polypeptide(s) employed in the systems described herein is a TCR-like fusion molecule. Non-limiting examples of TCR-like fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRUC receptors engaging the complete T cell receptor for potent anti-tumor response,” Nature Communications volume 10, Article number: 2087 (2019), which is incorporated by reference in its entirety).

In certain embodiments, the TCR-like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT-CAR” or “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 136) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 136 (Table 7).

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 135 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 137 (Table 7).

An exemplary nucleotide sequence encoding the amino acid sequence of SEQ ID NO: 137 is set forth in SEQ ID NO: 138 (Table 7).

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 139 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 139 (Table 7).

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 140 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 1408.

An exemplary nucleotide sequence encoding the amino acid sequence of SEQ ID NO: 140 is set forth in SEQ ID NO: 141 (Table 7).

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI GenBank ID: 28639, NG_001333.2, range 645749 to 647196 (TRBC1, SEQ ID NO: 142), NCBI GenBank ID: 28638, NG_001333.2 range 655095 to 656583 (TRBC2, SEQ ID NO: 143) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 142. In certain embodiments, the TRBC polypeptide comprises or consists of the amino acid sequence encoded by the nucleotide sequence of SEQ ID NO: 143 (Table 7).

In certain embodiments, the TCR-like fusion molecule comprises a hinge/spacer region that links the first antigen binding chain to the constant domain. In certain embodiments, the TCR-like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH₂CH₃ region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 144. In certain embodiments, the hinge/spacer region comprises or consists of amino acids 1 to 3 of the sequence set forth in SEQ ID NO: 144. An exemplary nucleic acid sequence encoding the amino acid sequence of SEQ ID NO: 144 is set forth in SEQ ID NO: 145 (Table 7).

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 146. In certain embodiments, the hinge/spacer region comprises or consists of amino acid 1 to 2 of the sequence set forth in SEQ ID NO: 146. An exemplary nucleic acid sequence encoding the amino acid sequence of SEQ ID NO: 146 is set forth in SEQ ID NO: 147. Table 7 shows exemplary T cell receptor and hinge/spacer sequences.

TABLE 7
Exemplary T Cell Receptor and Hinge/Spacer Sequences

| | | |
|---|---|---|
| TRAC | ATATCCAGAACCCTGACCCTGCCGTGTACCAGCTGAGAGACTCTAAA | 136 |
| | TCCAGTGACAAGTCTGTCTGCCTATTCACCGATTTTGATTCTCAAAC | |
| | AAATGTGTCACAAAGTAAGGATTCTGATGTGTATATCACAGACAAAA | |
| | CTGTGCTAGACATGAGGTCTATGGACTTCAAGAGCAACAGTGCTGTG | |
| | GCCTGGAGCAACAAATCTGACTTTGCATGTGCAAACGCCTTCAACAA | |
| | CAGCATTATTCCAGAAGACACCTTCTTCCCCAGCCCAGGTAAGGGCA | |
| | GCTTTGGTGCCTTCGCAGGCTGTTTCCTTGCTTCAGGAATGGCCAGG | |
| | TTCTGCCCAGAGCTCTGGTCAATGATGTCTAAAACTCCTCTGATTGG | |
| | TGGTCTCGGCCTTATCCATTGCCACCAAAACCCTCTTTTTACTAAGA | |
| | AACAGTGAGCCTTGTTCTGGCAGTCCAGAGAATGACACGGGAAAAAA | |
| | GCAGATGAAGAGAAGGTGGCAGGAGAGGGCACGTGGCCCAGCCTCAG | |
| | TCTCTCCAACTGAGTTCCTGCCTGCCTGCCTTTGCTCAGACTGTTTG | |
| | CCCCTTACTGCTCTTCTAGGCCTCATTCTAAGCCCCTTCTCCAAGTT | |
| | GCCTCTCCTTATTTCTCCCTGTCTGCCAAAAAATCTTTCCCAGCTCA | |
| | CTAAGTCAGTCTCACGCAGTCACTCATTAACCCACCAATCACTGATT | |
| | GTGCCGGCACATGAATGCACCAGGTGTTGAAGTGGAGGAATTAAAAA | |
| | GTCAGATGAGGGGTGTGCCCAGAGGAAGCACCATTCTAGTTGGGGGA | |
| | GCCCATCTGTCAGCTGGGAAAAGTCCAAATAACTTCAGATTGGAATG | |
| | TGTTTTAACTCAGGGTTGAGAAAACAGCTACCTTCAGGACAAAAGTC | |
| | AGGGAAGGGCTCTCTGAAGAAATGCTACTTGAAGATACCAGCCCTAC | |
| | CAAGGGCAGGGAGAGGACCCTATAGAGGCCTGGGACAGGAGCTCAAT | |
| | GAGAAAGGAGAAGAGCAGCAGGCATGAGTTGAATGAAGGAGGCAGGG | |
| | CCGGGTCACAGGGCCTTCTAGGCCATGAGAGGGTAGACAGTATTCTA | |
| | AGGACGCCAGAAAGCTGTTGATCGGCTTCAAGCAGGGGAGGGACACC | |
| | TAATTTGCTTTTCTTTTTTTTTTTTTTTTTTTTTTTTTTTTTTGAGA | |
| | TGGAGTTTTGCTCTTGTTGCCCAGGCTGGAGTGCAATGGTGCATCTT | |
| | GGCTCACTGCAACCTCCGCCTCCCAGGTTCAAGTGATTCTCCTGCCT | |
| | CAGCCTCCCGAGTAGCTGAGATTACAGGCACCCGCCACCATGCCTGG | |
| | CTAATTTTTTGTATTTTTAGTAGAGACAGGGTTTCACTATGTTGGCC | |
| | AGGCTGGTCTCGAACTCCTGACCTCAGGTGATCCACCCGCTTCAGCC | |
| | TCCCAAAGTGCTGGGATTACAGGCGTGAGCCACCACACCCGGCCTGC | |
| | TTTTCTTAAAGATCAATCTGAGTGCTGTACGGAGAGTGGGTTGTAAG | |
| | CCAAGAGTAGAAGCAGAAAGGGAGCAGTTGCAGCAGAGAGATGATGG | |
| | AGGCCTGGGCAGGGTGGTGGCAGGGAGGTAACCAACACCATTCAGGT | |
| | TTCAAAGGTAGAACCATGCAGGGATGAGAAAGCAAAGAGGGGATCAA | |
| | GGAAGGCAGCTGGATTTTGGCCTGAGCAGCTGAGTCAATGATAGTGC | |
| | CGTTTACTAAGAAGAAACCAAGGAAAAAATTTGGGGTGCAGGGATCA | |
| | AAACTTTTTGGAACATATGAAAGTACGTGTTTATACTCTTTATGGCC | |
| | CTTGTCACTATGTATGCCTCGCTGCCTCCATTGGACTCTAGAATGAA | |
| | GCCAGGCAAGAGCAGGGTCTATGTGTGATGGCACATGTGGCCAGGGT | |
| | CATGCAACATGTACTTTGTACAAACAGTGTATATTGAGTAAATAGAA | |
| | ATGGTGTCCAGGAGCCGAGGTATCGGTCCTGCCAGGGCCAGGGGCTC | |
| | TCCCTAGCAGGTGCTCATATGCTGTAAGTTCCCTCCAGATCTCTCCA | |
| | CAAGGAGGCATGGAAAGGCTGTAGTTGTTCACCTGCCCAAGAACTAG | |
| | GAGGTCTGGGGTGGGAGAGTCAGCCTGCTCTGGATGCTGAAAGAATG | |
| | TCTGTTTTTCCTTTTAGAAAGTTCCTGTGATGTCAAGCTGGTCGAGA | |
| | AAAGCTTTGAAACAGGTAAGACAGGGGTCTAGCCTGGGTTTGCACAG | |
| | GATTGCGGAAGTGATGAACCCGCAATAACCCTGCCTGGATGAGGGAG | |
| | TGGGAAGAAATTAGTAGATGTGGGAATGAATGATGAGGAATGGAAAC | |
| | AGCGGTTCAAGACCTGCCCAGAGCTGGGTGGGGTCTCTCCTGAATCC | |
| | CTCTCACCATCTCTGACTTTCCATTCTAAGCACTTTGAGGATGAGTT | |
| | TCTAGCTTCAATAGACCAAGGACTCTCTCCTAGGCCTCTGTATTCCT | |
| | TTCAACAGCTCCACTGTCAAGAGAGCCAGAGAGAGCTTCTGGGTGGC | |
| | CCAGCTGTGAAATTTCTGAGTCCCTTAGGGATAGCCCTAAACGAACC | |
| | AGATCATCCTGAGGACAGCCAAGAGGTTTTGCCTTCTTTCAAGACAA | |
| | GCAACAGTACTCACATAGGCTGTGGGCAATGGTCCTGTCTCTCAAGA | |
| | ATCCCCTGCCACTCCTCACACCCACCCTGGGCCCATATTCATTTCCA | |
| | TTTGAGTTGTTCTTATTGAGTCATCCTTCCTGTGGTAGCGGAACTCA | |
| | CTAAGGGGCCCATCTGGACCCGAGGTATTGTGATGATAAATTCTGAG | |
| | CACCTACCCCATCCCCAGAAGGGCTCAGAAATAAAATAAGAGCCAAG | |
| | TCTAGTCGGTGTTTCCTGTCTTGAAACACAATACTGTTGGCCCTGGA | |
| | AGAATGCACAGAATCTGTTTGTAAGGGGATATGCACAGAAGCTGCAA | |
| | GGGACAGGAGGTGCAGGAGCTGCAGGCCTCCCCCACCCAGCCTGCTC | |
| | TGCCTTGGGGAAAACCGTGGGTGTGTCCTGCAGGCCATGCAGGCCTG | |
| | GGACATGCAAGCCCATAACCGCTGTGGCCTCTTGGTTTTACAGATAC | |
| | GAACCTAAACTTTCAAAACCTGTCAGTGATTGGGTTCCGAATCCTCC | |
| | TCCTGAAAGTGGCCGGGTTTAATCTGCTCATGACGCTGCGGCTGTGG | |
| | TCCAGCTGAGGTGAGGGGCCTTGAAGCTGGGAGTGGGGTTTAGGGAC | |
| | GCGGGTCTCTGGGTGCATCCTAAGCTCTGAGAGCAAACCTCCCTGCA | |
| | GGGTCTTGCTTTTAAGTCCAAAGCCTGAGCCCACCAAACTCTCCTAC | |
| | TTCTTCCTGTTACAAATTCCTCTTGTGCAATAATAATGGCCTGAAAC | |
| | GCTGTAAAATATCCTCATTTCAGCCGCCTCAGTTGCACTTCTCCCCT | |
| | ATGAGGTAGGAAGAACAGTTGTTTAGAAACGAAGAAACTGAGGCCCC | |
| | ACAGCTAATGAGTGGAGGAAGAGAGACACTTGTGTACACCACATGCC | |
| | TTGTGTTGTACTTCTCTCACCGTGTAACCTCCTCATGTCCTCTCTCC | |
| | CCAGTACGGCTCTCTTAGCTCAGTAGAAAGAAGACATTACACTCATA | |
| | TTACACCCCAATCCTGGCTAGAGTCTCCGCACCCTCCTCCCCCAGGG | |
| | TCCCCAGTCGTCTTGCTGACAACTGCATCCTGTTCCATCACCATCAA | |
| | AAAAAAACTCCAGGCTGGGTGCGGGGGCTCACACCTGTAATCCCAGC | |
| | ACTTTGGGAGGCAGAGGCAGGAGGAGCACAGGAGCTGGAGACCAGCC | |
| | TGGGCAACACAGGGAGACCCCGCCTCTACAAAAAGTGAAAAAATTAA | |
| | CCAGGTGTGGTGCTGCACACCTGTAGTCCCAGCTACTTAAGAGGCTG | |
| | AGATGGGAGGATCGCTTGAGCCCTGGAATGTTGAGGCTACAATGAGC | |
| | TGTGATTGCGTCACTGCACTCCAGCCTGGAAGACAAAGCAAGATCCT | |
| | GTCTCAAATAATAAAAAAAATAAGAACTCCAGGGTACATTTGCTCCT | |
| | AGAACTCTACCACATAGCCCCAAACAGAGCCATCACCATCACATCCC | |
| | TAACAGTCCTGGGTCTTCCTCAGTGTCCAGCCTGACTTCTGTTCTTC | |
| | CTCATTCCAGATCTGCAAGATTGTAAGACAGCCTGTGCTCCCTCGCT | |
| | CCTTCCTCTGCATTGCCCCTCTTCTCCCTCTCCAAACAGAGGGAACT | |
| | CTCCTACCCCCAAGGAGGTGAAAGCTGCTACCACCTCTGTGCCCCCC | |
| | CGGCAATGCCACCAACTGGATCCTACCCGAATTTATGATTAAGATTG | |
| | CTGAAGAGCTGCCAAACACTGCTGCCACCCCCTCTGTTCCCTTATTG | |
| | CTGCTTGTCACTGCCTGACATTCACGGCAGAGGCAAGGCTGCTGCAG | |
| | CCTCCCCTGGCTGTGCACATTCCCTCCTGCTCCCCAGAGACTGCCTC | |
| | CGCCATCCCACAGATGATGGATCTTCAGTGGGTTCTCTTGGGCTCTA | |
| | GGTCCTGCAGAATGTTGTGAGGGGTTTATTTTTTTTTAATAGTGTTC | |
| | ATAAAGAAATACATAGTATTCTTCTTCTCAAGACGTGGGGGGAAATT | |
| | ATCTCATTATCGAGGCCCTGCTATGCTGTGTATCTGGGCGTGTTGTA | |
| | TGTCCTGCTGCCGATGCCTTC | |
| TRBC2 | DLKNVFPPEVAVFEPSEAEISHTQKATLVCLATGFYPDHVELSWWVN | 137 |
| | GKEVHSGVSTDPQPLKEQPALNDSRYCLSSRLRVSATFWQNPRNHFR | |
| | CQVQFYGLSENDEWTQDRAKPVTQIVSAEAWGRADCGFTSESYQQGV | |
| | LSATILYEILLGKATLYAVLVSALVLMAMVKRKDSRG | |
| TRBC2 | GATCTGAAAAACGTGTTCCCTCCTGAAGTGGCTGTCTTTGAACCATC | 138 |
| | CGAGGCCGAGATTTCCCATACCCAGAAAGCAACTCTGGTCTGTCTGG | |
| | CCACTGGATTCTACCCCGATCACGTGGAACTGTCTTGGTGGGTGAAC | |
| | GGCAAGGAAGTCCATTCCGGAGTCTCTACCGACCCTCAGCCCCTCAA | |
| | GGAGCAGCCTGCTCTCAACGATTCTCGGTACTGCCTGTCATCTCGAC | |
| | TGAGAGTGTCTGCCACCTTCTGGCAGAACCCTAGAAACCACTTTCGG | |
| | TGTCAGGTCCAGTTTTACGGCCTGAGCGAGAACGATGAGTGGACACA | |
| | GGATAGAGCCAAACCTGTGACACAGATTGTGAGCGCCGAGGCTTGGG | |
| | GACGAGCCGATTGTGGCTTCACATCCGAGTCTTACCAGCAGGGAGTG | |
| | CTGTCTGCTACAATCCTCTACGAAATTCTCCTGGGGAAGGCCACCCT | |
| | GTACGCTGTCCTCGTGTCTGCTCTGGTGCTCATGGCTATGGTCAAAC | |
| | GAAAGGACTCTAGAGGC | |
| TRBC1 (1) | LNKVFPPEVAVFEPSEAEISHTQKATLVCLATGFFPDHVELSWWVNG | 139 |
| | KEVHSGVSTDPQPLKEQPALNDSRYCLSSRLRVSATFWQNPRNHFRC | |
| | QVQFYGLSENDEWTQDRAKPVTQIVSAEAWGRADCGFTSVSYQQGVL | |
| | SATILYEILLGKATLYAVLVSALVLMAMVKRKDF | |
| TRBC1 (2) | DLNKVFPPEVAVFEPSEAEISHTQKATLVCLATGFFPDHVELSWWVN | 140 |
| | GKEVHSGVSTDPQPLKEQPALNDSRYCLSSRLRVSATFWQNPRNHFR | |
| | CQVQFYGLSENDEWTQDRAKPVTQIVSAEAWGRADCGFTSVSYQQGV | |
| | LSATILYEILLGKATLYAVLVSALVLMAMVKRKDF | |
| TRBC1 (2) | GACCTGAACAAGGTGTTCCCACCCGAGGTCGCTGTGTTTGAGCCATC | 141 |
| | AGAAGCAGAGATCTCCCACACCCAAAAGGCCACACTGGTGTGCCTGG | |
| | CCACAGGCTTCTTCCCCGACCACGTGGAGCTGAGCTGGTGGGTGAAT | |
| | GGGAAGGAGGTGCACAGTGGGGTCAGCACAGACCCGCAGCCCCTCAA | |
| | GGAGCAGCCCGCCCTCAATGACTCCAGATACTGCCTGAGCAGCCGCC | |
| | TGAGGGTCTCGGCCACCTTCTGGCAGAACCCCCGCAACCACTTCCGC | |
| | TGTCAAGTCCAGTTCTACGGGCTCTCGGAGAATGACGAGTGGACCCA | |
| | GGATAGGGCCAAACCCGTCACCCAGATCGTCAGCGCCGAGGCCTGGG | |
| | GTAGAGCAGACTGTGGCTTTACCTCGGTGTCCTACCAGCAAGGGGTC | |
| | CTGTCTGCCACCATCCTCTATGAGATCCTGCTAGGGAAGGCCACCCT | |
| | GTATGCTGTGCTGGTCAGCGCCCTTGTGTTGATGGCCATGGTCAAGA | |
| | GAAAGGATTTC | |
| TRBC | AGGACCTGAACAAGGTGTTCCCACCCGAGGTCGCTGTGTTTGAGCCA | 142 |
| | TCAGAAGCAGAGATCTCCCACACCCAAAAGGCCACACTGGTGTGCCT | |
| | GGCCACAGGCTTCTTCCCCGACCACGTGGAGCTGAGCTGGTGGGTGA | |
| | ATGGGAAGGAGGTGCACAGTGGGGTCAGCACAGACCCGCAGCCCCTC | |
| | AAGGAGCAGCCCGCCCTCAATGACTCCAGATACTGCCTGAGCAGCCG | |
| | CCTGAGGGTCTCGGCCACCTTCTGGCAGAACCCCCGCAACCACTTCC | |
| | GCTGTCAAGTCCAGTTCTACGGGCTCTCGGAGAATGACGAGTGGACC | |
| | CAGGATAGGGCCAAACCCGTCACCCAGATCGTCAGCGCCGAGGCCTG | |
| | GGGTAGAGCAGGTGAGTGGGGCCTGGGGAGATGCCTGGAGGAGATTA | |
| | GGTGAGACCAGCTACCAGGGAAAATGGAAAGATCCAGGTAGCAGACA | |
| | AGACTAGATCCAAAAAGAAAGGAACCAGCGCACACCATGAAGGAGAA | |
| | TTGGGCACCTGTGGTTCATTCTTCTCCCAGATTCTCAGCCCAACAGA | |
| | GCCAAGCAGCTGGGTCCCCTTTCTATGTGGCCTGTGTAACTCTCATC | |
| | TGGGTGGTGCCCCCCATCCCCCTCAGTGCTGCCACATGCCATGGATT | |
| | GCAAGGACAATGTGGCTGACATCTGCATGGCAGAAGAAAGGAGGTGC | |
| | TGGGCTGTCAGAGGAAGCTGGTCTGGGCCTGGGAGTCTGTGCCAACT | |
| | GCAAATCTGACTTTACTTTTAATTGCCTATGAAAATAAGGTCTCTCA | |
| | TTTATTTTCCTCTCCCTGCTTTCTTTCAGACTGTGGCTTTACCTCGG | |
| | GTAAGTAAGCCCTTCCTTTTCCTCTCCCTCTCTCATGGTTCTTGACC | |
| | TAGAACCAAGGCATGAAGAACTCACAGACACTGGAGGGTGGAGGGTG | |
| | GGAGAGACCAGAGCTACCTGTGCACAGGTACCCACCTGTCCTTCCTC | |
| | CGTGCCAACAGTGTCCTACCAGCAAGGGGTCCTGTCTGCCACCATCC | |
| | TCTATGAGATCCTGCTAGGGAAGGCCACCCTGTATGCTGTGCTGGTC | |
| | AGCGCCCTTGTGTTGATGGCCATGGTAAGCAGGAGGGCAGGATGGGG | |
| | CCAGCAGGCTGGAGGTGACACACTGACACCAAGCACCCAGAAGTATA | |
| | GAGTCCCTGCCAGGATTGGAGCTGGGCAGTAGGGAGGGAAGAGATTT | |
| | CATTCAGGTGCCTCAGAAGATAACTTGCACCTCTGTAGGATCACAGT | |
| | GGAAGGGTCATGCTGGGAAGGAGAAGCTGGAGTCACCAGAAAACCCA | |
| | ATGGATGTTGTGATGAGCCTTACTATTTGTGTGGTCAATGGGCCCTA | |
| | CTACTTTCTCTCAATCCTCACAACTCCTGGCTCTTAATAACCCCCAA | |
| | AACTTTCTCTTCTGCAGGTCAAGAGAAAGGATTTCTGA | |
| TRBC | AGGACCTGAAAAACGTGTTCCCACCCGAGGTCGCTGTGTTTGAGCCA | 143 |
| | TCAGAAGCAGAGATCTCCCACACCCAAAAGGCCACACTGGTATGCCT | |
| | GGCCACAGGCTTCTACCCCGACCACGTGGAGCTGAGCTGGTGGGTGA | |
| | ATGGGAAGGAGGTGCACAGTGGGGTCAGCACAGACCCGCAGCCCCTC | |
| | AAGGAGCAGCCCGCCCTCAATGACTCCAGATACTGCCTGAGCAGCCG | |
| | CCTGAGGGTCTCGGCCACCTTCTGGCAGAACCCCCGCAACCACTTCC | |
| | GCTGTCAAGTCCAGTTCTACGGGCTCTCGGAGAATGACGAGTGGACC | |
| | CAGGATAGGGCCAAACCCGTCACCCAGATCGTCAGCGCCGAGGCCTG | |
| | GGGTAGAGCAGGTGAGTGGGGCCTGGGGAGATGCCTGGAGGAGATTA | |
| | GGTGAGACCAGCTACCAGGGAAAATGGAAAGATCCAGGTAGCGGACA | |
| | AGACTAGATCCAGAAGAAAGCCAGAGTGGACAAGGTGGGATGATCAA | |
| | GGTTCACAGGGTCAGCAAAGCACGGTGTGCACTTCCCCCACCAAGAA | |
| | GCATAGAGGCTGAATGGAGCACCTCAAGCTCATTCTTCCTTCAGATC | |
| | CTGACACCTTAGAGCTAAGCTTTCAAGTCTCCCTGAGGACCAGCCAT | |
| | ACAGCTCAGCATCTGAGTGGTGTGCATCCCATTCTCTTCTGGGGTCC | |
| | TGGTTTCCTAAGATCATAGTGACCACTTCGCTGGCACTGGAGCAGCA | |
| | TGAGGGAGACAGAACCAGGGCTATCAAAGGAGGCTGACTTTGTACTA | |
| | TCTGATATGCATGTGTTTGTGGCCTGTGAGTCTGTGATGTAAGGCTC | |
| | AATGTCCTTACAAAGCAGCATTCTCTCATCCATTTTTCTTCCCCTGT | |
| | TTTCTTTCAGACTGTGGCTTCACCTCCGGTAAGTGAGTCTCTCCTTT | |
| | TTCTCTCTATCTTTCGCCGTCTCTGCTCTCGAACCAGGGCATGGAGA | |
| | ATCCACGGACACAGGGGCGTGAGGGAGGCCAGAGCCACCTGTGCACA | |
| | GGTGCCTACATGCTCTGTTCTTGTCAACAGAGTCTTACCAGCAAGGG | |
| | GTCCTGTCTGCCACCATCCTCTATGAGATCTTGCTAGGGAAGGCCAC | |
| | CTTGTATGCCGTGCTGGTCAGTGCCCTCGTGCTGATGGCCATGGTAA | |
| | GGAGGAGGGTGGGATAGGGCAGATGATGGGGGCAGGGGATGGAACAT | |
| | CACACATGGGCATAAAGGAATCTCAGAGCCAGAGCACAGCCTAATAT | |
| | ATCCTATCACCTCAATGAAACCATAATGAAGCCAGACTGGGGAGAAA | |
| | ATGCAGGGAATATCACAGAATGCATCATGGGAGGATGGAGACAACCA | |
| | GCGAGCCCTACTCAAATTAGGCCTCAGAGCCCGCCTCCCCTGCCCTA | |
| | CTCCTGCTGTGCCATAGCCCCTGAAACCCTGAAAATGTTCTCTCTTC | |
| | CACAGGTCAAGAGAAAGGATTCCAGAGGCTAG | |
| Hinge/ | IPNIQNPDPA | 144 |
| Spacer (1) | | |
| Hinge/ | ATTCCCAATATCCAGAACCCTGACCCTGCC | 145 |
| Spacer (1) | | |
| Hinge/ | LEDLKNVFPPE | 146 |
| Spacer (2) | | |
| Hinge/ | CTGGAGGATCTGAAAAACGTGTTCCCTCCTGAA | 147 |
| Spacer (2) | | |

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR-like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 109 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 109, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 109. In certain embodiments, the CD3% polypeptide comprises or consists of amino acids 52 to 164 of SEQ ID NO: 109.

In certain embodiments, the CD3ζ polypeptide comprises or consists of an amino acid sequence that is at least about 85%, about 90%, about 95%, about 96%, about 97%, about 98%, about 99% or about 100% homologous or identical to one or more of SEQ ID NOs: 109-111 or 113 or a fragment thereof, and/or may optionally comprise up to one or up to two or up to three conservative amino acid substitutions. In certain embodiments, the CD3ζ polypeptide comprises or consists of the amino acid sequence set forth in any one of SEQ ID NOs: 109-111 or 113.

In certain embodiments, the TCR-like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR-like fusion molecule is capable of associating with a CD3 complex (also known as “T-cell co-receptor”). In certain embodiments, the TCR-like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR-like fusion molecule replaces a native and/or an endogenous TCR in the CD3/TCR complex. In certain embodiments, the CD3 complex comprises a CD3γ chain, a CD3δ chain, and two CD3ε chains. In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 105) or a fragment thereof, and/or may optionally comprise up to one or up to two or up to three conservative amino acid substitutions.

In certain embodiments, the CD3δ chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 106) or a fragment thereof, or the amino acid sequence having a NCBI reference numbers: NP_001035741.1 (SEQ ID NO: 107) or a fragment thereof, and/or may optionally comprise up to one or up to two or up to three conservative amino acid substitutions.

In certain embodiments, the CD3ε chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 108) or a fragment thereof, and/or may optionally comprise up to one or up to two or up to three conservative amino acid substitutions.

In certain embodiments, the TCR-like fusion molecule exhibits a greater antigen sensitivity than a CAR targeting the same antigen. In certain embodiments, the TCR-like fusion molecule is capable of inducing an immune response when binding to an antigen that has a low density on the surface of a tumor cell. In certain embodiments, cells comprising the TCR-like fusion molecule can be used to treat a subject having tumor cells with a low expression level of a surface antigen, e.g., from a relapse of a disease, wherein the subject received treatment that leads to residual tumor cells. In certain embodiments, the tumor cells have a low density of a target molecule on the surface of the tumor cells. In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

Various TCR-like fusion molecules are disclosed in International Patent Application Publication No. WO2019/133969, which is incorporated by reference hereby in its entirety.

### 6.4. Antigens

In certain embodiments, the antigen bound by a modular polypeptide of the instant disclosure is a tumor antigen. Any tumor antigen (antigenic peptide) can be used in the tumor-related embodiments described herein. Sources of antigen include, but are not limited to, cancer polypeptides. The antigen can be expressed as a peptide or as an intact polypeptide or a portion thereof. The intact polypeptide or portion thereof can be native or mutagenized. Non-limiting examples of antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g., a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CRI, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PRI), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

In certain embodiments, the antigen is CD19. In certain embodiments, the antigen is expressed on an acute myeloid leukemia (AML) tissue. In certain embodiments, the antigen is expressed on an acute myeloid leukemia (AML) hematopoietic stem/progenitor cell (HSPC) and/or a leukemia stem cell (LSC). In certain embodiments, the AML HSPC expresses CD34. In certain embodiments, the antigen is expressed in a malignant hematopoietic stem cell and/or a malignant hematopoietic progenitor cell. In certain embodiments, the antigen is not expressed or expressed at a non-detectable level in a non-malignant hematopoietic stem cell and/or a non-malignant hematopoietic progenitor cell. In certain embodiments, the antigen is selected from the group consisting of CD70, IL1RAP, CD19, CD33, CLEC12A, ADGRE2, CD123, and combinations thereof.

In certain embodiments, the antigen is a pathogen antigen. Non-limiting examples of viruses include, Retroviridae (e.g., human immunodeficiency viruses, such as HIV-1 (also referred to as HDTV-III, LAVE or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronoviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Naira viruses); Arena viridae (hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), the agents of non-A, non-B hepatitis (class 1-internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g., M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 6.5. Vectors

Genetic modification of an immunoresponsive cell (e.g., a T cell) can be accomplished by transducing a substantially homogeneous cell composition with a recombinant DNA construct. In certain embodiments, a retroviral vector (is employed for the introduction of the DNA construct into the cell. For example, a polynucleotide encoding any polypeptide or system disclosed herein can be cloned into a retroviral vector and expression can be driven from its endogenous promoter, from the retroviral long terminal repeat, or from a promoter specific for a target cell type of interest. In certain embodiments, the retroviral vector is a gamma-retroviral vector. In certain embodiments, the retroviral vector is a lentiviral vector. Non-viral vectors may be used as well.

For initial genetic modification of an immunoresponsive cell to include a polypeptide and/or a system disclosed herein, a retroviral vector is generally employed for transduction, however any other suitable viral vector or non-viral delivery system can be used. The polypeptides and/or the system can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides, e.g., SEQ ID NOs: 23-26). Combinations of retroviral vector and an appropriate packaging line are also suitable, where the capsid polypeptides will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits a high efficiency of infection, stable integration into the host cell genome, and durable expression of the recombinant gene product(s) (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALENs nucleases, CRISPR). Transient expression may be obtained by RNA electroporation. In certain embodiments, recombinant receptors can be introduced by a transposon-based vector. In certain embodiments, the transposon-based vector comprises a transposon (a.k.a. a transposable element). In certain embodiments, the transposon can be recognized by a transposase. In certain embodiments, the transposase is a Sleeping Beauty transposase.

The resulting cells can be grown under conditions similar to those for unmodified cells, whereby the modified cells can be expanded and used for a variety of purposes.

### 6.6. Cells

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

### 6.7. Formulations and Administration

The presently disclosed subject matter provides compositions comprising presently disclosed cells. In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. in certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells)) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 6.8. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g., leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g., leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenström macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain embodiments, the myeloid disorder is acute myeloid leukemia (AML). In certain embodiments, the first and/or second antigens are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g., a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PRI), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

In certain embodiments, the myeloid disorder is acute myeloid leukemia (AML), and the first and/or second antigens are expressed on an AML hematopoietic stem/progenitor cell (HSPC) and/or a leukemia stem cell (LSC). In certain embodiments, the AML HSPC expresses CD34. In certain embodiments, the first antigen and/or the second antigen are not expressed or expressed at a non-detectable level in a non-hematopoietic stem cell and/or a non-hematopoietic progenitor cell. In certain embodiments, the first antigen and/or the second antigen are independently selected from the group consisting of CD70, IL1RAP, CD33, CLEC12A, ADGRE2, CD123, and combinations thereof.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an infectious disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into the presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion or eradicated at the earliest signs of toxicity.

### 6.9. Kits

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises compositions, buffers, nucleic acids, vectors, and reagents for producing the presently disclosed cells. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6.10. Exemplary Embodiments

In certain non-limiting embodiments, the presently disclosed subject matter is directed to systems comprising two nucleic acid constructs, wherein the first nucleic acid construct comprises a nucleic acid sequence encoding one of a complementary pair of N- and C-split inteins; the second nucleic acid construct comprises a nucleic acid sequence encoding the other of the complementary pair of N- and C-split inteins; and at least one nucleic acid construct encodes an endoplasmic reticulum (ER) retention motif. In certain non-limiting embodiments, such systems comprise a first nucleic acid construct comprising a nucleic acid sequence encoding: a first extein; and a first N-split intein of a first complementary pair of split inteins; as well as a second nucleic acid construct comprising a nucleic acid sequence encoding: a second extein; a first C-split intein of the first complementary pair split inteins; and a second N-split intein, wherein the second N-split intein is of a second complementary pair of split inteins; and a third nucleic acid construct comprising nucleic acid sequences encoding: a third extein; and a second C-split intein, wherein the second C-split intein is of the second complementary pair split inteins. In certain non-limiting embodiments, such systems comprise a first nucleic acid construct comprising a nucleic acid sequence encoding: a first extein; and a first leucine zipper motif; a second nucleic acid construct comprising a nucleic acid sequence encoding: a second leucine zipper motif; and an N-split intein of a complementary pair split inteins; and a third nucleic acid construct comprising a nucleic acid sequence encoding: a second extein; and a C-split intein of the complementary pair split inteins. In certain non-limiting embodiments, such systems comprise a first nucleic acid construct comprising a nucleic acid sequence encoding: a first extein; and a first N-split intein of a first complementary pair of split inteins; a second nucleic construct comprising a nucleic acid sequence encoding: a second extein; a first C-split intein of the first complementary pair split inteins; and a second N-split intein, wherein the second N-split intein is of a second complementary pair of split inteins; a third nucleic acid construct comprising a nucleic acid sequence encoding: a third extein; a second C-split intein, wherein the second C-split intein is of the second complementary split inteins; and a third N-split intein, wherein the third N-split intein is of a third complementary pair of split inteins; and a fourth nucleic acid construct comprising a nucleic acid sequence encoding: a fourth extein; and a third C-split intein, wherein the third C-split intein is of the third complementary pair of split inteins. In certain of the foregoing systems, the N-terminal amino acid of an N-split intein is linked to the C-terminal amino acid of an extein, or the N-terminal amino acid of a extein is linked to the C-terminal amino acid of a C-split intein. In certain of the foregoing systems, the complementary pair of split inteins are selected from the group consisting of a Cfa intein, a gp41-1 intein, gp41-8 intein, an Aes123 PolB1 Intein (Aes Intein), an NrdJ-1 intein, an IMPDH-1 intein, a SspGyrB intein, a DNA polymerase III (DnaE) intein, orthologs thereof, and variants thereof. In certain of the foregoing systems, the complementary pair of N- and C-split inteins comprise a splicing motif selected from the group consisting of CLS, CFN, CLD, HNS, SVV, SVYLN, and CLV. In certain of the foregoing systems, the N-split inteins have an amino acid sequence selected from SEQ ID NOs: 1, 3, 5, 7, and 9. In certain of the foregoing systems, the C-split inteins have an amino acid sequence selected from SEQ ID NOs: 2, 4, 6, 8, and 10. In certain of the foregoing systems, the N-split inteins have an amino acid sequence selected from SEQ ID NOs: 1, 3, 5, 7, and 9, and the complementary C-split inteins have an amino acid sequence selected from SEQ ID NOs: 2, 4, 6, 8, and 10 respectively. In certain of the foregoing systems, the ER retention motif is a KKXX motif, or an RXR motif. In certain of the foregoing systems, the E319K motif comprises SEQ ID No. 18. In certain of the foregoing systems, the RXR motif comprises a sequence selected from any one of SEQ ID NOs: 19-22. In certain of the foregoing systems, the N-terminal amino acid of the ER retention motif is linked to the C-terminal amino acid of a N-split intein. In certain of the foregoing systems, the ER retention motif is an RXR motif, the RXR motif flanked by sequences encoding an extein. In certain of the foregoing systems, at least one nucleic acid construct further comprises a nucleotide sequence encoding one or more of an affinity tag, a spacer, or a linker, wherein the amino acid sequence of the one or more of the affinity tag, the spacer, or the linker is disposed on the polypeptide chain, between the C-terminal amino acid of a extein and the N-terminal amino acid of a N-split intein, or between the N-terminal amino acid of a extein and the C-terminal amino acid of a C-split intein. In certain of the foregoing systems, the affinity tag is one or more of: a FLAG tag comprising an amino acid sequence DYKDDDDK; a Strep tag comprising the amino acid sequence of SEQ ID NO: 27; a V5 tag comprising the amino acid sequence of SEQ ID NO: 28; a CD34 tag comprising the amino acid sequence of SEQ ID NO: 29; a CD20 mimotope tag comprising the amino acid sequence of SEQ ID NO: 30; and a tagBFP comprising the amino acid sequence of SEQ ID NO: 31. In certain of the foregoing systems, the spacer is a CD8 spacer comprising the amino acid sequence of SEQ ID NO: 32, a CD28 spacer comprising the amino acid sequence of SEQ ID NO: 33, or a PD-1 spacer comprising the amino acid sequence of SEQ ID NO: 34. In certain of the foregoing systems, the linker has an amino acid sequence selected from any one of SEQ ID NOs: 11-17, or comprises the amino acid sequence, GGGGSGGGS, GGGGSGTG, GTSRAKRGS, ALGGSGGGS, GGGGSTS, SGGGGSD, GSSGSG, GSGGTR, GSGTR, TSGSG, GSGGS, GSS, GTG, LES, GSG, GSL, or ALG. In certain of the foregoing systems, the linker is a cleavable linker. In certain of the foregoing systems, the cleavable linker comprises a sequence selected from one or more of SEQ ID NOs: 23, 24, 25 or 26. In certain of the foregoing systems, at least one nucleic acid construct comprises a nucleotide sequence encoding a regulatable gene element. In certain of the foregoing systems, the regulatable gene element encodes a regulator motif that regulates expression of the extein. In certain of the foregoing systems, the regulator motif comprises a drug-stabilized signaling domain, a drug-destabilized degron domain, or a drug-regulatable self-cleaving domain. In certain of the foregoing systems, the drug-destabilized degron domain is a dihydrofolate reductase destabilization domain (DHFR-DD). In certain of the foregoing systems, the drug is trimethoprim or analogs thereof. In certain of the foregoing systems, the regulator motif comprises a non-structural 3 (NS3) protease cleavage site and a drug-regulated protease. In certain of the foregoing systems, the drug-regulated protease is a HCV protease that is regulated by asunaprevir, grazoprevir, or analogs thereof. In certain of the foregoing systems, the regulator motif comprises a dimerization domain that dimerizes in the presence of a drug. In certain of the foregoing systems, the N-terminal amino acid of the ER retention motif is linked to the C-terminal amino acid of the dimerization domain. In certain of the foregoing systems, the dimerization domain is selected from a FK506 binding protein 12 (FKBP12) and a FKBP-rapamycin-binding (FRB). In certain of the foregoing systems, the dimerization domain is regulated by rapamycin or analogs thereof. In certain of the foregoing systems, the regulatable gene element comprises a sequence selected from SEQ ID NOs: 38-47, or the sequence DEMEECSQH. In certain of the foregoing systems, the extein comprises an extracellular antigen binding domain, an extracellular cytokine, a transmembrane domain, a signaling domain, or a combination thereof. In certain of the foregoing systems, the extracellular antigen binding domain further comprises a hinge region. In certain of the foregoing systems, the hinge region comprises an amino acid sequence selected from SEQ ID NOs: 144-147. In certain of the foregoing systems, the extracellular antigen binding domain comprises a single chain variable fragment (scFv), a Fab, a F (ab) 2, or a single domain VHH antibody. In certain of the foregoing systems, the extein comprises a chimeric antigen receptor (CAR), a chimeric co-stimulating receptor (CCR), a T cell receptor (TCR), a TCR-like fusion molecule, orthologs thereof, or variants thereof. In certain of the foregoing systems, the extracellular antigen binding domain binds one or more antigens selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CRI, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11. In certain of the foregoing systems, the CAR further comprises a signal sequence. In certain of the foregoing systems, the signal sequence comprises an amino acid sequence of one of SEQ ID NOs: 128-135. In certain of the foregoing systems, the CAR comprises an extracellular antigen binding domain of an anti-CD19 antibody. In certain of the foregoing systems, the extracellular antigen binding domain of the CAR is an anti-CD19 scFv. In certain of the foregoing systems, the extracellular antigen binding domain of the CAR is an anti-CD19 scFv has the heavy chain variable domain (VH) of SEQ ID NO: 94 and the light chain variable domain (VL) of SEQ ID NO: 95. In certain of the foregoing systems, the extracellular antigen binding domain of the CAR is an anti-CD19 scFv having the sequence of SEQ ID NO: 96. In certain of the foregoing systems, the CAR comprises an extracellular antigen binding domain of an anti-CD20 antibody. In certain of the foregoing systems, the extracellular antigen binding domain of the CAR is an anti-CD20 scFv. In certain of the foregoing systems, the extracellular antigen binding domain of the CAR is an anti-CD20 scFv has the heavy chain variable domain (VH) of SEQ ID NO: 97 and the light chain variable domain (VL) of SEQ ID NO: 98. In certain of the foregoing systems, the extracellular antigen binding domain of the CAR is an anti-CD20 scFv having the sequence of SEQ ID NO: 99. In certain of the foregoing systems, the extracellular antigen binding domain of the CAR is a VHH antibody. In certain of the foregoing systems, the VHH antibody has the sequence of SEQ ID NO: 100. In certain of the foregoing systems, the TCR comprises an alpha chain constant (TRAC) region and a beta chain constant (TRBC) region, wherein: the TRAC is encoded by the nucleotide sequence of SEQ ID NO:136; the TRBC is encoded by the nucleotide sequence selected from any one of SEQ ID NOs: 138, 141, 142 or 143; and the TRBC has comprises an amino acid sequence selected from any one of SEQ ID NOs: 137, 139, or 140. In certain of the foregoing systems, the cytokine is selected from the group consisting of IL-7, IL-15, and IL-18. In certain of the foregoing systems, the signaling domain is one or more of CD3δ, CD3γ, CD3ε, CD3ζ, CD28, 4-1BB, ICOS, OX40, CD27, CD40, NKG2D, DAP-10, CD2, CD150, CD226, NKG2D, Zap70, orthologs thereof or variants thereof. In certain of the foregoing systems, the signaling domain further comprises a kinase. In certain of the foregoing systems, the kinase is one or more of a Src kinase, a Syk kinase, or a receptor tyrosine (RTK). In certain of the foregoing systems, the kinase is one or more of a Src kinase, a Syk kinase, or a receptor tyrosine (RTK). In certain of the foregoing systems, the signaling domain comprises a kinase domain from one or more of PDGFR, KIT, Abl, Arg, EGFR, Raf, VEGFR, PDGFR, Flt3, Abl, Arg, or ErbB2, or orthologs thereof. In certain of the foregoing systems, the CD3γ signaling domain comprises the amino acid sequence of SEQ ID No. 105; the CD3δ signaling domain comprises the amino acid sequence selected from any one of SEQ ID NOs: 106 or 107; the CD3δ signaling domain comprises the amino acid sequence of SEQ ID NO: 108; the CD3ζ signaling domain comprises the amino acid sequence selected from any one of SEQ ID NOs: 109-111, or 113; the CD28 signaling domain comprises the amino acid sequence selected from any one of SEQ ID NOs: 101-103; the 4-1BB signaling domain comprises the amino acid sequence of SEQ ID NO: 104; the Zap70 signaling domain comprises the amino acid sequence selected from any one of SEQ ID NOs: 114 or 115. In certain of the foregoing systems, the signaling domain further comprises an immunoreceptor tyrosine-based activation motif (ITAM). In certain of the foregoing systems, the ITAM comprises the amino acid sequence selected from any one of SEQ ID NOs: 116, 118, 120, 122, 124, 126. In certain of the foregoing systems, the nucleic acid construct encodes for an amino acid sequence selected from any one of SEQ ID NOs: 55-93.

In certain non-limiting embodiments, the presently disclosed subject matter is directed to systems comprising two or more nucleic acid constructs, each nucleic acid construct comprising a nucleotide sequence encoding: at least one extein; at least one of a complementary pair of N- and C-split inteins; at least one endoplasmic reticulum (ER) retention motif; and a regulator motif or a regulatable domain that regulates expression of the extein. In certain of such systems, the extein comprises one or more of an extracellular antigen binding domain, an extracellular cytokine, a transmembrane domain, or a signaling domain. In certain of such systems, the complementary pair of split inteins are selected from the group consisting of a Cfa intein, a gp41-1 intein, gp41-8 intein, an Aes123 PolB1 Intein (Aes Intein), an NrdJ-1 intein, an IMPDH-1 intein, a SspGyrB intein, a DNA polymerase III (DnaE) intein, orthologs thereof, and variants thereof. In certain of such systems, the N-split inteins have an amino acid sequence selected from SEQ ID NOs: 1, 3, 5, 7, and 9. In certain of such systems, the C-split inteins have an amino acid sequence selected from SEQ ID NOs: 2, 4, 6, 8, and 10. In certain of such systems, the N-split inteins have an amino acid sequence selected from SEQ ID NOs: 1, 3, 5, 7, and 9, and the complementary C-split inteins have an amino acid sequence selected from SEQ ID NOs: 2, 4, 6, 8, and 10 respectively. In certain of such systems, the ER retention motif is an E319K motif (KKXX), or an RXR motif. In certain of such systems, the E319K motif comprises the sequence the SEQ ID NO: 18. In certain of such systems, the RXR motif comprises a sequence selected from any one of SEQ ID NOs: 19-22. In certain of such systems, the N-terminal amino acid of the ER retention motif is linked to the C-terminal amino acid of a N-split intein. In certain of such systems, the ER retention motif is an RXR motif, the RXR motif flanked by sequences encoding an extein. In certain of such systems, the regulator motif or the regulatable domain comprises a drug-stabilized signaling domain, a drug-destabilized degron domain, or a drug-regulatable self-cleaving domain. In certain of such systems, the extein comprises one CAR domains and one CCR domains. In certain of such systems, the extein comprises at least two extracellular antigen binding CAR domains. In certain of such systems, the extein comprises at least two extracellular antigen binding CCR domains. In certain of such systems, the extein comprises two CAR domains and two CCR domains. In certain of such systems, the extein comprises at least one signaling domain. In certain of such systems, the extein comprises at least two signaling domains. In certain of such systems, the signaling domain further comprises a kinase. In certain of such systems, the signaling domain is one or more of CD3δ, CD3γ, CD3ε, CD3ζ, CD28, 4-1BB, ICOS, OX40, CD27, CD40, NKG2D, DAP-10, CD2, CD150, CD226, NKG2D, Zap70, orthologs thereof or variants thereof. In certain of such systems, the nucleic acid construct comprises: a nucleotide sequence encoding regulatory motif comprising a non-structural 3 (NS3) protease cleavage site and a drug-regulatable HCV protease; and a signaling domain comprising a kinase. In certain of such systems, the nucleic acid construct comprises: a nucleotide sequence encoding regulatory motif comprising a non-structural 3 (NS3) protease cleavage site and a drug-regulatable HCV protease; and a Zap 70 signaling domain. In certain of such systems, the nucleic acid construct comprises: a nucleotide sequence encoding regulatory motif comprising a dimerization domain that dimerizes in the presence of a drug; and at least one signaling domain, wherein the signaling domain comprises a kinase.

In certain non-limiting embodiments, the presently disclosed subject matter is directed to methods of modifying a cell comprising delivering to the cell, the system of the present disclosure. In certain of such methods, the cell is a mammalian cell. In certain of such methods, the mammalian cell is an immune cell. In certain of such methods, the immune cell is a T cell.

In certain non-limiting embodiments, the presently disclosed subject matter is directed to methods for enriching a population of modified cells where the enrichment comprises: modifying a population of cells as disclosed herein; culturing the population of cells; and enriching for the population of modified cells by selecting for the surface expression of an extein. In certain of such methods, the cell is a mammalian cell. In certain of such methods, the mammalian cell is an immune cell. In certain of such methods, the immune cell is a T cell.

In certain non-limiting embodiments, the presently disclosed subject matter is directed to methods for treating a disease comprising providing to a subject in need thereof, a population of modified cells comprising a system as described herein, or a cell modified according to a method of modification disclosed herein, or an enriched population of cells enriched according to a method disclosed herein. In certain of such embodiments, the subject is a human subject. In certain of such embodiments, the disease is a cancer, an autoimmune disease, an inflammatory disease, or a graft versus-host disease. In certain of such embodiments, the cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma. In certain of such embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain of such embodiments, the lymphoma is Hodgkin's lymphoma or non-Hodgkin's lymphoma. In certain of such embodiments, the non-Hodgkin's lymphoma is B-cell non-Hodgkin's lymphoma or T-cell non-Hodgkin's lymphoma. In certain of such embodiments, the cancer comprises cells expressing CD19 or CD20. In certain of such embodiments, the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MARTI, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

### 7. EXAMPLES

### 7.1. Materials and Methods

### 7.1.1. Plasmid Construction

Inteins and other proteins of interest (e.g., antigen binding domains, spacers, transmembrane domains, cytoplasmic signaling domains, drug regulation domains, reporter proteins) were combined into vector sequences using standard molecular cloning techniques and arranged into polycistronic configurations with self-splicing 2A peptides.

### 7.1.2. Retroviral Transduction

Retrovirus packaging lines were stably transfected with retroviral DNA plasmids encoding intein and other construct elements. Viral supernatants were produced and concentrated with polyethylene glycol. T cells were stimulated with plate-bound antibodies or antibody-coated beads. Following T cell stimulation, T cells were co-cultured with retroviral particles adsorbed on to recombinant human fibronectin (RETRONECTIN®, Takara Bio Inc.) and centrifuged into proximity on coated culture plates. Following transduction, T cells were assessed for construct expression by flow cytometry. Other methods for vector integration included, lentiviral or adeno associated virus (AAV) vectors or non-viral targeted integration of DNA using CRISPR/Cas-based integration. Transposon delivery and non-integrating lentiviral vector template delivery combined with site-specific CRISPR/Cas-based integration was also compatible with the intein-sorting system methodology.

### 7.1.3. Intein-Based Cell Sorting

T cells co-transduced with multiple vector components of the intein sorting system were incubated with anti-affinity tag magnetic beads (e.g., anti-FLAG, and hCD34) and positively selected on magnetic isolation columns (Miltenyi Biotec). Post-sorting cell purity and construct expression was confirmed by flow cytometry.

### 7.1.4. Luciferase-Based Target Lysis Assay

Target cells were genetically modified to express a luciferase gene (firefly or click beetle red) and subsequently incubated with CAR T cells at varying effector:target (E:T) ratios for 24 hours. Viable residual target cells were quantified by luminous flux following addition of D-Luciferin.

### 7.1.5. Live Cell Imaging

Target cells were genetically modified to express iRFP713 and incubated in an INCUCYTE® Live Cell imaging system with CAR T cells co-expressing EGFP at varying effector:target ratios. T cell and target cell counts were assessed via serial microscopic imaging.

### 7.2. Data Compression

A The use of degenerate inteins tagged to domains used in common for multiple CAR constructs (e.g., Zap70 signaling domain and 4-1BB costimulatory domain) can be multiplexed with multiple antigen binding domain-containing molecules to generate a greater number of trans-spliced CAR and CCR molecules than could be encoded with the same amount of DNA. DNA “data” savings increases with the size of the encoded domains and as the domains are reused in a greater number of molecules.

The quantity of DNA, Q_{C}, required to encode a series of conventionally encoded CAR and CCR molecules with common binding domains of size Bᵢ and signaling domains of size Dⱼ can be described by Equation 1;

$${Q}_{C} = d \sum {B}_{i} + b \sum {D}_{j} \qquad Equation 1$$

where d is the number of distinct signaling domains with size Dⱼ used and b is the number of distinct binding domains with size Bi used.

Whereas the quantity of DNA, Q_{I}, required to encode a series of intein-encoded CAR and CCR molecules can be described by Equation 2;

$${Q}_{I} = \sum {B}_{i} + \sum {D}_{j} + {bI}_{N} + {dI}_{C} \qquad Equation 2$$

where I_{N} is the size of DNA required to encode the N terminal split intein and I_{C} is the size of DNA required to encode the C terminal split intein.

The DNA savings (Δ=Q_{c}−Q_{I}) resulting from intein-based compression encoding can be determined by Equation 3

$$Δ = ( d - 1 ) \sum {B}_{i} + ( b - 1 ) \sum {D}_{j} - {bI}_{N} - {dI}_{C} \qquad Equation 3$$

where d is the number of signaling domains encoded, b is the number of binding stalk molecules encoded, Bᵢ is the size of each binding domain, Dⱼ is the size of each signaling domain, I_{N} is the size of the N terminal split intein, and I_{C} is the size of the C terminal intein. This is illustrated by one example of conventional encoding of two CAR and two CCR molecules utilizing an NS3 drug-regulated Zap70 signaling domain and a 4-1BB costimulatory domain with domain sizes; B1=1 kB, B2=1 kB, D1=2.2 kB, D2=0.1 kB. This would require per Equation 1:

$$Equation 4$$

$$2 * ( B 1 + B 2 ) + 2 * ( D 1 + D 2 ) = 2 * ( 1 + 1 ) + 2 * ( 2.2 + 0.1 ) = 8.6 kB$$

Rearranging by CAR and CCR molecules:

$$Equation 5$$

$${( B 1 + D 1 )}_{CAR 1} + {( B 2 + D 1 )}_{CAR 2} + {( B 1 + D 2 )}_{CCR 1} + {( B 2 + D 2 )}_{CCR 1} = 3.2 + 3.2 + 1.1 + 1.1 = 8.6 kB$$

In contrast, intein encoding where I_{N}=0.3 kB and I_{C}=0.1, would require the following DNA to sum encode:

$$Equation 5$$

$$( B1 + B 2 ) + ( D 1 + D 2 ) + 2 ( {I}_{N} ) + 2 ( {I}_{C} ) = 2 + 2.3 + 0.6 + 0.2 = 5.1 kB$$

which yields a 8.6 kB−5.1 kB=3.5 KB savings of DNA.

Confirming with Equation 3, data savings is:

$$1 * 2 + 1 * 2.3 - 0.6 - 0.2 = 3.5 kB \qquad Equation 6$$

This data savings increases the number of antigen binding domains B and signaling domains D, or the size of each domain increases.

Use of three signaling domains with sizes (2 kB, 0.3 kB, and 0.1 kB) and three antigen binding domains with size 1 kB, where I_{N}=0.3 kB and I_{C}=0.1 kB, would encode nine receptors and would yield the following DNA savings in intein encoding compared with conventional encoding:

$$Equation 7$$

$$2 * ( 1 + 1 + 1 ) + 2 * ( 2 + 0.3 + 0.1 ) - 3 * 0.3 - 3 * 0.1 = 9.6 kB .$$

### 7.3. Two-Vector Intein Sorting System-Secreted Affinity Tag with Intein Tag, Transmembrane Intein Capture Stalk

In the instant example, mouse T cells were transduced with the following 2 vectors (FIG. 2A): (Vector 1) encodes a secreted FLAG-tagged intein-tagged construct comprised of a FLAG affinity tag fused to a gp41-1 intein N terminus and also encodes a BFP reporter, while (Vector 2) encodes a capture intein construct comprised of a gp41-1 intein C terminus fused to an IgG1 hinge spacer, a CD28 transmembrane domain, and truncated CD3 zeta delta non-signaling cytoplasmic domain. Vector 2 also encodes a Thy1.1 reporter. The two intein-tagged constructs are covalently linked during trans-splicing of the gp41-1 intein, resulting in an extracellular splice junction. The FLAG-tagged intein-tagged construct is captured by the transmembrane intein-tagged construct only when constructs are expressed by the same cell, enabling the trans-splicing reaction. Cells transduced with both vectors were enriched via MACS sorting with anti-FLAG magnetic beads, yielding a highly pure BFP+/Thy1.1+ population (FIG. 2B).

### 7.4. Two-Vector and Three-Vector Intein Modular CAR and Sorting System-Extracellular scFv with Spacer Fused to Transmembrane Intein Capture Stalk Trans-Splicing with Cytoplasmic Signaling Domain Shuttle Intein and and Cytoplasmic Intein Adapter Molecules

The instant example is directed to an intein sorting system using an inward rectifier potassium (Kir6.2) RXR ER retention motif (FIG. 3A), where mouse T cells were transduced with the following 2 vectors: (Vector 1) encodes a Thy1.1 reporter and also encodes a transmembrane intein-tagged construct comprised of a human CD34 affinity tag (QBEND epitope) that is flanked by two human-CD20 affinity tags (rituximab binding mimotope) to create an RQR domain, a FLAG affinity tag, a CD8 hinge and transmembrane domain, a consensus sequence from this alignment (Cfa) intein N terminus, and a Kir6.2 RXR or E3 19K KKXX endoplasmic reticulum (ER) retention motif, while (Vector 2) encodes Cfa intein C terminus fused to a CD28 costimulatory domain, a CD3-zeta chain, and also encodes an EGFP reporter. Trans-splicing of the two Cfa intein-tagged constructs results in an intracellular splice junction and creates a transmembrane bound construct with both the RQR and FLAG affinity-tags on the cell surface. Surface expression of these affinity tags are markedly reduced due to ER retention of the transmembrane construct until the two inteins trans-splice, which removes the ER retention motif. Cells transduced with both vectors were enriched via MACS sorting with anti-FLAG magnetic beads, yielding a highly pure population of T cells expressing each affinity tag on the surface.

Mouse T cells were transduced with the following 2 vectors: (Vector 1) encodes a Thy1.1 reporter, and also encodes an antigen-binding intein-tagged construct comprising a CD19-targeting scFv fused to a hCD34 affinity tag, a CD8 transmembrane domain, a Cfa intein N terminus, and a Kir6.2 RXR or an E319K KKXX, ER retention motif, while (Vector 2) encodes Cfa intein C terminus fused to a CD28 costimulatory domain, a CD3-zeta chain, and also encodes an EGFP reporter. Trans-splicing of the two Cfa intein-tagged constructs results in an intracellular splice junction leading to formation of a full-length CD19 chimeric antigen receptor (CAR). Surface expression of the CD19 antigen-binding domain is markedly reduced due to the ER retention motif until the two inteins trans-splice, which removes the ER retention motif. Cells transduced with both vectors were enriched by MACS sorting with anti-hCD34 magnetic beads, yielding highly pure hCD34+CD19-CAR T cells (FIGS. 3B-3C). CD19-CAR T cells (effector cells) generated by trans-splicing of Cfa inteins, or control non-transduced T cells were cultured for 24 hours with CD19 expressing target cells (e.g., BM185 cells) at various E/T ratios. FIG. 3D demonstrates efficacy of the CD19-CAR T cells in killing the BM185-CD19 target cells.

FIG. 3E illustrates an exemplary three vector intein retention-based using an RXR or a E319K (KKXX) retention motifs. FIGS. 3F-3I demonstrates the ability of RXR-type ER retention motifs to function inside of a polypeptide chain (in this case a 2A peptide remnant remaining attached to the RXR retention motif). In contrast, the E3 19K KKXX-type motif fails in this setting due to the requirement of this retention motif to occupy the end of the polypeptide chain. In this example, T cells are transduced with a CD20-mimotope, hCD34-tag, and FLAG-tag containing sorting stalk molecule with cytoplasmic Cfa N intein and E319K retention motif (Vector 1). The cells are co-transduced with a second vector encoding a Cfa C intein fused to a gp41-1 N intein (cytoplasmic adapter intein) with a fused Kir6.2 RXR or E319K KKXX ER retention motifs followed by a 2A peptide and an EGFP reporter (Vector 2). The KKXX retention motif fails to effectively retain the spliced sorting stalk protein in the ER in this setting, while the RXR motif, which can tolerate appended C terminal amino acids, retains the protein inside the cells. For cells transduced with Vector 1 and Vector 2 encoding either RXR or KKXX motifs, co-transduction with Vector 3 encoding a gp41-1 C intein removes the ER retention motif and promotes strong surface trafficking of the fully spliced sorting stalk molecule. However, the RXR-type ER retention motif version enables high expression of this construct only in triple-transduced cells, enabling selective MACS sorting of triple-transduced cells. This methodology enables single-step MACS purification of cells incorporating two or more vectors with enhanced overall vector payload to generate effector cells with enhanced functionality. Furthermore, use of RXR-type ER retention motifs enables placement of ER-retained constructs in 5′ or 3′ locations in 2A peptide flanked polycistronic vectors containing multiple encoded proteins.

### 7.5. Two-Vector Dual Secreted Intein-Tagged Modular scFv Sorting System for Post-Translational CAR Assembly

### 7.5.1. Dual Secreted CAR Fused to a Cytoplasmic Signaling Domain

In this example, mouse T cells were transduced with the following 2 vectors (FIG. 4A): (Vector 1) encodes dual affinity-tagged intein-tagged scFvs (targeting CD19 or CD20) fused to a FLAG tag and gp41-1 intein N terminus and also encodes a BFP reporter. (Vector 2) encodes a transmembrane signaling domain shuttle comprised of a gp41-1 intein C terminus fused to a CD8 spacer and transmembrane domain, a CD28 costimulatory domain, and a CD3-zeta chain and also encodes a Thy1.1 reporter (FIG. 4A). Following splicing of the two gp41-1 intein-tagged constructs, both CD19 and CD20 binding-domains are displayed on the cell surface, recapitulating full-length CAR molecules (FIG. 4A). Cells containing both vectors were enriched by MACS sorting with anti-FLAG magnetic beads, resulting in high purity of FLAG-positive dual-CAR T cells (FIG. 4B). FIGS. 4C and 4D show efficacy of the CD19/CD20-targeted dual-CAR T cells respectively in killing the BM185-CD19 and BM185-CD20 cells. Unexpectedly, intein-scFv molecules appear to only splice inside the ER (possibly due to the reducing environment needed to maintain intein sulfhydryl groups in the reduced state vs. the extracellular oxidizing environment). This results in unique expression of affinity-tagged inteins-scFv-armed intein-CARs only in dual-transduced T cells and enables selective MACS sorting of dual-transduced T cells.

### 7.5.2. Dual Secreted CAR Fused to Zap70

In this example, mouse T cells were transduced with the following 2 vectors (FIG. 4E): (Vector 1) encodes two distinct antigen-binding scFv domain (e.g., recognizing CD19 or CD20 antigens) containing polypeptides each fused to transmembrane domains and degenerate cytoplasmic N terminus intein domains, and to a distinct affinity tag. (Vector 2) encodes a Zap70 domain shuttle comprised of a degenerate intein C terminus, and a EGFP protein tag. Following trans-splicing of combinations of two degenerate intein-tagged antigen-binding polypeptides with the Zap70 domain shuttle, a CD19 CAR and a CD20 CAR, each attached to a Zap70 domain are generated and expressed at the cell surface. Cells containing both vectors were enriched by MACS sorting with anti-FLAG magnetic beads, resulting in high purity of FLAG-positive dual-CAR T cells (FIG. 4F). T cells co-expressing trans-spliced CD19/Zap70 and CD20/Zap70 CARs (effector cells), or non-transduced T cells (control) were cultured for 24 hours with C1498 target cells, which express both CD19 and CD20 antigens. FIG. 4G shows efficacy of the effector T cells in killing target cells. This use of degenerate splicing of inteins sharing a common cytoplasmic signaling domain shuttle enables reuse of DNA encoding large structural elements (e.g., Zap70 interdomain and kinase), serving as a data compression algorithm. This enables greater amount of receptors to be generated per the amount of DNA delivered by the vector and is associated with very high expression of the trans-spliced CAR constructs. Selective sequestration of the FLAG-tagged CAR by the ER retention motif and subsequent surface trafficking following trans-splicing with the appropriate cytoplasmic signaling domain shuttle and removal of the ER retention motif enables selective MACS sorting of dual-transduced cells (molecular coincidence detector).

### 7.6. Three-Vector Intein CAR and Sorting System-Secreted Affinity-Tagged Intein-Tagged scFv, Transmembrane Intein Adapter with ER Retention Motif, and Cytoplasmic Signaling Domain Shuttle

In the instant example, mouse T cells were transduced with the following 3 vectors (FIG. 5A): (Vector 1) encodes dual CD19 and CD20-binding scFvs each fused to a FLAG-tag and gp41-1 intein N terminus and also encodes a BFP reporter, while (Vector 2) encodes a Thy1.1 reporter and a dual-intein transmembrane adapter comprised of a gp41-1 intein C terminus fused to CD8 extracellular and transmembrane domains, a Cfa intein N terminus, and an E319K ER retention motif, and (Vector 3) encodes a cytoplasmic signaling domain shuttle comprised of a Cfa intein C terminus fused to a CD28 costimulatory domain and a CD3-zeta chain and also encodes an EGFP reporter. The gp41-N intein-tagged scFv molecules trans-splice with the dual-intein transmembrane adapter in the ER, which can exit the ER following trans-splicing of Cfa inteins of the adapter and cytoplasmic shuttle constructs, which recapitulates a full-length CAR, with cleavage of the ER retention motif. Triple-transduced cells were selected by MACS sorting using anti-FLAG magnetic beads, resulting in high purity of FLAG-positive dual-CAR T cells (FIG. 5B). These CD19/CD20-targeted dual-CAR T cells demonstrate killing of BM185 target cells expressing either CD19 (FIG. 5C), or CD20 (FIG. 5D) in 24-hour co-culture experiments. Thus, this orthogonal two-intein, tri-molecular trans-splicing methodology enables selective MACS sorting of T cells incorporating three unique vectors, which can further encode additional transgenes to enhance effector cell functionality.

### 7.7. Modular Leucine Zipper—ZipR-CAR—Dual Antigen Specificity—Secreted Affinity-Tagged Leucine Zipper-Tagged scFv, Transmembrane Zipper-Intein Adapter

In the instant example, mouse T cells were transduced with the following 3 vectors (FIG. 6A): (Vector 1) encodes dual CD19 and CD20-binding scFvs each fused to a FLAG-tag and RR12EE345L leucine zipper and also encodes a BFP reporter, while (Vector 2) encodes a Thy1.1 reporter and a leucine zipper-intein transmembrane adapter comprised of a EE12RR345L leucine zipper fused an IgG1 hinge, CD28 transmembrane domain, a Cfa intein N terminus, and an E319K ER retention motif, and (Vector 3) encodes a cytoplasmic signaling domain shuttle comprised of a Cfa intein C terminus fused to a CD28 costimulatory domain and a CD3-zeta chain and also encodes an EGFP reporter. The leucine zipper-tagged scFv molecules bind to the capture zipper-intein transmembrane adapter in the ER, which can exit the ER following trans-splicing of Cfa inteins of the adapter and cytoplasmic shuttle constructs, which recapitulates a full-length CAR, with cleavage of the ER retention motif. Triple-transduced cells were enriched via MACS sorting with anti-FLAG magnetic beads, resulting in high purity of FLAG-positive dual-CAR T cells (FIG. 6B) These T cells demonstrate killing of CD19 or CD20 antigen expressing BM185 target cells after 24-hour co-culture (FIGS. 6C-6D). Thus, this methodology utilizes orthogonal inteins and leucine zippers to enable selective MACS sorting of T cells incorporating three unique vectors, which can further encode additional transgenes to enhance effector cell functionality.

### 7.8. Four-Vector Intein Sorting System

In the instant example, mouse T cells were transduced with the following 4 vectors (FIG. 8A): (Vector 1) encodes a secreted FLAG affinity-tagged gp41-1 intein N terminus with a BFP reporter, (Vector 2) encodes a Thy1.1 reporter and a dual-intein-tagged transmembrane intein adapter comprised of a V5 reporter tag, gp41-1 intein C terminus, a CD8 spacer, a CD28 transmembrane domain, a Cfa intein N terminus, and an E319K ER retention motif, (Vector 3) encodes an EGFP reporter and a dual-intein-tagged cytoplasmic intein adapter containing, a Cfa intein C terminus, a gp41-8 intein N terminus, and an E319K retention motif, and (Vector 4) encodes a truncated EGFR (EGFRt) reporter and a cytoplasmic chain terminator construct comprised of a gp41-8 intein C terminus fused to a CD3-zeta non-signaling delta stalk. Trans-splicing of the cognate orthogonal inteins results in assembly of a surface express molecule with removal of ER retention motifs. Quadruple-transduced cells were enriched via MACS sorting with anti-FLAG (or anti-CD34) magnetic beads, yielding a highly pure population of cells incorporating all four vectors (FIG. 8B). Additional transgenes can be encoded on each vector to boost engineered cell functionality. Antigen-binding domains and signaling domains can be added to convert the sorting system into a modular CAR system.

The example depicted in FIG. 8C is similar to FIG. 8B, except the secreted FLAG affinity tag on Vector 1 is replaced with a truncated hCD34 molecule serving as an antibody-binding epitope. Quadruple-transduced cells were enriched via MACS sorting with anti-hCD34 magnetic beads, yielding a highly pure population of cells incorporating all four vectors. Additional transgenes can be encoded on each vector to boost engineered cell functionality. Antigen-binding domains and signaling domains can be added to convert the sorting system into a modular CAR system.

In the example depicted in FIG. 8D, mouse T cells were transduced similar to the example depicted in FIG. 8B, except that Vector 3 (the cytoplasmic intein adapter) is omitted, thereby disabling the connection between constructs encoded by Vector 2 (the transmembrane intein adapter) and Vector 4 (intracellular chain terminator). The EGFP reporter is not expressed in the absence of Vector 3. In the absence of the cytoplasmic intein adapter encoded by Vector 3, the transmembrane intein adapter encoded by Vector 2 is retained in the ER, impairing FLAG expression on the cell surface (encoded by Vector 1). Cells positive for BFP/Thy1.1 (Vector 1 and Vector 2) express sufficient levels of FLAG only when Vector 3 is present. Thus, this orthogonal three-intein splicing methodology specifically generates a trans-spliced CAR or sorting stalk molecule in cells incorporating four unique vectors. This recursive strategy of nesting orthogonal inteins tagged with ER retention motifs in principle may be extended to greater than 4 vectors by adding additional orthogonal inteins. This strategy greatly increases the number of transgenes delivered to engineered cells due to the increased number of vectors integrated.

### 7.9. Two-Vector Intein Sorting System-Drug-Regulated CAR Expression

In the instant example, mouse T cells were transduced with the following 2 vectors (FIG. 9A): (Vector 1) encodes a Thy1.1 reporter and a drug-activated intein-tagged antigen-binding construct comprised of a CD19-targeting scFv fused to a hCD34 affinity-tag, a CD8 hinge and transmembrane domain, an AES CL low affinity intein N terminus, an FKBP12 drug-induced heterodimerization domain (DmrA), and an E319K ER retention motif, while (Vector 2) encodes an EGFP reporter and a cytoplasmic signaling shuttle comprised of a FRB drug-induced heterodimerization domain (DmrC), an AES CL low affinity intein C terminus, a CD28 costimulatory domain, and a CD3-zeta chain. In the absence of the dimerizing drug, hCD34 expression at the cell surface is low due to presence of the ER retention motif encoded by Vector1 (FIG. 9B). Addition of the A/C heterodimerizing drug (AP21967) at a concentration of 1-micromolar (μM) allows the FKBP12 (DmrA) and FRB (DmrB) domains of the two intein-tagged constructs to heterodimerize (i.e. A/C heterodimerization), facilitating drug-induced splicing of the low affinity AES CL inteins (FIG. 9B). Thus, this system enables drug-regulated splicing to regulate post-translational CAR assembly. Double-transduced cells were cultured with 1 μM A/C heterodimerizing drug and subsequently enriched by MACS sorting using anti-hCD34 magnetic beads to yield highly pure hCD34+CD19-CAR T cells (FIG. 9B). The ability of this system to enable regulated target cell killing through drug dependent cell surface expression of CAR is demonstrated using CD19 expressing BM185 cells as targets (FIG. 9C). This methodology improves upon previous FKBP/FRB-based drug-regulated CAR systems by eliminating the FKBP/FRB proteins following trans-splicing and generating a mature CAR molecule. This simplifies the CAR design and brings the signaling domains into a membrane proximal location where signaling is favored.

### 7.10. Two-Vector Intein Sorting System-Drug-Regulated CAR Expression, Dual-CAR, Dual Antigen Specificity

The instant example is similar in design to the example depicted in FIGS. 9A-9D, but is enabled for dual CD19 and CD20 antigen specificity. Mouse T cells were transduced with the following 2 vectors (FIG. 10A): (Vector 1) encodes two antigen binding constructs comprised of (1) a CD19-targeting scFv fused to a STREPTAG® affinity tag, a CD28 hinge and transmembrane domain, an AES intein N terminus, an FKBP12 heterodimerizing domain, and (2) a CD20-targeting VHH (variable heavy chain single domain binding domain) fused to a hCD34 tag, a CD8 hinge and transmembrane domain, an AES intein N terminus, an FKBP12 heterodimerization domain, and an E319K ER retention motif. (Vector 2) encodes a drug-regulated cytoplasmic signaling domain shuttle comprised of an FRB* heterodimerization domain, an AES intein C terminus, a CD28 costimulatory domain, a CD3-zeta chain, and also encodes an EGFP reporter. In absence of the dimerizer drug, hCD34 expression at the cell surface is low due to presence of the ER retention motif encoded fused to the cytoplasmic side of the CD20 antigen-binding construct (FIG. 10B). Addition of the A/C heterodimerizing drug (AP21967) at a concentration of 1 μM, or rapamycin (50 nM) enables FRB* encoded by Vector 2 to heterodimerize with the FKBP12 domain of the CD19 and CD20-targeting constructs encoded by Vector 1 (FIG. 10C). This enables the low-affinity AES inteins to splice efficiently due to dimerization-induced enhanced proximity, reconstituting full-length CD19 and CD20 CARs on the cell surface. The ER retention motif on the CD20-targeting construct enables hCD34 surface expression to reach sortable levels only after drug-induced splicing occurs. The CD19-targeting stalk constitutively traffics to the cell surface due to lack of an ER retention motif and enables identification of cells transduced with Vector 1. In the absence of heterodimerizing drug, the CD19-targeting stalk lacks a trans-spliced signaling domain and cannot direct T cell activation. Double-transduced cells were cultured in 1 μM A/C heterodimerizer and enriched by MACS sorting using anti-hCD34 magnetic beads yields highly pure hCD34+ CD19 CAR T cells (FIG. 10B). The efficacy of CD19/CD20-targeted dual-CAR T cells killing BM185-CD19 (FIG. 10C) and BM185-CD20 cells (FIG. 10D) in heterodimerizing drug dependent target cell killing is demonstrated through 24 hour co-culture studies. FIGS. 10E-10L demonstrates live cell microscopy demonstrating the kinetics of CD19+ or CD20+ target cell elimination by _CD19/CD20-targeted dual-CAR T cells in the presence of heterodimerizing drug. This methodology improves upon previous FKBP/FRB-based drug-regulated CAR systems by eliminating the FKBP/FRB proteins following trans-splicing and generating a mature CAR molecule. This simplifies the CAR design and brings the signaling domains into a membrane proximal location where signaling is favored. This design also facilitates generation of drug-regulated multi-antigen-targeting CAR T cells.

### 7.11. Two-Vector Intein Sorting System—Trans-Presented Cytokine

The instant example is directed to constitutive and drug-regulated presentation of IL-7 on the cell surface. For the Constitutive expression system, mouse T cells were transduced with the following 2 vectors: (Vector 1) encodes a Thy1.1 reporter and an IL-7 presentation construct comprised of the IL-7 molecule fused to a hCD34 affinity-tag, a CD8 hinge and transmembrane domain, a Cfa intein N terminus, and an E319K ER retention motif. (Vector 2) encodes a cytoplasmic chain terminator construct comprised of a Cfa intein C terminus, a CD3-zeta non-signaling delta stalk, and also encodes an EGFP reporter. The two Cfa inteins splice together intracellularly to remove the ER retention motif and allow the IL-7 to be presented on the cell surface. For the Drug-regulated expression system, mouse T cells were transduced with the following 2 vectors (FIG. 11A): (Vector 1) encodes a Thy1.1 reporter, an IL-7 presentation construct comprised of the IL-7 molecule fused to a hCD34 affinity-tag, a CD8 hinge and transmembrane domain, an AES intein N terminus, an FKBP12 heterodimerization domain (DmrA), and an E319K ER retention motif. (Vector 2) encodes a drug-regulated cytoplasmic chain terminator construct comprised of a FRB* heterodimerization domain (DmrC), an AES intein C terminus, and a CD3-zeta non-signaling delta stalk, and also encodes an EGFP reporter. Addition of the A/C heterodimerizing drug (AP21967) at a concentration of 1 μM allows the low-affinity AES inteins to splice due to drug-induced construct heterodimerization, resulting in removal of the ER retention motif and presentation of IL-7 on the cell surface. Both the constitutive and drug-regulated IL-7 trans-presenting cells were enriched by MACS sorting using anti-hCD34 magnetic beads, yielding highly pure IL-7 trans-presenting T cells (FIG. 11B). To assess expansion of these T cells, equal numbers of sorted and activated T cells were plated for 72 hours in the absence of IL-2. Drug-regulated cells were additionally maintained in the presence of a A/C heterodimerizing drug (1 μM) for 3 days. Cells were counted using flow cytometry. Both constitutive and drug-regulated trans-presenting IL-7 cells showed robust expansion compared to non-transduced T cells (FIG. 11C). This methodology utilizes ER retention motifs and drug-regulated trans-splicing to control surface expression of a tethered cytokine, which can promote cis and trans stimulation of T cells or other effector cells to enhance immune function.

### 7.12. Two-Vector Intein Modular CAR with Drug-Regulated Splicing—Single Antigen Specificity—Trimethoprim Regulation

In this example, mouse T cells were transduced with the following 2 vectors (FIG. 12A): (Vector 1) encodes a Thy1.1 reporter and an intein-tagged antigen-binding construct comprised of a CD19-targeting scFv fused to a hCD34 affinity-tag, a CD8 hinge and transmembrane domain, a Cfa intein N terminus, and an E319K ER retention motif. (Vector 2) encodes a cytoplasmic signaling domain shuttle comprised of a Cfa C terminus, a CD28 costimulatory domain, a CD3-zeta 1XX chain (ITAMs 2 and 3 with loss of function mutations), a dihydrofolate reductase destabilization domain (DHFR-DD), and a EGFP reporter. In the absence of the stabilizing drug, trimethoprim, the DHFR-DD domain is unstable, which results in construct degradation, thereby impairing expression of the hCD34-tagged CAR on the cell surface (FIG. 12B). Inclusion of trimethoprim (10 μM) stabilizes the DHFR-DD, thereby allowing the Cfa split intein domains to trans-splice with enhanced efficiency, reconstituting a full-length CD19 CAR (FIG. 12B). Double-transduced cells were cultured with 10 μM trimethoprim and subsequently enriched via MACS sorting with anti-hCD34 magnetic beads, yielding highly pure hCD34+CD19-CAR T cells (FIG. 12B). FIG. 12C shows killing of CD19 expressing BM185 target cells only in the presence trimethoprim. This methodology utilizes a drug-regulated degron (destabilized protein) to the cytoplasmic signaling shuttle intein to regulate intein expression inside cells and thereby control trans-splicing activity and CAR signaling.

### 7.13. Two-Vector Intein Modular CAR with Drug-Regulated Splicing—Single Antigen Specificity—Asunaprevir Regulation

7.13.1. hCD34 Affinity Tag

In this example, mouse T cells were transduced with the following 2 vectors (FIG. 13A): (Vector 1) encodes a Thy1.1 reporter and an intein-tagged antigen-binding construct comprised of a CD19-targeting scFv fused to an hCD34 tag, a CD8 hinge and transmembrane domain, a Cfa intein N terminus, and an E319K retention motif. (Vector 2) encodes a cytoplasmic signaling domain shuttle comprised of a Cfa intein C terminus, a CD28 costimulatory domain, a CD3-zeta 1XX chain (ITAMs 2 and 3 with loss of function mutations), a hepatitis C virus (HCV) NS3 protease cleavage site, and a degron-tagged asunaprevir-regulated HCV protease (Small Molecule-Assisted Shutoff, SMASh tag). Splicing of the two Cfa split intein domains reconstitutes a full-length CD19 CAR on the cell surface. In the absence of asunaprevir, the protease will cut the peptide at the cleavage site, allowing the degron to be removed (FIG. 13A) thus preventing degradation of the cytoplasmic signaling domain shuttle. Addition of 3 μM asunaprevir to culture media blocks the protease activity, which results in maintenance of the connection between the degron and cytoplasmic signaling domain shuttle, resulting in its degradation, thereby reducing generation of the mature trans-spliced CAR molecule (FIG. 13B). Double-transduced T cells cultured in the absence of asunaprevir were enriched using anti-hCD34 magnetic beads, yielding highly pure hCD34+CD19 CAR T cells (FIG. 13B).

### 7.13.2. FLAG Affinity Tag

In this example, —mouse T cells were transduced with the following 2 vectors: (Vector 1) encodes a Thy1.1 reporter and an intein-tagged antigen-binding construct comprised of a CD19-targeting scFv fused to a FLAG tag, a CD8 hinge and transmembrane domain, a Cfa intein N terminus, and an E319K retention motif and (Vector 2) encodes a cytoplasmic signaling domain shuttle comprised of a Cfa intein C terminus, a CD28 costimulatory domain, a CD3-zeta 1XX chain (ITAMs 2 and 3 with loss of function mutations), a hepatitis C virus (HCV) NS3 protease cleavage site, and a degron-tagged asunaprevir-regulated HCV protease (Small Molecule-Assisted Shutoff, SMASh tag). Splicing of the two Cfa inteins reconstitutes a full-length CD19 CAR on the cell surface. As discussed above, the cytoplasmic signaling domain shuttle will degraded in the presence of 3 μM asunaprevir but not in the absence of asunaprevir (FIG. 13C). Double-transduced T cells cultured in the absence of asunaprevir were enriched using anti-FLAG magnetic beads, yielding highly pure FLAG+ CD19 CAR T cells (FIG. 13C). The ability of the enriched T cells from FIG. 13C in killing CD19 expressing target cells were next examined. T cells that were not pre-cultured with asunaprevir demonstrated a higher efficacy for lysing CD 19 expressing BM185 cells than T cells that were pre-cultured with 3 μM asunaprevir (FIG. 13D).

In a second example, mouse T cells were transduced with the following 2 vectors: (Vector 1) encodes two intein-tagged antigen-binding constructs comprised of a CD20-targeting nanobody VHH domain fused to a hCD34 tag, a CD28 hinge and transmembrane domain, and a Cfa N terminus, followed by a CD19-targeting scFv fused to an FLAG tag, a CD8 hinge and transmembrane domain, a Cfa intein N terminus, and an E319K retention motif, and (Vector 2) encodes a cytoplasmic signaling domain shuttle comprised of a Cfa intein C terminus, a CD28 costimulatory domain, a CD3-zeta 1XX chain (ITAMs 2 and 3 with loss of function mutations), a hepatitis C virus (HCV) NS3 protease cleavage site, and a degron-tagged asunaprevir-regulated HCV protease (Small Molecule-Assisted Shutoff, SMASh tag). Splicing of the two Cfa inteins reconstituted full-length CD20 and CD19 CARs on the cell surface. As discussed for the examples above, the cytoplasmic signaling domain shuttle is degraded in the presence of 3 μM asunaprevir but not in the absence of asunaprevir (FIG. 13E). Thus, the retention motif on the CD19-targeting portion of Vector 1 reduces FLAG expression in the presence of drug. Double-transduced T cells were cultured with and without 3 μM asunaprevir for 4 days, and then co-cultured with BM185 expressing either CD19 or CD120. As shown in FIG. 13F and FIG. 13G, T cells exhibit specific killing against BM185 expressing either CD19 and CD20 respectively, and this lytic activity is eliminated when the T cells are precultured with asunaprevir. This methodology enables generation of T cells able to target two antigens simultaneously with coordinated drug-regulation and further enables single-step MACS purification of highly purified dual-transduced T cells incorporating two vectors.

### 7.13.3. NS3-Based Drug Regulated Zap70 CARs

In a third example, mouse T cells were transduced with two vectors (FIG. 14A): (Vector 1) encodes two distinct antigen-binding scFv transmembrane-domain-containing fusion polypeptides with degenerate cytoplasmic N terminus intein domains. Each scFv fusion polypeptide contains a distinct affinity tag and may additionally comprise an ER retention motif at the end of a vector construct at the C terminus of fusion polypeptide, and (Vector 2) encodes a drug regulatable zeta-chain-associated protein kinase 70 (Zap70) domain shuttle comprised of a degenerate intein C terminus, a hepatitis C virus (HCV) NS3 protease with flanking cleavage sites (FIG. 14B), and a tracer protein tag (e.g., EGFP tag). Splicing of the two Cfa inteins reconstitutes a full-length CD19 CAR on the cell surface. FIG. 14C shows efficacy of CD19/CD20-dual-targeting NS3-regulated Zap70-CAR T cells in killing CD19 expressing or CD20 expressing BM185 cells only in the presence of grazoprevir, which prevents NS3-based cleavage of the CAR constructs (FIG. 14B). This use of degenerate splicing of inteins sharing a common cytoplasmic signaling domain shuttle enables reuse of DNA encoding large structural elements (e.g., NS3 protease fused to Zap70 interdomain and kinase), serving as a data compression algorithm. This enables a greater number of receptors to be generated per the amount of DNA delivered by the vector and is associated with very high expression of the trans-spliced CAR constructs. Selective sequestration of the FLAG-tagged CAR by the ER retention motif and subsequent surface trafficking following trans-splicing with the cytoplasmic signaling domain shuttle and removal of the ER retention motif enables selective MACS sorting of dual-transduced cells (molecular coincidence detector).

### 7.14. NS3-Based Drug-Regulated CD28 Zeta Based Intein-CARs

### 7.14.1. Intein-Assisted NS3-Based Drug-Regulated CAR-Non-Nested Approach

In this example, mouse T cells were transduced with Vector 1 encoding a CD19 antigen binding stalk consisting of a CD19 scFv, CD8 hinge and transmembrane domain, Cfa intein, and E319K ER retention motif and Vector 2 encoding the NS4a cofactor fused to HCV NS3 protease and CD28 costimulatory domain and CD3 zeta signaling domains followed by a EGFP reporter. In different variants, the NS4a-NS3 fusion is flanked with combinations of 5A/5B and 4A/4B cut NS3 protease sites_(FIG. 15A). FIG. 15B illustrates the efficacy of CAR transfected T cells in killing BM185-CD19 cell targets selectively in the presence of grazoprevir (GZP). FIGS. 15C-15D illustrate live cell microscopy showing CD19/CD20-dual-targeting NS3-regulated Zap70-CAR T cells killing CD19 expressing or CD20 expressing BM185 cells only in the presence of grazoprevir, which prevents NS3-based cleavage of the CAR constructs. The high efficiency cleavage of the NS3 protease obviates “leaky” killing of targets by intein-CARs in the absence of the protease inhibitor. These T cells were generated by transducing with (Vector 1) a dual intein-tagged dual antigen-binding construct comprised of a CD20-targeting nanobody VHH domain fused to a hCD34 tag, a CD28 hinge and transmembrane domain, and a Cfa N terminus, followed by a CD19-targeting scFv fused to an FLAG tag, a CD8 hinge and transmembrane domain, a Cfa intein N terminus, and an E319K retention motif, and with (Vector 2), encoding the Cfa C 5A-5B NS4a NS3 4A-4B CD28z EGFP drug-regulated cytoplasmic signaling domain shuttle.

This use of degenerate splicing of inteins sharing a common cytoplasmic signaling domain shuttle enables reuse of DNA encoding large structural elements (e.g., NS3 protease fused to CD28 zeta), serving as a data compression algorithm. This enables a greater number of receptors to be generated per the amount of DNA delivered by the vector and is associated with very high expression of the trans-spliced CAR constructs. Selective sequestration of the FLAG-tagged CAR by the ER retention motif and subsequent surface trafficking following trans-splicing with the cytoplasmic signaling domain shuttle and removal of the ER retention motif enables selective MACS sorting of dual-transduced cells (molecular coincidence detector).

### 7.14.2. Intein-Assisted NS3-Based Drug-Regulated Car-Nested Approach

In this example, mouse T cells were transduced with Vector 1 encoding a Thy1.1 transduction reporter and a CD19-targeting antigen-binding stalk with CD8 hinge and transmembrane domains, nested cytoplasmic AES CL N intein, cytoplasmic Cfa N intein, and E3 19K ER retention motif and Vector 2 encoding a Cfa C terminal split intein fused to HCV NS4a-NS3 protease domain fusion flanked by 5A/5B and 4A/4B cut sties, AES CL C terminal intein, and CD28 zeta signaling domains (FIG. 16A). Following high affinity intermolecular Cfa intein trans-splicing, the low affinity AES CL inteins can splice to remove the intervening NS3 protease-based drug regulations system. FIGS. 16B and 16C illustrate the efficacy of CAR T cells generated by the non-nested (FIG. 15A) and nested approaches in killing BM185 target cells expressing CD19 at a high density and low density respectively. This methodology enables drug-regulated generation of actively signaling CARs with removal of the drug regulation polypeptides, which could impair CAR signal transduction by placing signaling domains in a membrane distal location. Removal of the drug regulation polypeptides can also prolong the activity of CAR in the absence of the HCV protease inhibitor.

### 7.15. Two-Vector Intein Modular CAR and Chimeric Costimulatory Receptor (CCR) Orthogonal Signaling Domain Regulation

In this example, mouse T cells were transduced with the following two vectors (FIG. 17A): (Vector 1) encodes a Thy1.1 reporter and an intein-tagged antigen-binding construct comprised of a CD19-targeting scFv fused to an FLAG tag, a CD8 hinge and transmembrane domain, a Cfa intein N terminus, and an E319K retention motif and (Vector 2) encodes a cytoplasmic signaling domain shuttle composed of a Cfa C intein terminus, a 4-1BB costimulatory domain fused to a BFP reporter, a 2A cleavage peptide, followed by a second Cfa C intein terminus, a CD28 costimulatory domain, a CD3-zeta 1XX chain (ITAMs 2 and 3 with loss of function mutations) fused to an EGFP reporter, and a hepatitis C virus (HCV) NS3 protease cleavage site, and a degron-tagged asunaprevir-regulated HCV protease (Small Molecule-Assisted Shutoff, SMASh tag). Splicing of the Cfa inteins reconstitutes either a full length CD19 CAR or a CD19 targeting chimeric costimulatory receptor (CCR). While the CD19 CAR enables the cells to kill CD19 targets, the CD19 CCR allows the T cells to expand and persist upon recognition of CD19. Double-transduced cells were enriched using anti-FLAG magnetic beads, yielding highly pure FLAG⁺ CD34⁺ CD19 CAR-CCR T cells (FIG. 17B). Control CD19 CD28 1XX intein CAR T cells lacking 4-1BB costimulation were also transduced and enriched with anti-FLAG beads to high purity (FIG. 17C). After sorting, 20,000 T cells were cultured in the absence of IL-2 and with or without 60,000 BM185 target cells expressing CD19. After 3 days of culture, cells were assessed by flow cytometry for T cell counts. T cells expressing both CD19 CAR and CCR were present in greater numbers after exposure to CD19-expressing targets, compared to T cells containing only CD19 CAR (FIG. 17D). FIG. 17E shows the results of flow cytometric analysis of dual-CAR (CD19/CD20)+dual-CCR dual stalk+CD28z+4-1BB (shown in FIG. 17F). FIG. 17G illustrates the efficacy of dual-CAR T cells to eliminate BM185-CD19 or BM185-CD20 target cells at the indicated E: T ratios. FIG. 17H shows the results of expansion studies for dual-CAR, dual-CCR T cells after exposure to BM185-CD19 or BM185-CD20 target cells at the indicated E: T ratios. This methodology enables trans-splicing combination to generate four possible combinations: CD19-CAR, CD20-CAR, CD19-CCR, CD20-CCR. This configuration represents a data compression algorithm whereby re-use of degenerate encoded DNA elements enables post-translational assembly of a greater number of receptors than could be encoded with the same amount of DNA. The additive CAR+CCR costimulation promotes enhanced T cell proliferation and enhanced target killing at low T cell E:T ratios, consistent with more active cell product.

### 7.16. Two-Vector Intein Modular Zap70-CAR and Chimeric Costimulatory Receptor (CCR) Expression

In this example, mouse T cells are transduced with two-vectors (FIG. 18A): (Vector 1) encodes two distinct antigen-binding scFv transmembrane-domain-containing fusion polypeptides (e.g., anti-CD19, anti-CD20) with degenerate cytoplasmic N terminus intein (I_{N}) domains. Each scFv fusion polypeptide contains a distinct affinity tag and may additionally comprise an ER retention motif (not shown) at the end of a vector construct at the C terminus of fusion polypeptide. (Vector 2) encodes a Zap70 domain shuttle comprising degenerate intein C terminus, and CCR (e.g., 4-1BB). FIGS. 18B and 18C show enhanced target killing and T cell proliferation with Zap70 intein-CAR+CCR combinations, targeting both CD19⁺ and CD20⁺ targets (dual-CAR/dual-CCR activity). The results demonstrate that co-expression of 4-1BB CCR improves activity of Zap70 CAR T cells. This methodology enables trans-splicing combination to generate four possible combinations: CD19-Zap70-CAR, CD20-Zap70-CAR, CD19-CCR, CD20-CCR. This use of degenerate splicing of inteins sharing a common cytoplasmic signaling domain shuttle enables reuse of DNA encoding large structural elements (e.g., Zap70 interdomain and kinase), serving as a data compression algorithm. This enables a greater number of receptors to be generated per the amount of DNA delivered by the vector and is associated with very high expression of the trans-spliced CAR constructs. Selective sequestration of the FLAG-tagged CAR by the ER retention motif and subsequent surface trafficking following trans-splicing with the cytoplasmic signaling domain shuttle and removal of the ER retention motif enables selective MACS sorting of dual-transduced cells (molecular coincidence detector).

What is claimed is:

1. A system comprising two nucleic acid constructs, wherein:
A. each nucleic acid construct comprises a nucleic acid sequence encoding an extein;
B. the first nucleic acid construct comprises a nucleic acid sequence encoding one of a complementary pair of N- and C-split inteins;
C. the second nucleic acid construct comprises a nucleic acid sequence encoding the other of the complementary pair of N- and C-split inteins; and
D. at least one nucleic acid construct encodes an endoplasmic reticulum (ER) retention motif.

2. The system of claim 1 comprising:
A. a first nucleic acid construct comprising a nucleic acid sequence encoding:
i. a first extein; and
ii. a first N-split intein of a first complementary pair of split inteins;
B. a second nucleic acid construct comprising a nucleic acid sequence encoding:
i. a second extein;
ii. a first C-split intein of the first complementary pair split inteins; and
iii. a second N-split intein, wherein the second N-split intein is of a second complementary pair of split inteins; and
C. a third nucleic acid construct comprising nucleic acid sequences encoding:
i. a third extein; and
ii. a second C-split intein, wherein the second C-split intein is of the second complementary pair split inteins.

3. The system of claim 1 comprising:
A. a first nucleic acid construct comprising a nucleic acid sequence encoding:
a. a first extein; and
b. a first leucine zipper motif;
B. a second nucleic acid construct comprising a nucleic acid sequence encoding:
a. a second leucine zipper motif; and
b. an N-split intein of a complementary pair split inteins; and
C. a third nucleic acid construct comprising a nucleic acid sequence encoding:
a. a second extein; and
b. a C-split intein of the complementary pair split inteins.

4. The system of claim 1 comprising:
A. a first nucleic acid construct comprising a nucleic acid sequence encoding:
a. a first extein; and
b. a first N-split intein of a first complementary pair of split inteins;
B. a second nucleic construct comprising a nucleic acid sequence encoding:
a. a second extein;
b. a first C-split intein of the first complementary pair split inteins; and
c. a second N-split intein, wherein the second N-split intein is of a second complementary pair of split inteins;
C. a third nucleic acid construct comprising a nucleic acid sequence encoding:
a. a third extein;
b. a second C-split intein, wherein the second C-split intein is of the second complementary split inteins; and
c. a third N-split intein, wherein the third N-split intein is of a third complementary pair of split inteins; and
D. a fourth nucleic acid construct comprising a nucleic acid sequence encoding:
a. a fourth extein; and
b. a third C-split intein, wherein the third C-split intein is of the third complementary pair of split inteins.

5. The system of claim 1, wherein the N-terminal amino acid of an N-split intein is linked to the C-terminal amino acid of an extein, or the N-terminal amino acid of a extein is linked to the C-terminal amino acid of a C-split intein.

6. The system of claim 1 wherein the complementary pair of split inteins are selected from the group consisting of a Cfa intein, a gp41-1 intein, gp41-8 intein, an Aes123 PolB1 Intein (Aes Intein), an NrdJ-1 intein, an IMPDH-1 intein, a SspGyrB intein, a DNA polymerase III (DnaE) intein, orthologs thereof, and variants thereof.

7. The system of claim 1 wherein the complementary pair of N- and C-split inteins comprise a splicing motif selected from the group consisting of CLS, CFN, CLD, HNS, SVV, SVYLN, and CLV.

8. The system of claim 1 wherein the N-split inteins have an amino acid sequence selected from SEQ ID NOs: 1, 3, 5, 7, and 9.

9. The system of claim 1 wherein the C-split inteins have an amino acid sequence selected from SEQ ID NOs: 2, 4, 6, 8, and 10.

10. The system of claim 1 wherein the N-split inteins have an amino acid sequence selected from SEQ ID NOs: 1, 3, 5, 7, and 9, and the complementary C-split inteins have an amino acid sequence selected from SEQ ID NOs: 2, 4, 6, 8, and 10 respectively.

11. The system of claim 1, wherein the ER retention motif is a KKXX motif, or an RXR motif.

12. The system of claim 1, wherein the N-terminal amino acid of the ER retention motif is linked to the C-terminal amino acid of a N-split intein.

13. The system of claim 1, wherein the ER retention motif is an RXR motif, the RXR motif flanked by sequences encoding an extein.

14. The system of claim 1, wherein at least one nucleic acid construct comprises a nucleotide sequence encoding a regulatable gene element.

15. The system of claim 22, wherein the regulatable gene element encodes a regulator motif that regulates expression of the extein.

16. The system of claim 1, wherein the extein comprises an extracellular antigen binding domain, an extracellular cytokine, a transmembrane domain, a signaling domain, or a combination thereof.

17. The system of claim 16, wherein the extein comprises a chimeric antigen receptor (CAR), a chimeric co-stimulating receptor (CCR), a T cell receptor (TCR), a TCR-like fusion molecule, orthologs thereof, or variants thereof.

18. A system comprising two or more nucleic acid constructs, each nucleic acid construct comprising a nucleotide sequence encoding:
A. at least one extein;
B. at least one of a complementary pair of N- and C-split inteins;
C. at least one endoplasmic reticulum (ER) retention motif; and
D. a regulator motif or a regulatable domain that regulates expression of the extein.

19. A method for treating a disease comprising providing to a subject in need thereof, a population of modified cells comprising the system of claim 1.

20. The method of claim 19, wherein the disease is a cancer, an autoimmune disease, an inflammatory disease, or a graft versus-host disease.


SEQUENCE LISTING (161 sequences)

SEQ ID NO: 1 - protein, 104 aa, synthetic construct
  REGION 1..104: note = Description of sequence: Cfa intein N-terminus
AEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 2 - protein, 39 aa, synthetic construct
  REGION 1..39: note = Description of sequence: Cfa intein C-terminus
MVKIISRKSLGTQNVYDIGVEKDHNFLLKNGLVASNCFN

SEQ ID NO: 3 - protein, 93 aa, synthetic construct
  REGION 1..93: note = Description of sequence: gp41-1 intein N-terminus
TRSGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 4 - protein, 42 aa, synthetic construct
  REGION 1..42: note = Description of sequence: gp41-1 intein C-terminus
MMLKKILKIEELDERELIDIEVSGNHLFYANDILTHNSSSDV

SEQ ID NO: 5 - protein, 94 aa, synthetic construct
  REGION 1..94: note = Description of sequence: gp41-8 intein N-terminus
SQLNRCLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 6 - protein, 51 aa, synthetic construct
  REGION 1..51: note = Description of sequence: gp41-8 intein C-terminus
MCEIFENEIDWDEIASIEYVGVEETIDINVTNDRLFFANGILTHNSSAVEE

SEQ ID NO: 7 - protein, 32 aa, synthetic construct
  REGION 1..32: note = Description of sequence: Aes intein N-terminus
YIDTDSVVGDTIIDVSGKKMTIAEFYDSTPDG

SEQ ID NO: 8 - protein, 134 aa, synthetic construct
  REGION 1..134: note = Description of sequence: Aes intein C-terminus
EARDWVKRVGGKTSLSVNTYSGEVERKNINYIMKHTVKKRMFKIKAGGKEVIVTADHSVMVKRDGKIIDVKPTEMKQTDRVVKWMLTGSHMIEFIEFEIEDLGVMEIDVYDIEVDGNHNFFGNDILVHNSVYLN

SEQ ID NO: 9 - protein, 110 aa, synthetic construct
  REGION 1..110: note = Description of sequence: Nrdj-1 N-terminus
GTNPCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 10 - protein, 46 aa, synthetic construct
  REGION 1..46: note = Description of sequence: Nrdj-1 C-terminus
MEAKTYIGKLKSRKIVSNEDTYDIQTSTHNFFANDILVHNSSEIVL

SEQ ID NO: 11 - protein, 37 aa, synthetic construct
  REGION 1..37: note = Description of sequence: Linker1
GGGGSGGGSELPTQGTFSNVSTNVSGGGGSGGGSRSG

SEQ ID NO: 12 - protein, 12 aa, synthetic construct
  REGION 1..12: note = Description of sequence: Linker2
TSGGGGSGGGSG

SEQ ID NO: 13 - protein, 14 aa, synthetic construct
  REGION 1..14: note = Description of sequence: Linker3
TSGGGGSGGGSGTG

SEQ ID NO: 14 - protein, 12 aa, synthetic construct
  REGION 1..12: note = Description of sequence: Linker4
GGGGSGGGSGTG

SEQ ID NO: 15 - protein, 11 aa, synthetic construct
  REGION 1..11: note = Description of sequence: Linker5
STGGGGSGGGS

SEQ ID NO: 16 - protein, 11 aa, synthetic construct
  REGION 1..11: note = Description of sequence: Linker6
GSGGGGSGGGS

SEQ ID NO: 17 - protein, 10 aa, synthetic construct
  REGION 1..10: note = Description of sequence: Linker7
GTSRAKRGSG

SEQ ID NO: 18 - protein, 27 aa, synthetic construct
  REGION 1..27: note = Description of sequence: ER retention motif - E319K
  DOMAIN 1..12: note = Linker
  DOMAIN 13..27: note = E3 19K motif
GGGGSGGGSGTGKYKSRRSFIEEKKMP

SEQ ID NO: 19 - protein, 48 aa, synthetic construct
  REGION 1..48: note = Description of sequence: ER retention motif-Kir6.2 RXR
  DOMAIN 1..12: note = Linker
  DOMAIN 13..48: note = RXR containing motif
GGGGSGGGSGTGLLDALTLASSRGPLRKRSVAVAKAKPKFSISPDSLS

SEQ ID NO: 20 - protein, 42 aa, synthetic construct
  REGION 1..42: note = Description of sequence: kainite 5 RXR motif
EFIWSTRRSAESEEVSVCQEMLQELRHAVSCRKTSRSRRRRR

SEQ ID NO: 21 - protein, 35 aa, synthetic construct
  REGION 1..35: note = Description of sequence: Na+ channel Nav1.8 RXR motif
EVLAALGIDTTSLHSHNGSPLTSKNASERRHRIKP

SEQ ID NO: 22 - protein, 33 aa, synthetic construct
  REGION 1..33: note = Description of sequence: SAP97 RXR motif
RKSGRAEPDPKKKATFRAITSTLASSFKRRRSS

SEQ ID NO: 23 - protein, 20 aa, synthetic construct
  REGION 1..20: note = Description of sequence: E2A
QCTNYALLKLAGDVESNPGP

SEQ ID NO: 24 - protein, 19 aa, synthetic construct
  REGION 1..19: note = Description of sequence: P2A
ATNFSLLKQAGDVEENPGP

SEQ ID NO: 25 - protein, 18 aa, synthetic construct
  REGION 1..18: note = Description of sequence: T2A
EGRGSLLTCGDVEENPGP

SEQ ID NO: 26 - protein, 22 aa, synthetic construct
  REGION 1..22: note = Description of sequence: F2A
VKQTLNFDLLKLAGDVESNPGP

SEQ ID NO: 27 - protein, 28 aa, synthetic construct
  REGION 1..28: note = Description of sequence: Tandem Strep-tag-II
WSHPQFEKGGGSGGGSGGSAWSHPQFEK

SEQ ID NO: 28 - protein, 14 aa, synthetic construct
  REGION 1..14: note = Description of sequence: V5 Tag
GKPIPNPLLGLDST

SEQ ID NO: 29 - protein, 16 aa, synthetic construct
  REGION 1..16: note = Description of sequence: CD34 tag
ELPTQGTFSNVSTNVS

SEQ ID NO: 30 - protein, 41 aa, synthetic construct
  REGION 1..41: note = Description of sequence: Tandem CD20 mimotope tag Linker (e.g., SGGGGSD, GGGGSGGGS) underlined Mimotope in bold
  DOMAIN 1..8: note = Linker
  DOMAIN 9..19: note = Mimotope
  DOMAIN 20..25: note = Linker
  DOMAIN 26..32: note = Mimotope
  DOMAIN 33..41: note = Linker
SGGGGSDCPYSNPSLCSGGGGSDCPYSNPSLCGGGGSGGGS

SEQ ID NO: 31 - protein, 233 aa, synthetic construct
  REGION 1..233: note = Description of sequence: tagBFP
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 32 - protein, 44 aa, synthetic construct
  REGION 1..44: note = Description of sequence: CD8 spacer (mouse)
STTTKPVLRTPSPVHPTGTSQPQRPEDCRPRGSVKGTGLDFACD

SEQ ID NO: 33 - protein, 36 aa, synthetic construct
  REGION 1..36: note = Description of sequence: CD28 spacer (mouse)
IEFMYPPPYLDNERSNGTIIHIKEKHLCHTQSSPKL

SEQ ID NO: 34 - protein, 25 aa, synthetic construct
  REGION 1..25: note = Description of sequence: PD-1 spacer (mouse)
TERILETSTRYPSPSPKPEGRFQGM

SEQ ID NO: 35 - protein, 13 aa, synthetic construct
  REGION 1..13: note = Description of sequence: IgG1 hinge (mouse)
VPRDCGCKPCICT

SEQ ID NO: 36 - protein, 47 aa, synthetic construct
  REGION 1..47: note = Description of sequence: Leucine Zipper (RR12EE345L)
LEIRAAFLRQRNTALRTEVAELEQEVQRLENEVSQYETRYGPLGGGK

SEQ ID NO: 37 - protein, 47 aa, synthetic construct
  REGION 1..47: note = Description of sequence: Leucine Zipper (EE12RR345L)
LEIEAAFLERENTALETRVAELRQRVQRLRNRVSQYRTRYGPLGGGK

SEQ ID NO: 38 - protein, 107 aa, synthetic construct
  REGION 1..107: note = Description of sequence: FKBP12
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 39 - protein, 94 aa, synthetic construct
  REGION 1..94: note = Description of sequence: FRB*
MILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 40 - protein, 158 aa, synthetic construct
  REGION 1..158: note = Description of sequence: DHFR destabilization domain
ISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 41 - protein, 304 aa, synthetic construct
  REGION 1..304: note = Description of sequence: Degron (SMASh tag)
GTGGSGDEMEECSQHLPGAGSSGDIMGSSGTGSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLYGS

SEQ ID NO: 42 - protein, 186 aa, synthetic construct
  REGION 1..186: note = Description of sequence: HCV NS3 protease
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 43 - protein, 261 aa, synthetic construct
  REGION 1..261: note = Description of sequence: HCV NS3 protease
EDVVCCHSIYGKKKGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQH

SEQ ID NO: 44 - protein, 250 aa, synthetic construct
  REGION 1..250: note = Description of sequence: HCV NS3 protease
GTGGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQH

SEQ ID NO: 45 - protein, 252 aa, synthetic construct
  REGION 1..252: note = Description of sequence: HCV NS3 protease
EDVVCCHSIYGKKKGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEF

SEQ ID NO: 46 - protein, 14 aa, synthetic construct
  REGION 1..14: note = Description of sequence: NS4 cofactor
TGCVVIVGRIVLSG

SEQ ID NO: 47 - protein, 14 aa, synthetic construct
  REGION 1..14: note = Description of sequence: HCV 5A/5B cut site
EDVVCCHSIYGKKK

SEQ ID NO: 48 - protein, 143 aa, synthetic construct
  REGION 1..143: note = Description of sequence: Thy1.1 mouse
QKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 49 - protein, 335 aa, synthetic construct
  REGION 1..335: note = Description of sequence: Truncated epidermal growth factor receptor
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 50 - protein, 239 aa, synthetic construct
  REGION 1..239: note = Description of sequence: EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 51 - protein, 17 aa, synthetic construct
  REGION 1..17: note = Description of sequence: Truncated CD3z
RAKFSRSAETAANLQDT

SEQ ID NO: 52 - protein, 12 aa, synthetic construct
  REGION 1..12: note = Description of sequence: Truncated CD3z
FDALHMQTLAPR

SEQ ID NO: 53 - protein, 345 aa, synthetic construct
  REGION 1..345: note = Description of sequence: Zap70 Interdomain + kinase domain (mouse)
PNSSASAAVAAPTLPAHPSTFTQPQRRVDTLNSDGYTPEPARLASSTDKPRPMPMDTSVYESPYSDPEELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEAFSYGQKPYKKMKGPEVLDFIKQGKRMECPPECPPEMYALMSDCWIYKWEDRPDFLTVEQRMRNYYYSL

SEQ ID NO: 54 - protein, 346 aa, synthetic construct
  REGION 1..346: note = Description of sequence: Zap70 Interdomain + kinase domain (human)
PNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 55 - protein, 400 aa, synthetic construct
  REGION 1..400: note = Description of sequence: FLAG gp41-1 N BFP From drawing(s): FIGs. 2B 8B
  DOMAIN 1..400: note = From FIGs. 2B 8B
  DOMAIN 23..25: note = Linker
  DOMAIN 26..134: note = Intein
  DOMAIN 135..137: note = Linker
  DOMAIN 138..141: note = Furin cleavage site
MASPLTRFLSLNLLLLGESIILGSGEAEFTDYKDDDDKGSGTRSGYCLDLKTQVQTPQGMKEISNIQVGDLVLSNTGYNEVLNVFPKSKKKSYKITLEDGKEIICSEEHLFPTQTGEMNISGGLKEGMCLYVKEGSGRAKRATNFSLLKQAGDVEENPGPTRTGLESMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 56 - protein, 360 aa, synthetic construct
  REGION 1..360: note = Description of sequence: gp41-1 C myc CD8EC CD3z delta E2A Thy1.1 From drawing(s): FIG. 2B
  DOMAIN 1..360: note = From FIG. 2B
  DOMAIN 21..62: note = Intein
METDTLLLWVLLLWVPGSTGMMLKKILKIEELDERELIDIEVSGNHLFYANDILTHNSSSDVEQKLISEEDLAAASTTTKPVLRTPSPVHPTGTSQPQRPEDCRPRGSVKGTGLDFACDIYIWAPLAGICVALLLSLIITLICYRAKFSRSAETAANLQDTYDALHMQTLAPRGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 57 - protein, 518 aa, synthetic construct
  REGION 1..518: note = Description of sequence: Thy1.1 P2A RQR FLAG CD8EC CD8TM tCD3z AEY Cfa N G4S2 Kir RXR From drawing(s): FIG. 3B
  DOMAIN 1..518: note = From FIG. 3B
  DOMAIN 362..364: note = Linker
  DOMAIN 365..468: note = Intein
  DOMAIN 469..482: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMETDTLLLWVLLLWVPGSTGCPYSNPSLCSGGGGSELPTQGTFSNVSTNVSPAKPTTTACPYSNPSLCGGGGSGGGSDYKDDDDKAAASTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGLLDALTLASSRGPLRKRSVAVAKAKPKFSISPDSLS

SEQ ID NO: 58 - protein, 461 aa, synthetic construct
  REGION 1..461: note = Description of sequence: Cfa C CD28z EGFP From drawing(s): FIGs. 3B 5C-5D 6C-6D
  DOMAIN 1..461: note = From FIGs. 3B 5C-5D 6C-6D
  DOMAIN 1..39: note = Intein
  DOMAIN 40..42: note = Linker
MVKIISRKSLGTQNVYDIGVEKDHNFLLKNGLVASNCFNGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 59 - protein, 708 aa, synthetic construct
  REGION 1..708: note = Description of sequence: Thy1.1 P2A 1D3 CD19 hCD34 CD8EC CD8TM tCD3z AEY Cfa N G4S2 EEKKMP E3 19K From drawing(s): FIGs. 3C-3D 12C
  DOMAIN 1..708: note = From FIGs. 3C-3D 12C
  DOMAIN 556..572: note = Truncated CD3z
  DOMAIN 573..575: note = Linker
  DOMAIN 576..679: note = Intein
  DOMAIN 680..693: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRGGGGSELPTQGTFSNVSTNVSGGGGSGGGSGSLSTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 60 - protein, 1041 aa, synthetic construct
  REGION 1..1041: note = Description of sequence: CD19 scFv gp41-1N CD20 scFv gp41-1N FLAG-BFP From drawing(s): FIGs. 4C-4D 5C-5D
  DOMAIN 1..1041: note = From FIGs. 4C-4D 5C-5D
  DOMAIN 278..280: note = Linker
  DOMAIN 281..373: note = Intein
  DOMAIN 374..376: note = Linker
  DOMAIN 677..679: note = Linker
  DOMAIN 680..772: note = Intein
  DOMAIN 773..775: note = Linker
MASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREFTDYKDDDDKGSGTRSGYCLDLKTQVQTPQGMKEISNIQVGDLVLSNTGYNEVLNVFPKSKKKSYKITLEDGKEIICSEEHLFPTQTGEMNISGGLKEGMCLYVKEGSGRAKRATNFSLLKQAGDVEENPGPTRTMETDTLLLWVLLLWVPGSTGQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSRSGDYKDDDDKGSGTRSGYCLDLKTQVQTPQGMKEISNIQVGDLVLSNTGYNEVLNVFPKSKKKSYKITLEDGKEIICSEEHLFPTQTGEMNISGGLKEGMCLYVKEGSGRAKRQCTNYALLKLAGDVESNPGPGSLGSGLESMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 61 - protein, 485 aa, synthetic construct
  REGION 1..485: note = Description of sequence: gp41-1C CD8EC CD28 CD3z E2A Thy1.1 From drawing(s): FIGs. 4C-4D
  DOMAIN 1..485: note = From FIGs. 4C-4D
  DOMAIN 21..62: note = Intein
METDTLLLWVLLLWVPGSTGMMLKKILKIEELDERELIDIEVSGNHLFYANDILTHNSSSDVEQKLISEEDLAAASTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 62 - protein, 891 aa, synthetic construct
  REGION 1..891: note = Description of sequence: 2MC57 CD34 CD28EC CD28TM tCD3z AEY Cfa N P2A 1D3 CD19 FLAG CD8EC CD8TM tCD3z AEY Cfa N G4S2 EEKKMP E3 19K From drawing(s): FIGs. 4G 13F-13G 14A 15C-15D 17E-17G
  DOMAIN 1..891: note = From FIGs. 4G 13F-13G 14A 15C-15D 17E-17G
  DOMAIN 241..257: note = Truncated CD3z
  DOMAIN 258..260: note = Linker
  DOMAIN 261..364: note = Intein
  DOMAIN 365..367: note = Linker
  DOMAIN 739..755: note = Truncated CD3z
  DOMAIN 756..758: note = Linker
  DOMAIN 759..862: note = Intein
  DOMAIN 863..876: note = Linker
METDTLLLWVLLLWVPGSTGQVQLQESGGGLVQAGGSLRLSCAASGRDFATYSMAWFRQAPGKERESVATISWSGQRTRYADSVKGRFTISRDNAKNTVYLQMNSLKPEDTAVYYCAMPRTWGEFPPTQYDSWGQGTQVTVSSGGGGSELPTQGTFSNVSTNVSGGGGSGGGSEARLIEFMYPPPYLDNERSNGTIIHIKEKHLCHTQSSPKLFWALVVVAGVLFCYGLLVTVALCVIWTRAKFSRSAETAANLQDTGSGAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGSGATNFSLLKQAGDVEENPGPEFTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRGSGDYKDDDDKGSLSTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 63 - protein, 659 aa, synthetic construct
  REGION 1..659: note = Description of sequence: Cfa C Zap70 E2A EGFP From drawing(s): FIG. 4G
  DOMAIN 1..659: note = From FIG. 4G
  DOMAIN 1..39: note = Intein
  DOMAIN 40..42: note = Linker
MVKIISRKSLGTQNVYDIGVEKDHNFLLKNGLVASNCFNGTGPNSSASAAVAAPTLPAHPSTFTQPQRRVDTLNSDGYTPEPARLASSTDKPRPMPMDTSVYESPYSDPEELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEAFSYGQKPYKKMKGPEVLDFIKQGKRMECPPECPPEMYALMSDCWIYKWEDRPDFLTVEQRMRNYYYSLGTSRAKRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 64 - protein, 486 aa, synthetic construct
  REGION 1..486: note = Description of sequence: Thy1.1 P2A V5 gp41-1 CD8EC CD8TM tCD3z AEY Cfa N E3 19K From drawing(s): FIGs. 5C/D
  DOMAIN 1..486: note = From FIGs. 5C/D
  DOMAIN 222..230: note = Linker
  DOMAIN 231..272: note = Intein
  DOMAIN 273..275: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMETDTLLLWVLLLWVPGSTGGKPIPNPLLGLDSTGGGGSGGGSMMLKKILKIEELDERELIDIEVSGNHLFYANDILTHNSSSDVGSLSTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGTGKYKSRRSFIDEKKMP

SEQ ID NO: 65 - protein, 946 aa, synthetic construct
  REGION 1..946: note = Description of sequence: CD19/CD20 scFv FLAG-RR12EE345L -BFP From drawing(s): FIGs. 6C/D
  DOMAIN 1..946: note = From FIGs. 6C/D
  DOMAIN 278..324: note = Leucine zipper
  DOMAIN 325..327: note = Linker
  DOMAIN 628..674: note = Leucine zipper
  DOMAIN 675..680: note = Linker
MASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREFTDYKDDDDKLEIRAAFLRQRNTALRTEVAELEQEVQRLENEVSQYETRYGPLGGGKGSGRAKRATNFSLLKQAGDVEENPGPTRTMETDTLLLWVLLLWVPGSTGQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSRSGDYKDDDDKLEIRAAFLRQRNTALRTEVAELEQEVQRLENEVSQYETRYGPLGGGKGSGGSGRAKRQCTNYALLKLAGDVESNPGPGSLGSGLESMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 66 - protein, 468 aa, synthetic construct
  REGION 1..468: note = Description of sequence: Thy1.1 P2A V5 EE12RR345L IgG1-hinge tCD3z AEY Cfa N E3 19K From drawing(s): FIGs. 6C/D
  DOMAIN 1..468: note = From FIGs. 6C/D
  DOMAIN 234..280: note = Leucine zipper
  DOMAIN 344..346: note = Linker
  DOMAIN 347..450: note = Intein
  DOMAIN 451..453: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMETDTLLLWVLLLWVPGSTGGSGGKPIPNPLLGLDSTGGGGSGGGSLEIEAAFLERENTALETRVAELRQRVQRLRNRVSQYRTRYGPLGGGKAAAVPRDCGCKPCICTGSLFWALVVVAGVLFCYGLLVTVALCVIWTRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGTGKYKSRRSFIDEKKMP

SEQ ID NO: 67 - protein, 502 aa, synthetic construct
  REGION 1..502: note = Description of sequence: Thy1.1 P2A V5 gp41-1 CD8EC CD28TM tCD3z AEY Cfa N G4S2 E3 19K EEKKMP From drawing(s): FIG. 8B
  DOMAIN 1..502: note = From FIG. 8B
  DOMAIN 220..230: note = Linker
  DOMAIN 231..272: note = Intein
  DOMAIN 273..275: note = Linker
  DOMAIN 272..274: note = Linker
  DOMAIN 275..473: note = Intein
  DOMAIN 474..487: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMETDTLLLWVLLLWVPGSTGGKPIPNPLLGLDSTGGGGSGGGSMMLKKILKIEELDERELIDIEVSGNHLFYANDILTHNSSSDVGSLSTTTKPVLRTPSPVHPTGTSQPQRPEDCRPRGSVKGTGLDFACDGSLFWALVVVAGVLFCYGLLVTVALCVIWTRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 68 - protein, 449 aa, synthetic construct
  REGION 1..449: note = Description of sequence: EGFP Cfa C CD3z delta GP41-8 N EEKKMP E3 19K From drawing(s): FIG. 8B
  DOMAIN 1..449: note = From FIG. 8B
  DOMAIN 267..269: note = Linker
  DOMAIN 270..308: note = Intein
  DOMAIN 312..325: note = Truncated alternate CD3z
  DOMAIN 326..328: note = Linker
  DOMAIN 329..422: note = Intein
  DOMAIN 423..434: note = Linker
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGQCTNYALLKLAGDVESNPGPRAKRGSGMVKIISRKSLGTQNVYDIGVEKDHNFLLKNGLVASNCFNGTGFDALHMQTLAPRTSGSGSQLNRCLSLDTMVVTNGKAIEIRDVKVGDWLESECGPVQVTEVLPIIKQPVFEIVLKSGKKIRVSANHKFPTKDGLKTINSGLKVGDFLRSRAKGGGGSGGGSGTGKYKSRRSFIEEKKMP

SEQ ID NO: 69 - protein, 454 aa, synthetic construct
  REGION 1..454: note = Description of sequence: gp41-8 C CD3z delta EGFRt From drawing(s): FIG. 8B 3E-3I
  DOMAIN 1..454: note = From FIG. 8B 3E-3I
  DOMAIN 1..51: note = Intein
  DOMAIN 52..54: note = Linker
MCEIFENEIDWDEIASIEYVGVEETIDINVTNDRLFFANGILTHNSSAVEEGTGFDALHMQTLAPRGSGRAKRGSGQCTNYALLKLAGDVESNPGPLESMYSMQLASCVTLTLVLLVNSRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 70 - protein, 445 aa, synthetic construct
  REGION 1..445: note = Description of sequence: Q2 gp41-1 N BFP From drawing(s): FIG. 8C
  DOMAIN 1..445: note = From FIG. 8C
  DOMAIN 51..86: note = Linker
  DOMAIN 87..179: note = Intein
  DOMAIN 180..182: note = Linker
MASPLTRFLSLNLLLLGESIILGSGEAEFTGSGELPTQGTFSNVSTNVSGGGGSGGGSELPTQGTFSNVSTNVSGGGGSGGGSRSGTRSGYCLDLKTQVQTPQGMKEISNIQVGDLVLSNTGYNEVLNVFPKSKKKSYKITLEDGKEIICSEEHLFPTQTGEMNISGGLKEGMCLYVKEGSGRAKRATNFSLLKQAGDVEENPGPTRTGLESMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 71 - protein, 751 aa, synthetic construct
  REGION 1..751: note = Description of sequence: Thy1.1 P2A 1D3 CD19 hCD34 CD8EC CD8TM tCD3z AES CL N FKBP12-DmRA G4S2 EEKKMP E3 19K From drawing(s): FIG. 9C
  DOMAIN 1..751: note = From: FIG. 9C
  DOMAIN 556..572: note = Truncated CD3z
  DOMAIN 573..578: note = Linker
  DOMAIN 579..609: note = Intein
  DOMAIN 610..615: note = Linker
  DOMAIN 616..722: note = FKBP12
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRGGGGSELPTQGTFSNVSTNVSGGGGSGGGSGSLSTTTKPVLRTPSPVHPTGTSQPQRPEDCRPRGSVKGTGLDFACDIYIWAPLAGICVALLLSLIITLICYRAKFSRSAETAANLQDTGSSGSGYIDTDSVVGDTIIDVSGKKMTIAEFYDSTPDGSGGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSGGGGSGGGSGTGKYKSRRSFIEEKKMP

SEQ ID NO: 72 - protein, 666 aa, synthetic construct
  REGION 1..666: note = Description of sequence: FRB AES CL C CD28z EGFP From drawing(s): FIG. 9C
  DOMAIN 1..666: note = From: FIG. 9C
  DOMAIN 1..94: note = FKB
  DOMAIN 95..105: note = Linker
  DOMAIN 106..239: note = Intein
  DOMAIN 240..247: note = Linker
MILWHEMWHEGLEEASRLYFGERNVKGMFEVLEPLHAMMERGPQTLKETSFNQAYGRDLMEAQEWCRKYMKSGNVKDLLQAWDLYYHVFRRISKGSGGGGSGGGSEARDWVKRVGGKTSLSVNTYSGEVERKNINYIMKHTVKKRMFKIKAGGKEVIVTADHSVMVKRDGKIIDVKPTEMKQTDRVVKWMLTGSHMIEFIEFEIEDLGVMEIDVYDIEVDGNHNFFGNDILVHNSVYLNGGGGSGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 73 - protein, 1007 aa, synthetic construct
  REGION 1..1007: note = Description of sequence: 1D3 CD19 ST CD28EC CD28TM AES CL N FKBP12 P2A 2MC27 CD20 VHH hCD34 CD8EC CD8TM tCD3z AES CL N FKBP12 G4S2 EEKKMP E3 19K From drawing(s): FIGs. 10C/D
  DOMAIN 1..1007: note = From: FIGs. 10C/D
  DOMAIN 366..381: note = Truncated CD3z
  DOMAIN 382..388: note = Linker
  DOMAIN 389..419: note = Intein
  DOMAIN 420..425: note = Linker
  DOMAIN 426..532: note = FKBP12
  DOMAIN 812..828: note = FKBP12
  DOMAIN 829..834: note = Linker
  DOMAIN 835..865: note = Intein
  DOMAIN 866..871: note = Linker
  DOMAIN 872..978: note = FKBP12
METDTLLLWVLLLWVPGSTGEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTRAKFSRSAETAANLQDTGSSGSGYIDTDSVVGDTIIDVSGKKMTIAEFYDSTPDGSGGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGRSSGATNFSLLKQAGDVEENPGPEFTMASPLTRFLSLNLLLLGESIILGSGEAQVQLQESGGGLVQAGGSLRLSCAASGRDFATYSMAWFRQAPGKERESVATISWSGQRTRYADSVKGRFTISRDNAKNTVYLQMNSLKPEDTAVYYCAMPRTWGEFPPTQYDSWGQGTQVTVSSGGGGSELPTQGTFSNVSTNVSGGGGSGGGSGSLSTTTKPVLRTPSPVHPTGTSQPQRPEDCRPRGSVKGTGLDFACDIYIWAPLAGICVALLLSLIITLICYRAKFSRSAETAANLQDTGSSGSGYIDTDSVVGDTIIDVSGKKMTIAEFYDSTPDGSGGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSGGGGSGGGSGTGKYKSRRSFIEEKKMP

SEQ ID NO: 74 - protein, 593 aa, synthetic construct
  REGION 1..593: note = Description of sequence: Thy1.1 P2A IL-7 hCD34 CD8EC CD8TM tCD3z AEY Cfa N G4S2 EEKKMP E3 19K From drawing(s): FIG. 11C
  DOMAIN 1..593: note = From: FIG. 11C
  DOMAIN 441..457: note = Truncated CD3z
  DOMAIN 458..460: note = Linker
  DOMAIN 461..564: note = Intein
  DOMAIN 565..578: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGGGGRSELPTQGTFSNVSTNVSGGGGSGSLSTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 75 - protein, 751 aa, synthetic construct; identical to SEQ ID NO: 71
  REGION 1..751: note = Description of sequence: Thy1.1 P2A IL-7 hCD34 CD8EC CD8TM tCD3z AES CL N FKBP12-DmRA G4S2 EEKKMP E3 19K From drawing(s): FIG. 11C
  DOMAIN 1..751: note = From: FIG. 11C
  DOMAIN 556..572: note = Truncated CD3z
  DOMAIN 573..578: note = Linker
  DOMAIN 579..609: note = Intein
  DOMAIN 610..615: note = Linker
  DOMAIN 616..722: note = FKBP12

SEQ ID NO: 76 - protein, 624 aa, synthetic construct
  REGION 1..624: note = Description of sequence: Cfa C CD28 1XX DHFR-DD RGI EGFP From drawing(s): FIG. 12C
  DOMAIN 1..624: note = From: FIG. 12C
  DOMAIN 1..39: note = Intein
  DOMAIN 40..42: note = Linker
  DOMAIN 203..360: note = DHFR-DD
MVKIISRKSLGTQNVYDIGVEKDHNFLLKNGLVASNCFNGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGTEARLISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 77 - protein, 767 aa, synthetic construct
  REGION 1..767: note = Description of sequence: Cfa C CD28 1XX AI EGFP From drawing(s): FIGs. 13B/C
  DOMAIN 1..767: note = From: FIGs. 13B/C
  DOMAIN 1..39: note = Intein
  DOMAIN 40..42: note = Linker
  DOMAIN 197..201: note = Linker
  DOMAIN 202..503: note = SMASh-tag
MVKIISRKSLGTQNVYDIGVEKDHNFLLKNGLVASNCFNGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGTRGTGGSGDEMEECSQHLPGAGSSGDIMGSSGTGSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLYGSQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - protein, 923 aa, synthetic construct
  REGION 1..923: note = Description of sequence: Cfa C 5A-5B NS4a NS3 4A-4B Zap70 E2A EGFP From drawing(s): FIG. 14A
  DOMAIN 1..923: note = From: FIG. 14A
  DOMAIN 1..39: note = Intein
  DOMAIN 40..42: note = Linker
  DOMAIN 43..303: note = HCV NS3
  DOMAIN 504..506: note = Linker
  DOMAIN 507..651: note = Zap 70
  DOMAIN 652..661: note = Linker
MVKIISRKSLGTQNVYDIGVEKDHNFLLKNGLVASNCFNGTGEDVVCCHSIYGKKKGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHALGPNSSASAAVAAPTLPAHPSTFTQPQRRVDTLNSDGYTPEPARLASSTDKPRPMPMDTSVYESPYSDPEELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEAFSYGQKPYKKMKGPEVLDFIKQGKRMECPPECPPEMYALMSDCWIYKWEDRPDFLTVEQRMRNYYYSLGTSRAKRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 79 - protein, 689 aa, synthetic construct
  REGION 1..689: note = Description of sequence: Thy1.1 P2A 1D3 CD19 FLAG CD8EC CD8TM tCD3z AEY Cfa N G4S2 EEKKMP E3 19K From drawing(s): FIG. 15B
  DOMAIN 1..689: note = From: FIG. 15B
  DOMAIN 554..556: note = Linker
  DOMAIN 557..660: note = Intein
  DOMAIN 661..674: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRGSGDYKDDDDKGSLSTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 80 - protein, 725 aa, synthetic construct
  REGION 1..725: note = Description of sequence: Cfa C 5A-5B NS4a NS3 4A-4B CD28z EGFP From drawing(s): FIGs. 15B-15D
  DOMAIN 1..725: note = From: FIGs. 15B-15D
  DOMAIN 1..39: note = Intein
  DOMAIN 40..42: note = Linker
  DOMAIN 43..303: note = HCV NS3
  DOMAIN 304..306: note = Linker
MVKIISRKSLGTQNVYDIGVEKDHNFLLKNGLVASNCFNGTGEDVVCCHSIYGKKKGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHALGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 81 - protein, 711 aa, synthetic construct
  REGION 1..711: note = Description of sequence: Cfa C NS4a NS3 4A-4B CD28z EGFP From drawing(s): FIGs. 15B-15D
  DOMAIN 1..711: note = From: FIGs. 15B-15D
  DOMAIN 1..39: note = Intein
  DOMAIN 43..289: note = HCV NS3
  DOMAIN 290..292: note = Linker
MVKIISRKSLGTQNVYDIGVEKDHNFLLKNGLVASNCFNGTGGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHALGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 82 - protein, 716 aa, synthetic construct
  REGION 1..716: note = Description of sequence: Cfa C 5A-5B NS4a NS3 CD28z EGFP From drawing(s): FIGs. 15B-15D
  DOMAIN 1..716: note = From: FIGs. 15B-15D
  DOMAIN 1..39: note = Intein
  DOMAIN 40..42: note = Linker
  DOMAIN 43..294: note = HCV NS3
  DOMAIN 295..297: note = Linker
MVKIISRKSLGTQNVYDIGVEKDHNFLLKNGLVASNCFNGTGEDVVCCHSIYGKKKGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFALGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 83 - protein, 748 aa, synthetic construct
  REGION 1..748: note = Description of sequence: Thy1.1 P2A 1D3 CD19 hCD34 CD8EC CD8TM tCD3z AEY AES CL N Cfa N G4S2 EEKKMP E3 19K From drawing(s): FIGs. 16A-16C
  DOMAIN 1..748: note = From: FIGs. 16A-16C
  DOMAIN 556..572: note = Truncated CD3z
  DOMAIN 573..578: note = Linker
  DOMAIN 569..609: note = Intein
  DOMAIN 610..614: note = Linker
  DOMAIN 616..719: note = Intein
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRGGGGSELPTQGTFSNVSTNVSGGGGSGGGSGSLSTTTKPVLRTPSPVHPTGTSQPQRPEDCRPRGSVKGTGLDFACDIYIWAPLAGICVALLLSLIITLICYRAKFSRSAETAANLQDTGSSGSGYIDTDSVVGDTIIDVSGKKMTIAEFYDSTPDGSGGSGAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 84 - protein, 872 aa, synthetic construct
  REGION 1..872: note = Description of sequence: Cfa C 5A-5B NS4a NS3 4A-4B AES CL C CD28z EGFP From drawing(s): FIGs. 16A-16C
  DOMAIN 1..872: note = From: FIGs. 16A-16C
  DOMAIN 1..39: note = Intein
  DOMAIN 40..42: note = Linker
  DOMAIN 43..303: note = HCV NS3
  DOMAIN 304..312: note = Linker
  DOMAIN 313..446: note = Intein
  DOMAIN 447..453: note = Linker
MVKIISRKSLGTQNVYDIGVEKDHNFLLKNGLVASNCFNGTGEDVVCCHSIYGKKKGGGGSGGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHALGGSGGGSEARDWVKRVGGKTSLSVNTYSGEVERKNINYIMKHTVKKRMFKIKAGGKEVIVTADHSVMVKRDGKIIDVKPTEMKQTDRVVKWMLTGSHMIEFIEFEIEDLGVMEIDVYDIEVDGNHNFFGNDILVHNSVYLNGGGGSTSNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 85 - protein, 689 aa, synthetic construct; identical to SEQ ID NO: 79
  REGION 1..689: note = Description of sequence: Thy1.1 P2A 1D3 CD19 FLAG CD8EC CD8TM tCD3z AEY Cfa N G4S2 EEKKMP E3 19K From drawing(s): FIG. 17D
  DOMAIN 1..689: note = From: FIG. 17D
  DOMAIN 537..553: note = Truncated CD3z
  DOMAIN 554..556: note = Linker
  DOMAIN 557..660: note = Intein
  DOMAIN 661..674: note = Linker

SEQ ID NO: 86 - protein, 1102 aa, synthetic construct
  REGION 1..1102: note = Description of sequence: Cfa C BB BFP T2A CD28 1XX EGFP AI From drawing(s): FIG. 17D
  DOMAIN 1..1102: note = From: FIG. 17D
  DOMAIN 1..39: note = Intein
  DOMAIN 353..391: note = Intein
  DOMAIN 392..394: note = Linker
  DOMAIN 799..1100: note = SMASh-tag
MVKIISRKSLGTQNVYDIGVEKDHNFLLKNGLVASNCFNKRGRKKLLYIFKQPFMRPVQTTQEEDGCSCRFPEEEEGGCELGGGGSMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGSRSGGSGRAKRALGEGRGSLLTCGDVEENPGPMVKIISRKSLGTQNVYDIGVEKDHNFLLKNGLVASNCFNGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGGGGSGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKTRGTGGSGDEMEECSQHLPGAGSSGDIMGSSGTGSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLYGS

SEQ ID NO: 87 - protein, 303 aa, synthetic construct
  REGION 1..303: note = Description of sequence: Cfa C CD28z E2A Cfa BB From drawing(s): FIGs. 17E-17G
  DOMAIN 1..303: note = From: FIGs. 17E-17G
  DOMAIN 1..39: note = Intein
  DOMAIN 40..42: note = Linker
  DOMAIN 220..222: note = Linker
  DOMAIN 223..261: note = Intein
MVKIISRKSLGTQNVYDIGVEKDHNFLLKNGLVASNCFNGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMVKIISRKSLGTQNVYDIGVEKDHNFLLKNGLVASNCFNKRGRKKLLYIFKQPFMRPVQTTQEEDGCSCRFPEEEEGGCEL

SEQ ID NO: 88 - protein, 221 aa, synthetic construct
  REGION 1..221: note = Description of sequence: Cfa C CD28z E2A From drawing(s): FIGs. 17E-17G
  DOMAIN 1..221: note = From: FIGs. 17E-17G
  DOMAIN 1..39: note = Intein
  DOMAIN 40..42: note = Linker
MVKIISRKSLGTQNVYDIGVEKDHNFLLKNGLVASNCFNGTGNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 89 - protein, 501 aa, synthetic construct
  REGION 1..501: note = Description of sequence: Cfa C Zap70 E2A Cfa BB From drawing(s): FIGs. 17A-17C
  DOMAIN 1..501: note = From: FIGs. 17A-17C
  DOMAIN 1..39: note = Intein
  DOMAIN 40..42: note = Linker
  DOMAIN 43..387: note = Zap70
  DOMAIN 388..397: note = Linker
  DOMAIN 418..420: note = Linker
  DOMAIN 421..459: note = Intein
MVKIISRKSLGTQNVYDIGVEKDHNFLLKNGLVASNCFNGTGPNSSASAAVAAPTLPAHPSTFTQPQRRVDTLNSDGYTPEPARLASSTDKPRPMPMDTSVYESPYSDPEELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEAFSYGQKPYKKMKGPEVLDFIKQGKRMECPPECPPEMYALMSDCWIYKWEDRPDFLTVEQRMRNYYYSLGTSRAKRGSGQCTNYALLKLAGDVESNPGPLESMVKIISRKSLGTQNVYDIGVEKDHNFLLKNGLVASNCFNKRGRKKLLYIFKQPFMRPVQTTQEEDGCSCRFPEEEEGGCEL

SEQ ID NO: 90 - protein, 419 aa, synthetic construct
  REGION 1..419: note = Description of sequence: Cfa C Zap70 E2A From drawing(s): FIGs. 17A-17C
  DOMAIN 1..419: note = From: FIGs. 17A-17C
  DOMAIN 1..39: note = Intein
  DOMAIN 40..42: note = Linker
  DOMAIN 43..387: note = Zap70
  DOMAIN 388..396: note = Linker
MVKIISRKSLGTQNVYDIGVEKDHNFLLKNGLVASNCFNGTGPNSSASAAVAAPTLPAHPSTFTQPQRRVDTLNSDGYTPEPARLASSTDKPRPMPMDTSVYESPYSDPEELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEAFSYGQKPYKKMKGPEVLDFIKQGKRMECPPECPPEMYALMSDCWIYKWEDRPDFLTVEQRMRNYYYSLGTSRAKRGSGQCTNYALLKLAGDVESNPGPLE

SEQ ID NO: 91 - protein, 497 aa, synthetic construct
  REGION 1..497: note = Description of sequence: Thy1.1 P2A RQR FLAG CD8EC CD8TM tCD3z AEY Cfa N G4S2 EEKKMP E3 19K From drawing(s): FIGs. 3E-3I
  DOMAIN 1..497: note = From: FIGs. 3E-3I
  DOMAIN 345..361: note = Truncated CD3z
  DOMAIN 362..364: note = Linker
  DOMAIN 365..468: note = Intein
  DOMAIN 469..482: note = Linker
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPEFTMETDTLLLWVLLLWVPGSTGCPYSNPSLCSGGGGSELPTQGTFSNVSTNVSPAKPTTTACPYSNPSLCGGGGSGGGSDYKDDDDKAAASTTTKPVLRTPSPVHPTGTSQPQRPEDCRPRGSVKGTGLDFACDIYIWAPLAGICVALLLSLIITLICYRAKFSRSAETAANLQDTGSSAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTSGGGGSGGGSGTGKYKSRRSFIEEKKMP

SEQ ID NO: 92 - protein, 477 aa, synthetic construct
  REGION 1..477: note = Description of sequence: Cfa C CD3z delta GP41-8 N RXR E2A EGFP From drawing(s): FIGs. 3E-3I
  DOMAIN 1..477: note = From: FIGs. 3E-3I
  DOMAIN 1..39: note = Intein
  DOMAIN 40..42: note = Linker
  DOMAIN 55..59: note = Linker
  DOMAIN 60..153: note = Intein
  DOMAIN 154..166: note = Linker
  DOMAIN 167..202: note = Kir6.2 RXR
MVKIISRKSLGTQNVYDIGVEKDHNFLLKNGLVASNCFNGTGFDALHMQTLAPRTSGSGSQLNRCLSLDTMVVTNGKAIEIRDVKVGDWLESECGPVQVTEVLPIIKQPVFEIVLKSGKKIRVSANHKFPTKDGLKTINSGLKVGDFLRSRAKGGGGSGGGSGSTGLLDALTLASSRGPLRKRSVAVAKAKPKFSISPDSLSALGGSGRAKRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 93 - protein, 452 aa, synthetic construct
  REGION 1..452: note = Description of sequence: Cfa C CD3z delta GP41-8 N EEKKMP EGFP From drawing(s): FIGs. 3E-3I
  DOMAIN 1..452: note = From: FIGs. 3E-3I
  DOMAIN 1..39: note = Intein
  DOMAIN 40..42: note = Linker
  DOMAIN 55..59: note = Linker
  DOMAIN 60..153: note = Intein
  DOMAIN 154..165: note = Linker
  DOMAIN 166..180: note = KKXX
  DOMAIN 181..183: note = Linker
MVKIISRKSLGTQNVYDIGVEKDHNFLLKNGLVASNCFNGTGFDALHMQTLAPRTSGSGSQLNRCLSLDTMVVTNGKAIEIRDVKVGDWLESECGPVQVTEVLPIIKQPVFEIVLKSGKKIRVSANHKFPTKDGLKTINSGLKVGDFLRSRAKGGGGSGGGSGTGKYKSRRSFIEEKKMPGSGRAKRGSGQCTNYALLKLAGDVESNPGPLESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 94 - protein, 116 aa, synthetic construct
  REGION 1..116: note = Description of sequence: CD 19 VH (1D3)
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 95 - protein, 108 aa, synthetic construct
  REGION 1..108: note = Description of sequence: CD19 VL (1D3)
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKR

SEQ ID NO: 96 - protein, 239 aa, synthetic construct
  REGION 1..239: note = Description of sequence: CD19 scFv (VH-Spacer-VL)
  DOMAIN 117..130: note = Spacer
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKR

SEQ ID NO: 97 - protein, 121 aa, synthetic construct
  REGION 1..121: note = Description of sequence: CD20 VH (18B12)
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSS

SEQ ID NO: 98 - protein, 107 aa, synthetic construct
  REGION 1..107: note = Description of sequence: CD20 VL (18B12)
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKR

SEQ ID NO: 99 - protein, 243 aa, synthetic construct
  REGION 1..243: note = Description of sequence: CD20 scFv (VL-Spacer-VH)
  DOMAIN 108..122: note = Spacer
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSS

SEQ ID NO: 100 - protein, 123 aa, synthetic construct
  REGION 1..123: note = Description of sequence: CD20 VHH (2MC57)
QVQLQESGGGLVQAGGSLRLSCAASGRDFATYSMAWFRQAPGKERESVATISWSGQRTRYADSVKGRFTISRDNAKNTVYLQMNSLKPEDTAVYYCAMPRTWGEFPPTQYDSWGQGTQVTVSS

SEQ ID NO: 101 - protein, 220 aa, synthetic construct
  REGION 1..220: note = Description of sequence: CD28 (1)
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 102 - protein, 123 aa, synthetic construct
  REGION 1..123: note = Description of sequence: CD28 (1) - encoding amino acids 180-220
AGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCC

SEQ ID NO: 103 - protein, 218 aa, synthetic construct
  REGION 1..218: note = Description of sequence: CD28 (mouse)
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 104 - protein, 255 aa, synthetic construct
  REGION 1..255: note = Description of sequence: 4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 105 - protein, 182 aa, synthetic construct
  REGION 1..182: note = Description of sequence: CD3-Gamma
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 106 - protein, 171 aa, synthetic construct
  REGION 1..171: note = Description of sequence: CD3-Delta (1)
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 107 - protein, 127 aa, synthetic construct
  REGION 1..127: note = Description of sequence: CD3-Delta (2)
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 108 - protein, 207 aa, synthetic construct
  REGION 1..207: note = Description of sequence: CD3e
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 109 - protein, 164 aa, synthetic construct
  REGION 1..164: note = Description of sequence: CD3-Zeta (1)
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 110 - protein, 112 aa, synthetic construct
  REGION 1..112: note = Description of sequence: CD3-Zeta (2)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 112 aa, synthetic construct
  REGION 1..112: note = Description of sequence: CD3-Zeta (3)
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 112 - protein, 336 aa, synthetic construct
  REGION 1..336: note = Description of sequence: CD3-Zeta (3)
AGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTTCAATGAACTGCAGAAAGATAAGATGGCGGAGGCCTTCAGTGAGATTGGGATGAAAGGCGAGCGCCGGAGGGGCAAGGGGCACGATGGCCTTTTCCAGGGGCTCAGTACAGCCACCAAGGACACCTTCGACGCCCTTCACATGCAGGCCCTGCCCCCTCGC

SEQ ID NO: 113 - protein, 164 aa, synthetic construct
  REGION 1..164: note = Description of sequence: CD3-Zeta (mouse)
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 114 - protein, 619 aa, synthetic construct
  REGION 1..619: note = Description of sequence: Zap70 human Interdomain + kinase domain in lower case underlined
  DOMAIN 255..600: note = Kinase domain
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 115 - protein, 618 aa, synthetic construct
  REGION 1..618: note = Description of sequence: Zap70 mouse Interdomain + kinase domain in lower case underlined
  DOMAIN 255..599: note = Kinase domain
MPDPAAHLPFFYGSISRAEAEEHLKLAGMADGLFLLRQCLRSLGGYVLSLVHDVRFHHFPIERQLNGTYAIAGGKAHCGPAELCQFYSQDPDGLPCNLRKPCNRPPGLEPQPGVFDCLRDAMVRDYVRQTWKLEGDALEQAIISQAPQVEKLIATTAHERMPWYHSSLTREEAERKLYSGQQTDGKFLLRPRKEQGTYALSLVYGKTVYHYLISQDKAGKYCIPEGTKFDTLWQLVEYLKLKADGLIYRLKEVCPNSSASAAVAAPTLPAHPSTFTQPQRRVDTLNSDGYTPEPARLASSTDKPRPMPMDTSVYESPYSDPEELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEAFSYGQKPYKKMKGPEVLDFIKQGKRMECPPECPPEMYALMSDCWIYKWEDRPDFLTVEQRMRNYYYSLASRAEGPPQCEQVAEAACG

SEQ ID NO: 116 - protein, 22 aa, synthetic construct
  REGION 1..22: note = Description of sequence: ITAM1
QNQLYNELNLGRREEYDVLDKR

SEQ ID NO: 117 - protein, 66 aa, synthetic construct
  REGION 1..66: note = Description of sequence: ITAM1
CAGAACCAGCTCTATAACGAGCTCAATCTAGGACGAAGAGAGGAGTACGATGTTTTGGACAAGAGA

SEQ ID NO: 118 - protein, 22 aa, synthetic construct
  REGION 1..22: note = Description of sequence: ITAM1 variant
QNQLFNELNLGRREEFDVLDKR

SEQ ID NO: 119 - protein, 66 aa, synthetic construct
  REGION 1..66: note = Description of sequence: ITAM1 variant
CAGAACCAGCTCTTTAACGAGCTCAATCTAGGACGAAGAGAGGAGTTCGATGTTTTGGACAAGAGA

SEQ ID NO: 120 - protein, 23 aa, synthetic construct
  REGION 1..23: note = Description of sequence: ITAM2
QEGLYNELQKDKMAEAYSEIGMK

SEQ ID NO: 121 - protein, 69 aa, synthetic construct
  REGION 1..69: note = Description of sequence: ITAM2
CAGGAAGGCCTGTACAATGAACTGCAGAAAGATAAGATGGCGGAGGCCTACAGTGAGATTGGGATGAAA

SEQ ID NO: 122 - protein, 23 aa, synthetic construct
  REGION 1..23: note = Description of sequence: ITAM2 variant
QEGLFNELQKDKMAEAFSEIGMK

SEQ ID NO: 123 - protein, 69 aa, synthetic construct
  REGION 1..69: note = Description of sequence: ITAM2 variant
CAGGAAGGCCTGTTCAATGAACTGCAGAAAGATAAGATGGCGGAGGCCTTCAGTGAGATTGGGATGAAA

SEQ ID NO: 124 - protein, 22 aa, synthetic construct
  REGION 1..22: note = Description of sequence: ITAM3
HDGLYQGLSTATKDTYDALHMQ

SEQ ID NO: 125 - protein, 66 aa, synthetic construct
  REGION 1..66: note = Description of sequence: ITAM3
CACGATGGCCTTTACCAGGGTCTCAGTACAGCCACCAAGGACACCTACGACGCCCTTCACATGCAG

SEQ ID NO: 126 - protein, 22 aa, synthetic construct
  REGION 1..22: note = Description of sequence: ITAM3 variant
HDGLFQGLSTATKDTFDALHMQ

SEQ ID NO: 127 - protein, 66 aa, synthetic construct
  REGION 1..66: note = Description of sequence: ITAM3 variant
CACGATGGCCTTTTCCAGGGGCTCAGTACAGCCACCAAGGACACCTTCGACGCCCTTCACATGCAG

SEQ ID NO: 128 - protein, 20 aa, synthetic construct
  REGION 1..20: note = Description of sequence: Signal sequence 1
MYRMQLLSCIALSLALVTNS

SEQ ID NO: 129 - protein, 20 aa, synthetic construct
  REGION 1..20: note = Description of sequence: Signal sequence 2
MYSMQLASCVTLTLVLLVNS

SEQ ID NO: 130 - protein, 20 aa, synthetic construct
  REGION 1..20: note = Description of sequence: Signal sequence 3
METPAQLLFLLLLWLPDTTG

SEQ ID NO: 131 - protein, 20 aa, synthetic construct
  REGION 1..20: note = Description of sequence: Signal sequence 4
METDTLLLWVLLLWVPGSTG

SEQ ID NO: 132 - protein, 21 aa, synthetic construct
  REGION 1..21: note = Description of sequence: Signal sequence 5
MALPVTALLLPLALLLHAARP

SEQ ID NO: 133 - protein, 18 aa, synthetic construct
  REGION 1..18: note = Description of sequence: Signal sequence 6
MALPVTALLLPLALLLHA

SEQ ID NO: 134 - protein, 16 aa, synthetic construct
  REGION 1..16: note = Description of sequence: Signal sequence 7
MKWVTFISLLFSSAYS

SEQ ID NO: 135 - protein, 30 aa, synthetic construct
  REGION 1..30: note = Description of sequence: Signal sequence 8
MDSKGSSQKGSRLLLLLVVSNLLLCQGVVS

SEQ ID NO: 136 - DNA, 4627 bp, synthetic construct
  misc_feature 1..4627: note = Description of sequence: TRAC
  source 1..4627: mol_type = other DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 137 - protein, 178 aa, synthetic construct
  REGION 1..178: note = Description of sequence: TRBC2
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 138 - DNA, 534 bp, synthetic construct
  misc_feature 1..534: note = Description of sequence: TRBC2
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 139 - protein, 175 aa, synthetic construct
  REGION 1..175: note = Description of sequence: TRBC1 (1)
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 140 - protein, 176 aa, synthetic construct
  REGION 1..176: note = Description of sequence: TRBC1 (2)
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 141 - DNA, 528 bp, synthetic construct
  misc_feature 1..528: note = Description of sequence: TRBC1 (2)
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 142 - DNA, 1448 bp, synthetic construct
  misc_feature 1..1448: note = Description of sequence: TRBC
  source 1..1448: mol_type = other DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 143 - DNA, 1489 bp, synthetic construct
  misc_feature 1..1489: note = Description of sequence: TRBC
  source 1..1489: mol_type = other DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 144 - protein, 10 aa, synthetic construct
  REGION 1..10: note = Description of sequence: Hinge/spacer
IPNIQNPDPA

SEQ ID NO: 145 - DNA, 30 bp, synthetic construct
  misc_feature 1..30: note = Description of sequence: Hinge/spacer
  source 1..30: mol_type = other DNA
attcccaatatccagaaccctgaccctgcc

SEQ ID NO: 146 - protein, 11 aa, synthetic construct
  REGION 1..11: note = Description of sequence: Hinge/spacer
LEDLKNVFPPE

SEQ ID NO: 147 - DNA, 33 bp, synthetic construct
  misc_feature 1..33: note = Description of sequence: Hinge/spacer
  source 1..33: mol_type = other DNA
ctggaggatctgaaaaacgtgttccctcctgaa

SEQ ID NO: 148 - protein, 9 aa, synthetic construct
  REGION 1..9: note = Description of sequence: Linker8
GGGGSGGGS

SEQ ID NO: 149 - protein, 8 aa, synthetic construct
  REGION 1..8: note = Description of sequence: Linker9
GGGGSGTG

SEQ ID NO: 150 - protein, 9 aa, synthetic construct
  REGION 1..9: note = Description of sequence: Linker10
GTSRAKRGS

SEQ ID NO: 151 - protein, 9 aa, synthetic construct
  REGION 1..9: note = Description of sequence: Linker11
ALGGSGGGS

SEQ ID NO: 152 - protein, 7 aa, synthetic construct
  REGION 1..7: note = Description of sequence: Linker12
GGGGSTS

SEQ ID NO: 153 - protein, 7 aa, synthetic construct
  REGION 1..7: note = Description of sequence: Linker13
SGGGGSD

SEQ ID NO: 154 - protein, 6 aa, synthetic construct
  REGION 1..6: note = Description of sequence: Linker14
GSSGSG

SEQ ID NO: 155 - protein, 6 aa, synthetic construct
  REGION 1..6: note = Description of sequence: Linker15
GSGGTR

SEQ ID NO: 156 - protein, 5 aa, synthetic construct
  REGION 1..5: note = Description of sequence: Linker16
GSGTR

SEQ ID NO: 157 - protein, 5 aa, synthetic construct
  REGION 1..5: note = Description of sequence: Linker17
TSGSG

SEQ ID NO: 158 - protein, 5 aa, synthetic construct
  REGION 1..5: note = Description of sequence: Linker18
GSGGS

SEQ ID NO: 159 - protein, 4 aa, synthetic construct
  REGION 1..4: note = Description of sequence: Furin cleavage site
RAKR

SEQ ID NO: 160 - protein, 8 aa, synthetic construct
  REGION 1..8: note = Description of sequence: FLAG tag
DYKDDDDK

SEQ ID NO: 161 - protein, 9 aa, synthetic construct
  REGION 1..9: note = Description of sequence: HCV 4A/4B cut site
DEMEECSQH